(19) United States
(12) Patent Application Publication
(10) Pub. No.: US 2025/0177362 A1
(43) Pub. Date: Jun. 5, 2025

(54) HETEROCYCLIC GLP-1 AGONISTS

(71) Applicant: Gasherbrum Bio, Inc., South San Francisco, CA (US)

(72) Inventors: Qinghua MENG, Shanghai (CN); Weiqiang XING, Shanghai (CN)

(21) Appl. No.: 18/837,152

(22) PCT Filed: Feb. 8, 2023
(86) PCT No.: PCT/CN2023/074936

§ 371 Date: Aug. 8, 2024

(30) Foreign Application Priority Data
Feb. 9, 2022 (WO) PCT/CN2022/075604

(51) Int. Cl. A61K 31/437; C07D 405/14; C07D 413/14; C07D 417/14; C07D 471/04; C07D 491/08

(52) U.S. Cl. CPC A61K 31/437; C07D 405/14; C07D 413/14; C07D 417/14; C07D 471/04; C07D 491/08

(57) ABSTRACT

The present disclosure relates generally to GLP-1 agonists and pharmaceutical compositions comprising the same, as well as methods for treating a GLP-1 associated disease, disorder, or condition.

### CROSS REFERENCE TO RELATED APPLICATIONS

The application claims the benefit of International Patent Application Number PCT/CN2022/075604, filed on Feb. 9, 2022, which is incorporated herein by reference in its entirety.

### FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

In one aspect, provided is a compound of Formula I:

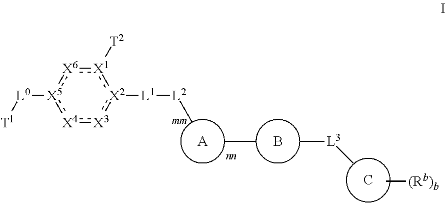

or a pharmaceutically acceptable salt or solvate thereof, wherein:
X¹, X², and X⁵ are independently C or N;
X³ and X⁴ are independently selected from the group consisting of: N, NR^{x}, CR^{y}, C(O), O, and S;
X⁶ is selected from the group consisting of: a bond, N, NR^{x}, CR^{y}, and C(O);
each [symbol] is a single bond or a double bond, provided that at least one of X¹-X⁶ is an independently selected heteroatom or heteroatomic group; at least one of X¹-X⁶ is C or CR^{y}; and the ring including X¹-X⁶ is aromatic;
each R^{x} is independently selected from the group consisting of: hydrogen, (C₁-C₆)alkyl, (C₁-C₆)haloalkyl, (C₃-C₆)cycloalkyl, C(O)(C₁-C₅)alkyl, S(O)₂(C₁-C₆)alkyl, and C(O)O(C₁-C₆)alkyl;
each R^{y} is independently selected from the group consisting of: hydrogen, —OH, (C₁-C₆)alkyl, (C₁-C₆)haloalkyl, (C₃-C₆)cycloalkyl, (C₁-C₅)alkoxy, (C₁-C₆)haloalkoxy, CN, and halogen;
L⁰ is a bond or #—P⁰-P¹, wherein # represents the point of attachment to X⁵;
—P⁰ is a bond, —NH—, —N(C₁-C₆ alkyl)-, —O—, or S(O)₀₋₂;
—P¹ is selected from the group consisting of: (C₁-C₅)alkylene, (C₂-C₆)alkenylene, (C₂-C₆)alkynylene, (C₃-C₈)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH or a carboxylic acid bioisostere; or
the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₅)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
T² is:
   hydrogen, CN, (C₁-C₆)haloalkyl, (C₁-C₆)hydroxyalkyl or (C₁-C₆)alkyl which is optionally substituted with (C₁-C₅)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₅)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
   —NH(C₁-C₆)alkyl, —NH—(C₃-C₆)cycloalkyl, —CH₂—S(O)₂—(C₃-C₅)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
provided the compound is not:

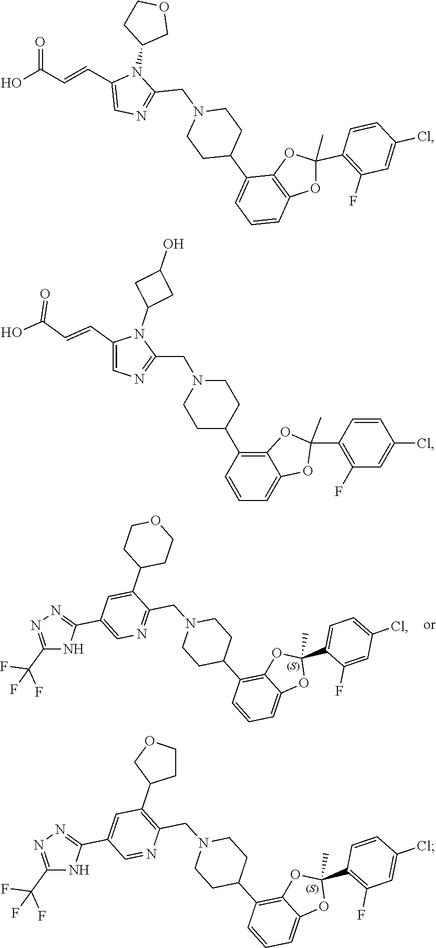

each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)—C₁-C₆ alkyl, S(O)₂(C₁-C₆ alkyl) and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₅)cycloalkyl ring;
Ring A is selected from the group consisting of:

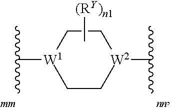

wherein n1 is 0, 1, or 2, W¹ is CR^{Y1} or N, and W² is CR^{Y2} or N;

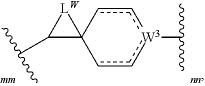

wherein W³ is C, CR^{Y3}, or N, L^{w} is (C₁-C₃)alkylene, and each [symbol] is independently a single bond or a double bond, as allowed by valence;
phenylene optionally substituted with 1-4 R^{Y};
5- to 6-membered heteroarylene optionally substituted with 1-3 R^{Y};
(C₅-C₈)cycloalkylene optionally substituted with 1-4 R^{Y};
partially unsaturated monocyclic (C₅-C₈)cycloalkylene optionally substituted with 1-4 R^{y}; and
partially unsaturated monocyclic 5- to 8-membered heterocycloalkylene optionally substituted with 1-4 R^{Y};
wherein mm represents the point of attachment to L², and nn represents the point of attachment to Ring B;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1}, R^{Y2}, and R^{Y3} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together can form (C₁-C₄)alkylene, wherein one of the CH₂ units of the (C₁-C₄)alkylene is optionally replaced by a heteroatom selected from the group consisting of O, S, NH, and N(C₁₋₃)alkyl;
Ring B is selected from the group consisting of: (B-I), (B-II), (B-III), (B-IV), (B-V), (B-VI), and (B-VII):

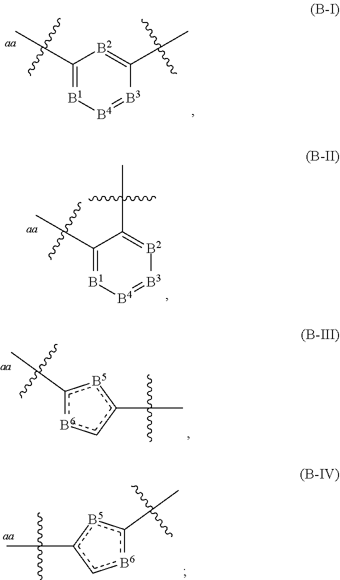

wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl;

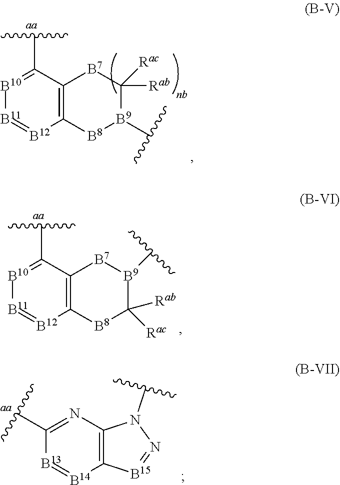

wherein aa represents the point of attachment to Ring A;
B⁷ and B⁸ are independently selected from the group consisting of: —O—, —NR^{N}—, and —C(R′)₂—;
B⁹ is N or CR^{aa};
nb is 0 or 1;
B¹⁰, B¹¹, and B¹² are independently selected from the group consisting of CR¹ and N;
B¹¹, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₅)cycloalkyl, (C₁-C₃)alkyl(3- to 5-membered heterocycloalkyl), and —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or —Z¹-Z²—*, wherein * represents the point of attachment to Ring C;
—Z¹ is a bond, NH, N(C₁-C₆ alkyl), O, or S(O)₀₋₂;
—Z² is C₁₋₃ alkylene optionally substituted with 1-2 R^{c};
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3;
provided at least one of the following occurs:
1) at least one R^{b} is —C(O)NH₂ or —C(O)OH;
2) Ring A is (C₅-C₈)cycloalkylene optionally substituted with 1-4 R^{Y};
3) Ring B is

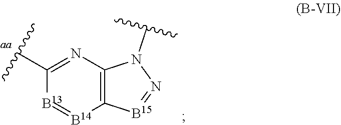

4) the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}.

This disclosure also provides pharmaceutical compositions comprising one or more compound as disclosed herein, or a pharmaceutically acceptable salt or solvate thereof, and a pharmaceutically acceptable excipient.

Also provided herein are pharmaceutical compositions comprising a compound disclosed herein, or a pharmaceutically acceptable salt, stereoisomer, mixture of stereoisomers, or prodrug thereof, and a pharmaceutically acceptable excipient.

Also provided herein are methods for treating type 2 diabetes mellitus in a patient in need thereof, the methods comprising administering to the patient a therapeutically effective amount of a compound disclosed herein, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound disclosed herein, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound disclosed herein, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound disclosed herein, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound disclosed herein, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound disclosed herein, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound disclosed herein, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound disclosed herein, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Before the present compounds and methods are described, it is to be understood that the disclosure is not limited to the methodologies, protocols, cell lines, assays, and reagents described, as these may vary. It is also to be understood that the terminology used herein is intended to describe embodiments of the present disclosure, and is in no way intended to limit the scope of the present disclosure as set forth in the appended claims.

### Definitions

It must be noted that as used herein, and in the appended claims, the singular forms “a,” “an,” and “the” include plural references unless the context clearly dictates otherwise.

Unless defined otherwise, all technical, and scientific terms used herein have the same meanings as commonly understood by one of ordinary skill in the art to which this disclosure pertains. Although any methods and materials similar or equivalent to those described herein can be used in the practice or testing of the present disclosure, exemplary methods, devices, and materials are now described. All publications cited herein are incorporated herein by reference in their entirety for the purpose of describing and disclosing the methodologies, reagents, and tools reported in the publications that might be used in connection with the disclosure.

Provided herein are heterocyclic GLP-1 agonists for use in the management of T2DM and other conditions where activation of GLP-1 activity is useful.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁₋₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “(C₁₋₃)alkylene” refers to a divalent alkyl having one to three carbon atoms (e.g., —CH₂—, —CH(CH₃)—, —CH₂CH₂—, or —CH₂CH₂CH₂—). Similarly, the terms “cycloalkylene,” “heterocyclylene,” “arylene,” and “heteroarylene” mean divalent cycloalkyl, heterocycloalkyl, aryl, and heteroaryl, respectively.

As used herein, the term “alkenyl” refers to a linear or branched mono-unsaturated hydrocarbon chain, containing the indicated number of carbon atoms. For example, “(C₂₋₆)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂₋₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃₋₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the terms “heterocyclyl” and “heterocycloalkyl” refer to a mon-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, the heteroatoms selected from O, N, S, or S(O)₁₂ (e.g., carbon atoms and 1-3, 1-6, or 1-9 heteroatoms of N, O, S, or S(O)₁₋₂ if monocyclic, bicyclic, or tricyclic, respectively), wherein 0, 1, 2 or 3 atoms of each ring may be substituted by a substituent. Examples of heterocycloalkyl groups include piperazinyl, pyrrolidinyl, dioxanyl, morpholinyl, tetrahydrofuranyl, and the like. Heterocycloalkyl groups may be partially unsaturated. Non-limiting examples of partially unsaturated heterocycloalkyl include dihydropyrrolyl, dihydropyridinyl, tetrahydropyridinyl, dihydrofuranyl, dihydropyranyl, and the like. Heterocycloalkyl may include multiple fused and bridged rings. Non-limiting examples of fused/bridged heterocycloalkyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocycloalkyl also includes spirocyclic rings (e.g., spirocyclic bicycle wherein two rings are connected through just one atom). Non-limiting examples of spirocyclic heterocycloalkyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more hydrogen atoms (e.g., 1 to 5, or 1 to 3) is replaced with one or more halogen atoms (e.g., 1 to 5, or 1 to 3). Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, the term “amino” refers to an amine of formula —N(R^{N1})₂, where each R^{N} is independently hydrogen, alkyl, alkenyl, alkynyl, haloalkyl, cycloalkyl, heterocycloalkyl, aryl, or heteroaryl, wherein each is optionally substituted, such as by one or more (e.g., 1 to 5, or 1 to 3) substituents (e.g., independently selected from halo, cyano, hydroxy, —NH₂, —NH(alkyl), —N(alkyl)₂, alkyl, alkenyl, alkynyl, haloalkyl, alkoxy, or haloalkoxy).

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic,” it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thiazole, isoxazole, isothiazole, and the like. When a ring system comprising at least two rings is described as “aromatic,” it means the ring system comprises one or more aromatic ring(s). Accordingly, when a ring system comprising at least two rings is described as “non-aromatic,” none of the constituent rings of the ring system is aromatic.

As used herein, when a ring is described as being “partially unsaturated,” it means the ring has one or more additional degrees of unsaturation (in addition to the degree of unsaturation attributed to the ring itself, e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated,” it means the ring system comprises one or more partially unsaturated ring(s), provided that none of the constituent rings of the ring system is aromatic.

As used herein, the term “carboxylic acid bioisostere” means a group which has chemical and physical similarities producing broadly similar biological properties to a carboxylic acid (see Lipinski, Annual Reports in Medicinal Chemistry, 1986, 21, p 283 “Bioisosterism In Drug Design”; Yun, Hwahak Sekye, 1993, 33, pages 576-579 “Application Of Bioisosterism To New Drug Design”; Zhao, Huaxue Tongbao, 1995, pages 34-38, 25 “Bioisosteric Replacement And Development Of Lead Compounds In Drug Design”; Graham, Theochem, 1995, 343, pages 105-109 “Theoretical Studies Applied To Drug Design:ab initio Electronic Distributions In Bioisosteres”). Examples of a suitable carboxylic acid bioisostere include: sulfo, phosphono, alkylsulfonylcarbamoyl, tetrazolyl, arylsulfonylcarbamoyl, heteroarylsulfonylcarbamoyl, N-methoxycarbamoyl, 3-hydroxy-3-cyclobutene-1,2-dione, 3,5-dioxo-1,2,4-oxadiazolidinyl or heterocyclic phenols such as 3-hydroxyisoxazolyl and 3-hydoxy-1-methylpyrazolyl.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 receptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocycloalkyl, aryl, or heteroaryl, wherein each is optionally substituted, such as by one or more (e.g., 1 or 5 or 1 or 3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

It is understood that the substituents as defined herein are not intended to include impermissible substitution patterns (e.g., methyl substituted with 5 fluoro groups or a hydroxy group attached to an ethenylic or acetylenic carbon atom). Such impermissible substitution patterns are well known to the skilled artisan. Likewise, the term “substituted” refers to one or more (e.g., one to five) chemical moieties as disclosed herein (such as those defined above), which may then be further substituted.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound disclosed herein, or a pharmaceutically acceptable salt or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21^{st} ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound disclosed herein, or a pharmaceutically acceptable salt or solvate thereof as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing,” as used herein, is the prevention of the onset, recurrence or spread, in whole or in part, of the disease or condition as described herein, or a symptom thereof.

The terms “subject,” “patient,” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the disclosure.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation,” as used herein, refers to a regulation or an adjustment (e.g., increase or decrease) and can include, for example agonism, partial agonism or antagonism.

### Compounds

Provided herein is a compound of Formula I:

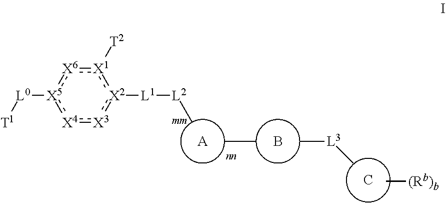

or a pharmaceutically acceptable salt or solvate thereof, wherein:
X¹, X², and X⁵ are independently C or N;
X³ and X⁴ are independently selected from the group consisting of: N, NR^{x}, CR^{y}, C(O), O, and S;
X⁶ is selected from the group consisting of: a bond, N, NR^{x}, CR^{y}, and C(O);
each [symbol] is a single bond or a double bond, provided that at least one of X¹-X⁶ is an independently selected heteroatom or heteroatomic group; at least one of X¹-X⁶ is C or CR^{y}; and the ring including X¹-X⁶ is aromatic;
each R^{x} is independently selected from the group consisting of: hydrogen, (C₁-C₆)alkyl, (C₁-C₆)haloalkyl, (C₃-C₆)cycloalkyl, C(O)(C₁-C₆)alkyl, S(O)₂(C₁-C₆)alkyl, and C(O)O(C₁-C₆)alkyl;
each R^{y} is independently selected from the group consisting of: hydrogen, —OH, (C₁-C₆)alkyl, (C₁-C₆)haloalkyl, (C₃-C₆)cycloalkyl, (C₁-C₆)alkoxy, (C₁-C₆)haloalkoxy, CN, and halogen; L⁰ is a bond or #—P⁰-P¹, wherein # represents the point of attachment to X⁵;
—P⁰ is a bond, —NH—, —N(C₁-C₆ alkyl)-, —O—, or S(O)₀₋₂;
—P¹ is selected from the group consisting of: (C₁-C₆)alkylene, (C₂-C₆)alkenylene, (C₂-C₆)alkynylene, (C₃-C₆)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH or a carboxylic acid bioisostere; or
the moiety -L⁰-T¹ is:
   —C(O)NH—(C₁-C₆)alkyl or —C(O)NH—(C₃-C₅)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
T² is:
   hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
—NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₅)cycloalkyl, —O—(C₃-C₅)cycloalkyl, (C₁-C₅)alkoxy, (C₃-C₅)cycloalkyl, or 3-10 membered heterocycloalkyl is optionally substituted with 1-4 R^{T};
each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)—C₁-C₆ alkyl, S(O)₂(C₁-C₆ alkyl) and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

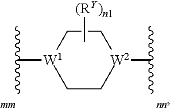

wherein n1 is 0, 1, or 2, W is CR^{Y1} or N, and W² is CR^{Y2} or N;

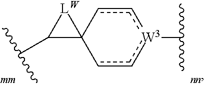

wherein W³ is C, CR^{Y3}, or N, L is (C₁-C₃)alkylene, and each [symbol] is independently a single bond or a double bond, as allowed by valence;
phenylene optionally substituted with 1-4 R^{Y};
5- to 6-membered heteroarylene optionally substituted with 1-3 R^{Y};
(C₅-C₈)cycloalkylene optionally substituted with 1-4 R^{y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to Ring B;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1}, R^{Y2}, and R^{Y3} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together can form (C₁-C₄)alkylene, wherein one of the CH₂ units of the (C₁-C₄)alkylene is optionally replaced by a heteroatom selected from the group consisting of O, S, NH, and N(C₁₋₃)alkyl;
Ring B is selected from the group consisting of: (B-I), (B-II), (B-Ill), (B-IV), (B-V), (B-VI), and (B-VII):

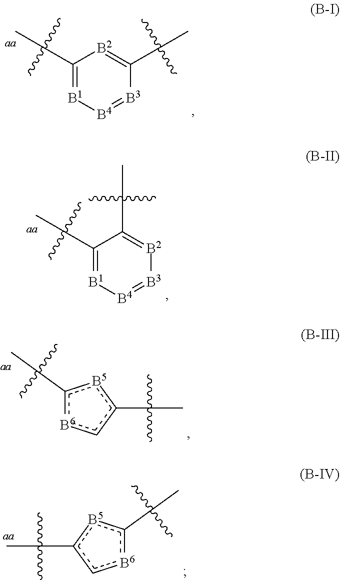

wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl;

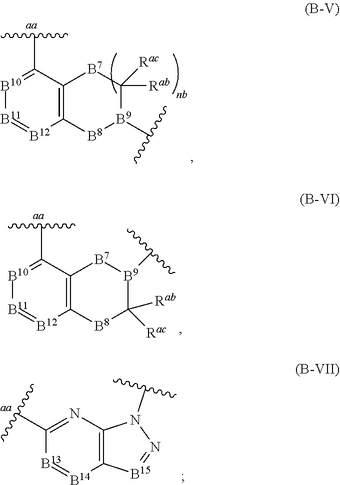

wherein aa represents the point of attachment to Ring A;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
B¹³, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or —Z¹-Z²—*, wherein * represents the point of attachment to Ring C;
—Z¹ is a bond, NH, N(C₁-C₆ alkyl), O, or S(O)₀₋₂;
—Z² is C₁₋₃ alkylene optionally substituted with 1-2 R¹;
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3.

In some embodiments, at least one of the following occurs:
1) at least one R^{b} is —C(O)NH₂ or —C(O)OH;
2) Ring A is (C₅-C₈)cycloalkylene optionally substituted with 1-4 R^{Y};
3) Ring B is

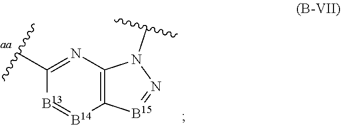

4) the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}.

In some embodiments, at least one R^{x} (C₃-C₆)cycloalkyl.

In some embodiments, at least one R^{y} is (C₃-C₆)cycloalkyl.

In some embodiments, the compound is not:

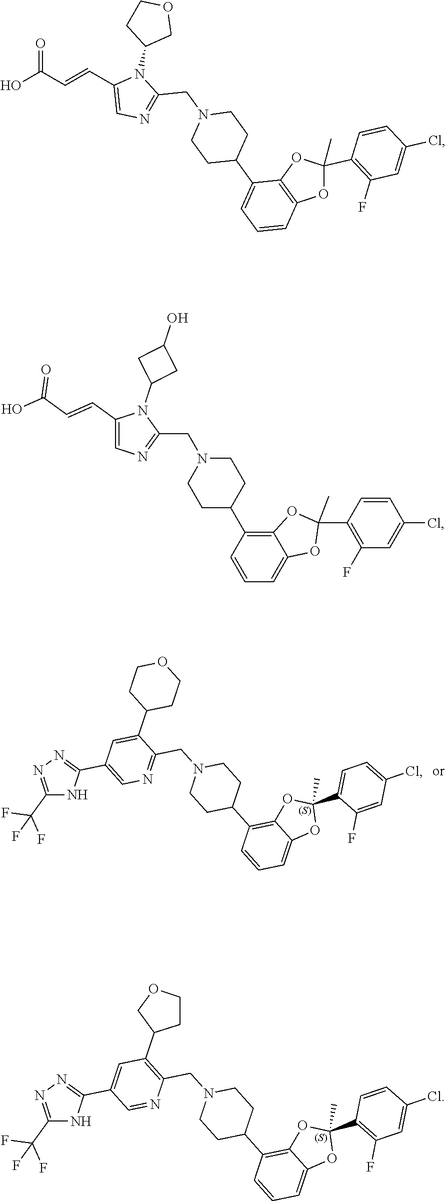

In some embodiments, provided herein is a compound of Formula I:

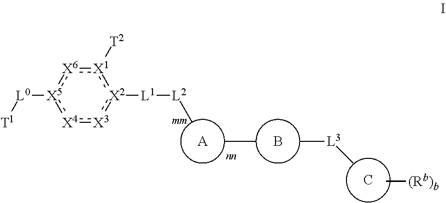

or a pharmaceutically acceptable salt or solvate thereof, wherein:
X¹, X², and X⁵ are independently C or N;
X³ and X⁴ are independently selected from the group consisting of: N, NR^{x}, CR^{y}, C(O), O, and S;
X⁶ is selected from the group consisting of: a bond, N, NR^{x}, CR^{y}, and C(O);
each [symbol] is a single bond or a double bond, provided that at least one of X¹-X⁶ is an independently selected heteroatom or heteroatomic group; at least one of X¹-X⁶ is C or CR^{y}; and the ring including X¹-X⁶ is aromatic;
each R^{x} is independently selected from the group consisting of: hydrogen, (C₁-C₆)alkyl, (C₁-C₆)haloalkyl, (C₃-C₆)cycloalkyl, C(O)(C₁-C₆)alkyl, S(O)₂(C₁-C₆)alkyl, and C(O)O(C₁-C₆)alkyl;
each R^{y} is independently selected from the group consisting of: hydrogen, —OH, (C₁-C₆)alkyl, (C₁-C₆)haloalkyl, (C₃-C₆)cycloalkyl, (C₁-C₆)alkoxy, (C₁-C₆)haloalkoxy, CN, and halogen; L⁰ is a bond or #—P⁰-P¹, wherein # represents the point of attachment to X⁵;
—P⁰ is a bond, —NH—, —N(C₁-C₆ alkyl)-, —O—, or S(O)₀₋₂;
—P¹ is selected from the group consisting of: (C₁-C₆)alkylene, (C₂-C₆)alkenylene, (C₂-C₆)alkynylene, (C₃-C₈)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH, sulfo, phosphono, alkylsulfonylcarbamoyl, tetrazolyl, arylsulfonylcarbamoyl, heteroarylsulfonylcarbamoyl, N-methoxycarbamoyl, 3-hydroxy-3-cyclobutene-1,2-dione, 3,5-dioxo-1,2,4-oxadiazolidinyl or heterocyclic phenol such as 3-hydroxyisoxazolyl or 3-hydoxy-1-methylpyrazolyl; or
the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₅)alkyl, —C(O)NH(C₁-C₅)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₅)alkyl, —C(O)NH—(C₃-C₆)cycloalkyl, 3- to 6-membered heterocycloalkyl, and 6-membered heteroaryl is optionally substituted with 1-4 R^{T};
T² is:
   hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
   —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)— C₁-C₆ alkyl, S(O)₂(C₁-C₆ alkyl) and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

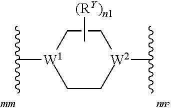

wherein n1 is 0, 1, or 2, W¹ is CR^{Y1} or N, and W² is CR^{Y2} or N;

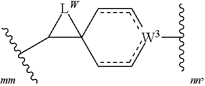

wherein W³ is C, CR^{Y3}, or N, L^{w} is (C₁-C₃)alkylene, and each [symbol] is independently a single bond or a double bond, as allowed by valence;
phenylene optionally substituted with 1-4 R^{Y};
5- to 6-membered heteroarylene optionally substituted with 1-3 R^{Y};
(C₅-C₈)cycloalkylene optionally substituted with 1-4 R^{y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to Ring B;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1}, R^{Y2}, and R^{Y3} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together can form (C₁-C₄)alkylene, wherein one of the CH₂ units of the (C₁-C₄)alkylene is optionally replaced by a heteroatom selected from the group consisting of O, S, NH, and N(C₁₋₃)alkyl;
Ring B is selected from the group consisting of: (B-I), (B-II), (B-III), (B-IV), (B-V), (B-VI), and (B-VII):

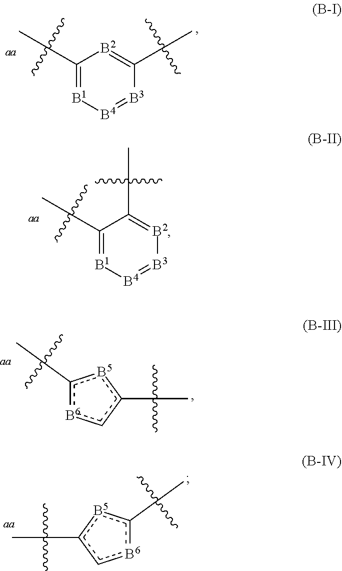

wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl;

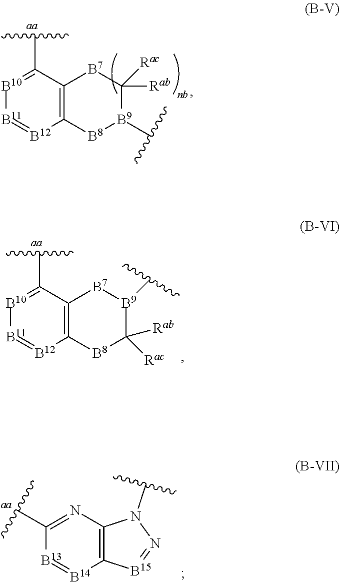

wherein aa represents the point of attachment to Ring A;
B⁷ and B⁸ are independently selected from the group consisting of: —O—, —NR^{N}—, and —C(R′)₂—;
B⁹ is N or CR^{aa};
nb is 0 or 1;
B¹⁰, B¹¹, and B¹² are independently selected from the group consisting of CR¹ and N;
B¹³, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or Z¹-Z²—*, wherein * represents the point of attachment to Ring C;
—Z¹ is a bond, NH, N(C₁-C₆ alkyl), O, or S(O)₀₋₂;
—Z² is C₁₋₃ alkylene optionally substituted with 1-2 R^{c};
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3;
provided at least one of the following occurs:
1) at least one R^{b} is —C(O)NH₂ or —C(O)OH;
2) Ring A is (C₅-C₈)cycloalkylene optionally substituted with 1-4 R^{y};
3) Ring B is

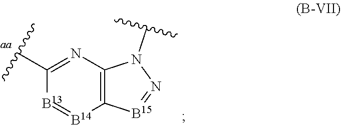

4) the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}.

In some embodiments, the compound is not:

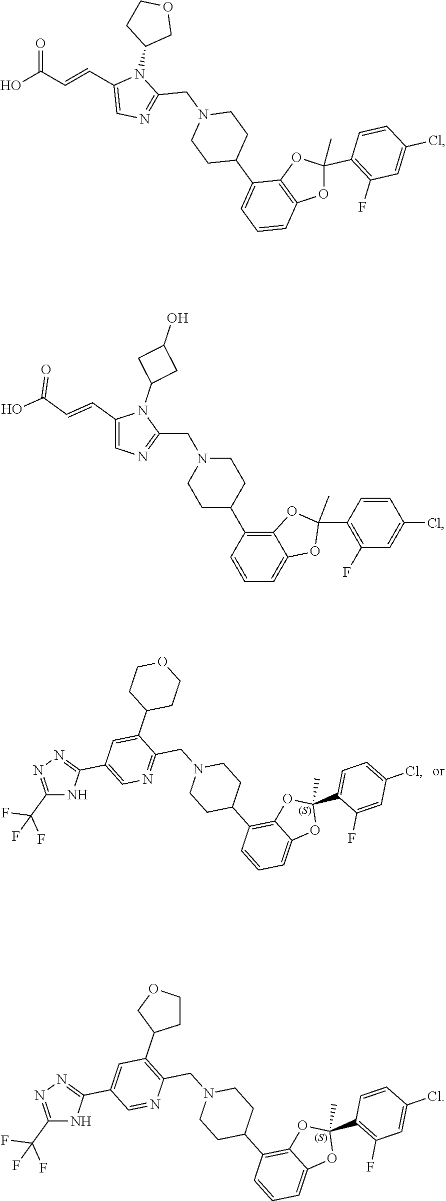

In some embodiments, each R^{x} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl. In some embodiments, each R^{x} is independently selected from the group consisting of hydrogen, (C₁-C₆)alkyl, (C₁-C₆)haloalkyl, and (C₃-C₆)cycloalkyl. In some embodiments, each R^{x} is independently selected from the group consisting of hydrogen, (C₁-C₆)alkyl, and (C₁-C₆)haloalkyl.

In some embodiments, each R^{y} is independently selected from the group consisting of hydrogen, —OH, (C₁-C₆)alkyl, (C₁-C₆)haloalkyl, (C₁-C₆)alkoxy, (C₁-C₆)haloalkoxy, CN, and halogen.

In some embodiments, X³ is N.

In some embodiments, X⁶ is a bond. For avoidance of doubt, when X⁶ is a bond, X¹ is directly attached to X⁵ (e.g., via a single bond or double bond), thereby providing a 5-membered heteroaromatic ring.

In some embodiments, X³ is N; and X⁶ is a bond.

In some embodiments, X³ and X⁴ are independently selected from the group consisting of: N, NR^{x}, CR^{y}, O, and S.

In some embodiments, X⁶ is selected from the group consisting of: a bond, N, NR^{x}, and CR^{y}.

In some embodiments, the ring including X¹-X⁶ is:

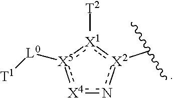

In some embodiments, X² is C. In some embodiments, X¹ is N. In some embodiments, X⁵ is C. In some embodiments, X⁴ is CR^{y}. In some embodiments, X⁴ is CH. In some embodiments, X⁴ is N.

In some embodiments, X¹ is N; X² is C; and X⁵ is C. In some embodiments, the ring including X¹-X⁶ is

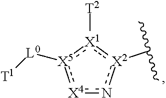

wherein X¹ is N; and X² is C. In some embodiments, X⁴ is CH. In some embodiments, X⁴ is N.

In some embodiments, X¹ is N; X² is C; X³ is N; X⁴ is CR^{y} or N; X⁵ is C; and X⁶ is a bond. In some embodiments, X⁴ is CH or N. As a non-limiting example of the foregoing embodiments, X¹ is N; X² is C; X³ is N; X⁴ is CH; X⁵ is C; and X⁶ is a bond. As another non-limiting example, X¹ is N; X² is C; X³ is N; X⁴ is N; X⁵ is C; and X⁶ is a bond.

In some embodiments, X⁶ is selected from the group consisting of: N, NR^{x}, CR^{y}, and C(O). In some embodiments, X³ is N; and X⁶ is selected from the group consisting of: N, NR^{x}, CR^{y}, and C(O).

In some embodiments, the ring including X¹—X⁶ is:

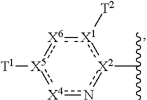

wherein X⁶ is selected from the group consisting of: N, NR^{x}, CR^{y}, and C(O). In some embodiments, X¹ is C. In some embodiments, X² is C. In some embodiments, X⁵ is C. In some embodiments, X⁶ is CR^{y}. For example, X⁶ can be CH. In some embodiments, X⁴ is CR^{y}. For example, X⁴ can be CH.

In some embodiments, X¹, X², and X⁵ are C; X⁴ and X⁶ are independently N or CR^{y}. In some embodiments, X⁴ and X⁶ are independently selected CR^{y}. As a non-limiting example of the foregoing embodiments, X⁴ and X⁶ can be CH.

In some embodiments, provided herein is a compound of Formula II:

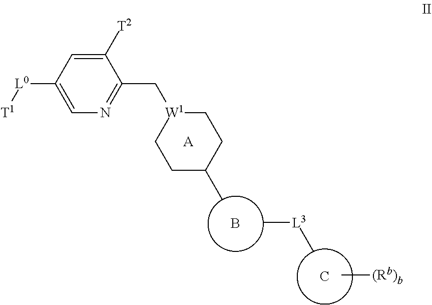

wherein:
or a pharmaceutically acceptable salt or solvate thereof, wherein:
L⁰ is a bond or —P⁰-P¹;
—P⁰ is a bond, —NH—, —N(C₁-C₆ alkyl)-, —O—, or S(O)₀₋₂;
—P¹ is selected from the group consisting of: (C₁-C₆)alkylene, (C₂-C₆)alkenylene, (C₂-C₆)alkynylene, (C₃-C₆)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH or a carboxylic acid bioisostere; or
the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
T² is:
hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
—NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)— C₁-C₆ alkyl, S(O)₂(C₁-C₆ alkyl) and di(C₁-C₆)alkylamino;
W¹ is CH or N;
Ring B is selected from the group consisting of: (B-I), (B-II), (B-III), (B-IV), (B-V), (B-VI), and (B-VII):

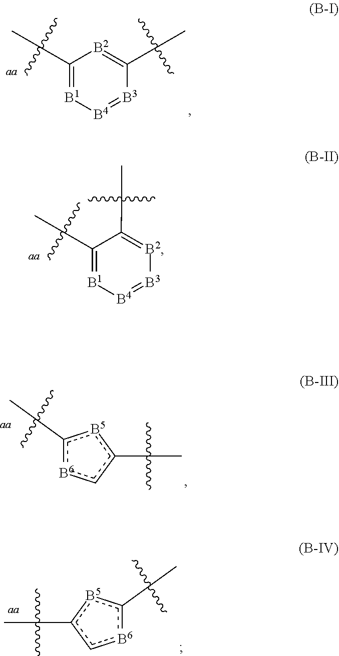

wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl;

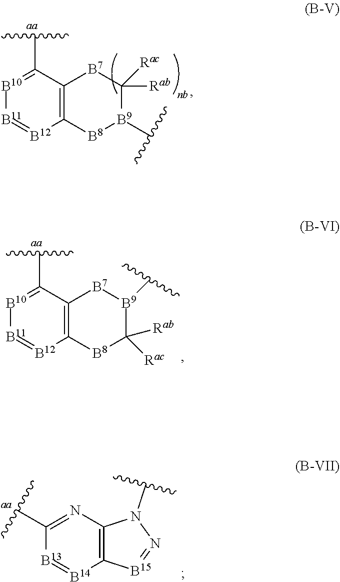

wherein aa represents the point of attachment to Ring A;
B⁷ and B⁸ are independently selected from the group consisting of: —O—, —NR^{N}—, and —C(R′)₂—;
B⁹ is N or CR^{aa};
nb is 0 or 1;
B¹⁰, B¹¹, and B¹² are independently selected from the group consisting of CR¹ and N;
B¹³, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and
   —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or Z¹-Z²—*, wherein * represents the point of attachment to Ring C;
—Z¹ is a bond, NH, N(C₁-C₆ alkyl), O, or S(O)₀₋₂;
—Z² is C₁₋₃ alkylene optionally substituted with 1-2 R^{c};
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3;
provided at least one of the following occurs:
1) at least one R^{b} is —C(O)NH₂ or —C(O)OH;
2) W¹ is CH;
3) Ring B is

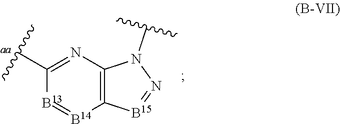

4) the moiety -L⁰-T¹ is:
   —C(O)NH—(C₁-C₆)alkyl or —C(O)NH—(C₃-C₆)cycloalkyl; wherein each is optionally substituted with 1-4 R^{T}; or
   a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}.

In some embodiments, the compound is not:

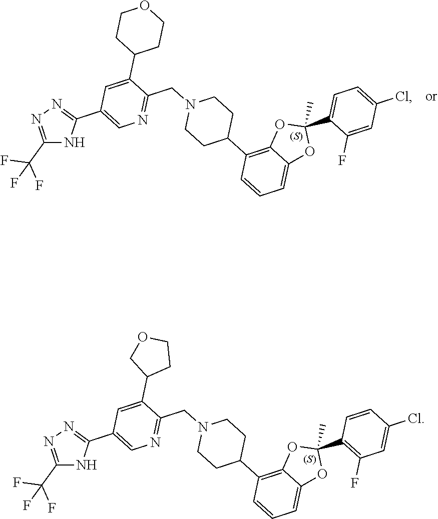

In some embodiments, L⁰ is a bond. In some embodiments, L⁰ is #—P⁰-P¹; and P⁰ is a bond.

In some embodiments, the moiety -L⁰-T¹ is —C(O)NH—(C₁-C₆)alkyl or —C(O)NH—(C₃-C₆)cycloalkyl; wherein each is optionally substituted with 1-4 R^{T}. In some embodiments, the moiety -L⁰-T¹ is —C(O)NH—(C₁-C₆)alkyl optionally substituted with 1-4 R^{T}. In some embodiments, the moiety -L⁰-T¹ is —C(O)NH—(C₃-C₆)cycloalkyl optionally substituted with 1-4 R^{T}.

In some embodiments, provided is a compound of Formula IID:

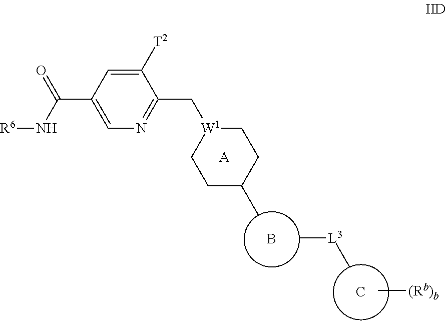

or a pharmaceutically acceptable salt or solvate thereof, wherein R⁶ is —(C₁-C₆)alkyl or —(C₃-C₆)cycloalkyl; and wherein the —(C₁-C₆)alkyl or —(C₃-C₆)cycloalkyl is optionally substituted with 1-4 R^{T}.

In some embodiments, R⁶ is —(C₁-C₆cycloalkyl wherein the —(C₁-C₆)cycloalkyl is optionally substituted with 1-4 R^{T}. In some embodiments, R⁶ is —(C₁-C₆)alkyl wherein the —(C₁-C₆)alkyl is optionally substituted with 1-4 R^{T}.

In some embodiments, the compound of Formula I, II, or IID is compound D-1, D-2, D-3, D-4, D-5, D-6, or D-7, as depicted in Table 1, or a pharmaceutically acceptable salt or solvate thereof.

In some embodiments, the moiety -L⁰-T¹ is a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T}.

In some embodiments, provided is a compound of Formula IIE:

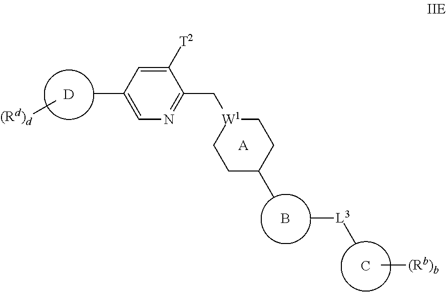

or a pharmaceutically acceptable salt or solvate thereof, wherein:
Ring D is a 5- to 6-membered heteroaryl;
d is 0 or 1; and
R^{d} is —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein the —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl is optionally substituted with 1-4 R^{T}.

In some embodiments, the compound is not:

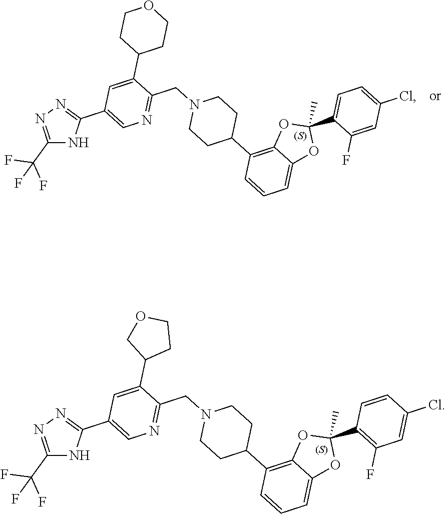

In some embodiments, d is 0. In some embodiments, d is 1.

In some embodiments, Ring D is triazolyl, thiazolyl, pyrazolyl, oxadiazolyl, pyridinyl, or thiadiazolyl.

In some embodiments, the compound of Formula I, II, or IIE is compound E-2, E-3, E-4, E-5, E-6, E-7, E-8, E-9, E-10, E-11, E-12, E-13, E-14, E-15, E-16, E-17, E-18, or E-19, as depicted in Table 1, or a pharmaceutically acceptable salt or solvate thereof.

In some embodiments, L⁰ is #—P⁰-P¹; and P⁰ is —NH—, —N(C₁-C₆ alkyl)-, —O—, or S(O)₀₋₂. In some embodiments, P⁰ is —O—. In some embodiments, P⁰ is —NH—.

In some embodiments, P¹ is (C₁-C₆)alkylene which is optionally substituted with 1-3 R⁰. In some embodiments, P¹ is (C₁-C₃)alkylene which is optionally substituted with 1-3 R⁰. In some embodiments, P¹ is (C₁-C₃)alkylene. In some embodiments, P¹ is —CH₂CH₂—, —CH₂—, —CH(Me)- or —C(Me)₂-. For example, P¹ can be —CH₂CH₂—. As another non-limiting example, P¹ can be —CH₂—. As further non-limiting examples, P¹ can be —CH(Me)- or —C(Me)₂-.

In some embodiments, P¹ is (C₂-C₆)alkenylene or (C₂-C₆)alkynylene, each of which is optionally substituted with 1-3 R⁰. In some embodiments, P¹ is (C₂-C₆)alkenylene which is optionally substituted with 1-3 R⁰. In some embodiments, P¹ is (C₂-C₄)alkenylene (e.g., (C₂-C₃)alkenylene, such as C₂ alkenylene which is optionally substituted with 1-3 R⁰.

In some embodiments, P¹ is

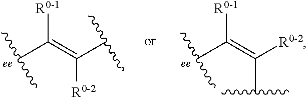

wherein R⁰⁻¹ and R⁰⁻² are independently H or R⁰; and ee is the point of attachment to T¹. In some embodiments, P¹ is

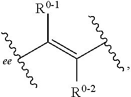

wherein R⁰⁻¹ and R⁰⁻² are independently H or R⁰; and ee is the point of attachment to T¹. In some embodiments, P¹ is

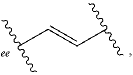

wherein ee is the point of attachment to T¹. In some embodiments, P¹ is

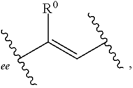

wherein ee is the point of attachment to T¹. For example, P¹ can be

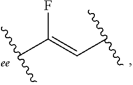

wherein ee is the point of attachment to T¹. As another non-limiting example, P¹ can be

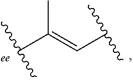

wherein ee is the point of attachment to T¹. In some embodiments, P¹ is

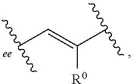

wherein ee is the point of attachment to T¹. For example, P¹ can be

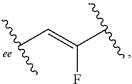

wherein ee is the point of attachment to T¹. As another non-limiting example, P¹ can be

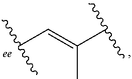

wherein ee is the point of attachment to T¹.

In some embodiments, P¹ is

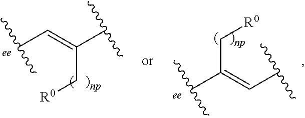

wherein np is 1, 2, or 3; and ee is the point of attachment to T¹. In some embodiments, P¹ is

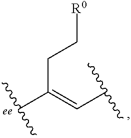

wherein ee is the point of attachment to T¹. For example, P¹ can be

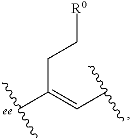

wherein R⁰ is (C₁-C₅)alkoxy or (C₁-C₆)haloalkoxy; and ee is the point of attachment to T¹.

In some embodiments, P¹ is selected from the group consisting of: (C₃-C₈)cycloalkylene, and 4- to 8-membered heterocycloalkylene, each of which is optionally substituted with 1-3 R⁰.

In some embodiments, P¹ is (C₃-C₈)cycloalkylene, which is optionally substituted with 1-3 R⁰. In some embodiments, P¹ is (C₃-C₆)cycloalkylene, which is optionally substituted with 1-3 R⁰. In some embodiments, P¹ is (C₃-C₄)cycloalkylene. As a non-limiting example of the foregoing embodiments, P¹ can be

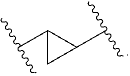 [SMILES: *C1CC1*]

In some embodiments, P¹ is 4- to 8-membered heterocycloalkylene, which is optionally substituted with 1-3 R⁰. In some embodiments, P¹ is

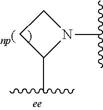

which is optionally substituted with 1-2 R⁰, wherein np is 1, 2, or 3; and ee is the point of attachment to T¹. In some embodiments, P¹ is

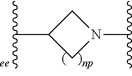

which is optionally substituted with 1-2 R⁰, wherein np is 1, 2, or 3; and ee is the point of attachment to T.

In some embodiments, L⁰ is #—P⁰-P¹; P⁰ is a bond; and P¹ is (C₁-C₃)alkylene which is optionally substituted with 1-3 R⁰. In some embodiments, P¹ is (C₁-C₃)alkylene. For example, P¹ can be CH₂CH₂. As another example, P¹ can be CH₂.

In some embodiments, L⁰ is #—P⁰-P¹; P⁰ is —NH—, —N(C₁-C₃ alkyl) or —O—; and P¹ is (C₁-C₃)alkylene which is optionally substituted with 1-3 R⁰. In some embodiments, P¹ is (C₁-C₃)alkylene. As non-limiting examples of the foregoing embodiments, P¹ can be CH₂, CH(Me), or C(Me)₂.

In some embodiments, L⁰ is #—P⁰-P¹; P⁰ is a bond; and P¹ is (C₂-C₄)alkenylene which is optionally substituted with 1-3 R⁰. In some embodiments, P¹ is

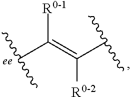

wherein R⁰⁻¹ and R⁰⁻² are independently H or R⁰; and ee is the point of attachment to T¹. As non-limiting examples of the foregoing embodiments, P¹ can be

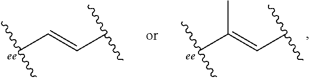

wherein ee is the point of attachment to T¹.

In some embodiments, L⁰ is #—P⁰-P¹; P⁰ is a bond; and P¹ is (C₃-C₆)cycloalkylene, which is optionally substituted with 1-3 R⁰. In some embodiments, P¹ is

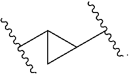 [SMILES: *C1CC1*]

In some embodiments, L⁰ is #—P⁰-P¹; P⁰ is a bond; and P¹ is

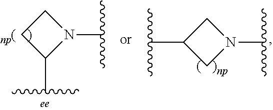

each of which is optionally substituted with 1-2 R⁰, wherein np is 1, 2, or 3; and ee is the point of attachment to T¹.

In some embodiments, T¹ is C(O)OH.

In some embodiments, T¹ is a carboxylic acid bioisostere.

In some embodiments, T¹ is tetrazolyl optionally substituted with (C₁-C₃)alkyl.

In some embodiments, T¹ is tetrazolyl, which is optionally substituted with from 1-2 substituents each independently selected from the group consisting of hydroxy, (C₁-C₅)alkyl, (C₁-C₅)haloalkyl, and halogen. For example, T¹ is selected from the group consisting of:

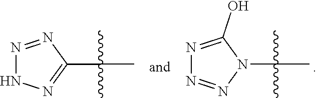 [SMILES: *c1nn[nH]n1 ; *n1nnnc1O]

In some embodiments, T¹ is triazolyl or oxadiazolyl, which is optionally substituted with from 1-2 substituents each independently selected from (C₁-C₆)alkyl and hydroxy. For example, T¹ is

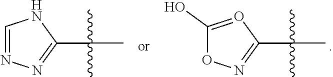

In some embodiments, T¹ is triazolyl, which is optionally substituted with from 1-2 substituents each independently selected from (C₁-C₆)haloalkyl, CN. For example, T¹ is

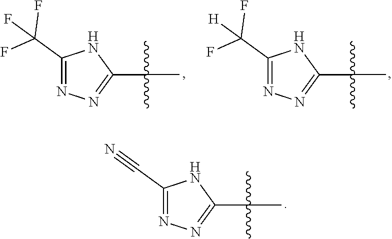 [SMILES: *c1nnc(C(F)(F)F)[nH]1 ; *c1nnc(C(F)F)[nH]1 ; *c1nnc(C#N)[nH]1]

In some embodiments, T¹ is a ring (e.g., a 4-6 membered ring, e.g., a 5-membered ring) including from 0-3 heteroatoms each independently selected from the group consisting of N, O, and S, wherein the ring is substituted with from 1-2 oxo and further optionally substituted from 1-2 substituent each independently selected from the group consisting of hydroxy, (C₁-C₆)alkyl, (C₁-C₆)haloalkyl, and halogen. For example, in some embodiments T¹ is

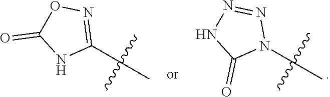 [SMILES: *c1noc(=O)[nH]1 ; *n1nn[nH]c1=O]

In some embodiments, T¹ is (C₁-C₆)alkyl which is substituted with from 1-3 hydroxy and further optionally substituted with from 1-10 fluoro. In some embodiments, T¹ is (C₁-C₆)alkyl which is substituted with from 1-3 hydroxy and further substituted with from 1-10 fluoro. For example, in some embodiments T¹ is

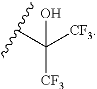 [SMILES: *C(O)(C(F)(F)F)C(F)(F)F]

In some embodiments, T¹ is C(O)NHS(O)₂(C₁-C₄)alkyl. For example, in some embodiments, T¹ is C(O)NHS(O)₂Me.

In some embodiments, T¹ is selected from the group consisting of the following:

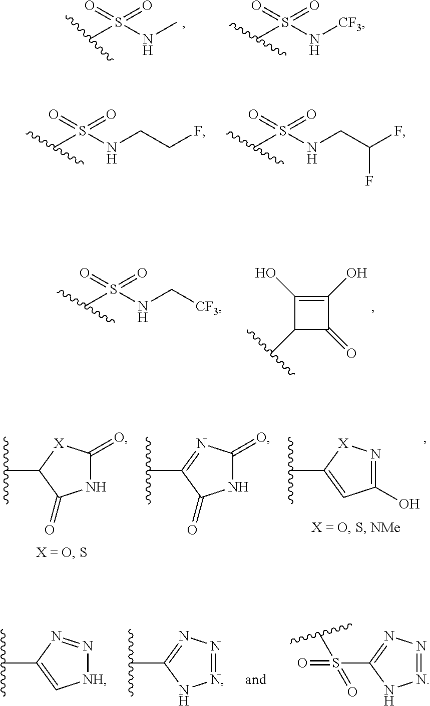

In some embodiments, T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments, each R^{T} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino.

In some embodiments, T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; and each R^{T} is independently selected from the group consisting of OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₅)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino.

In some embodiments, T² is hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments, each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)—C₁-C₆ alkyl, S(O)₂(C₁-C₆ alkyl) and di(C₁-C₆)alkylamino.

In some embodiments, T² is hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; and each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆alkyl)-C(O)— C₁-C₆ alkyl, S(O)₂(C₁-C₆ alkyl) and di(C₁-C₆)alkylamino.

In some embodiments, T² is hydrogen.

In some embodiments, T² is CN.

In some embodiments, T² is (C₁-C₆)alkyl. In some embodiments, T² is methyl.

In some embodiments, T² is (C₁-C₆)alkyl which is substituted with (C₁-C₆)alkoxy. In some embodiments, T² is (C₁-C₃)alkyl which is substituted with (C₁-C₃)alkoxy. For example, T² can be

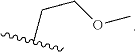 [SMILES: *CCOC]

In some embodiments, T² is (C₁-C₆)alkyl which is substituted with S(O)₂(C₁-C₆ alkyl). In some embodiments, T² is (C₁-C₃)alkyl which is substituted with S(O)₂(C₁-C₃ alkyl). For example, T² can be

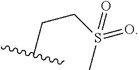 [SMILES: *CCS(C)(=O)=O]

In some embodiments, T² is (C₁-C₆)alkyl which is substituted with (C₃-C₆)cycloalkyl. In some embodiments, T² is (C₁-C₃)alkyl which is substituted with (C₃-C₆)cycloalkyl. In some embodiments, T² is (C₁-C₃)alkyl which is substituted with cyclobutyl. For example, T² can be

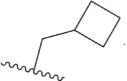 [SMILES: *CC1CCC1]

In some embodiments, T² is (C₁-C₆)alkyl which is substituted with 3- to 6-membered heterocycloalkyl. In some embodiments, T² is (C₁-C₃)alkyl which is substituted with 3- to 5-membered heterocycloalkyl. In some embodiments, T² is (C₁-C₃)alkyl which is substituted with oxetanyl. For example, T² can be

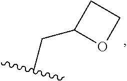 [SMILES: *CC1CCO1]

optionally wherein the stereogenic center in

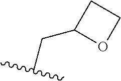 [SMILES: *CC1CCO1]

has (S)-configuration. In some embodiments, T² is (C₁-C₃)alkyl which is substituted with tetrahydrofuranyl. For example, T² can be

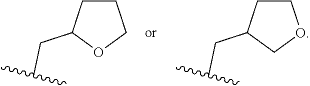 [SMILES: *CC1CCCO1 ; *CC1CCOC1]

In some embodiments, T² is (C₁-C₅)alkyl which is substituted with 5- to 6-membered heteroaryl, wherein the 5- to 6-membered heteroaryl is optionally substituted with 1-4 R^{T}. In some embodiments, T² is (C₁-C₃)alkyl which is substituted with 5-membered heteroaryl, wherein the 5-membered heteroaryl is optionally substituted with 1-2 R^{T}. In some embodiments, T² is (C₁-C₃)alkyl which is substituted with imidazolyl, wherein the imidazolyl is optionally substituted with R^{T}. For example, T² can be

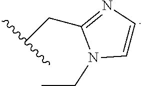 [SMILES: *Cc1nccn1CC]

As another non-limiting example, T² can be

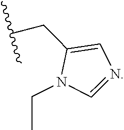 [SMILES: *Cc1cncn1CC]

In some embodiments, T² is (C₁-C₆)alkyl which is substituted with (C₃-C₆)cycloalkoxy.

In some embodiments, T² is (C₁-C₆)alkyl which is substituted with 3- to 6-membered heterocycloalkyl, wherein the 3- to 6-membered heterocycloalkyl is optionally substituted with 1-4 R^{T}. In certain of these embodiments, R^{T} is S(O)₂(C₁-C₆ alkyl).

In some embodiments, T² is (C₁-C₅)alkyl, which is substituted with —N(C₁-C₆ alkyl)-S(O)₂(C₁-C₆ alkyl).

In some embodiments, T² is (C₁-C₅)alkyl, which is substituted with —NH—S(O)₂(C₁-C₆ alkyl).

In some embodiments, T² is (C₁-C₆)hydroxyalkyl.

In some embodiments, T² is (C₁-C₅)haloalkyl.

In some embodiments, T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₅)alkyl, —NH—(C₃-C₅)cycloalkyl, —CH₂—S(O)₂—(C₃-C₆)cycloalkyl, —O—(C₃-C₆)cycloalkyl, (C₁-C₅)alkoxy, (C₃-C₅)cycloalkyl, or 3-10 membered heterocycloalkyl is optionally substituted with 1-4 R^{T}.

In some embodiments, T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered nitrogen-containing heterocycloalkyl; wherein each —NH(C₁-C₅)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₅)cycloalkyl, or 3-10 membered nitrogen-containing heterocycloalkyl is optionally substituted with 1-4 R^{T}.

In some embodiments, T² is (C₃-C₆)cycloalkyl or 3-10 membered heterocycloalkyl; wherein each (C₃-C₆)cycloalkyl or 3-10 membered heterocycloalkyl is optionally substituted with 1-4 R^{T}. In some embodiments, T² is (C₃-C₆)cycloalkyl or 3-10 membered nitrogen-containing heterocycloalkyl; wherein each (C₃-C₆)cycloalkyl or 3-10 membered nitrogen-containing heterocycloalkyl is optionally substituted with 1-4 R^{T}.

In some embodiments, T² is (C₃-C₅)cycloalkyl optionally substituted with 1-4 R^{T}. In some embodiments, T² is 3-10 membered heterocycloalkyl optionally substituted with 1-4 R^{T}. In some embodiments, T² is 3-10 membered nitrogen-containing heterocycloalkyl optionally substituted with 1-4 R^{T}.

In some embodiments, provided is a compound of Formula IIF:

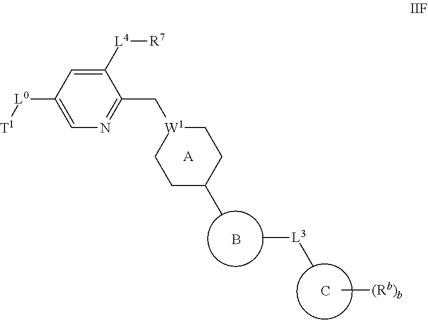

or a pharmaceutically acceptable salt or solvate thereof, wherein:
L⁴ is a bond, —NH—, —CH₂—S(O)₂, or —O—; and
R⁷ is (C₁-C₆)alkyl, (C₃-C₆)cycloalkyl, or 3-10 membered heterocycloalkyl, wherein each (C₁-C₆)alkyl, (C₃-C₆)cycloalkyl, or 3-10 membered heterocycloalkyl is optionally substituted with 1-4 R^{T}; provided that when L⁴ is a bond, then R⁷ is substituted or unsubstituted (C₃-C₆)cycloalkyl, or substituted or unsubstituted 3-10 membered heterocycloalkyl.

In some embodiments, the compound is not:

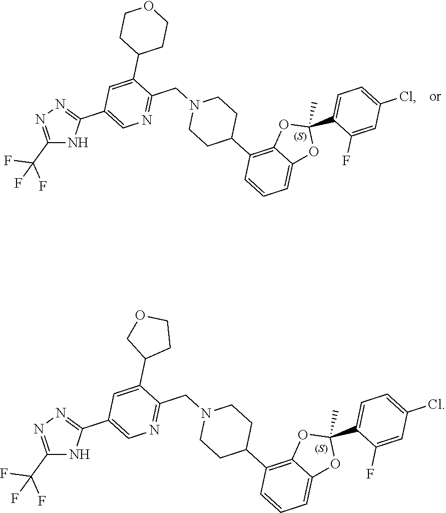

In some embodiments, R⁷ is (C₃-C₆)cycloalkyl, or 3-10 membered heterocycloalkyl, wherein each (C₃-C₅)cycloalkyl, or 3-10 membered heterocycloalkyl is optionally substituted with 1-4 R^{T}. In some embodiments, R⁷ is (C₃-C₆)cycloalkyl optionally substituted with 1-4 R^{T}. In some embodiments, R¹ is 3-10 membered heterocycloalkyl optionally substituted with 1-4 R^{T}. In some embodiments, R¹ is 3-10 membered nitrogen-containing heterocycloalkyl optionally substituted with 1-4 R^{T}.

In some embodiments, L⁴ is a bond, —NH—, —CH₂—S(O)₂, or —O—; and R⁷ is (C₁-C₆)alkyl, (C₃-C₆)cycloalkyl, or a 3-10 membered nitrogen-containing heterocycloalkyl, wherein the (C₁-C₆)alkyl, (C₃-C₆)cycloalkyl, or 3-10 membered nitrogen-containing heterocycloalkyl is optionally substituted with 1-4 R^{T}; provided that when L⁴ is a bond, then R⁷ is substituted or unsubstituted (C₃-C₆)cycloalkyl, or substituted or unsubstituted 3-10 membered nitrogen-containing heterocycloalkyl.

In some embodiments, the compound of Formula I, II, or IIF is compound F-1, F-2, F-3, F-4, F-5, F-6, F-7, F-8, F-9, F-10, F-11, F-12, F-13, F-14, F-15, F-16, F-17, F-18, F-19, or F-20, as depicted in Table 1, or a pharmaceutically acceptable salt or solvate thereof.

In some embodiments, L² is a bond.

In some embodiments, L¹ is CH₂. In some embodiments, L¹ is a bond.

In some embodiments, L² is a bond; and L¹ is CH₂.

In some embodiments, L¹ is a bond; and L² is a bond.

In some embodiments, Ring A is

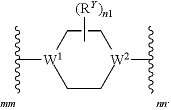

In some embodiments, W¹ is N. In some embodiments, W² is CR^{Y2}. In some embodiments, R^{Y2} is hydrogen. In some embodiments, W² is N. In some embodiments, n1 is 0. For example, Ring A can be

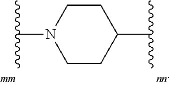 [SMILES: *C1CCN(*)CC1]

As another non-limiting example, Ring A can be

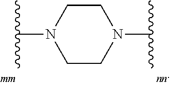 [SMILES: *N1CCN(*)CC1]

In some embodiments, n1 is 1. For example, Ring A can be

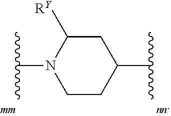

In some embodiments, Ring A is

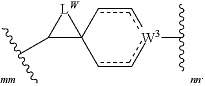

In some embodiments, Ring A is

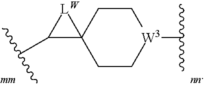

In some embodiments, L^{W} is CH₂. In some embodiments, W³ is N. As a non-limiting example of the foregoing embodiments, Ring A can be

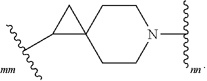 [SMILES: *C1CC12CCN(*)CC2]

In some embodiments, L² is a bond; L¹ is CH₂; and Ring A is

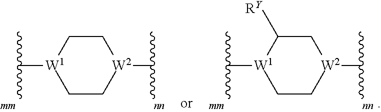

In some embodiments, Ring A is

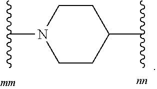 [SMILES: *C1CCN(*)CC1]

In some embodiments, Ring A is

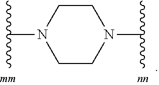 [SMILES: *N1CCN(*)CC1]

In some embodiments, L² is a bond; L¹ is a bond; and Ring A is

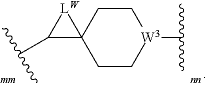

In some embodiments, Ring A is

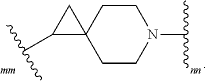 [SMILES: *C1CC12CCN(*)CC2]

In some embodiments, Ring A is (C₅-C₈)cycloalkylene optionally substituted with 1-4 R^{Y}. In some embodiments, Ring A is

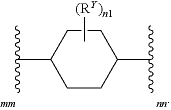

In some embodiments, provided is a compound of Formula IIB:

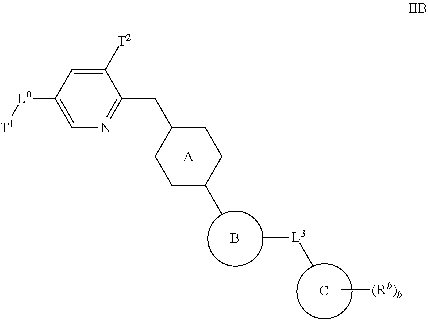

or a pharmaceutically acceptable salt or solvate thereof.

In some embodiments, the compound of Formula I, II, or IIB is compound B-1, as depicted in Table 1, or a pharmaceutically acceptable salt or solvate thereof.

In some embodiments, Ring B is

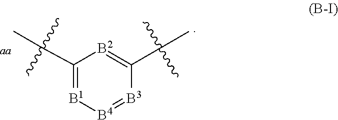

In some embodiments, B⁴ is CR¹. For example, B⁴ can be CH. In some embodiments, B¹ is CR¹. For example, B¹ can be CH. In some embodiments, B³ is CR¹. For example, B³ can be CH. In some embodiments, B² is N. In some embodiments, Ring B is

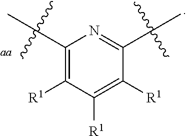

For example, Ring B can be

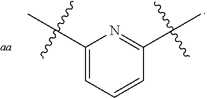 [SMILES: *c1cccc(*)n1]

In some embodiments, Ring B is

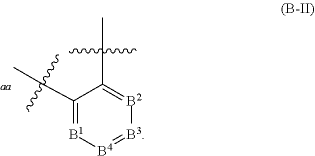

In some embodiments, B⁴ and B³ are independently selected CR¹. As non-limiting examples of the foregoing embodiments, B⁴ and B³ can be CH. In some embodiments, B¹ is CR¹. For example, B¹ can be CH. In some embodiments, B² is N. In some embodiments, Ring B is

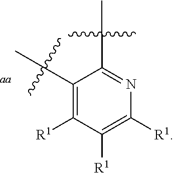

For example, Ring B can be

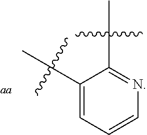 [SMILES: *c1cccnc1*]

In some embodiments, Ring B is

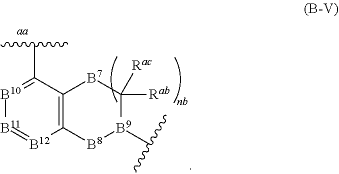

In some embodiments, B⁹ is CR^{aa}. In some embodiments, R^{aa} is H. In some embodiments, R^{aa} is (C₁-C₆)alkyl. In some embodiments, R^{aa} is (C₁-C₃)alkyl. For example, R^{aa} can be methyl. In some embodiments, nb is 0. In some embodiments, nb is 1. In some embodiments, when B⁹ is CR^{aa}, the carbon atom to which B⁸ and R^{aa} are both attached has (R)-configuration. In some embodiments, when B⁹ is CR^{aa}, the carbon atom to which B⁸ and R^{aa} are both attached has (S)-configuration.

In some embodiments, Ring B is

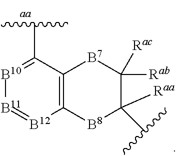

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₆)alkyl. In some embodiments, R^{aa} is (C₁-C₃)alkyl. For example, R^{aa} can be methyl. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, B¹⁰ is CR¹. For example, B¹⁰ can be CH. In some embodiments, B¹¹ is CR¹. For example, B¹¹ can be CH. In some embodiments, B¹² is CR¹. For example, B¹² can be CH. In some embodiments, B¹⁰, B¹¹, and B¹² are each independently selected CR¹. In some embodiments, B¹⁰, B¹¹, and B¹² are CH. In some embodiments, the carbon atom to which B⁸ and R^{aa} are both attached has (R)-configuration. In some embodiments, the carbon atom to which B⁸ and R^{aa} are both attached has (S)-configuration.

In some embodiments, B is

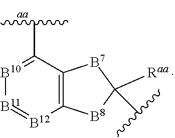

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₆)alkyl. In some embodiments, R^{aa} is (C₁-C₃)alkyl. For example, R^{aa} can be methyl. In some embodiments, B¹⁰ is CR¹. For example, B¹⁰ can be CH. In some embodiments, B¹¹ is CR¹. For example, B¹¹ can be CH. In some embodiments, B¹² is CR¹. For example, B¹² can be CH. In some embodiments, B¹⁰, B¹¹, and B¹² are each independently selected CR¹. In some embodiments, B¹⁰, B¹¹, and B¹² are CH. In some embodiments, the carbon atom to which B⁸ and R^{aa} are both attached has (R)-configuration. In some embodiments, the carbon atom to which B⁸ and R^{aa} are both attached has (S)-configuration.

In some embodiments, Ring B is

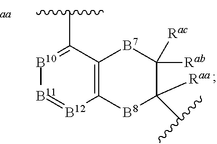

B⁷ and B⁸ are —O—; and R^{aa} is H or (C₁-C₃)alkyl, such as H or methyl. In some embodiments, R^{ab} and R^{ac} are H. In some embodiments, R^{aa} is H. In some embodiments, R^{aa} is (C₁-C₃)alkyl, such as methyl. In some embodiments, B¹⁰, B¹¹, and B¹² are each independently selected CR¹. In some embodiments, B¹⁰, B¹¹, and B¹² are CH. In some embodiments, the carbon atom to which B⁸ and R^{aa} are both attached has (R)-configuration. In some embodiments, the carbon atom to which B⁸ and R^{aa} are both attached has (S)-configuration.

In some embodiments, Ring B is

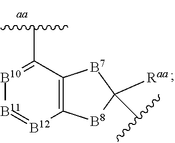

B⁷ and B⁸ are —O—; and R^{aa} is H or (C₁-C₃)alkyl. In some embodiments, R^{aa} is H. In some embodiments, R^{aa} is (C₁-C₃)alkyl, such as methyl. In some embodiments, B¹⁰, B¹¹, and B¹² are each independently selected CR¹. In some embodiments, B¹⁰, B¹¹, and B¹² are CH. In some embodiments, the carbon atom to which B⁸ and R^{aa} are both attached has (R)-configuration. In some embodiments, the carbon atom to which B⁸ and R^{aa} are both attached has (S)-configuration.

In some embodiments, Ring B is

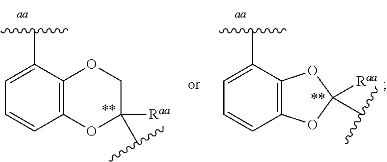

and the carbon atom labelled with ** has (R)-configuration.

In some embodiments, Ring B is

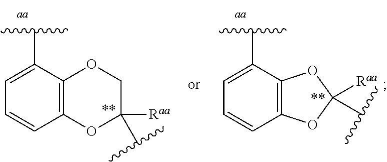

and the carbon atom labelled with ** has (S)-configuration.

In some embodiments, Ring B is

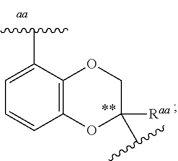

and the carbon atom labelled with ** has (R)-configuration.

In some embodiments, Ring B is

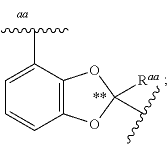

and the carbon atom labelled with ** has (S)-configuration. In some embodiments, R^{aa} is (C₁-C₃)alkyl. For example, R^{aa} can be methyl. For example, Ring B can be

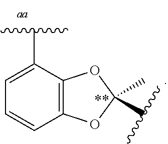 [SMILES: *c1cccc2c1O[C@](*)(C)O2]

In some embodiments, Ring B is

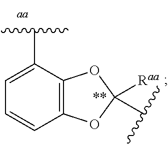

and the carbon atom labelled with ** has (R)-configuration. In some embodiments, R^{aa} is (C₁-C₃)alkyl. For example, R^{aa} can be methyl. For example, Ring B can be

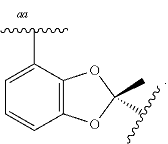 [SMILES: *c1cccc2c1O[C@@](*)(C)O2]

In some embodiments, Ring B is

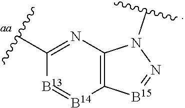

In some embodiments, Ring B is

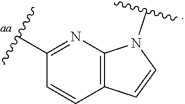 [SMILES: *c1ccc2ccn(*)c2n1]

In some embodiments, provided is a compound of Formula IIC:

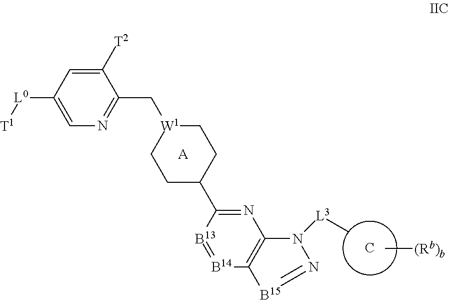

or a pharmaceutically acceptable salt or solvate thereof.

In some embodiments, the compound of Formula I, II, or IIC is compound AC-1, as depicted in Table 1, or a pharmaceutically acceptable salt or solvate thereof.

In some embodiments, L³ is a bond. In some embodiments, Ring B is selected from the group consisting of (B-V) and (B-VI); and L³ is a bond. In some embodiments, Ring B is (B-V) (e.g., Ring B is as defined in one or more embodiments, supra); and L³ is a bond.

In some embodiments, L³ is —Z¹-Z²—*, wherein * represents the point of attachment to Ring C. In some embodiments, Ring B is selected from the group consisting of (B-I), (B-II), (B-III), and (B-IV), L³ is —Z¹-Z²—*, wherein * represents the point of attachment to Ring C. For example, Ring B is (B-I) or (B-II); and L³ is —Z¹-Z²—*.

In some embodiments, Z¹ is —O—.

In some embodiments, Z² is —CH₂— optionally substituted with 1-2 R^{c}. In some embodiments, Z² is —CH₂—.

In some embodiments, L³ is O—CH₂—*, wherein * represents the point of attachment to Ring C.

In some embodiments, Ring C is selected from the group consisting of: phenyl, 5- to 6-membered heteroaryl, and 5- to 10-membered bicycloheteroaryl. In some embodiments, Ring C is selected from the group consisting of: phenyl and 6-membered heteroaryl (e.g., pyridyl).

In some embodiments, b is 1-3. In some embodiments, b is 2. In some embodiments, b is 1. In some embodiments, b is 0.

In some embodiments, Ring C is phenyl. In some embodiments, Ring C is phenyl; and b is 2. In some embodiments,

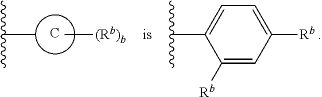

In some embodiments, Ring C is phenyl; and b is 1. In some embodiments,

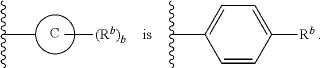

In some embodiments, Ring C is phenyl; and b is 0.

In some embodiments, each occurrence of R^{b} is independently selected from the group consisting of: (C₁-C₆)alkyl, (C₁-C₅)haloalkyl, (C₁-C₅)alkoxy, (C₁-C₆)haloalkoxy, halogen, and CN. In some embodiments, each occurrence of R^{b} is independently selected from the group consisting of —F, —Cl, CF₃, and CN.

In some embodiments, at least one R^{b} is —C(O)NH₂ or —C(O)OH. In some embodiments, at least one R^{b} is —C(O)NH₂ or —C(O)OH; and the remaining R^{b} are independently selected from the group consisting of (C₁-C₆)alkyl, (C₁-C₆)haloalkyl, (C₁-C₆)alkoxy, (C₁-C₆)haloalkoxy, halogen, and CN.

In some embodiments, provided is a compound of Formula IIA:

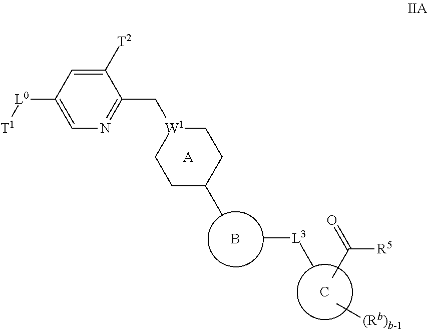

or a pharmaceutically acceptable salt or solvate thereof, wherein R⁵ is —OH or —NH₂, and each of the remaining variables are independently as defined herein.

In some embodiments, the compound of Formula I, II, or IIA is compound A-1 or A-3, as depicted in Table 1, or a pharmaceutically acceptable salt or solvate thereof.

In some embodiments, the compound is a compound of Formula (I-A1) or a pharmaceutically acceptable salt thereof:

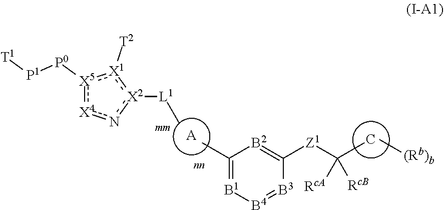

wherein R^{cA} and R^{cB} are independently selected from the group consisting of H and R^{c}.

In some embodiments of Formula (I-A1), the ring containing B¹, B², B³, and B⁴ is

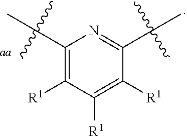

In some embodiments, the ring containing B¹, B², B³, and B⁴ can be

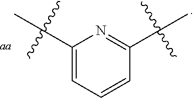 [SMILES: *c1cccc(*)n1]

In some embodiments, the compound is a compound of Formula (I-A2) or a pharmaceutically acceptable salt thereof:

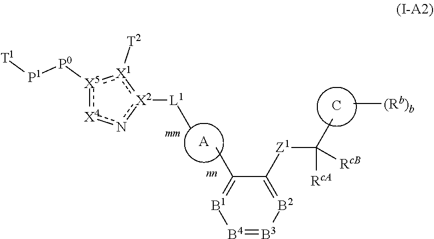

wherein R^{cA} and R^{cB} are independently selected from the group consisting of H and R^{c}.

In some embodiments of Formula (I-A2), the ring containing B¹, B², B³, and B⁴ is

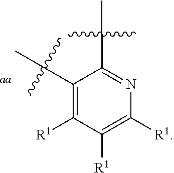

In some embodiments, the ring containing B¹, B², B³, and B⁴ can be

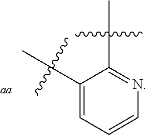 [SMILES: *c1cccnc1*]

In some embodiments of Formulae (I-A1) or (I-A2), Z¹ is —O—. In some embodiments of Formulae (I-A1) or (I-A2), R^{cA} is H. In some embodiments of Formulae (I-A1) or (I-A2), R^{cB} is H.

In some embodiments, the compound is a compound of Formula (I-A3):

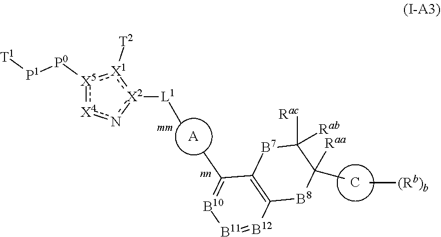

or a pharmaceutically acceptable salt thereof.

In some embodiments of Formula (I-A3), R^{ab} and R^{ac} are H.

In some embodiments, the compound is a compound of Formula (I-A4):

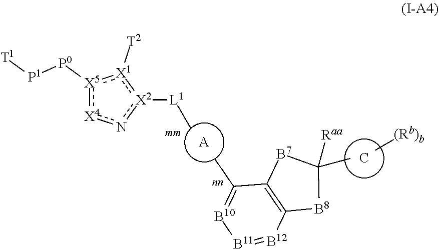

or a pharmaceutically acceptable salt thereof.

In some embodiments of Formula (I-A4), B⁷ is —O—; and B⁸ is —O—.

In some embodiments of Formulae (I-A3) or (I-A4), R^{aa} is H. In some embodiments, R^{a} is (C₁-C₃)alkyl. For example, R^{a}a can be methyl.

In some embodiments of Formulae (I-A3) or (I-A4), B¹⁰, B¹¹, and B¹² are independently selected CR¹. In some embodiments, B¹⁰, B¹¹, and B¹² are CH.

In some embodiments of Formulae (I-A1), (I-A2), (I-A3), or (I-A4), X¹ is N.

In some embodiments of Formulae (I-A1), (I-A2), (I-A3), or (I-A4), X² is C.

In some embodiments of Formulae (I-A1), (I-A2), (I-A3), or (I-A4), X⁵ is C.

In some embodiments of Formulae (I-A1), (I-A2), (I-A3), or (I-A4), X⁴ is N. In some embodiments of Formulae (I-A1), (I-A2), (I-A3), or (I-A4), X⁴ is CR^{y}, such as CH.

In some embodiments of Formulae (I-A1), (I-A2), (I-A3), or (I-A4), the

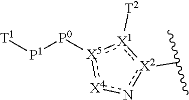

moiety is

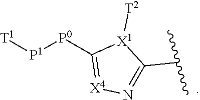

In some embodiments, X⁴ is N. In some embodiments, X⁴ is CR^{y}. For example, X⁴ can be CH.

In some embodiments, the compound is a compound of Formula (I-B1), or a pharmaceutically acceptable salt thereof:

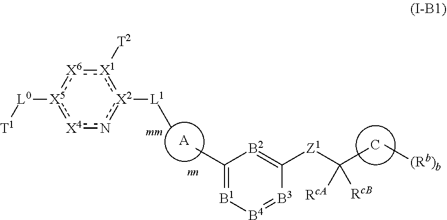

wherein X⁶ is selected from the group consisting of: N, NR^{x}, CR^{y}, and C(O); and R^{cA} and R^{cB} are independently selected from the group consisting of H and R^{c}.

In some embodiments of Formula (I-B1), X¹ is C. In some embodiments of Formula (I-B1), X² and X⁵ are C. In some embodiments of Formula (I-B1), X⁴ and X⁶ are independently selected CR^{y}. For example, X⁴ and X⁶ can be CH.

In some embodiments of Formula (I-B1), the

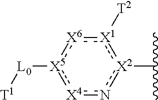

moiety is

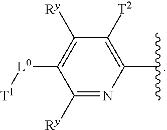

In some embodiments, each R^{y} is H.

In some embodiments of Formula (I-B1), L⁰ is #—P⁰-P¹.

In some embodiments of Formula (I-B1), Z¹ is —O—. In some embodiments of Formula (I-B1), R^{cA} is H; and R^{cB} is H.

In some embodiments of Formulae (I-A1), (I-A2), (I-A3), (I-A4), or (I-B1), P⁰ is a bond; and P¹ is (C₁-C₃)alkylene which is optionally substituted with 1-3 R⁰. In some embodiments, P¹ is (C₁-C₃)alkylene. For example, P¹ can be —CH₂CH₂—.

In some embodiments of Formulae (I-A1), (I-A2), (I-A3), (I-A4), or (I-B1), P⁰ is —NH—, —N(C₁-C₃ alkyl) or —O—; and P¹ is (C₁-C₃)alkylene which is optionally substituted with 1-3 R⁰. In some embodiments, P¹ is (C₁-C₃)alkylene. For example, P¹ can be CH₂, CH(Me), or C(Me)₂.

In some embodiments of Formulae (I-A1), (I-A2), (I-A3), (I-A4), or (I-B1), P⁰ is a bond; and P¹ is (C₂-C₄)alkenylene which is optionally substituted with 1-3 R⁰. In some embodiments, P¹ is

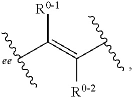

wherein R⁰⁻¹ and R⁰⁻² are independently H or R⁰; and ee is the point of attachment to T¹. In some embodiments, P¹ is

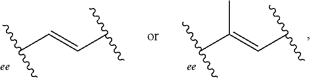

wherein ee is the point of attachment to T¹.

In some embodiments of Formulae (I-A1), (I-A2), (I-A3), (I-A4), or (I-B1), P⁰ is a bond; and P¹ is (C₃-C₆)cycloalkylene, which is optionally substituted with 1-3 R⁰. In some embodiments, P¹ is

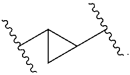 [SMILES: *C1CC1*]

In some embodiments of Formulae (I-A1), (I-A2), (I-A3), (I-A4), or (I-B1), P⁰ is a bond; and P¹ is

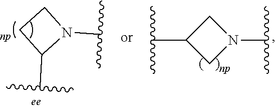

each of which is optionally substituted with 1-2 R⁰, wherein np is 1, 2, or 3; and ee is the point of attachment to T¹.

In some embodiments of Formulae (I-A1), (I-A2), (I-A3), (I-A4), or (I-B1), T¹ is C(O)OH.

In some embodiments of Formulae (I-A1), (I-A2), (I-A3), (I-A4), or (I-B1), T² is H or (C₁-C₃)alkyl.

In some embodiments of Formulae (I-A1), (I-A2), (I-A3), (I-A4), or (I-B1), T² is (C₁-C₃)alkyl which is substituted with (C₁-C₃)alkoxy. For example, T² can be

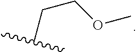 [SMILES: *CCOC]

In some embodiments of Formulae (I-A1), (I-A2), (I-A3), (I-A4), or (I-B1), T² is (C₁-C₃)alkyl which is substituted with S(O)₂(C₁-C₃ alkyl). For example, T² can be

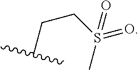 [SMILES: *CCS(C)(=O)=O]

In some embodiments of Formulae (I-A1), (I-A2), (I-A3), (I-A4), or (I-B1), T² is (C₁-C₃)alkyl which is substituted with (C₃-C₅)cycloalkyl. For example, T² can be

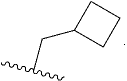 [SMILES: *CC1CCC1]

In some embodiments of Formulae (I-A1), (I-A2), (I-A3), (I-A4), or (I-B1), T² is (C₁-C₃)alkyl which is substituted with 3- to 5-membered heterocycloalkyl. In some embodiments, T² is (C₁-C₃)alkyl which is substituted with oxetanyl. For example, T² can be

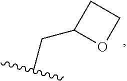 [SMILES: *CC1CCO1]

optionally wherein the stereogenic center in

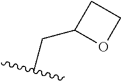 [SMILES: *CC1CCO1]

has (S)-configuration. In some embodiments, T² is (C₁-C₃)alkyl which is substituted with tetrahydrofuranyl. In some embodiments, T² is

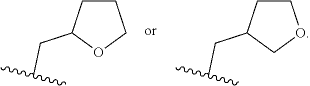 [SMILES: *CC1CCCO1 ; *CC1CCOC1]

In some embodiments of Formulae (I-A1), (I-A2), (I-A3), (I-A4), or (I-B1), T² is (C₁-C₃)alkyl which is substituted with 5-membered heteroaryl, wherein the 5-membered heteroaryl is optionally substituted with 1-2 R^{T}. In some embodiments, T² is (C₁-C₃)alkyl which is substituted with imidazolyl, wherein the imidazolyl is optionally substituted with R^{T}. For example, T² can be

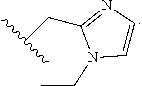 [SMILES: *Cc1nccn1CC]

As another non-limiting example, T² can be

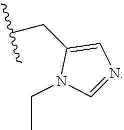 [SMILES: *Cc1cncn1CC]

In some embodiments of Formulae (I-A1), (I-A2), (I-A3), (I-A4), or (I-B1), L¹ is CH₂; and Ring A is

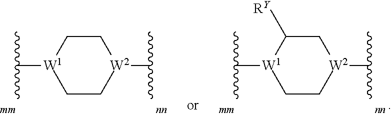

In some embodiments of Formulae (I-A1), (I-A2), (I-A3), (I-A4), or (I-B1), L¹ is CH₂; and Ring A is

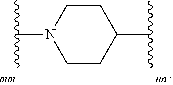 [SMILES: *C1CCN(*)CC1]

In some embodiments of Formulae (I-A1), (I-A2), (I-A3), (I-A4), or (I-B1), L¹ is CH₂; and Ring A is

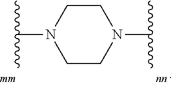 [SMILES: *N1CCN(*)CC1]

In some embodiments of Formulae (I-A1), (I-A2), (I-A3), (I-A4), or (I-B1), L¹ is a bond; and Ring A is

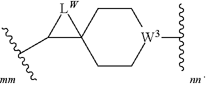

In some embodiments, Ring A is

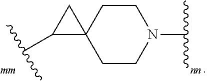 [SMILES: *C1CC12CCN(*)CC2]

In some embodiments of Formulae (I-A1), (I-A2), (I-A3), (I-A4), or (I-B1), Ring C is selected from the group consisting of: phenyl and 6-membered heteroaryl (e.g., pyridyl such as 2-pyridyl, 3-pyridyl, or 4-pyridyl).

In some embodiments of Formulae (I-A1), (I-A2), (I-A3), (I-A4), or (I-B1),

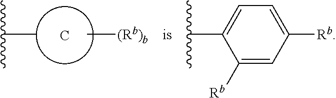

In some embodiments of Formulae (I-A1), (I-A2), (I-A3), (I-A4), or (I-B1), is

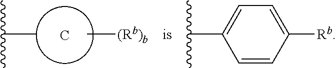

In some embodiments of Formulae (I-A1), (I-A2), (I-A3), (I-A4), or (I-B1), each occurrence of R^{b} is independently selected from the group consisting of: (C₁-C₆)alkyl, (C₁-C₆)haloalkyl, (C₁-C₅)alkoxy, (C₁-C₅)haloalkoxy, halogen, and CN.

In some embodiments of Formulae (I-A1), (I-A2), (I-A3), (I-A4), or (I-B1), R^{b} is independently selected from the group consisting of —F, —Cl, CF₃, and CN.

In some embodiments, the compound of Formula (I) is a compound of Formula (I-A4-1), or a pharmaceutically acceptable salt thereof:

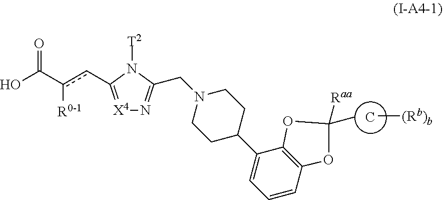

wherein:
[symbol] is a single bond or a double bond;
R⁰⁻¹ is selected from the group consisting of hydrogen and (C₁-C₃)alkyl;
X⁴ is selected from the group consisting of N and CR^{y};
   T² is (C₁-C₃)alkyl which is substituted with a substituent selected from the group consisting of (C₁-C₃)alkoxy, S(O)₂(C₁-C₃ alkyl), (C₃-C₆)cycloalkyl, 3- to 6-membered heterocycloalkyl, phenyl, and 5- to 6-membered heteroaryl, wherein the phenyl or 5- to 6-membered heteroaryl are each optionally substituted with halogen or (C₁-C₃)alkyl;
   R^{aa} is selected from the group consisting of hydrogen and (C₁-C₃)alkyl;
   Ring C is selected from the group consisting of: phenyl and 6-membered heteroaryl;
   b is 0, 1, or 2; and
   each occurrence of R^{b} is independently selected from the group consisting of: (C₁-C₅)alkyl, (C₁-C₅)haloalkyl, (C₁-C₆)alkoxy, (C₁-C₆)haloalkoxy, halogen, and CN.

In some embodiments of Formula (I-A4-1), [symbol] is a double bond.

In some embodiments of Formula (I-A4-1), [symbol] is a single bond.

In some embodiments of Formula (I-A4-1), R⁰⁻¹ is hydrogen.

In some embodiments of Formula (I-A4-1), R⁰⁻¹ is (C₁-C₃)alkyl. For example, R⁰⁻¹ can be methyl.

In some embodiments of Formula (I-A4-1), [symbol] is a double bond; and R⁰⁻¹ is hydrogen.

In some embodiments of Formula (I-A4-1), [symbol] is a double bond; and R⁰⁻¹ is methyl.

In some embodiments of Formula (I-A4-1), [symbol] is a single bond; and R⁰⁻¹ is hydrogen.

In some embodiments of Formula (I-A4-1), X⁴ is CH.

In some embodiments of Formula (I-A4-1), X⁴ is N.

In some embodiments of Formula (I-A4-1), T² is (C₁-C₃)alkyl which is substituted with 3- to 6-membered heterocycloalkyl. In some embodiments, T² is (C₁-C₃)alkyl which is substituted with oxetanyl. For example, T² can be

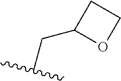 [SMILES: *CC1CCO1]

(e.g., the stereogenic center in T² can have (S)-configuration). In some embodiments, T² is (C₁-C₃)alkyl which is substituted with tetrahydrofuranyl. For example, T² can be or

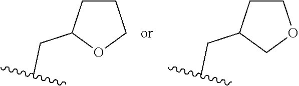 [SMILES: *CC1CCCO1 ; *CC1CCOC1]

(e.g., the stereogenic center in T² can have (S)-configuration).

In some embodiments of Formula (I-A4-1), T² is (C₁-C₃)alkyl which is substituted with 5-membered heteroaryl, wherein the 5-membered heteroaryl is optionally substituted with (C₁-C₃)alkyl. In some embodiments, T² is (C₁-C₃)alkyl which is substituted with imidazolyl, wherein the imidazolyl is optionally substituted with (C₁-C₃)alkyl. For example, T² can be

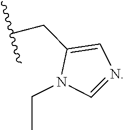 [SMILES: *Cc1cncn1CC]

As another non-limiting example, T² can be

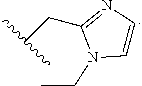 [SMILES: *Cc1nccn1CC]

In some embodiments of Formula (I-A4-1), T² is (C₁-C₃)alkyl which is substituted with (C₁-C₃)alkoxy. For example, T² can be

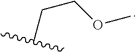 [SMILES: *CCOC]

In some embodiments of Formula (I-A4-1), R^{aa} is (C₁-C₃)alkyl. For example, R^{aa} is methyl.

In some embodiments of Formula (I-A4-1), R^{aa} is hydrogen.

In some embodiments of Formula (I-A4-1), the carbon to which both R^{aa} and Ring C are attached has (S)-configuration.

In some embodiments of Formula (I-A4-1), R^{aa} is (C₁-C₃)alkyl; and the carbon to which both R^{aa} and Ring C are attached has (S)-configuration. In some embodiments, R^{aa} is methyl.

In some embodiments of Formula (I-A4-1), Ring C is phenyl.

In some embodiments of Formula (I-A4-1), b is 1 or 2.

In some embodiments of Formula (I-A4-1),

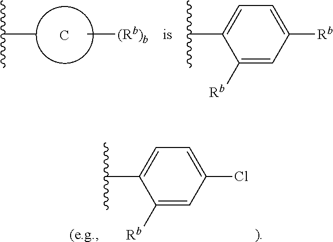

In some embodiments of Formula (I-A4-1),

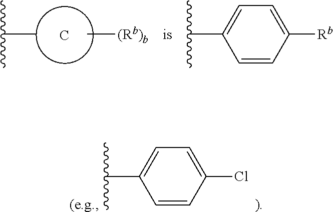

In some embodiments of Formula (I-A4-1), each R^{b} is independently selected from the group consisting of: —F, —Cl, and CN. In some embodiments, each R^{b} is independently selected from the group consisting of: —F and —Cl. For example, each R^{b} can be independently —F or —Cl.

In some embodiments of Formula (I-A4-1):
R⁰⁻¹ is hydrogen or methyl;
X⁴ is N or CH;
T² is selected from the group consisting of:

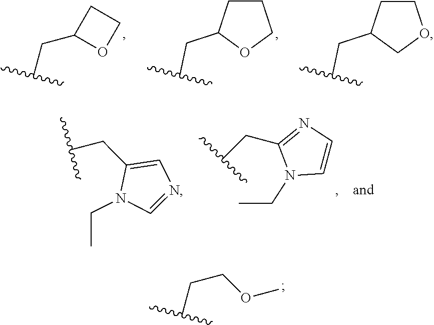

R^{aa} is (C₁-C₃)alkyl, wherein the carbon to which both R^{aa} and Ring C are attached has (S)-configuration;
Ring C is phenyl; and
b is 1 or 2.

In some embodiments, R^{aa} is methyl. In some embodiments, X⁴ is CH. In some embodiments, X⁴ is N. In some embodiments, each R^{b} is independently —F or —Cl.

In some embodiments, the compound is a compound of Formula (I-A1) or a pharmaceutically acceptable salt thereof:

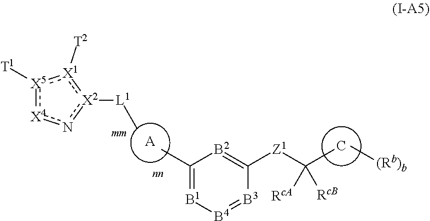

wherein R^{cA} and R^{cB} are independently selected from the group consisting of H and R^{c}.

In some embodiments of Formula (I-A5), the ring containing B¹, B², B³, and B⁴ is

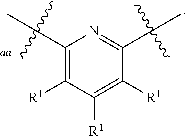

For example, the ring containing B¹, B², B³, and B⁴ can be

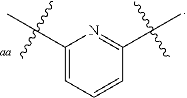 [SMILES: *c1cccc(*)n1]

In some embodiments, the compound is a compound of Formula (I-A2) or a pharmaceutically acceptable salt thereof:

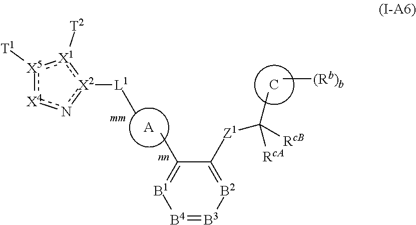

wherein R^{cA} and R^{cB} are independently selected from the group consisting of H and R^{c}.

In some embodiments of Formula (I-A6), the ring containing B¹, B², B³, and B⁴ is

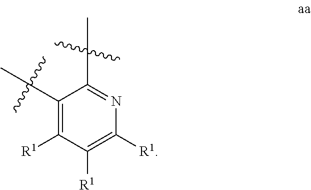

For example, the ring containing B¹, B², B³, and B⁴ can be

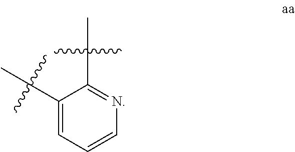 [SMILES: *c1cccnc1*]

In some embodiments of Formulae (I-A1) or (I-A2), Z¹ is —O—. In some embodiments of Formulae (I-A1) or (I-A2), R^{cA} is H. In some embodiments of Formulae (I-A1) or (I-A2), R^{cB} is H.

In some embodiments, the compound is a compound of Formula (I-A7):

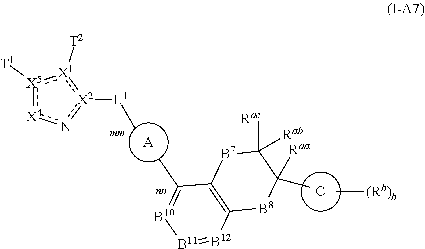

or a pharmaceutically acceptable salt thereof.

In some embodiments of Formula (I-A7), R^{ab} and R^{ac} are H.

In some embodiments, the compound is a compound of Formula (I-A8):

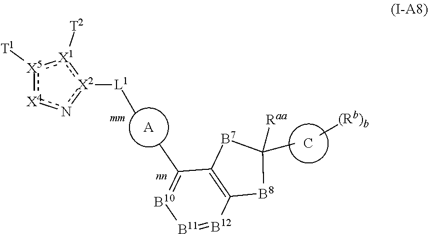

or a pharmaceutically acceptable salt thereof.

In some embodiments of Formula (I-A8), B⁷ is —O—; and B⁸ is —O—.

In some embodiments of Formulae (I-A7) or (I-A8), R^{aa} is H. In some embodiments, R^{aa} is (C₁-C₃)alkyl. For example, R^{aa} can be methyl.

In some embodiments of Formulae (I-A7) or (I-A8), B¹⁰, B¹¹, and B¹² are independently selected CR¹. In some embodiments, B¹⁰, B¹¹, and B¹² are CH.

In some embodiments of Formulae (I-A5), (I-A6), (I-A7), or (I-A8), X¹ is N.

In some embodiments of Formulae (I-A5), (I-A6), (I-A7), or (I-A8), X² is C.

In some embodiments of Formulae (I-A5), (I-A6), (I-A7), or (I-A8), X⁵ is C.

In some embodiments of Formulae (I-A5), (I-A6), (I-A7), or (I-A8), X⁴ is N. In some embodiments of Formulae (I-A5), (I-A6), (I-A7), or (I-A8), X⁴ is CR^{y}, such as CH.

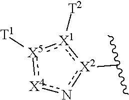

In some embodiments of Formulae (I-A5), (I-A6), (I-A7), or (I-A8), the moiety is

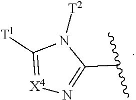

In some embodiments, X⁴ is N. In some embodiments, X⁴ is CR^{y}. For example, X⁴ can be CH.

In some embodiments, the compound is a compound of Formula (I-B2), or a pharmaceutically acceptable salt thereof:

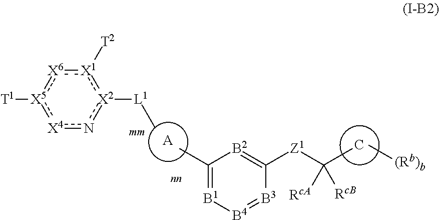

wherein X⁶ is selected from the group consisting of: N, NR^{x}, CR^{y}, and C(O); and
R^{cA} and R^{cB} are independently selected from the group consisting of H and R^{c}.

In some embodiments of Formula (I-B2), X¹ is C. In some embodiments of Formula (I-B2), X² and X⁵ are C. In some embodiments of Formula (I-B2), X⁴ and X⁶ are independently selected CR^{y}. For example, X⁴ and X⁶ can be CH.

In some embodiments of Formula (I-B2), the

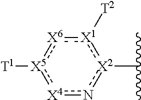

moiety is

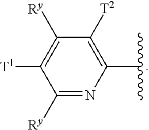

In some embodiments, each R^{y} is H.

In some embodiments of Formula (I-B2), L⁰ is #—P⁰-P¹.

In some embodiments of Formula (I-B2), Z¹ is —O—. In some embodiments of Formula (I-B2), R^{cA} is H; and R^{cB} is H.

In some embodiments of Formula (I-A5), (I-A6), (I-A7), (I-A8), or (I-B2), T¹ is C(O)OH.

In some embodiments of Formula (I-A5), (I-A6), (I-A7), (I-A8), or (I-B2), T¹ is a carboxylic acid bioisostere (e.g., tetrazolyl, optional substituted triazolyl).

In some embodiments of Formula (I-A5), (I-A6), (I-A7), (I-A8), or (I-B2), T² is H or (C₁-C₃)alkyl.

In some embodiments of Formula (I-A5), (I-A6), (I-A7), (I-A8), or (I-B2), T² is (C₁-C₃)alkyl which is substituted with (C₁-C₃)alkoxy. For example, T² can be

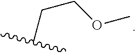 [SMILES: *CCOC]

In some embodiments of Formula (I-A5), (I-A6), (I-A7), (I-A8), or (I-B2), T² is (C₁-C₃)alkyl which is substituted with S(O)₂(C₁-C₃ alkyl). For example, T² can be

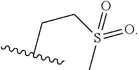 [SMILES: *CCS(C)(=O)=O]

In some embodiments of Formula (I-A5), (I-A6), (I-A7), (I-A8), or (I-B2), T² is (C₁-C₃)alkyl which is substituted with (C₃-C₅)cycloalkyl. For example, T² can be

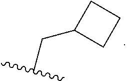 [SMILES: *CC1CCC1]

In some embodiments of Formula (I-A5), (I-A6), (I-A7), (I-A8), or (I-B2), T² is (C₁-C₃)alkyl which is substituted with 3- to 5-membered heterocycloalkyl. In some embodiments, T² is (C₁-C₃)alkyl which is substituted with oxetanyl. For example, T² can be

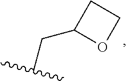 [SMILES: *CC1CCO1]

optionally wherein the stereogenic center in

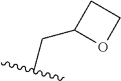 [SMILES: *CC1CCO1]

has (S)-configuration. In some embodiments, T² is (C₁-C₃)alkyl which is substituted with tetrahydrofuranyl. In some embodiments, T² is or

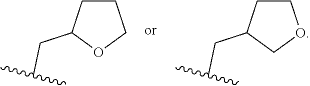 [SMILES: *CC1CCCO1 ; *CC1CCOC1]

In some embodiments of Formula (I-A5), (I-A6), (I-A7), (I-A8), or (I-B2), T² is (C₁-C₃)alkyl which is substituted with 5-membered heteroaryl, wherein the 5-membered heteroaryl is optionally substituted with 1-2 R^{T}. In some embodiments, T² is (C₁-C₃)alkyl which is substituted with imidazolyl, wherein the imidazolyl is optionally substituted with R^{T}. For example, T² can be

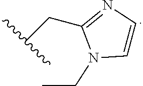 [SMILES: *Cc1nccn1CC]

As another non-limiting example, T² can be

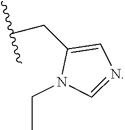 [SMILES: *Cc1cncn1CC]

In some embodiments of Formula (I-A5), (I-A6), (I-A7), (I-A8), or (I-B2), L¹ is CH₂; and Ring A is

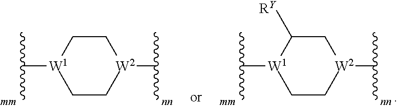

In some embodiments of Formula (I-A5), (I-A6), (I-A7), (I-A8), or (I-B2), L¹ is CH₂; and Ring A is

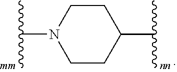 [SMILES: *C1CCN(*)CC1]

In some embodiments of Formula (I-A5), (I-A6), (I-A7), (I-A8), or (I-B2), L¹ is CH₂; and Ring A is

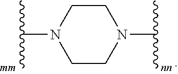 [SMILES: *N1CCN(*)CC1]

In some embodiments of Formula (I-A5), (I-A6), (I-A7), (I-A8), or (I-B2), L¹ is a bond; and Ring A is

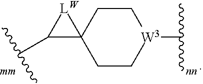

In some embodiments, Ring A is

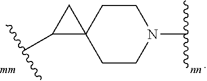 [SMILES: *C1CC12CCN(*)CC2]

In some embodiments of Formula (I-A5), (I-A6), (I-A7), (I-A8), or (I-B2), Ring C is selected from the group consisting of: phenyl and 6-membered heteroaryl (e.g., pyridyl such as 2-pyridyl, 3-pyridyl, or 4-pyridyl).

In some embodiments of Formula (I-A5), (I-A6), (I-A7), (I-A8), or (I-B2),

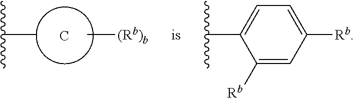

In some embodiments of Formula (I-A5), (I-A6), (I-A7), (I-A8), or (I-B2),

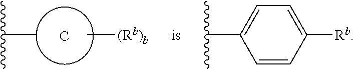

In some embodiments of Formula (I-A5), (I-A6), (I-A7), (I-A8), or (I-B2), each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

In some embodiments of Formula (I-A5), (I-A6), (I-A7), (I-A8), or (I-B2), R^{b} is independently selected from the group consisting of F, —Cl, CF₃, and CN.

In some embodiments, provided is a compound selected from Table 1, or a pharmaceutically acceptable salt or solvate thereof.

TABLE 1

| No. | Structure |
|---|---|
| A-1 | 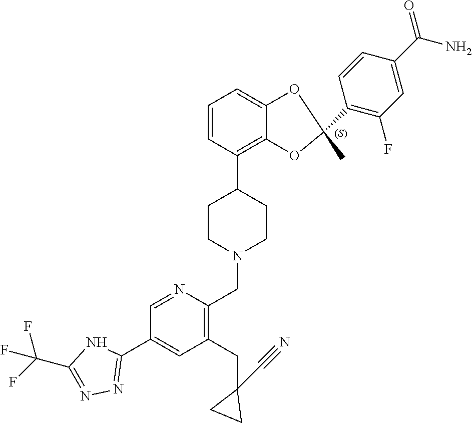 |
| A-3 | 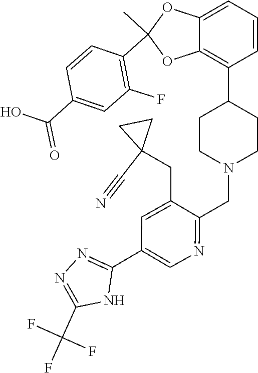 [SMILES: CC1(c2ccc(C(=O)O)cc2F)Oc2cccc(C3CCN(Cc4ncc(-c5nnc(C(F)(F)F)[nH]5)cc4CC4(C#N)CC4)CC3)c2O1] |
| B-1 | 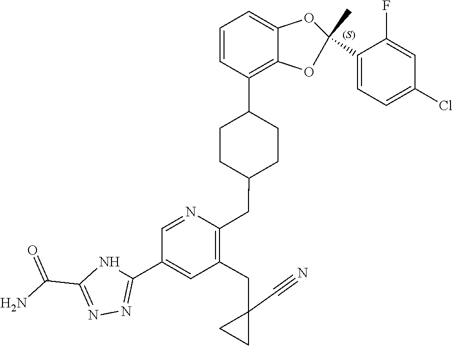 |
| C-1 | 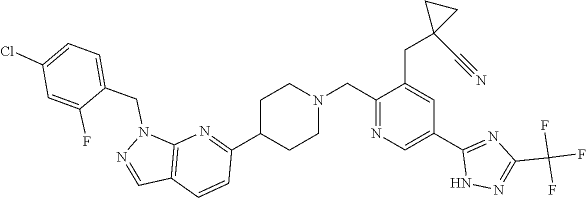 [SMILES: N#CC1(Cc2cc(-c3nc(C(F)(F)F)n[nH]3)cnc2CN2CCC(c3ccc4cnn(Cc5ccc(Cl)cc5F)c4n3)CC2)CC1] |
| D-1 | 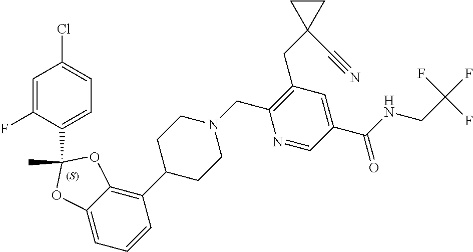 |
| D-2 | 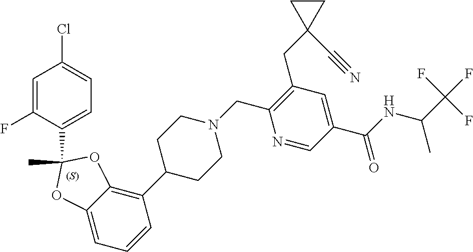 |
| D-3 | 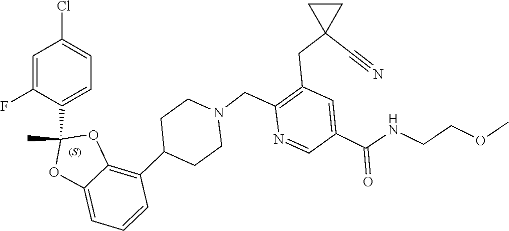 |
| D-4 | 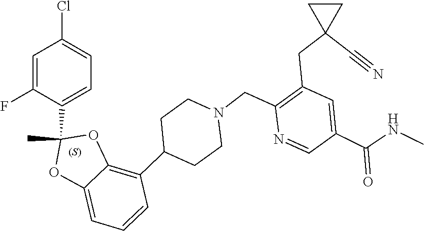 |
| D-5 | 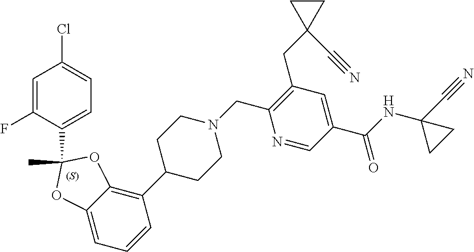 |
| D-6 | 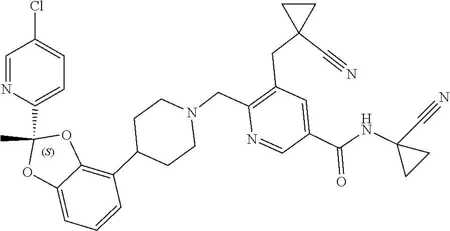 |
| D-7 | 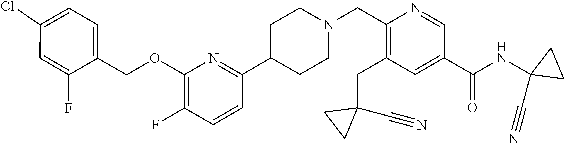 [SMILES: N#CC1(Cc2cc(C(=O)NC3(C#N)CC3)cnc2CN2CCC(c3ccc(F)c(OCc4ccc(Cl)cc4F)n3)CC2)CC1] |
| E-2 | 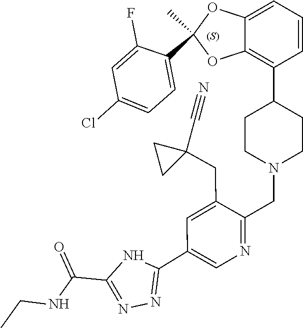 |
| E-3 | 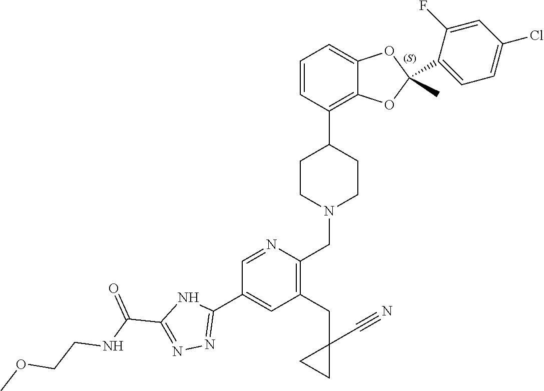 |
| E-4 | 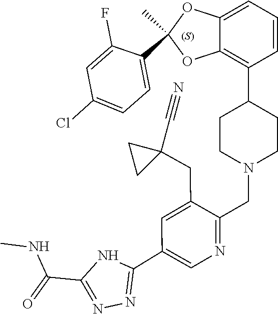 |
| E-5 | 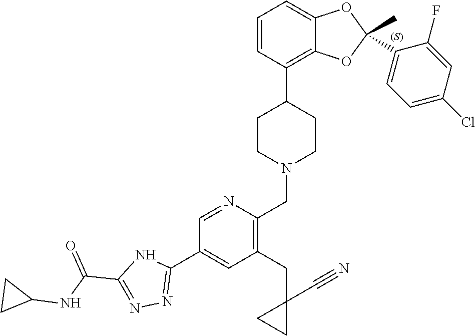 |
| E-6 | 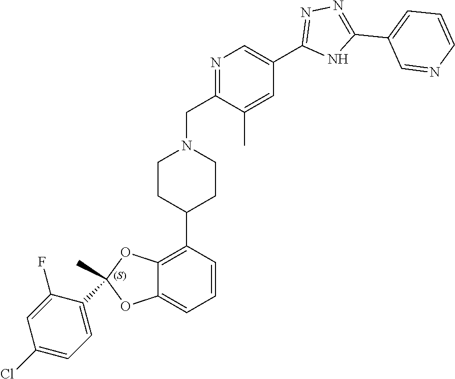 |
| E-7 | 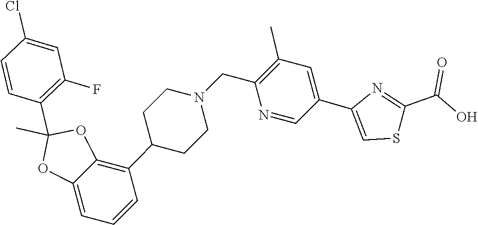 [SMILES: Cc1cc(-c2csc(C(=O)O)n2)cnc1CN1CCC(c2cccc3c2OC(C)(c2ccc(Cl)cc2F)O3)CC1] |
| E-8 | 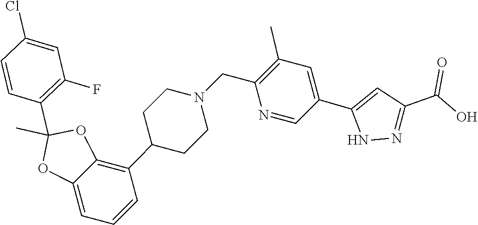 [SMILES: Cc1cc(-c2cc(C(=O)O)n[nH]2)cnc1CN1CCC(c2cccc3c2OC(C)(c2ccc(Cl)cc2F)O3)CC1] |
| E-9 | 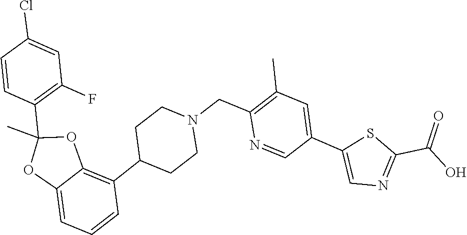 [SMILES: Cc1cc(-c2cnc(C(=O)O)s2)cnc1CN1CCC(c2cccc3c2OC(C)(c2ccc(Cl)cc2F)O3)CC1] |
| E-10 | 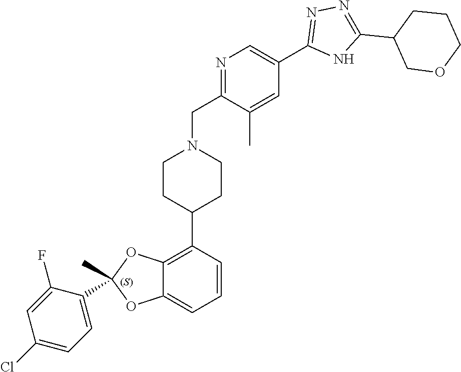 |
| E-11 | 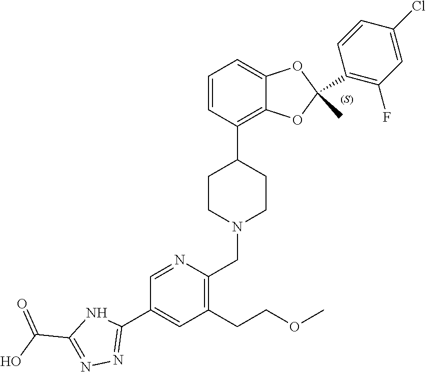 |
| E-12 | 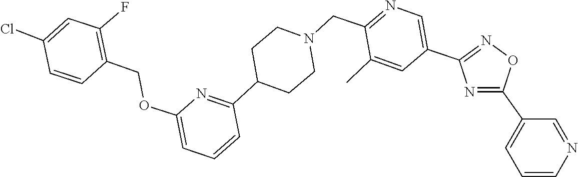 [SMILES: Cc1cc(-c2noc(-c3cccnc3)n2)cnc1CN1CCC(c2cccc(OCc3ccc(Cl)cc3F)n2)CC1] |
| E-13 | 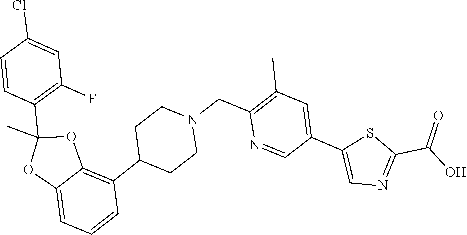 [SMILES: Cc1cc(-c2cnc(C(=O)O)s2)cnc1CN1CCC(c2cccc3c2OC(C)(c2ccc(Cl)cc2F)O3)CC1] |
| E-14 | 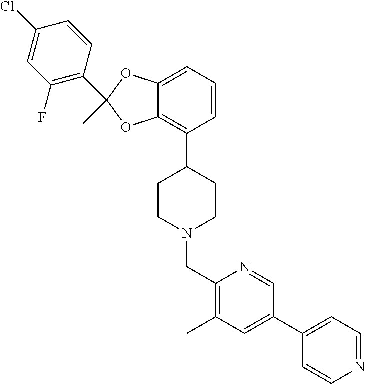 [SMILES: Cc1cc(-c2ccncc2)cnc1CN1CCC(c2cccc3c2OC(C)(c2ccc(Cl)cc2F)O3)CC1] |
| E-15 | 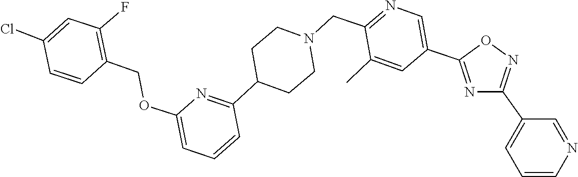 [SMILES: Cc1cc(-c2nc(-c3cccnc3)no2)cnc1CN1CCC(c2cccc(OCc3ccc(Cl)cc3F)n2)CC1] |
| E-16 | 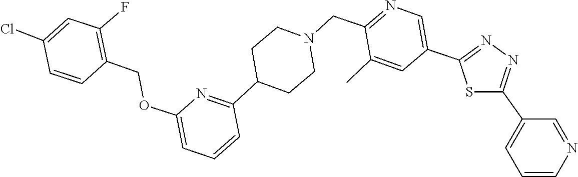 [SMILES: Cc1cc(-c2nnc(-c3cccnc3)s2)cnc1CN1CCC(c2cccc(OCc3ccc(Cl)cc3F)n2)CC1] |
| E-17 | 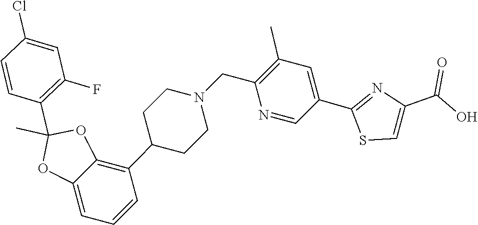 [SMILES: Cc1cc(-c2nc(C(=O)O)cs2)cnc1CN1CCC(c2cccc3c2OC(C)(c2ccc(Cl)cc2F)O3)CC1] |
| E-18 | 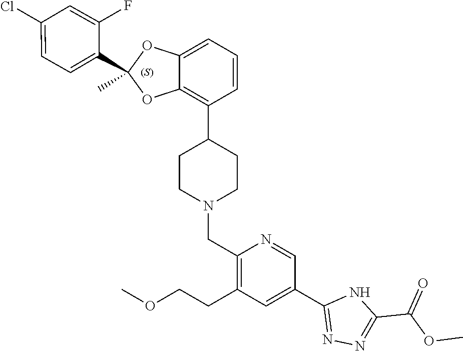 |
| E-19 | 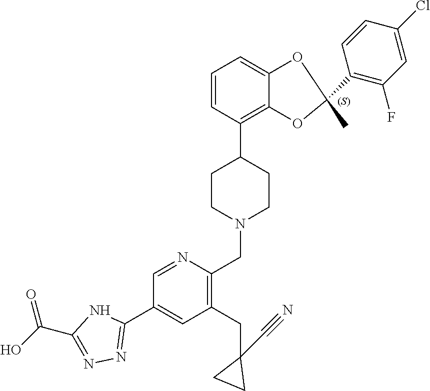 |
| F-1 | 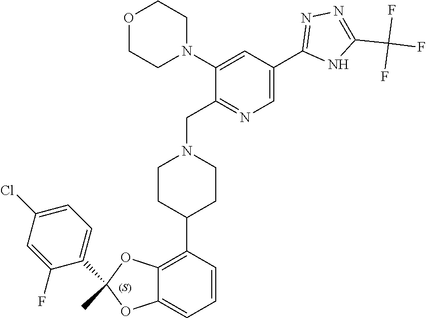 |
| F-2 | 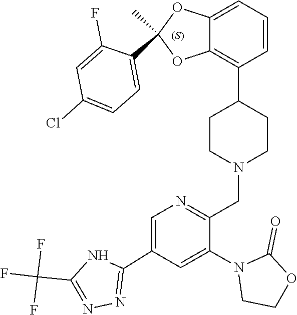 |
| F-3 | 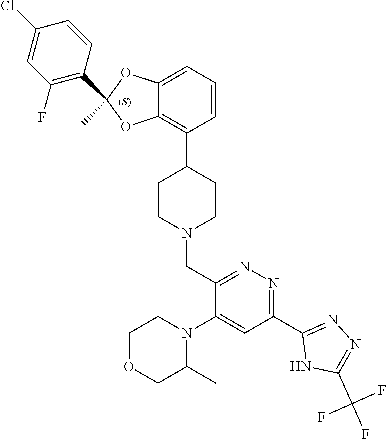 |
| F-4 | 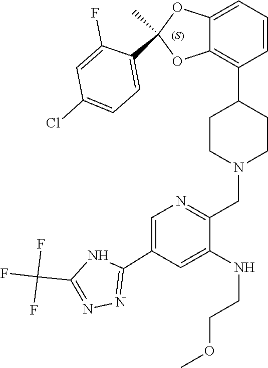 |
| F-5 | 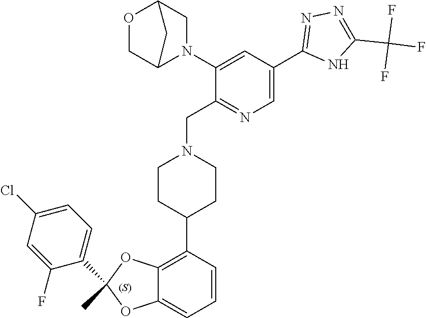 |
| F-6 | 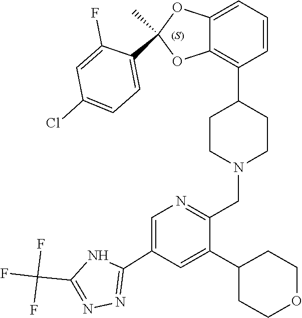 |
| F-7 | 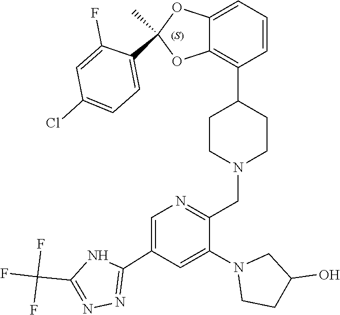 |
| F-8 | 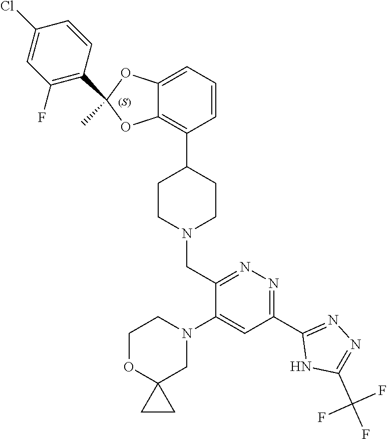 |
| F-9 | 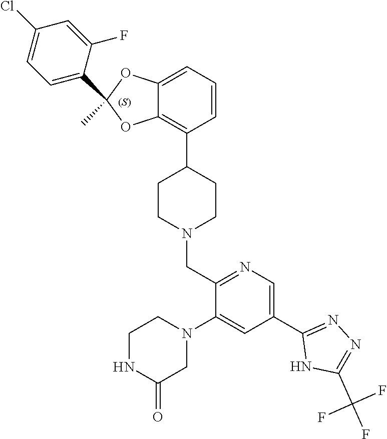 |
| F-10 | 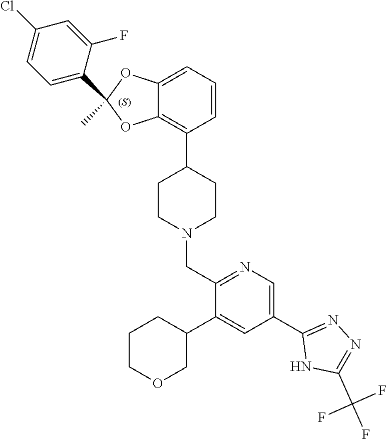 |
| F-11 | 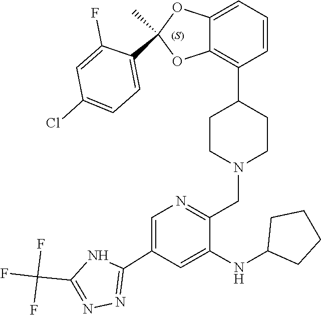 |
| F-12 | 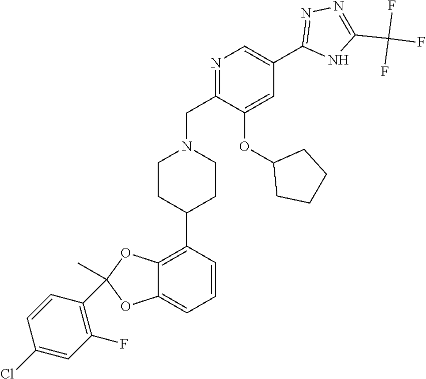 [SMILES: CC1(c2ccc(Cl)cc2F)Oc2cccc(C3CCN(Cc4ncc(-c5nnc(C(F)(F)F)[nH]5)cc4OC4CCCC4)CC3)c2O1] |
| F-13 | 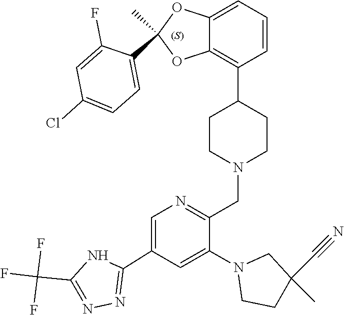 |
| F-14 | 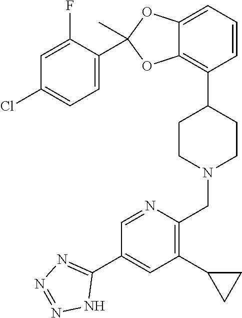 [SMILES: CC1(c2ccc(Cl)cc2F)Oc2cccc(C3CCN(Cc4ncc(-c5nnn[nH]5)cc4C4CC4)CC3)c2O1] |
| F-15 | 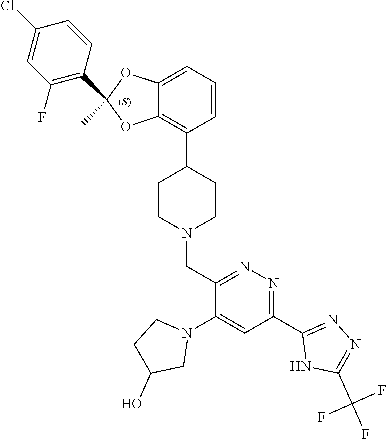 |
| F-16 | 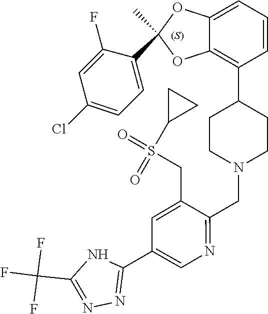 |
| F-17 | 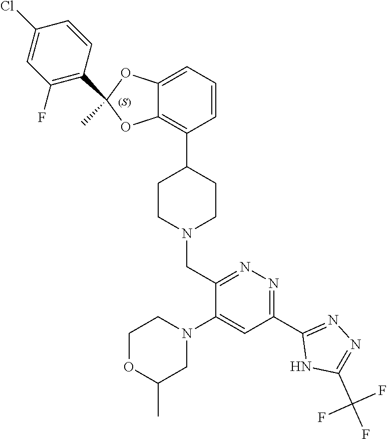 |
| F-18 | 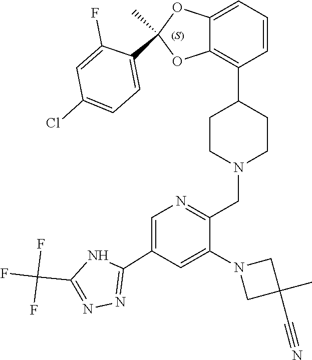 |
| F-19 | 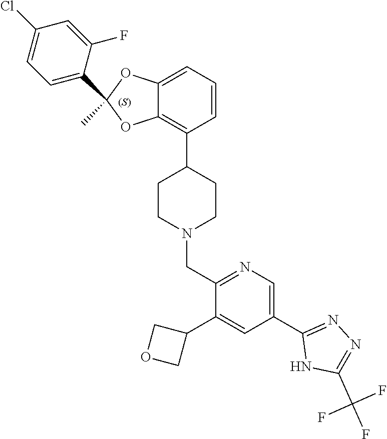 |
| F-20 | 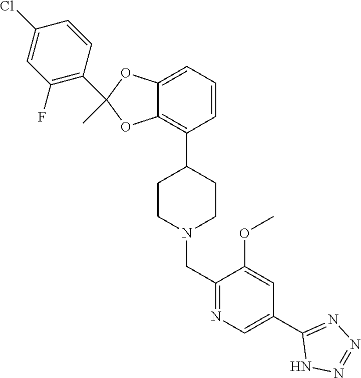 [SMILES: COc1cc(-c2nnn[nH]2)cnc1CN1CCC(c2cccc3c2OC(C)(c2ccc(Cl)cc2F)O3)CC1] |

In some embodiments, provided is a compound selected from Table 2, or a pharmaceutically acceptable salt or solvate thereof.

TABLE 2
Structure

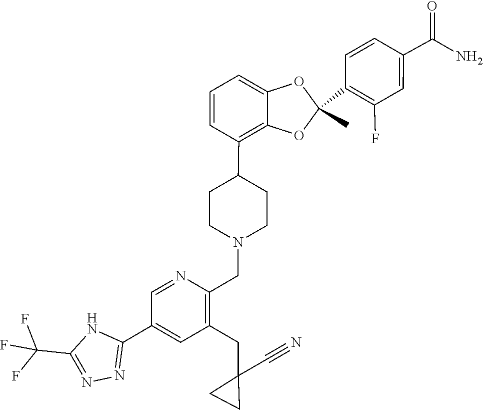 [SMILES: C[C@]1(c2ccc(C(N)=O)cc2F)Oc2cccc(C3CCN(Cc4ncc(-c5nnc(C(F)(F)F)[nH]5)cc4CC4(C#N)CC4)CC3)c2O1]

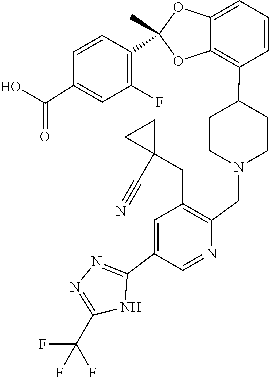 [SMILES: C[C@@]1(c2ccc(C(=O)O)cc2F)Oc2cccc(C3CCN(Cc4ncc(-c5nnc(C(F)(F)F)[nH]5)cc4CC4(C#N)CC4)CC3)c2O1]

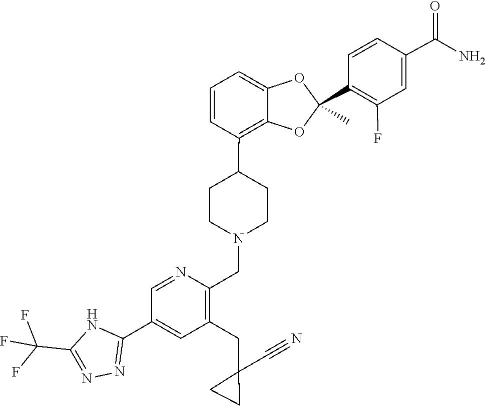 [SMILES: C[C@@]1(c2ccc(C(N)=O)cc2F)Oc2cccc(C3CCN(Cc4ncc(-c5nnc(C(F)(F)F)[nH]5)cc4CC4(C#N)CC4)CC3)c2O1]

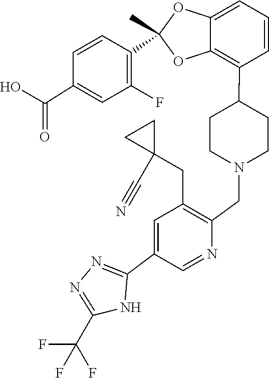 [SMILES: C[C@@]1(c2ccc(C(=O)O)cc2F)Oc2cccc(C3CCN(Cc4ncc(-c5nnc(C(F)(F)F)[nH]5)cc4CC4(C#N)CC4)CC3)c2O1]

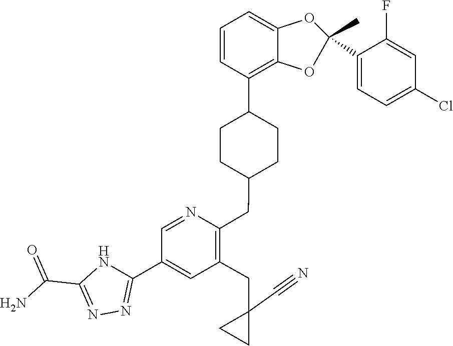 [SMILES: C[C@]1(c2ccc(Cl)cc2F)Oc2cccc(C3CCC(Cc4ncc(-c5nnc(C(N)=O)[nH]5)cc4CC4(C#N)CC4)CC3)c2O1]

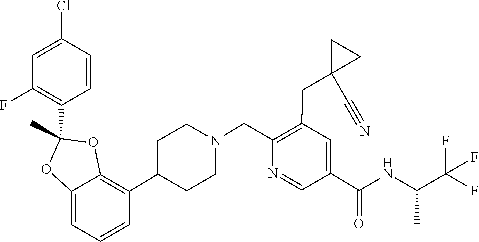 [SMILES: C[C@H](NC(=O)c1cnc(CN2CCC(c3cccc4c3O[C@@](C)(c3ccc(Cl)cc3F)O4)CC2)c(CC2(C#N)CC2)c1)C(F)(F)F]

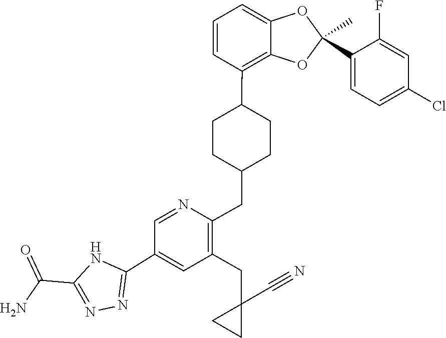 [SMILES: C[C@@]1(c2ccc(Cl)cc2F)Oc2cccc(C3CCC(Cc4ncc(-c5nnc(C(N)=O)[nH]5)cc4CC4(C#N)CC4)CC3)c2O1]

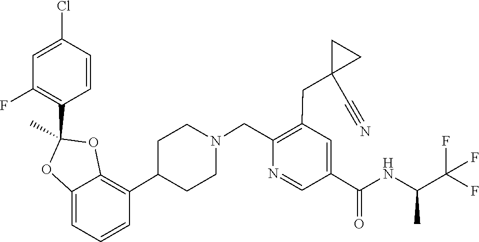 [SMILES: C[C@@H](NC(=O)c1cnc(CN2CCC(c3cccc4c3O[C@](C)(c3ccc(Cl)cc3F)O4)CC2)c(CC2(C#N)CC2)c1)C(F)(F)F]

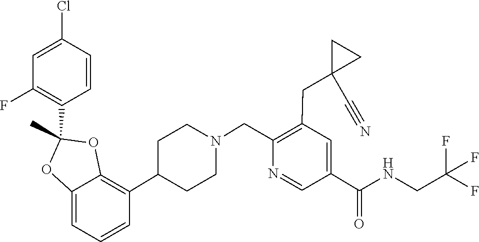 [SMILES: C[C@]1(c2ccc(Cl)cc2F)Oc2cccc(C3CCN(Cc4ncc(C(=O)NCC(F)(F)F)cc4CC4(C#N)CC4)CC3)c2O1]

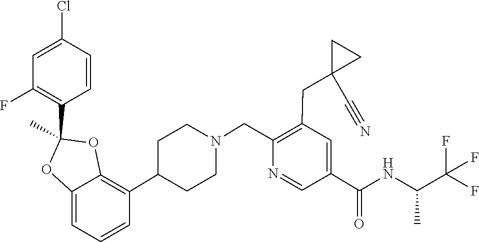 [SMILES: C[C@H](NC(=O)c1cnc(CN2CCC(c3cccc4c3O[C@](C)(c3ccc(Cl)cc3F)O4)CC2)c(CC2(C#N)CC2)c1)C(F)(F)F]

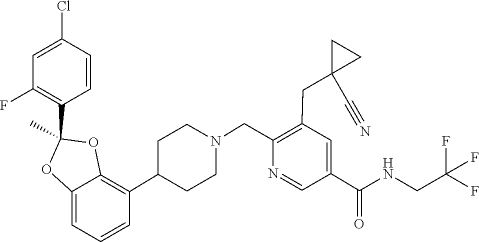 [SMILES: C[C@@]1(c2ccc(Cl)cc2F)Oc2cccc(C3CCN(Cc4ncc(C(=O)NCC(F)(F)F)cc4CC4(C#N)CC4)CC3)c2O1]

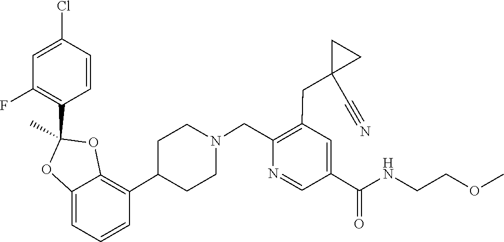 [SMILES: COCCNC(=O)c1cnc(CN2CCC(c3cccc4c3O[C@](C)(c3ccc(Cl)cc3F)O4)CC2)c(CC2(C#N)CC2)c1]

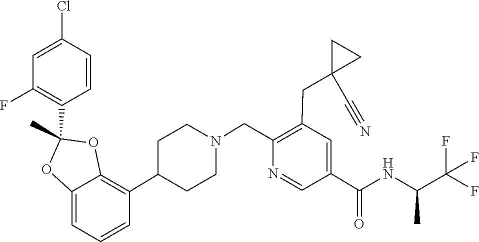 [SMILES: C[C@@H](NC(=O)c1cnc(CN2CCC(c3cccc4c3O[C@@](C)(c3ccc(Cl)cc3F)O4)CC2)c(CC2(C#N)CC2)c1)C(F)(F)F]

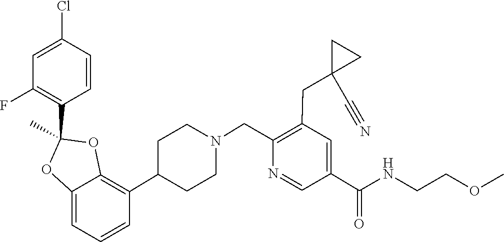 [SMILES: COCCNC(=O)c1cnc(CN2CCC(c3cccc4c3O[C@](C)(c3ccc(Cl)cc3F)O4)CC2)c(CC2(C#N)CC2)c1]

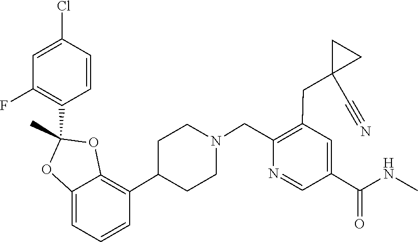 [SMILES: CNC(=O)c1cnc(CN2CCC(c3cccc4c3O[C@@](C)(c3ccc(Cl)cc3F)O4)CC2)c(CC2(C#N)CC2)c1]

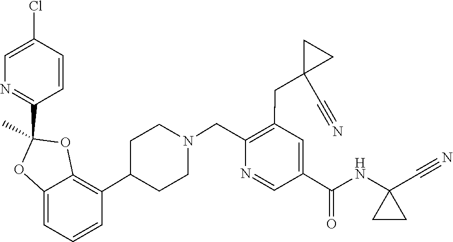 [SMILES: C[C@@]1(c2ccc(Cl)cn2)Oc2cccc(C3CCN(Cc4ncc(C(=O)NC5(C#N)CC5)cc4CC4(C#N)CC4)CC3)c2O1]

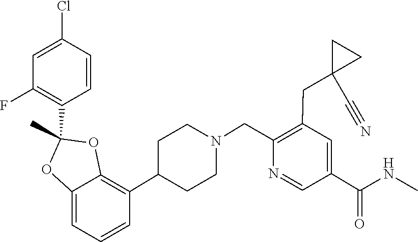 [SMILES: CNC(=O)c1cnc(CN2CCC(c3cccc4c3O[C@@](C)(c3ccc(Cl)cc3F)O4)CC2)c(CC2(C#N)CC2)c1]

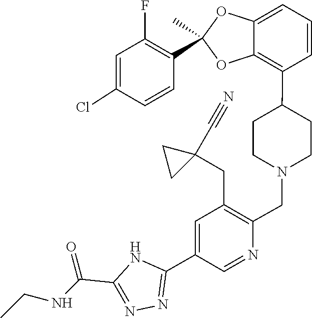 [SMILES: CCNC(=O)c1nnc(-c2cnc(CN3CCC(c4cccc5c4O[C@@](C)(c4ccc(Cl)cc4F)O5)CC3)c(CC3(C#N)CC3)c2)[nH]1]

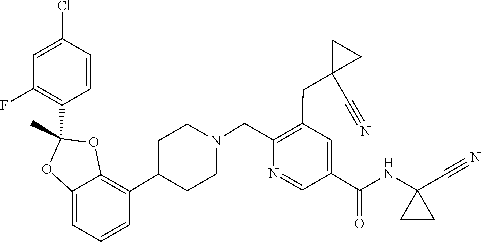 [SMILES: C[C@]1(c2ccc(Cl)cc2F)Oc2cccc(C3CCN(Cc4ncc(C(=O)NC5(C#N)CC5)cc4CC4(C#N)CC4)CC3)c2O1]

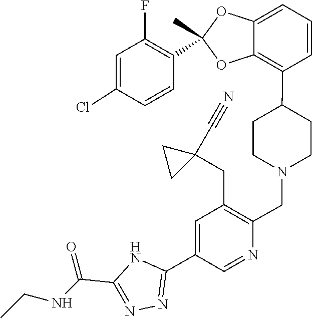 [SMILES: CCNC(=O)c1nnc(-c2cnc(CN3CCC(c4cccc5c4O[C@](C)(c4ccc(Cl)cc4F)O5)CC3)c(CC3(C#N)CC3)c2)[nH]1]

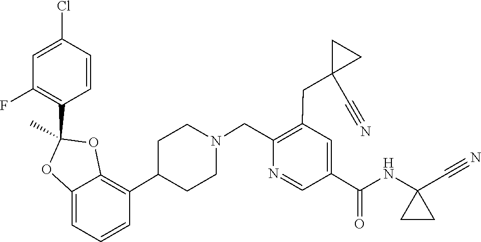 [SMILES: C[C@@]1(c2ccc(Cl)cc2F)Oc2cccc(C3CCN(Cc4ncc(C(=O)NC5(C#N)CC5)cc4CC4(C#N)CC4)CC3)c2O1]

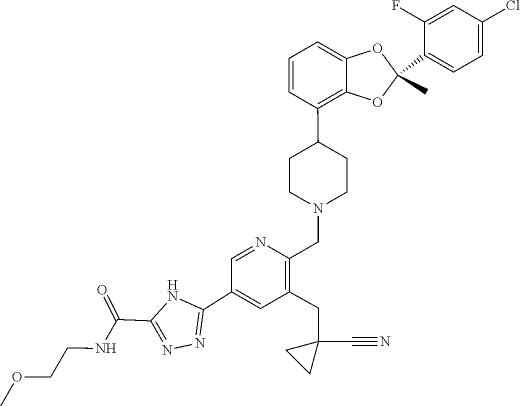 [SMILES: COCCNC(=O)c1nnc(-c2cnc(CN3CCC(c4cccc5c4O[C@@](C)(c4ccc(Cl)cc4F)O5)CC3)c(CC3(C#N)CC3)c2)[nH]1]

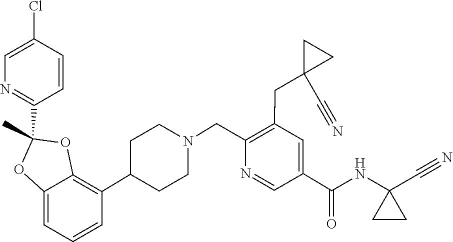 [SMILES: C[C@]1(c2ccc(Cl)cn2)Oc2cccc(C3CCN(Cc4ncc(C(=O)NC5(C#N)CC5)cc4CC4(C#N)CC4)CC3)c2O1]

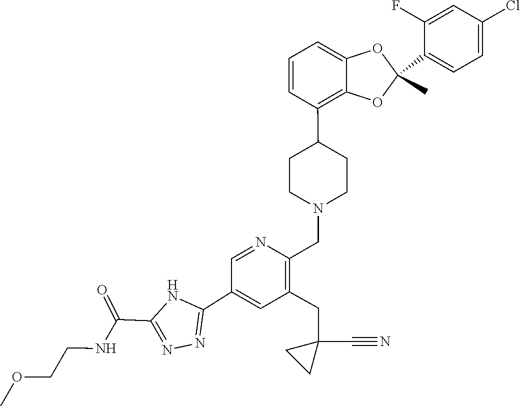 [SMILES: COCCNC(=O)c1nnc(-c2cnc(CN3CCC(c4cccc5c4O[C@@](C)(c4ccc(Cl)cc4F)O5)CC3)c(CC3(C#N)CC3)c2)[nH]1]

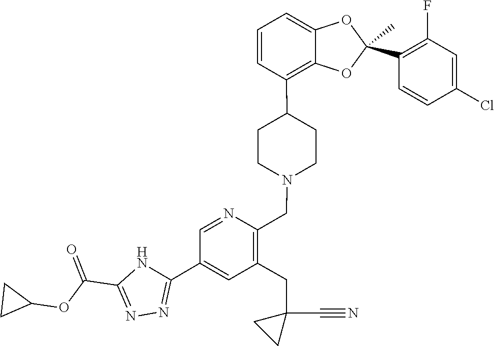 [SMILES: C[C@@]1(c2ccc(Cl)cc2F)Oc2cccc(C3CCN(Cc4ncc(-c5nnc(C(=O)OC6CC6)[nH]5)cc4CC4(C#N)CC4)CC3)c2O1]

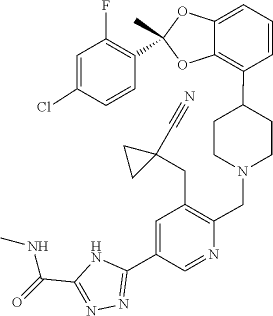 [SMILES: CNC(=O)c1nnc(-c2cnc(CN3CCC(c4cccc5c4O[C@](C)(c4ccc(Cl)cc4F)O5)CC3)c(CC3(C#N)CC3)c2)[nH]1]

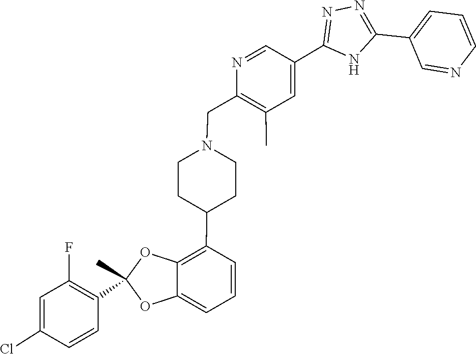 [SMILES: Cc1cc(-c2nnc(-c3cccnc3)[nH]2)cnc1CN1CCC(c2cccc3c2O[C@@](C)(c2ccc(Cl)cc2F)O3)CC1]

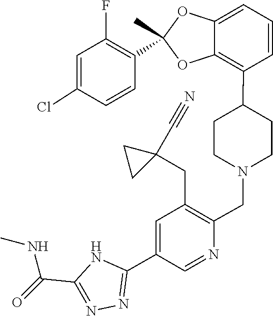 [SMILES: CNC(=O)c1nnc(-c2cnc(CN3CCC(c4cccc5c4O[C@](C)(c4ccc(Cl)cc4F)O5)CC3)c(CC3(C#N)CC3)c2)[nH]1]

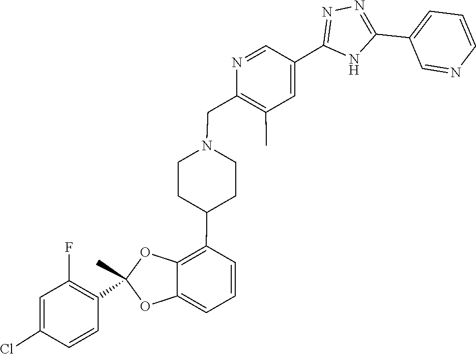 [SMILES: Cc1cc(-c2nnc(-c3cccnc3)[nH]2)cnc1CN1CCC(c2cccc3c2O[C@@](C)(c2ccc(Cl)cc2F)O3)CC1]

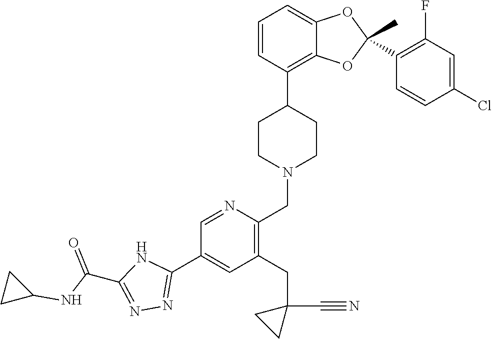 [SMILES: C[C@]1(c2ccc(Cl)cc2F)Oc2cccc(C3CCN(Cc4ncc(-c5nnc(C(=O)NC6CC6)[nH]5)cc4CC4(C#N)CC4)CC3)c2O1]

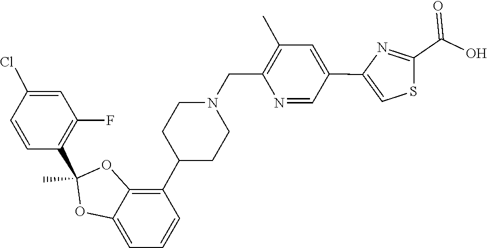 [SMILES: Cc1cc(-c2csc(C(=O)O)n2)cnc1CN1CCC(c2cccc3c2O[C@](C)(c2ccc(Cl)cc2F)O3)CC1]

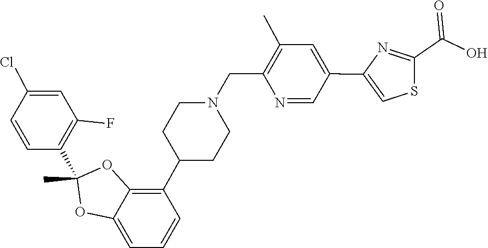 [SMILES: Cc1cc(-c2csc(C(=O)O)n2)cnc1CN1CCC(c2cccc3c2O[C@@](C)(c2ccc(Cl)cc2F)O3)CC1]

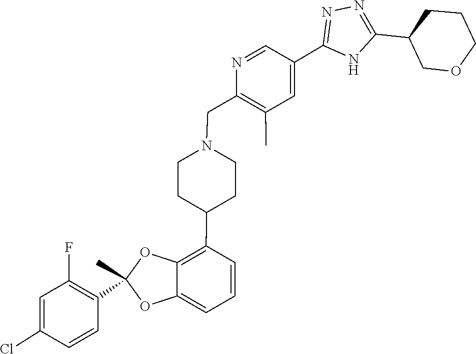 [SMILES: Cc1cc(-c2nnc([C@H]3CCCOC3)[nH]2)cnc1CN1CCC(c2cccc3c2O[C@@](C)(c2ccc(Cl)cc2F)O3)CC1]

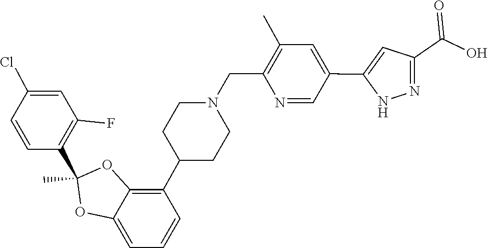 [SMILES: Cc1cc(-c2cc(C(=O)O)n[nH]2)cnc1CN1CCC(c2cccc3c2O[C@](C)(c2ccc(Cl)cc2F)O3)CC1]

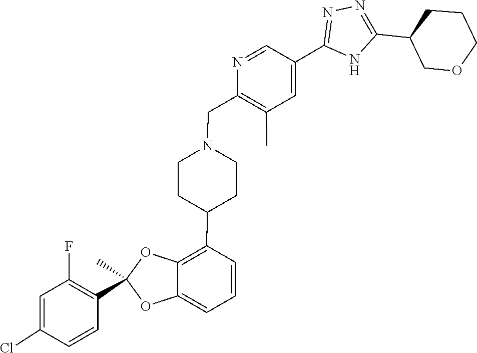 [SMILES: Cc1cc(-c2nnc([C@H]3CCCOC3)[nH]2)cnc1CN1CCC(c2cccc3c2O[C@](C)(c2ccc(Cl)cc2F)O3)CC1]

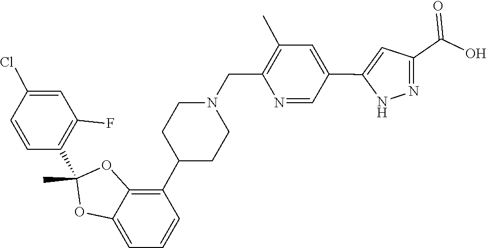 [SMILES: Cc1cc(-c2cc(C(=O)O)n[nH]2)cnc1CN1CCC(c2cccc3c2O[C@@](C)(c2ccc(Cl)cc2F)O3)CC1]

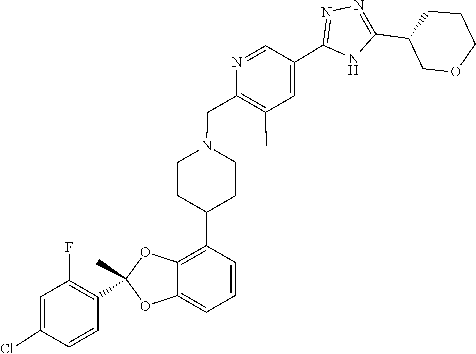 [SMILES: Cc1cc(-c2nnc([C@@H]3CCCOC3)[nH]2)cnc1CN1CCC(c2cccc3c2O[C@@](C)(c2ccc(Cl)cc2F)O3)CC1]

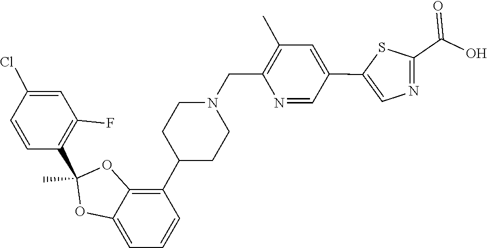 [SMILES: Cc1cc(-c2cnc(C(=O)O)s2)cnc1CN1CCC(c2cccc3c2O[C@](C)(c2ccc(Cl)cc2F)O3)CC1]

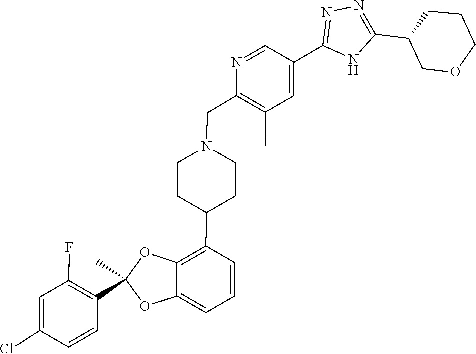 [SMILES: Cc1cc(-c2nnc([C@@H]3CCCOC3)[nH]2)cnc1CN1CCC(c2cccc3c2O[C@](C)(c2ccc(Cl)cc2F)O3)CC1]

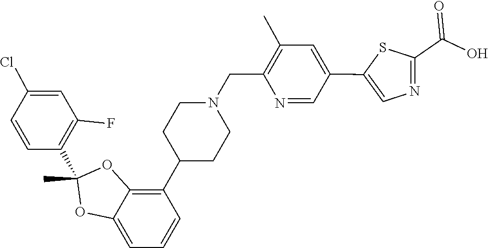 [SMILES: Cc1cc(-c2cnc(C(=O)O)s2)cnc1CN1CCC(c2cccc3c2O[C@@](C)(c2ccc(Cl)cc2F)O3)CC1]

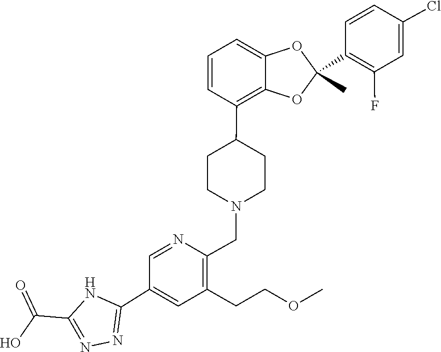 [SMILES: COCCc1cc(-c2nnc(C(=O)O)[nH]2)cnc1CN1CCC(c2cccc3c2O[C@@](C)(c2ccc(Cl)cc2F)O3)CC1]

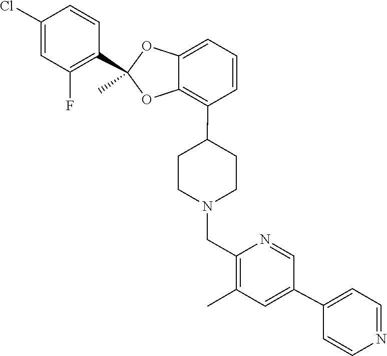 [SMILES: Cc1cc(-c2ccncc2)cnc1CN1CCC(c2cccc3c2O[C@@](C)(c2ccc(Cl)cc2F)O3)CC1]

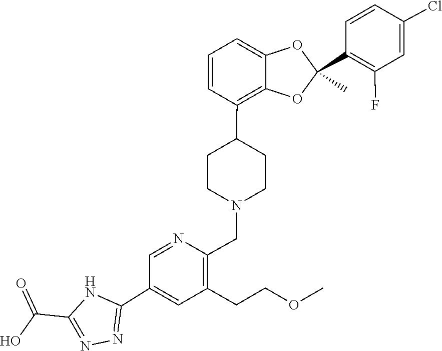 [SMILES: COCCc1cc(-c2nnc(C(=O)O)[nH]2)cnc1CN1CCC(c2cccc3c2O[C@](C)(c2ccc(Cl)cc2F)O3)CC1]

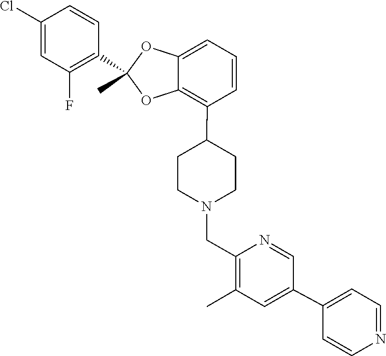 [SMILES: Cc1cc(-c2ccncc2)cnc1CN1CCC(c2cccc3c2O[C@](C)(c2ccc(Cl)cc2F)O3)CC1]

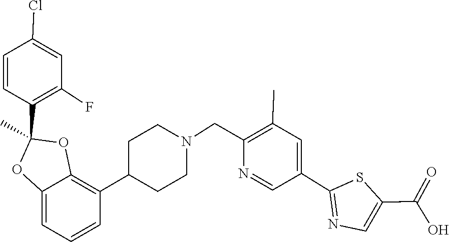 [SMILES: Cc1cc(-c2ncc(C(=O)O)s2)cnc1CN1CCC(c2cccc3c2O[C@](C)(c2ccc(Cl)cc2F)O3)CC1]

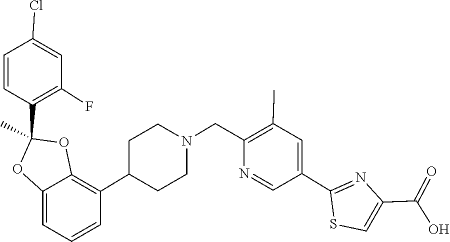 [SMILES: Cc1cc(-c2nc(C(=O)O)cs2)cnc1CN1CCC(c2cccc3c2O[C@](C)(c2ccc(Cl)cc2F)O3)CC1]

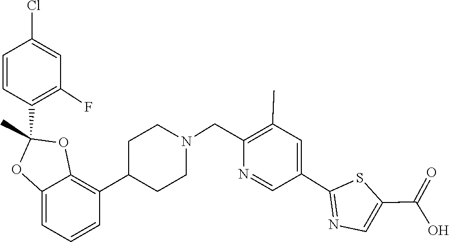 [SMILES: Cc1cc(-c2ncc(C(=O)O)s2)cnc1CN1CCC(c2cccc3c2O[C@@](C)(c2ccc(Cl)cc2F)O3)CC1]

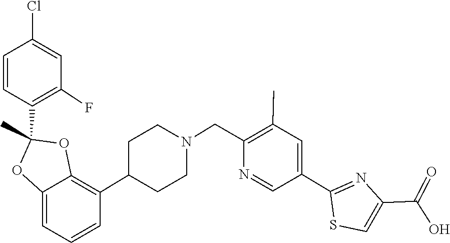 [SMILES: Cc1cc(-c2nc(C(=O)O)cs2)cnc1CN1CCC(c2cccc3c2O[C@@](C)(c2ccc(Cl)cc2F)O3)CC1]

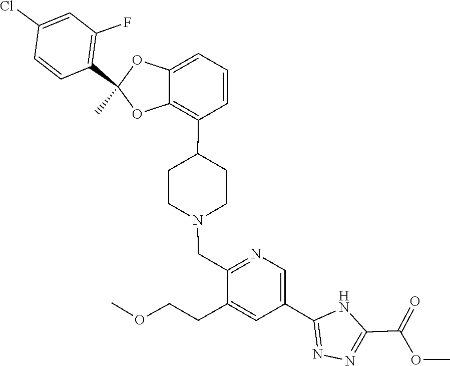 [SMILES: COCCc1cc(-c2nnc(C(=O)OC)[nH]2)cnc1CN1CCC(c2cccc3c2O[C@@](C)(c2ccc(Cl)cc2F)O3)CC1]

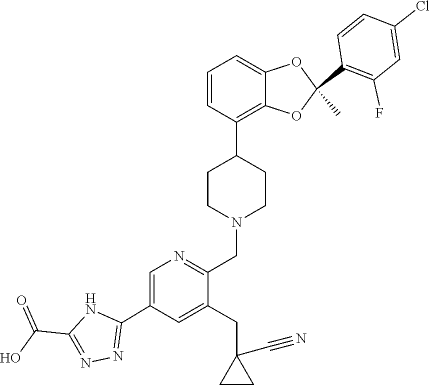 [SMILES: C[C@@]1(c2ccc(Cl)cc2F)Oc2cccc(C3CCN(Cc4ncc(-c5nnc(C(=O)O)[nH]5)cc4CC4(C#N)CC4)CC3)c2O1]

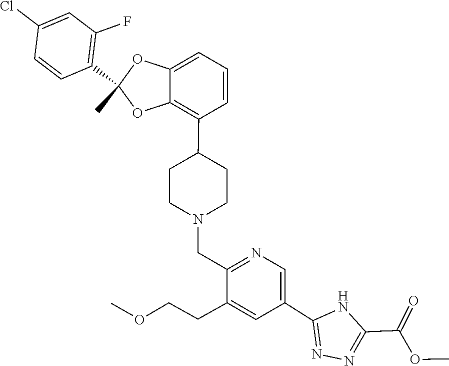 [SMILES: COCCc1cc(-c2nnc(C(=O)OC)[nH]2)cnc1CN1CCC(c2cccc3c2O[C@](C)(c2ccc(Cl)cc2F)O3)CC1]

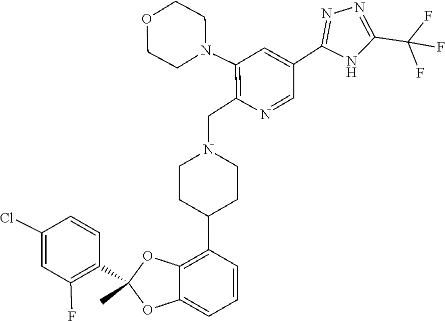 [SMILES: C[C@]1(c2ccc(Cl)cc2F)Oc2cccc(C3CCN(Cc4ncc(-c5nnc(C(F)(F)F)[nH]5)cc4N4CCOCC4)CC3)c2O1]

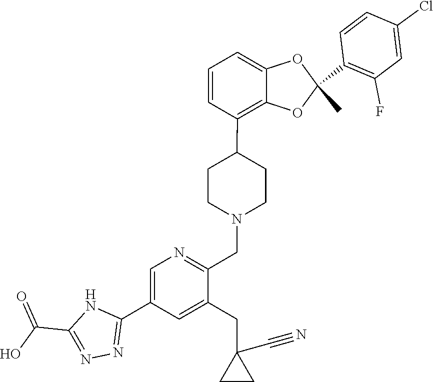 [SMILES: C[C@]1(c2ccc(Cl)cc2F)Oc2cccc(C3CCN(Cc4ncc(-c5nnc(C(=O)O)[nH]5)cc4CC4(C#N)CC4)CC3)c2O1]

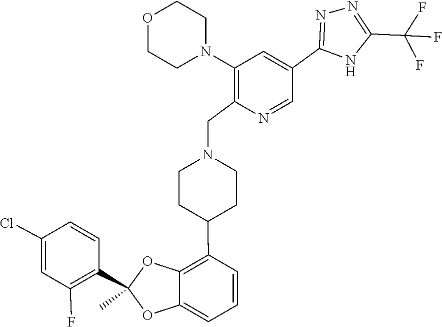 [SMILES: C[C@@]1(c2ccc(Cl)cc2F)Oc2cccc(C3CCN(Cc4ncc(-c5nnc(C(F)(F)F)[nH]5)cc4N4CCOCC4)CC3)c2O1]

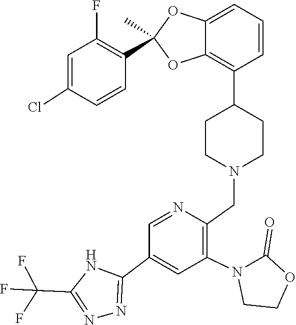 [SMILES: C[C@]1(c2ccc(Cl)cc2F)Oc2cccc(C3CCN(Cc4ncc(-c5nnc(C(F)(F)F)[nH]5)cc4N4CCOC4=O)CC3)c2O1]

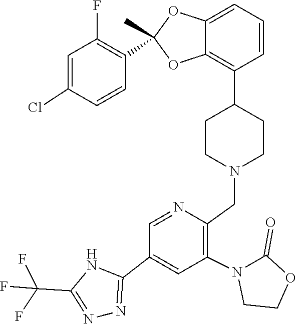 [SMILES: C[C@@]1(c2ccc(Cl)cc2F)Oc2cccc(C3CCN(Cc4ncc(-c5nnc(C(F)(F)F)[nH]5)cc4N4CCOC4=O)CC3)c2O1]

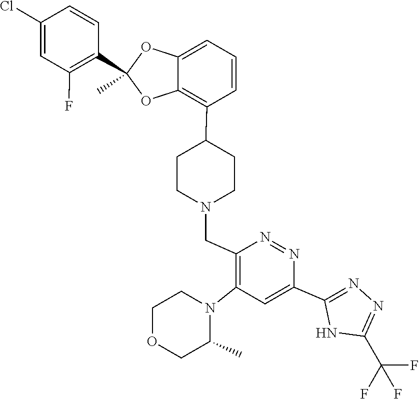 [SMILES: C[C@@H]1COCCN1c1cc(-c2nnc(C(F)(F)F)[nH]2)nnc1CN1CCC(c2cccc3c2O[C@@](C)(c2ccc(Cl)cc2F)O3)CC1]

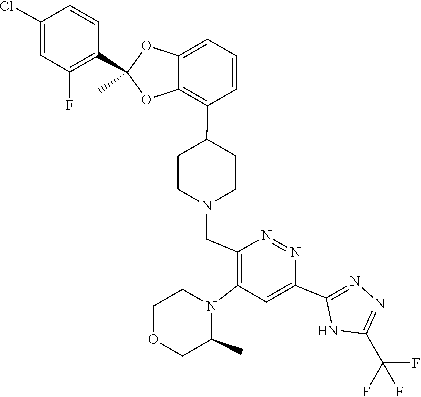 [SMILES: C[C@H]1COCCN1c1cc(-c2nnc(C(F)(F)F)[nH]2)nnc1CN1CCC(c2cccc3c2O[C@@](C)(c2ccc(Cl)cc2F)O3)CC1]

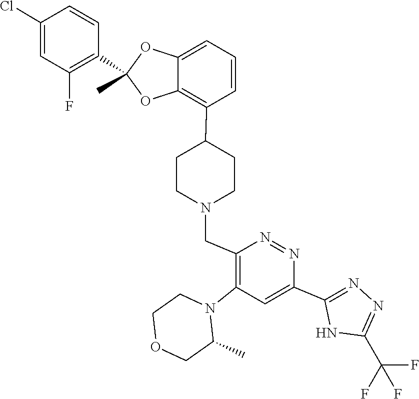 [SMILES: C[C@@H]1COCCN1c1cc(-c2nnc(C(F)(F)F)[nH]2)nnc1CN1CCC(c2cccc3c2O[C@](C)(c2ccc(Cl)cc2F)O3)CC1]

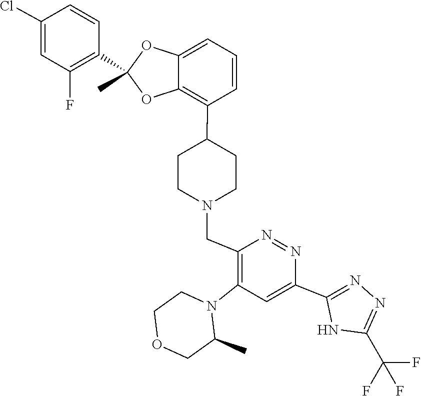 [SMILES: C[C@H]1COCCN1c1cc(-c2nnc(C(F)(F)F)[nH]2)nnc1CN1CCC(c2cccc3c2O[C@](C)(c2ccc(Cl)cc2F)O3)CC1]

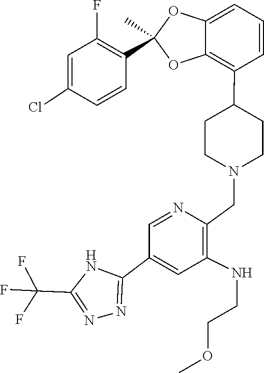 [SMILES: COCCNc1cc(-c2nnc(C(F)(F)F)[nH]2)cnc1CN1CCC(c2cccc3c2O[C@@](C)(c2ccc(Cl)cc2F)O3)CC1]

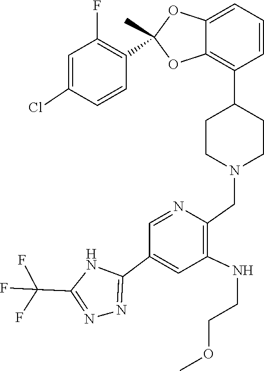 [SMILES: COCCNc1cc(-c2nnc(C(F)(F)F)[nH]2)cnc1CN1CCC(c2cccc3c2O[C@](C)(c2ccc(Cl)cc2F)O3)CC1]

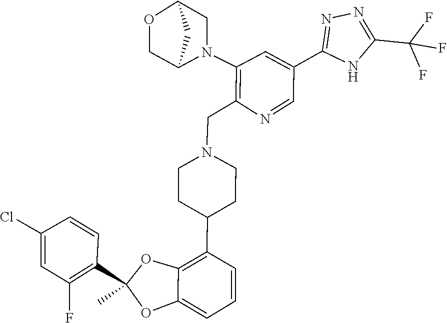 [SMILES: C[C@@]1(c2ccc(Cl)cc2F)Oc2cccc(C3CCN(Cc4ncc(-c5nnc(C(F)(F)F)[nH]5)cc4N4C[C@H]5C[C@@H]4CO5)CC3)c2O1]

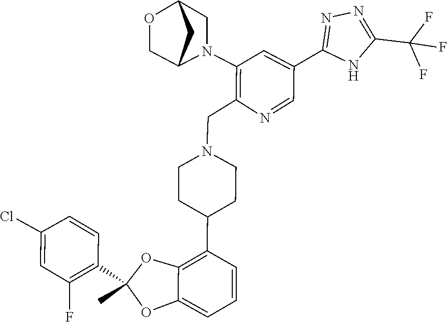 [SMILES: C[C@]1(c2ccc(Cl)cc2F)Oc2cccc(C3CCN(Cc4ncc(-c5nnc(C(F)(F)F)[nH]5)cc4N4C[C@@H]5C[C@H]4CO5)CC3)c2O1]

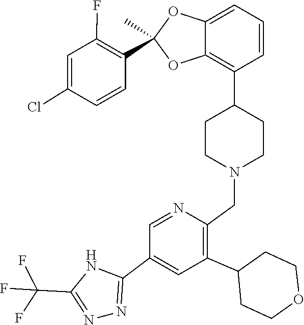 [SMILES: C[C@]1(c2ccc(Cl)cc2F)Oc2cccc(C3CCN(Cc4ncc(-c5nnc(C(F)(F)F)[nH]5)cc4C4CCOCC4)CC3)c2O1]

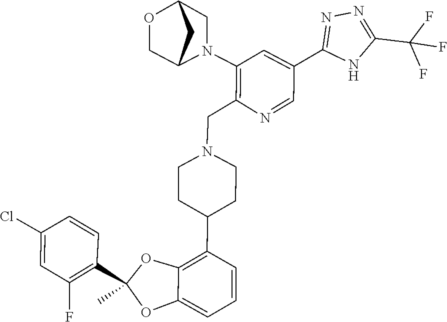 [SMILES: C[C@@]1(c2ccc(Cl)cc2F)Oc2cccc(C3CCN(Cc4ncc(-c5nnc(C(F)(F)F)[nH]5)cc4N4C[C@@H]5C[C@H]4CO5)CC3)c2O1]

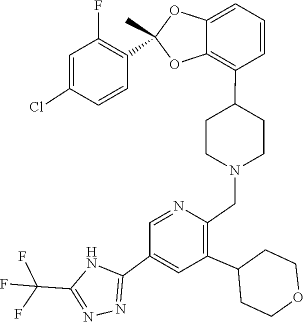 [SMILES: C[C@@]1(c2ccc(Cl)cc2F)Oc2cccc(C3CCN(Cc4ncc(-c5nnc(C(F)(F)F)[nH]5)cc4C4CCOCC4)CC3)c2O1]

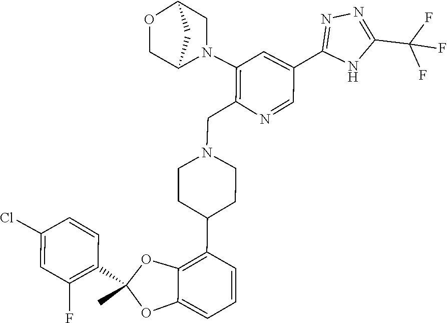 [SMILES: C[C@]1(c2ccc(Cl)cc2F)Oc2cccc(C3CCN(Cc4ncc(-c5nnc(C(F)(F)F)[nH]5)cc4N4C[C@H]5C[C@@H]4CO5)CC3)c2O1]

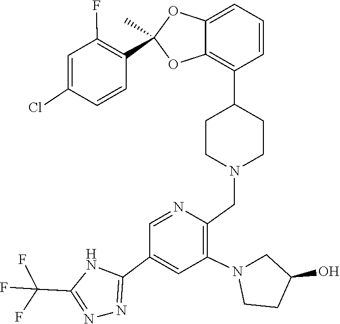 [SMILES: C[C@]1(c2ccc(Cl)cc2F)Oc2cccc(C3CCN(Cc4ncc(-c5nnc(C(F)(F)F)[nH]5)cc4N4CC[C@H](O)C4)CC3)c2O1]

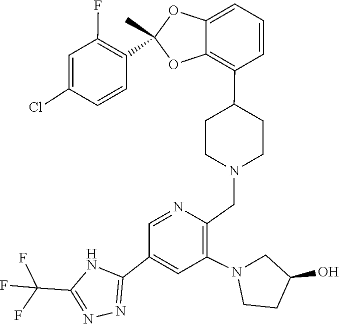 [SMILES: C[C@@]1(c2ccc(Cl)cc2F)Oc2cccc(C3CCN(Cc4ncc(-c5nnc(C(F)(F)F)[nH]5)cc4N4CC[C@H](O)C4)CC3)c2O1]

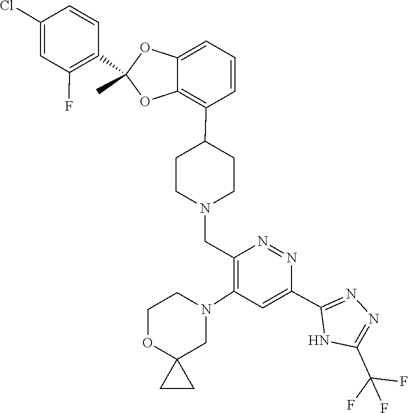 [SMILES: C[C@@]1(c2ccc(Cl)cc2F)Oc2cccc(C3CCN(Cc4nnc(-c5nnc(C(F)(F)F)[nH]5)cc4N4CCOC5(CC5)C4)CC3)c2O1]

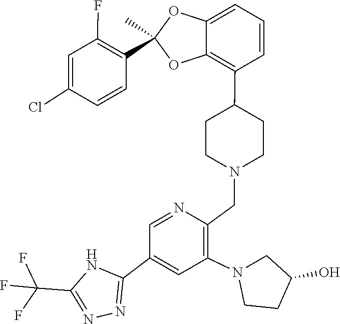 [SMILES: C[C@]1(c2ccc(Cl)cc2F)Oc2cccc(C3CCN(Cc4ncc(-c5nnc(C(F)(F)F)[nH]5)cc4N4CC[C@@H](O)C4)CC3)c2O1]

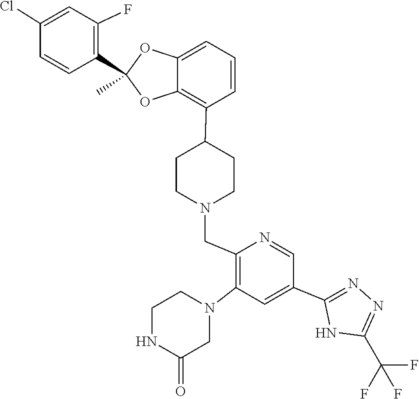 [SMILES: C[C@]1(c2ccc(Cl)cc2F)Oc2cccc(C3CCN(Cc4ncc(-c5nnc(C(F)(F)F)[nH]5)cc4N4CCNC(=O)C4)CC3)c2O1]

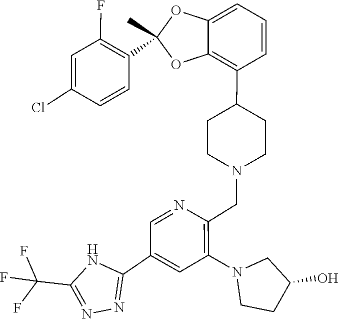 [SMILES: C[C@@]1(c2ccc(Cl)cc2F)Oc2cccc(C3CCN(Cc4ncc(-c5nnc(C(F)(F)F)[nH]5)cc4N4CC[C@@H](O)C4)CC3)c2O1]

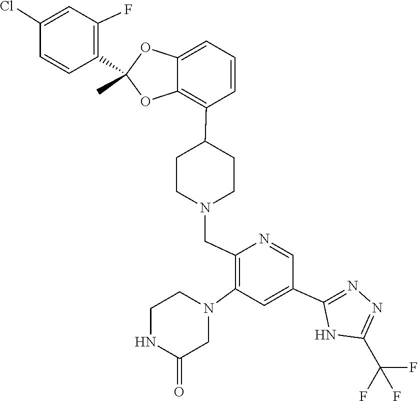 [SMILES: C[C@@]1(c2ccc(Cl)cc2F)Oc2cccc(C3CCN(Cc4ncc(-c5nnc(C(F)(F)F)[nH]5)cc4N4CCNC(=O)C4)CC3)c2O1]

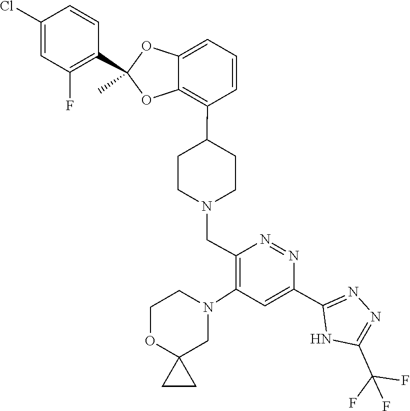 [SMILES: C[C@]1(c2ccc(Cl)cc2F)Oc2cccc(C3CCN(Cc4nnc(-c5nnc(C(F)(F)F)[nH]5)cc4N4CCOC5(CC5)C4)CC3)c2O1]

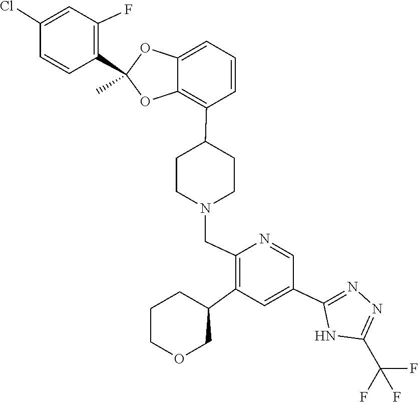 [SMILES: C[C@]1(c2ccc(Cl)cc2F)Oc2cccc(C3CCN(Cc4ncc(-c5nnc(C(F)(F)F)[nH]5)cc4[C@@H]4CCCOC4)CC3)c2O1]

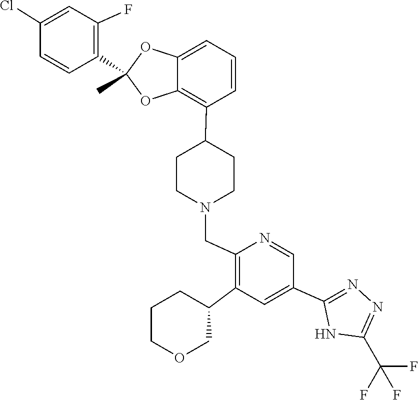 [SMILES: C[C@@]1(c2ccc(Cl)cc2F)Oc2cccc(C3CCN(Cc4ncc(-c5nnc(C(F)(F)F)[nH]5)cc4[C@H]4CCCOC4)CC3)c2O1]

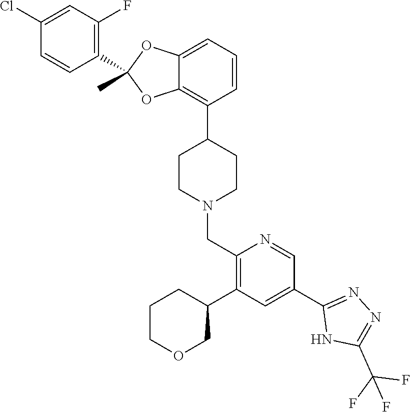 [SMILES: C[C@@]1(c2ccc(Cl)cc2F)Oc2cccc(C3CCN(Cc4ncc(-c5nnc(C(F)(F)F)[nH]5)cc4[C@@H]4CCCOC4)CC3)c2O1]

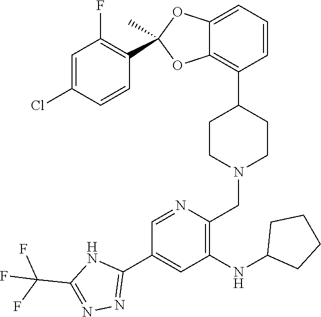 [SMILES: C[C@]1(c2ccc(Cl)cc2F)Oc2cccc(C3CCN(Cc4ncc(-c5nnc(C(F)(F)F)[nH]5)cc4NC4CCCC4)CC3)c2O1]

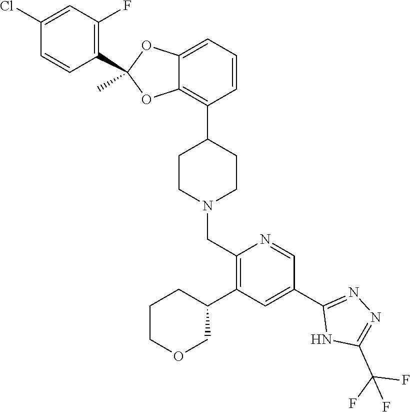 [SMILES: C[C@]1(c2ccc(Cl)cc2F)Oc2cccc(C3CCN(Cc4ncc(-c5nnc(C(F)(F)F)[nH]5)cc4[C@H]4CCCOC4)CC3)c2O1]

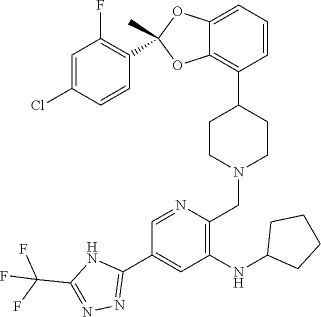 [SMILES: C[C@@]1(c2ccc(Cl)cc2F)Oc2cccc(C3CCN(Cc4ncc(-c5nnc(C(F)(F)F)[nH]5)cc4NC4CCCC4)CC3)c2O1]

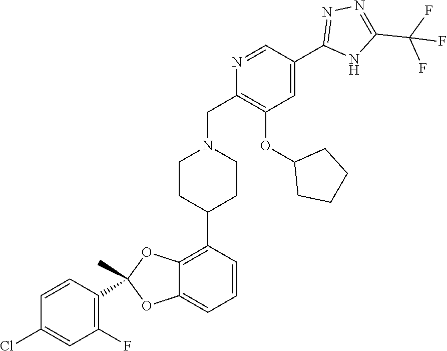 [SMILES: C[C@]1(c2ccc(Cl)cc2F)Oc2cccc(C3CCN(Cc4ncc(-c5nnc(C(F)(F)F)[nH]5)cc4OC4CCCC4)CC3)c2O1]

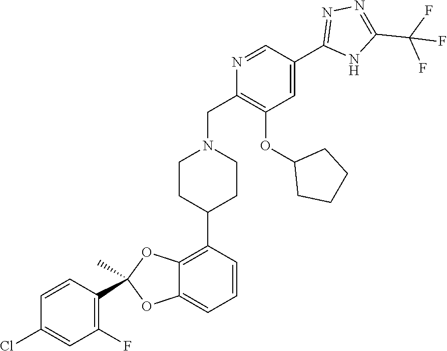 [SMILES: C[C@@]1(c2ccc(Cl)cc2F)Oc2cccc(C3CCN(Cc4ncc(-c5nnc(C(F)(F)F)[nH]5)cc4OC4CCCC4)CC3)c2O1]

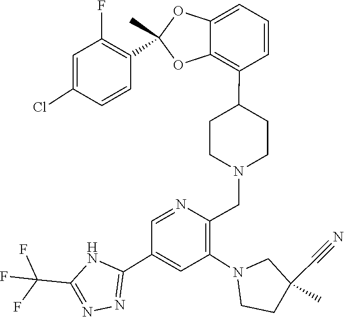 [SMILES: C[C@]1(C#N)CCN(c2cc(-c3nnc(C(F)(F)F)[nH]3)cnc2CN2CCC(c3cccc4c3O[C@](C)(c3ccc(Cl)cc3F)O4)CC2)C1]

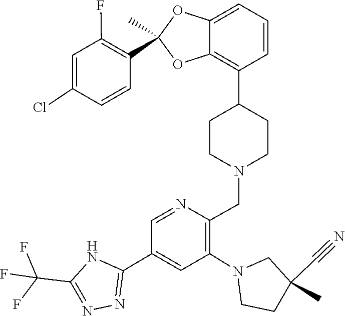 [SMILES: C[C@@]1(C#N)CCN(c2cc(-c3nnc(C(F)(F)F)[nH]3)cnc2CN2CCC(c3cccc4c3O[C@@](C)(c3ccc(Cl)cc3F)O4)CC2)C1]

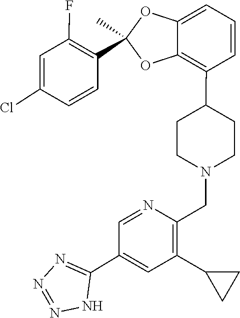 [SMILES: C[C@]1(c2ccc(Cl)cc2F)Oc2cccc(C3CCN(Cc4ncc(-c5nnn[nH]5)cc4C4CC4)CC3)c2O1]

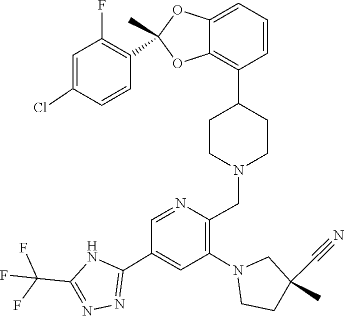 [SMILES: C[C@@]1(C#N)CCN(c2cc(-c3nnc(C(F)(F)F)[nH]3)cnc2CN2CCC(c3cccc4c3O[C@](C)(c3ccc(Cl)cc3F)O4)CC2)C1]

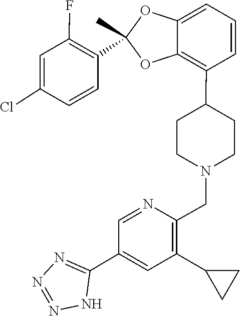 [SMILES: C[C@@]1(c2ccc(Cl)cc2F)Oc2cccc(C3CCN(Cc4ncc(-c5nnn[nH]5)cc4C4CC4)CC3)c2O1]

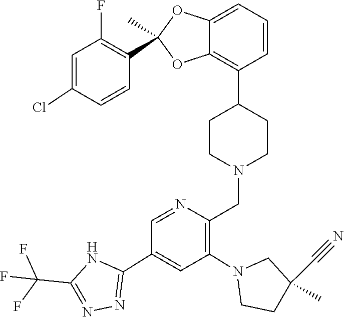 [SMILES: C[C@]1(C#N)CCN(c2cc(-c3nnc(C(F)(F)F)[nH]3)cnc2CN2CCC(c3cccc4c3O[C@@](C)(c3ccc(Cl)cc3F)O4)CC2)C1]

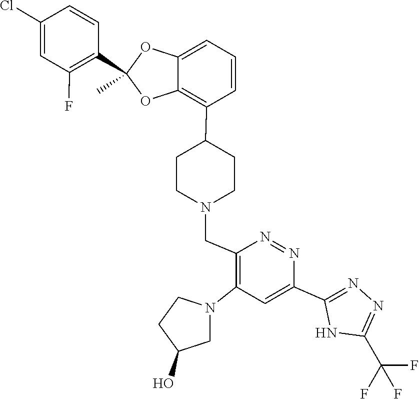 [SMILES: C[C@]1(c2ccc(Cl)cc2F)Oc2cccc(C3CCN(Cc4nnc(-c5nnc(C(F)(F)F)[nH]5)cc4N4CC[C@H](O)C4)CC3)c2O1]

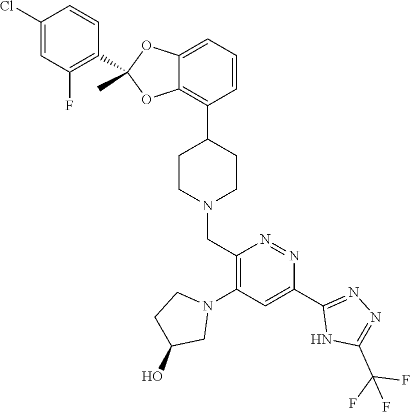 [SMILES: C[C@@]1(c2ccc(Cl)cc2F)Oc2cccc(C3CCN(Cc4nnc(-c5nnc(C(F)(F)F)[nH]5)cc4N4CC[C@H](O)C4)CC3)c2O1]

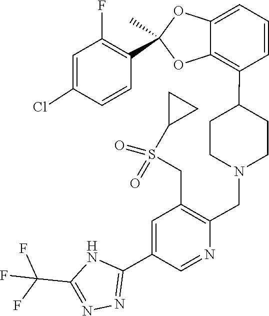 [SMILES: C[C@]1(c2ccc(Cl)cc2F)Oc2cccc(C3CCN(Cc4ncc(-c5nnc(C(F)(F)F)[nH]5)cc4CS(=O)(=O)C4CC4)CC3)c2O1]

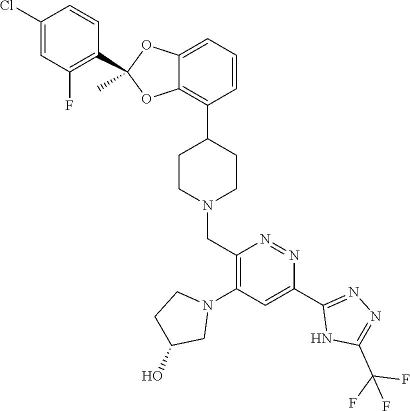 [SMILES: C[C@]1(c2ccc(Cl)cc2F)Oc2cccc(C3CCN(Cc4nnc(-c5nnc(C(F)(F)F)[nH]5)cc4N4CC[C@@H](O)C4)CC3)c2O1]

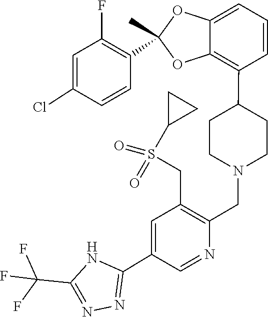 [SMILES: C[C@@]1(c2ccc(Cl)cc2F)Oc2cccc(C3CCN(Cc4ncc(-c5nnc(C(F)(F)F)[nH]5)cc4CS(=O)(=O)C4CC4)CC3)c2O1]

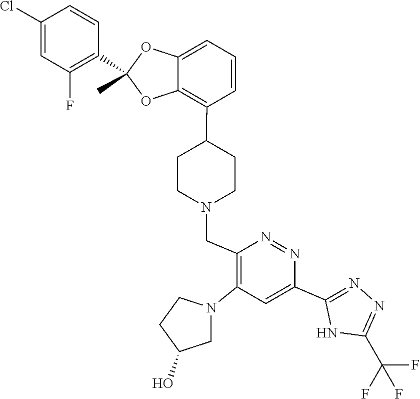 [SMILES: C[C@@]1(c2ccc(Cl)cc2F)Oc2cccc(C3CCN(Cc4nnc(-c5nnc(C(F)(F)F)[nH]5)cc4N4CC[C@@H](O)C4)CC3)c2O1]

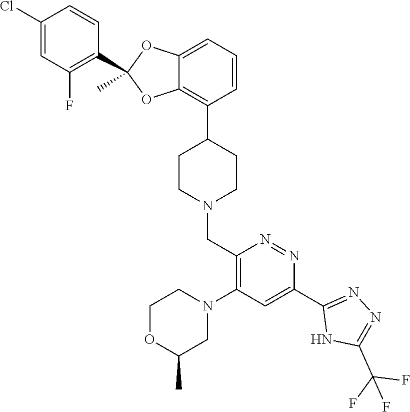 [SMILES: C[C@@H]1CN(c2cc(-c3nnc(C(F)(F)F)[nH]3)nnc2CN2CCC(c3cccc4c3O[C@@](C)(c3ccc(Cl)cc3F)O4)CC2)CCO1]

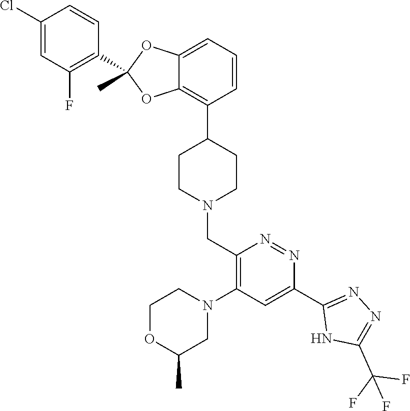 [SMILES: C[C@@H]1CN(c2cc(-c3nnc(C(F)(F)F)[nH]3)nnc2CN2CCC(c3cccc4c3O[C@](C)(c3ccc(Cl)cc3F)O4)CC2)CCO1]

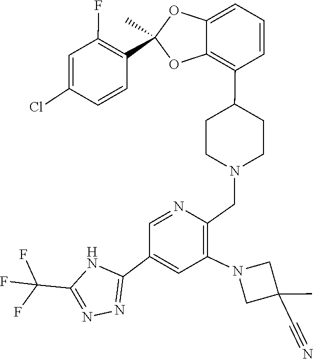 [SMILES: CC1(C#N)CN(c2cc(-c3nnc(C(F)(F)F)[nH]3)cnc2CN2CCC(c3cccc4c3O[C@@](C)(c3ccc(Cl)cc3F)O4)CC2)C1]

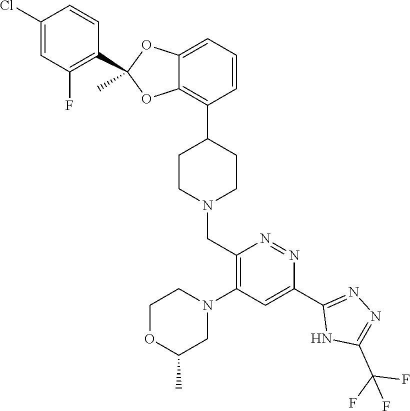 [SMILES: C[C@H]1CN(c2cc(-c3nnc(C(F)(F)F)[nH]3)nnc2CN2CCC(c3cccc4c3O[C@@](C)(c3ccc(Cl)cc3F)O4)CC2)CCO1]

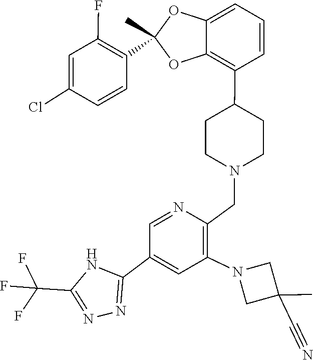 [SMILES: CC1(C#N)CN(c2cc(-c3nnc(C(F)(F)F)[nH]3)cnc2CN2CCC(c3cccc4c3O[C@](C)(c3ccc(Cl)cc3F)O4)CC2)C1]

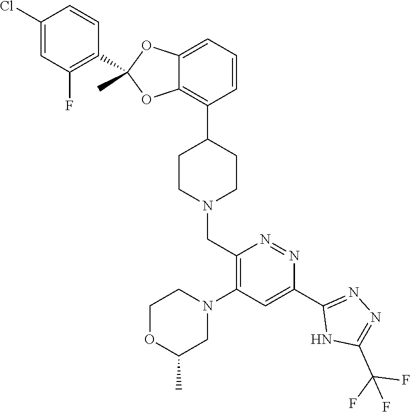 [SMILES: C[C@H]1CN(c2cc(-c3nnc(C(F)(F)F)[nH]3)nnc2CN2CCC(c3cccc4c3O[C@](C)(c3ccc(Cl)cc3F)O4)CC2)CCO1]

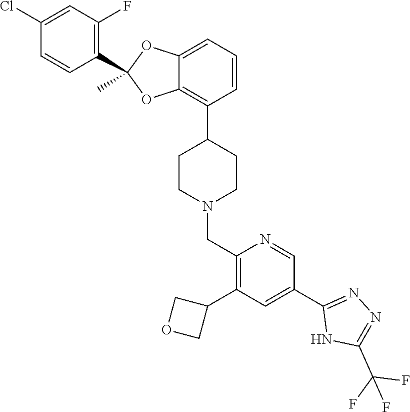 [SMILES: C[C@]1(c2ccc(Cl)cc2F)Oc2cccc(C3CCN(Cc4ncc(-c5nnc(C(F)(F)F)[nH]5)cc4C4COC4)CC3)c2O1]

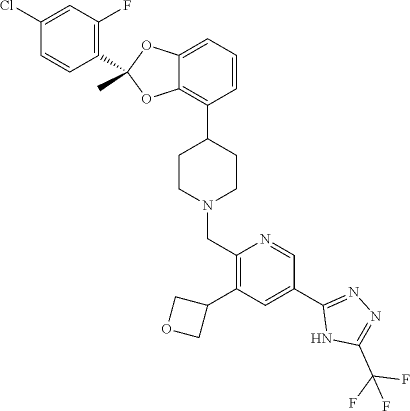 [SMILES: C[C@@]1(c2ccc(Cl)cc2F)Oc2cccc(C3CCN(Cc4ncc(-c5nnc(C(F)(F)F)[nH]5)cc4C4COC4)CC3)c2O1]

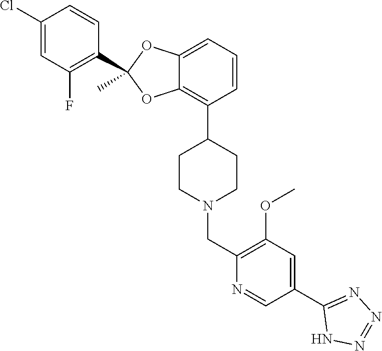 [SMILES: COc1cc(-c2nnn[nH]2)cnc1CN1CCC(c2cccc3c2O[C@@](C)(c2ccc(Cl)cc2F)O3)CC1]

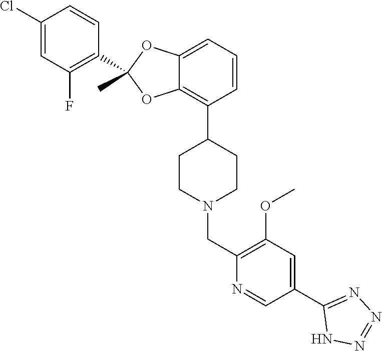 [SMILES: COc1cc(-c2nnn[nH]2)cnc1CN1CCC(c2cccc3c2O[C@](C)(c2ccc(Cl)cc2F)O3)CC1]

The compounds disclosed herein include pharmaceutically acceptable salts thereof. In addition, the compounds disclosed herein also include other salts of such compounds which are not necessarily pharmaceutically acceptable salts, and which may be useful as intermediates for preparing and/or purifying a compound disclosed herein and/or for separating enantiomers of compounds disclosed herein. Non-limiting examples of pharmaceutically acceptable salts of compounds disclosed herein include trifluoroacetic acid salts.

It will further be appreciated that the compounds disclosed herein or their salts may be isolated in the form of solvates, and accordingly that any such solvate is included within the scope of the present disclosure. A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. For example, compounds disclosed herein and salts thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., a compound of Formula I, or a pharmaceutically acceptable salt or solvate thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound disclosed herein, or a pharmaceutically acceptable salt or solvate thereof, in combination with one or more pharmaceutically acceptable excipients (carriers). For example, a pharmaceutical composition prepared using a compound of disclosed herein, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound disclosed herein, or a pharmaceutically acceptable salt or solvate thereof with a pharmaceutically acceptable excipient. Pharmaceutical compositions containing a compound disclosed herein, or a pharmaceutically acceptable salt or solvate thereof as the active ingredient can be prepared by intimately mixing the compound disclosed herein, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral, etc.). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, a compound disclosed herein, or a pharmaceutically acceptable salt or solvate thereof as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound disclosed herein, or a pharmaceutically acceptable salt or solvate thereof in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound disclosed herein, or a pharmaceutically acceptable salt or solvate thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound disclosed herein, or a pharmaceutically acceptable salt or solvate thereof, as described herein or a pharmaceutical composition thereof is formulated for local delivery to the digestive or GI tract by way of oral administration (e.g., solid or liquid dosage forms.).

In some embodiments, solid dosage forms for oral administration include capsules, tablets, pills, powders, and granules. In some embodiments, a compound disclosed herein, or a pharmaceutically acceptable salt or solvate thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound disclosed herein, or a pharmaceutically acceptable salt or solvate thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound disclosed herein, or a pharmaceutically acceptable salt or solvate thereof as described herein or a pharmaceutical composition thereof is formulated for ocular administration. In some embodiments, ocular compositions can include, without limitation, one or more of any of the following: viscogens (e.g., carboxymethylcellulose, glycerin, polyvinylpyrrolidone, polyethylene glycol); stabilizers (e.g., pluronic (triblock copolymers), cyclodextrins); preservatives (e.g., benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, a compound disclosed herein, or a pharmaceutically acceptable salt or solvate thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet Formulation

The following ingredients are mixed intimately and pressed into single scored tablets.

| Ingredient | Quantity per tablet, mg |
|---|---|
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| Ingredient | Quantity per capsule, mg |
|---|---|
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for a compound disclosed herein, or a pharmaceutically acceptable salt or solvate thereof, is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound disclosed herein, or a pharmaceutically acceptable salt or solvate thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound disclosed herein, or a pharmaceutically acceptable salt or solvate thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound disclosed herein, or a pharmaceutically acceptable salt or solvate thereof as described herein is administered as a dosage of about 100 mg/Kg.

In some embodiments, the foregoing dosages of a compound disclosed herein, or a pharmaceutically acceptable salt or solvate thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound disclosed herein, or a pharmaceutically acceptable salt or solvate thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound disclosed herein, or a pharmaceutically acceptable salt or solvate thereof is administered to a patient for a period of time followed by a separate period of time where administration of the compound disclosed herein, or a pharmaceutically acceptable salt or solvate thereof is stopped. In some embodiments, a compound disclosed herein, or a pharmaceutically acceptable salt or solvate thereof is administered for a first period and a second period following the first period, with administration stopped during the second period, followed by a third period where administration of the compound disclosed herein, or a pharmaceutically acceptable salt or solvate thereof is started and then a fourth period following the third period where administration is stopped. For example, the period of administration of a compound disclosed herein, or a pharmaceutically acceptable salt or solvate thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound disclosed herein, or a pharmaceutically acceptable salt or solvate thereof, is orally administered to the patient one or more times per day (e.g., one time per day, two times per day, three times per day, four times per day per day or a single daily dose).

In some embodiments, a compound disclosed herein, or a pharmaceutically acceptable salt or solvate thereof, is administered by parenteral administration to the patient one or more times per day (e.g., 1 to 4 times, one time per day, two times per day, three times per day, four times per day or a single daily dose).

In some embodiments, a compound disclosed herein, or a pharmaceutically acceptable salt or solvate thereof, is administered by parenteral administration to the patient weekly.

### Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound disclosed herein, or a pharmaceutically acceptable salt or solvate thereof, or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, kidney disease (e.g., acute kidney disorder, tubular dysfunction, proinflammatory changes to the proximal tubules), adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of a reduction of blood glucose levels (e.g., reduce blood glucose levels), a reduction of blood hemoglobin A1c (HbA1c) levels, a promotion of insulin synthesis, a stimulation of insulin secretion, an increase in the mass of β-cells, a modulation of gastric acid secretion, a modulation of gastric emptying, a decrease in the body mass index (BMI), and/or a decrease in glucagon production (e.g., level). In some embodiments, the compounds and pharmaceutical compositions and methods for treating a patient described herein can reduce blood glucose levels, reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), decrease glucagon production (e.g., level), or any combination thereof. In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound disclosed herein, or a pharmaceutically acceptable salt or solvate thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound disclosed herein, or a pharmaceutically acceptable salt or solvate thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or P-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound disclosed herein, or a pharmaceutically acceptable salt or solvate thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound disclosed herein, or a pharmaceutically acceptable salt or solvate thereof, or a pharmaceutical composition as disclosed herein.

Also provided herein is a method of treating type 2 diabetes mellitus in a patient in need thereof, the method comprising administering to the patient a therapeutically effective amount of a compound disclosed herein, or a pharmaceutically acceptable salt or solvate thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90).

Other non-limiting examples of disorders in metabolically important tissues include joint disorders (e.g., osteoarthritis, secondary osteoarthritis), steatosis (e.g., in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome.

In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include idiopathic intracranial hypertension (IIH), brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., U.S. Publication No. 20060275288A1.

In some embodiments, the condition, disease or disorder is idiopathic intracranial hypertension. Idiopathic intracranial hypertension is characterized by increased intracranial pressure and papilloedema. See, e.g., Virdee et al. Ophthalmol Ther. 2020; 9(4):767-781. In some embodiments, the compounds and pharmaceutical compositions and methods described herein reduce cerebrospinal fluid secretion in a patient with idiopathic intracranial hypertension. In some embodiments, the compounds and pharmaceutical compositions and methods described herein reduce intracranial pressure in a patient with idiopathic intracranial hypertension. In some embodiments, the compounds and pharmaceutical compositions and methods described herein reduce one or more symptoms in a patient with idiopathic intracranial hypertension. Symptoms of idiopathic intracranial hypertension can include severe headaches and visual impairment. In some embodiments, the patient with idiopathic intracranial hypertension is female. In some embodiments, the patient with idiopathic intracranial hypertension is about 20 to about 30 years old. In some embodiments, the patient with idiopathic intracranial hypertension is obese.

In some embodiments, the condition, disease or disorder is Wolfram syndrome. Wolfram syndrome is caused by biallelic mutations of the Wolframin ER transmembrane glycoprotein (Wfs1) gene. See, e.g., Seppa et al. Sci Rep 9, 15742 (2019). Wolfram syndrome can first appear as diabetes mellitus, followed by optic nerve atrophy, deafness, and symptoms of neurodegeneration. Patients with Wolfram syndrome can have symptoms of ataxia, sleep apnea, dysphagia, hearing loss, and loss of taste due to brainstem atrophy. In some embodiments, the compounds and pharmaceutical compositions and methods described herein reduce neuroinflammation in a patient with Wolfram syndrome. In some embodiments, the neuroinflammation is reduced in the inferior olive in the patient. In some embodiments, the compounds and pharmaceutical compositions and methods described herein reduce retinal ganglion cell death in a patient with Wolfram syndrome. In some embodiments, the compounds and pharmaceutical compositions and methods described herein reduce axonal degeneration in a patient with Wolfram syndrome. In some embodiments, the compounds and pharmaceutical compositions and methods described herein reduce one or more symptoms (e.g., any of the symptoms described herein) in a patient with Wolfram syndrome.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., U.S. Publication No. 20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related Conditions and Disorders

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., U.S. Publication No. 20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking. In some embodiments, the weight gain is induced by the use of steroids or antipsychotics.

In some embodiments, the condition, disease or disorder is an eating disorder, such as hyperphagia, binge eating, bulimia, compulsive eating, or syndromic obesity such as Prader-Willi and Bardet-Biedl syndromes.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds disclosed herein, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), peptide YY or an analogue thereof, cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498, naltrexone), orexin receptor antagonists, melanocortin 4 receptor agonists, 11p3-hydroxysteroid dehydrogenase inhibitors (e.g., AZD-4017, BVT-3498, INCB-13739), pancreatic lipase inhibitors (e.g., orlistat, cetilistat), β agonists (e.g., N-5984), diacylglycerol acyltransferase 1 (DGAT1) inhibitors, acetylCoA carboxylase (ACC) inhibitors (e.g., compounds described in International Publication Nos. WO 2020/234726, WO 2020/044266, and U.S. Pat. No. 8,859,577), stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin, empagliflozin, canagliflozin, ipragliflozin, tofogliflozin, sergliflozin etabonate, remogliflozin etabonate, or ertugliflozin), SGLT-1 inhibitors, MCR-4 agonists, monoamine reuptake inhibitors, melanocytestimulating hormone analogs, 5HT2c agonists, galanin antagonists, anorectic agents (such as a bombesin agonist), thyromimetic agents, dehydroepiandrosterone or analogs thereof, human agouti-related protein (AGRP) inhibitors, neuromedin U agonists, NFK inhibitors (e.g., HE-3286), PPAR agonists (e.g., GFT-505, DRF-11605, gemfibrozil, fenofibrate, balaglitazone, ciglitazone, darglitazone, englitazone, isaglitazone, pioglitazone, rosiglitazone, CLX-0940, GW-1536, GW-1 929, GW-2433, KRP-297, L-796449, LR-90, MK-0767, and SB-21 9994), phosphotyrosine phosphatase inhibitors (e.g., sodium vanadate, trodusquemin), GPR119 agonists (e.g., PSN-821, MBX-2982, APD597, compounds described in International Publication Nos. WO 2010/140092, WO 2010/128425, WO 2010/128414, WO 2010/106457), glucokinase activators (e.g., piragliatin, AZD-1656, AZD6370, TTP-355, TTP-399, TTP547, ARRY403, MK-0599, TAK-329, AZD5658 or GKM-001 compounds described in International Publication Nos. WO 2010/103437, WO 2010/103438, WO 2010/013161, WO 2007/122482, WO 2006/112549, WO 2007/028135, WO 2008/047821, WO 2008/050821, WO 2008/136428 and WO 2008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), melanocortin receptor 4 agonist (e.g., setmelanotide), melanin concentrating hormone receptor 1 antagonist, serotonergic agents (e.g. sibutramine, lorcaserin), farnesoid X receptor (FXR) agonist (e.g., obeticholic acid, tropifexor, cilofexor, LY2562175, Met409, TERN-101, EDP305, compounds described in International Publication Nos. WO 2020/234726 and WO 2020/044266), phentermine, zonisamide, norepinephrine/dopamine reuptake inhibitor (e.g., bupropion), GDF-15 analog, methionine aminopeptidase 2 (MetAP2) inhibitor (e.g., beloranib or ZGN-1061), diethylpropion, phendimetrazine, benzphetamine, fibroblast growth factor receptor (FGFR) modulator, biotin, a MAS receptor modulator, glucagon receptor agonist, CCKa agonists (e.g., compounds described in International Publication No. WO 2005/116034 and U.S. Publication No. 2005/0287100), and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS—1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, glimepiride, tolbutamide, glibenclamide, gliclazide, acetohexamide, glyclopyramide, glybuzole, glyburide, glipizide), thiazolidinedione agents (e.g. rosiglitazone, lobeglitazone, troglitazone, balaglitazone, rivoglitazone, lobeglitazone or pioglitazone), glitazars (e.g., aleglitazar, chiglitazar, saroglitazar, muraglitazar, tesaglitazar), SGLT2 inhibitors (e.g., JNJ-28431754, dapagliflozin, AVE2268, TS-033, YM543, TA-7284, ASP1941, THR1474, TS-071, ISIS388626, LX4211, remogliflozin, empagliflozin, canagliflozin, ipragliflozin, tofogliflozin, sergliflozin etabonate, remogliflozin etabonate, ertugliflozin, compounds described in WO 2010/023594), GPR40 agonists (e.g., a FFAR1/FFA1 agonist, e.g. fasiglifam), α-glucosidase inhibitors (e.g., adiposin, camiglibose, pradimicin-Q, salbostatin, voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, anagliptin (SK-0403), teneligliptin, omarigliptin, BI1356, GRC8200, MP-513, PF-00734200, PHX1149, ALS2-0426, TA-6666, TS-021, KRP-104, trelagliptin).

In some embodiments, the one or more additional therapeutic agents include those useful, for example, for treating NAFL and NASH. Non-limiting examples include FXR agonists (e.g., obeticholic acid), PF-05221304, PPAR a/6 agonists (e.g., elafibranor), a synthetic fatty acid-bile conjugate (e.g., aramchol), an anti-lysyl oxidase homologue 2 (LOXL2) monoclonal antibody (e.g., simtuzumab), a caspase inhibitor (e.g., emricasan), a MAPK5 inhibitor, a galectin 3 inhibitor (e.g., GR-MD-02), a fibroblast growth factor 21 (FGF21) (e.g., BMS-986036), a niacin analogue (e.g., ARJ 3037MO), a leukotriene D4 (LTD4) receptor antagonist (e.g., tipelukast), an acetyl-CoA carboxylase (ACC) inhibitor (e.g., NDI 010976 and compounds described in International Publication Nos. WO 2009/144554, WO 2003/072197, WO 2009/144555, and WO 2008/065508), a ketohexokinase (KHK) inhibitor; (e.g., compounds described in WO 2020/234726), an apoptosis signal-regulating kinase 1 (ASK1) inhibitor (selonsertib), an ileal bile acid transporter (IBAT) inhibitor, a dual antagonist of chemokine receptor 2 (CCR2) and CCR5 (e.g., cenicriviroc), diacylglyceryl acyltransferase 2 (DGAT2) inhibitor (e.g., compounds described in WO 2020/234726 and U.S. Publication No. 20180051012), a CB1 receptor antagonist, an anti-CB1R antibody, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone, balaglitazone, rivoglitazone, lobeglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (y-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, zofenopril, fbsinopril, enalapril, ceranopril, cilazopril, delapril, pentopril, quinapril, ramipril, lisinopril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol). Further non-limiting examples of antihypertensive agents include: diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone), alpha adrenergic blockers, beta adrenergic blockers, calcium channel blockers (e.g., diltiazem, verapamil, nifedipine and amlodipine), vasodilators (e.g., hydralazine), renin inhibitors, AT-1 receptor antagonists (e.g., losartan, irbesartan, valsartan), ET receptor antagonists (e.g., sitaxsentan, atrsentan, compounds disclosed in U.S. Pat. Nos. 5,612,359 and 6,043,265), dual ET/All antagonist (e.g., compounds disclosed in WO 2000/01389), neutral endopeptidase (NEP) inhibitors, If channel blocker ivabradinand, vasopepsidase inhibitors (dual NEP-ACE inhibitors) (e.g., gemopatrilat and nitrates).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran, boroarginine derivatives, boropeptides, heparins, hirudin, and melagatran), FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., anistreplase, streptokinase, tenecteplase (TNK), lanoteplase (nPA), urokinase, tisokinase, alteplase, nateplase, monteplase, pamiteplase, factor VIIa inhibitors, PAI-1 inhibitors, alpha2-antiplasmin inhibitors, and anisoylated plasminogen streptokinase activator complex), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin)-pravastatin, pitavastatin, lovastatin, atorvastatin, simvastatin, fluvastatin, itavastatin, ZD-4522), HMG-CoA synthase inhibitors, cholesterol-lowering agents, bile acid sequestrants (e.g., cholestyramine, questran, colestipol, and colesevelam), cholesterol absorption inhibitors (e.g. plant sterols such as phytosterols), cholesteryl ester transfer protein (CETP) inhibitors, inhibitors of the ileal bile acid transport system (IBAT inhibitors), diacylglyceryl acyltransferase 1 (DGAT1) inhibitors (e.g., AZD7687, LCQ908, compounds described in WO 2009/016462, WO 2010/086820), monoacylglycerol O-acyltransferase inhibitors, α-amylase inhibitors (e.g., tendamistat, trestatin, AL-3688), α-glucoside hydrolase inhibitors, SIRT-1 activators, c-Jun N-terminal kinase (JNK) inhibitors, a VPAC2 receptor agonist, TGR5 receptor modulators (e.g., compounds described in), GPBAR1 receptor modulators, GPR120 modulators, high affinity nicotinic acid receptor (HM74A) activators, carnitine palmitoyl transferase enzyme inhibitors, mineralocorticoid receptor inhibitors, inhibitors of TORC2, fatty acid synthetase inhibitors, serine palmitoyl transferase inhibitors, GPR81 modulators, GPR39 modulators, GPR43 modulators, GPR41 modulators, GPR105 modulators, Kv1.3 modulators, retinol binding protein 4 modulators, somatostain receptor modulators, PDHK2 modulators, PDHK4 modulators, MAP4K4 inhibitors, IL1 family modulators (e.g., ILI beta modulators), ACAT inhibitors, MTP inhibitors (e.g., diriotapide, mitratapide, and implitapide), lipooxygenase inhibitors, PCSK9 modulators (e.g., alirocumab and evolocumab), RXRalpha modulators, cysteamine, cystamine, an RNA antisense construct to inhibit protein tyrosine phosphatase PTPRU, vitamin B complex, pentraxin proteins, a protein tyrosine phosphatase-1 B (PTP-1 B) inhibitor (e.g., trodusquemine, hyrtiosal extract, and compounds described by Zhang et al. Drug Discovery Today. 2007, 12(9-10): 373-381), ezitimbe, betaine, pentoxifylline, alpha delta-9 desaturase, BCKDK inhibitors, branched-chain alpha keto acid dehydrogenase kinase (BCBK) inhibitors, PNPLA3 inhibitors, FGF1 9 analogs, SCD1 inhibitors, bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropyridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone, spironolactone), centrally acting adrenergic drugs, such as central alpha agonists (e.g. clonidine), diuretic agents (e.g. furosemide), torsemide, bemetanide, ethacrynic acid, thiazide-type diuretics (e.g., chlorothiazide, hydrochlorothiazide, benzthiazide, hydroflumethiazide, bendroflumethiazide, methychlorthiazide, polythiazide, trichloromethiazide, indapamide), phthalimidine-type diuretics (e.g., chlorthalidone, metolazone), quinazoline-type diuretics (e.g., quinethazone), potassium-sparing diuretics (e.g., triamterene and amiloride), thyroid receptor agonists (e.g., compounds described in WO 2020/117987), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin), non-steroidal anti-inflammatory drugs (NSAIDS), thromboxane-A2-receptor antagonists (e.g., ifetroban), thromboxane-A2-synthetase inhibitors, PDE inhibitors (e.g., Pletal, dipyridamole)), antagonists of purinergic receptors (e.g., P2Y1 and P2Y12), adenosine diphosphate (ADP) receptor inhibitors (e.g. clopidogrel), phosphodiesterase inhibitors (e.g. cilostazol), glycoprotein IIB/IIA inhibitors (e.g. tirofiban, eptifibatide, and abcixima), adenosine reuptake inhibitors (e.g. dipyridamole), noradrenergic agents (e.g. phentermine), serotonergic agents (e.g. sibutramine, lorcaserin), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO 2007/013694, WO 2007/018314, WO 2008/093639 and WO 2008/099794, GPR40 agonists (e.g., fasiglifam or a hydrate thereof, compounds described in WO 2004/041266, WO 2004/106276, WO 2005/063729, WO 2005/063725, WO 2005/087710, WO 2005/095338, WO 2007/013689 and WO 2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., K⁺ channel openers, mexiletine, propafenone, metoprolol, atenolol, carvadiol, propranolol, sotalol, dofetilide, amiodarone, azimilide, ibutilide, ditiazem, and verapamil), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), metaglidasen, AMG-131, balaglitazone, MBX-2044, rivoglitazone, aleglitazar, chiglitazar, saroglitazar, muraglitazar, tesaglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide-, piroxicam, NO donating agents (e.g., organonitrates), NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). While not wishing to be bound by theory, it is believed that administering one or more anti-emetic agents in combination with the compounds described herein may allow higher dosages of the compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, and cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+ netupitant), quetiapine, qamosetron, ramosteron, ricasetron, risperidone, ziprasidone, and zatosetron.

In certain embodiments, the 5HT-3-receptor antagonist is Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y—25130 hydrochloride, MDL 72222, Tropanyl-3,5-dimethylbenzoate 3-(4-AIIyI-piperazin-1-yl)-2-quinoxalinecarbonitrile maleate, Zacopride hydrochloride and Mirtazepine.

In certain embodiments, the 5HT-3-receptor antagonist is Granisetron, Dolasetron, Ondansetron hydrochloride, Tropisetron, Ramosetron, Palonosetron, Alosetron, Bemesetron, and Zatisetron.

In certain embodiments, the 5HT-3-receptor antagonist is Granisetron, Dolasetron and Ondansetron.

In certain embodiments, the 5HT-3-receptor antagonist is Granisetron.

In certain embodiments, the 5HT-3-receptor antagonist is Ondansetron.

In some embodiments, the anti-emetic agent is an antihistamine. Non-limiting examples of antihistamines include: piperazine derivatives (e.g., cyclizine, meclizine, and cinnarizine); Promethazine; Dimenhydrinate (Dramamine, Gravol); Diphenhydramine; Hydroxyzine; Buclizine; and Meclizine hydrochloride (Bonine, Antivert), doxylamine, and mirtazapine.

In some embodiments, the anti-emetic agent is an anticholinergic agent (Inhibitors of the acetylcholine receptors). Non-limiting examples of anticholinergic agents include: atropine, Scopolamine, Glycopyrron, Hyoscine, Artane (Trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, Dexamethasone, Methylprednisolone, Prednisone®, and Trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples ofNKl-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH₂OCO]—(R)-alpha-MeTrp-(S)—NHCH(CH₃)Ph), FK888, and (D-Pro4, D-Trp7,9,10, Phe11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine,); benzamides (e.g., Metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. Nos. 10,071,088 B2; 6,673,792 B1; 6,197,329 B1; 10,828,297 B2; 10,322,106 B2; 10,525,033 B2; WO 2009080351 A1; WO 2019203753 A2; WO 2002020001 A2; U.S. Pat. Nos. 8,119,697 B2; 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m2 to greater than or about 100 kg/m2. In some embodiments, the BMI of the patient is greater than or about 30 kg/m2 to greater than or about 90 kg/m2. In some embodiments, the BMI of the patient is greater than or about 40 kg/m2 to greater than or about 80 kg/m2. In some embodiments, the BMI of the patient is greater than or about 50 kg/m2 to greater than or about 70 kg/m2.

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

### General Synthetic Methods

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3^{rd} Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme I illustrates a general method which can be employed for the synthesis of compounds described herein, where Ring A, Ring B, Ring C, mm, nn, b, T¹, T², L⁰, L¹, L², L³, R^{b}, X¹, X², X³, X⁴, X⁵, and X⁶ are each as defined herein, and LG is a leaving group, such as halo (e.g., Cl, Br, or I) or with -L¹-L² forms an oxo.

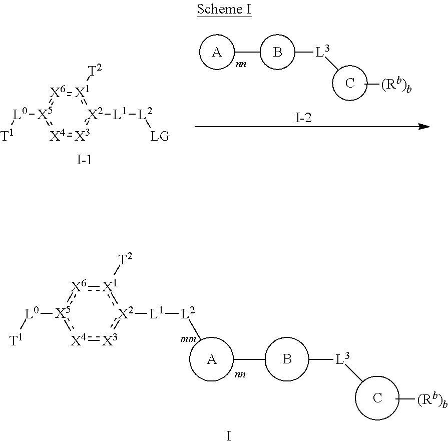

Compounds of Formula I can be provided by coupling compound I-1 with compound I-2 under suitable coupling reaction conditions. Exemplary suitable reaction conditions include, but are not limited to, a polar aprotic solvent (e.g., acetonitrile), optionally in the presence of a base (e.g., potassium carbonate), or reductive amination conditions.

For any compound shown in Scheme I, it should be understood that various derivatives can be provided by functional group interconversion at any step. For example with -L⁰-T¹ or T¹, various compounds of Formula I can be provided via functional group interconversion using methods known to one of skill in the art. Likewise, various compounds of Formula I can be prepared by contacting compounds where an R^{b} is a leaving group (e.g., halo, such as Cl, Br, or I, or a pseudohalide, such as a triflate, sulfonate, or phosphate), with a compound of Formula R^{b}—B, wherein B is a suitable functional group such as, but not limited to, a boronic acid or a derivative thereof, such as a boronic ester, zinc or magnesium halide, an organotin compound, such as tributylstannane or trimethylstannane, fluorosulfonyl esters, tin, sodium, hydrogen, and the like. Such reactions are commonly utilized for aromatic functionalization, and are typically conducted in the presence of suitable catalyst such as, but not limited to, a palladium catalyst including [1,1′-bis(diphenylphosphino)ferrocene]palladium(II) dichloride, Pd(OAc)₂, Pd(PPh₃)₄, PdCl₂(PPh₃)₂ or tris(dibenzylideneacetone)dipalladium(0), and the like, or a copper catalyst such as CuCl or CuI, and if required suitable mediator, co-catalyst and/or base known to one skilled in the art using suitable solvents/solvent mixtures. Upon reaction completion, compounds of Formula I can be recovered by conventional techniques such as neutralization, extraction, precipitation, chromatography, filtration and the like. In certain embodiments, when control of stereochemistry is desired, proper control of reaction conditions and selection of substituents for the reagents can at least partially dictate or preserve the formation of the various stereoisomers.

In some embodiments, the various substituents of Formula I-1, I-2, or I-3 (e.g., Ring A, Ring B, Ring C, mm, nn, b, T¹, T², L⁰, L¹, L², L³, R^{b}, X¹, X², X³, X⁴, X⁵, and X⁶) are as defined herein. However, derivatization of compounds I, I-1, or I-2 prior to reacting in any step, and/or further derivatization of the resulting reaction product, provides various compounds of Formula I. Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration, and the like. Other modifications to arrive at compounds of this disclosure are within the skill of the art.

### EXAMPLES

General information: All evaporations were carried out in vacuo with a rotary evaporator. Analytical samples were dried in vacuo (1-5 mm Hg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. 1H chemical shifts are reported in 6 values in ppm with the deuterated solvent as the internal standard. Data are reported as follows: chemical shift, multiplicity (s singlet, d doublet, t triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

This disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

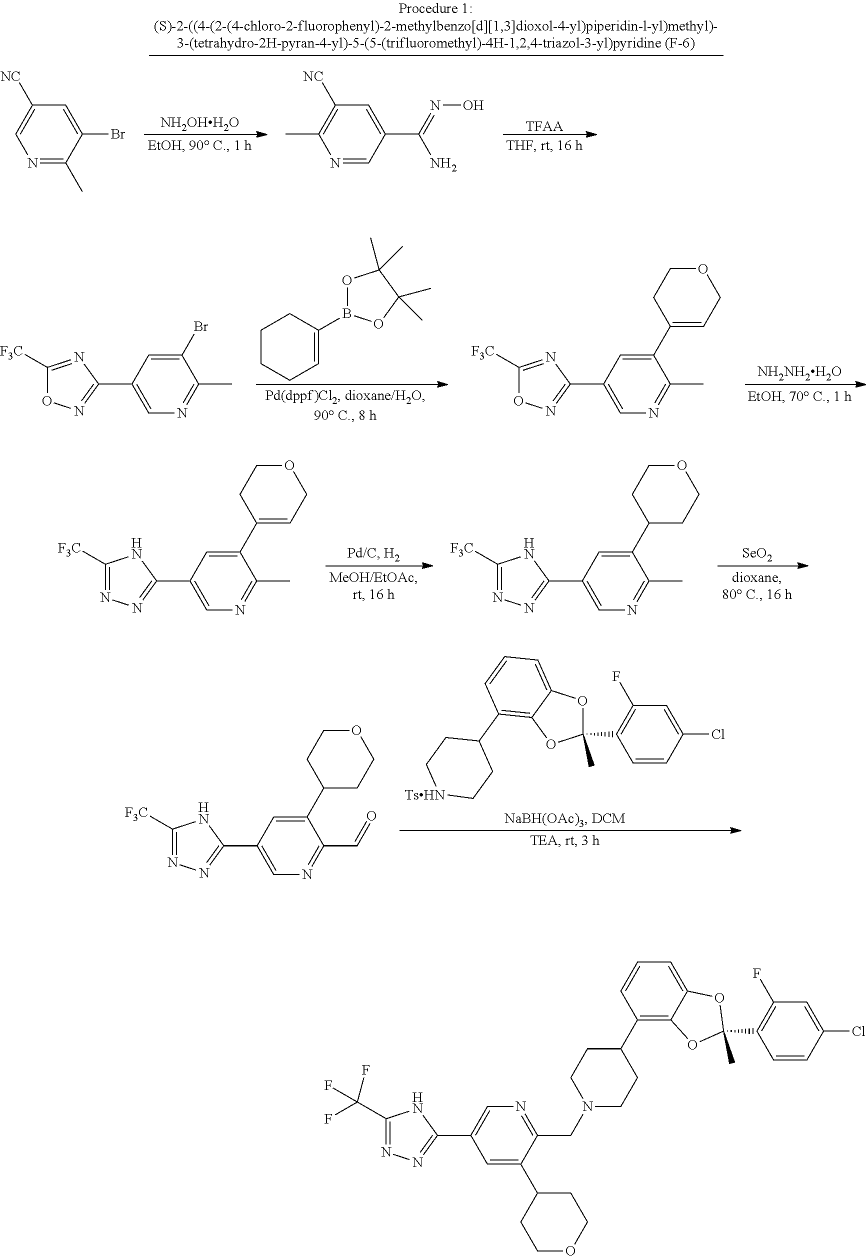

### Step A: (Z)-5-bromo-N′-hydroxy-6-methylnicotinimidamide

A mixture of 5-bromo-6-methylnicotinonitrile (60 mg, 0.31 mmol) and 50% NH₂OH aq in water (202 mg, 3.1 mmol) in EtOH (3 mL) was stirred at 90° C. for 1 h. The mixture was concentrated to obtain (Z)-5-bromo-N′-hydroxy-6-methylnicotinimidamide (60 mg, yield: 87%). MS Calcd: 229.0. MS Found: 230.0 [M+H]⁺

### Step B: 3-(5-bromo-6-methylpyridin-3-yl)-5-(trifluoromethyl)-1,2,4-oxadiazole

To a solution of (Z)-5-bromo-N′-hydroxy-6-methylnicotinimidamide (60 mg, 0.26 mmol) in THF (3 mL) was added dropwise TFAA (550 mg, 2.6 mmol) at 0° C. The mixture was warmed to rt for stirring 16 h. The NaHCO₃ aq and EtOAc were added to the mixture to adjust pH above 7, the organic phase was washed by brine, dried with Na₂SO₄, the solvent was concentrated to dryness to give the residue, which was purified by prep-TLC to obtain 3-(5-bromo-6-methylpyridin-3-yl)-5-(trifluoromethyl)-1,2,4-oxadiazole (80 mg). MS Calcd: 307.0. MS Found: 307.9 [M+H]⁺

### Step C: 3-(5-(3,6-dihydro-2H-pyran-4-yl)-6-methylpyridin-3-yl)-5-(trifluoromethyl)-1,2,4-oxadiazole

A mixture of 3-(5-bromo-6-methylpyridin-3-yl)-5-(trifluoromethyl)-1,2,4-oxadiazole (80 mg, 0.26 mmol) in dioxane/H₂O (4 mL/1 mL) was added 2-(3,6-dihydro-2H-pyran-4-yl)-4,4,5,5-tetramethyl-1,3,2-dioxaborolane (101 mg, 0.52 mmol), Pd(dppf)Cl₂ (21 mg, 0.03 mmol) under N₂. The mixture was stirred at 90° C. for 8 hours. The water and EtOAc were added to the mixture, the organic phase was washed by brine, dried with Na₂SO₄, the residue was purified by prep-TLC to obtain 3-(5-(3,6-dihydro-2H-pyran-4-yl)-6-methylpyridin-3-yl)-5-(trifluoromethyl)-1,2,4-oxadiazole (11 mg, yield: 13.5%). MS Calcd: 311.1. MS Found: 312.1 [M+H]⁺.

### Step D: 3-(3,6-dihydro-2H-pyran-4-yl)-2-methyl-5-(5-(trifluoromethyl)-4H-1,2,4-triazol-3-yl)pyridine

A mixture of 3-(5-(3,6-dihydro-2H-pyran-4-yl)-6-methylpyridin-3-yl)-5-(trifluoromethyl)-1,2,4-oxadiazole (21 mg, 0.068 mmol) and 85% NH₂NH₂·H₂O aq in water (0.2 mL) in EtOH (0.5 mL) was stirred at 70° C. for 1 h. The crude product was obtained by freeze-drying with the solvent. The crude product was purified by prep-TLC to 3-(3,6-dihydro-2H-pyran-4-yl)-2-methyl-5-(5-(trifluoromethyl)-4H-1,2,4-triazol-3-yl)pyridine (16 mg, yield: 76.5%). MS Calcd: 310.1. MS Found: 311.1[M+H]⁺

### Step E: 2-methyl-3-(tetrahydro-2H-pyran-4-yl)-5-(5-(trifluoromethyl)-4H-1,2,4-triazol-3-yl)pyridine

A mixture of 3-(3,6-dihydro-2H-pyran-4-yl)-2-methyl-5-(5-(trifluoromethyl)-4H-1,2,4-triazol-3-yl)pyridine (16 mg, 0.052 mmol) in EtOAc/MeOH (5 mL/0.5 mL) at rt was added Pd/C wet. 10% content (10 mg) under H₂ balloons for stirring 16 hours. The mixture was purified by prep-TLC to obtain 2-methyl-3-(tetrahydro-2H-pyran-4-yl)-5-(5-(trifluoromethyl)-4H-1,2,4-triazol-3-yl)pyridine (14 mg, yield: 86.9%). MS Calcd: 312.1. MS Found: 313.0 [M+H]⁺.

### Step F: 5-(tetrahydro-2H-pyran-4-yl)-3-(5-(trifluoromethyl)-4H-1,2,4-triazol-3-yl)picolinaldehyde

A mixture of 2-methyl-3-(tetrahydro-2H-pyran-4-yl)-5-(5-(trifluoromethyl)-4H-1,2,4-triazol-3-yl)pyridine (14 mg, 0.045 mmol) in dioxane (3 mL) was added SeO₂ (50 mg, 0.448 mmol) at 80° C. for stirring 16 hours. The mixture was filtered, and the filtrate was concentrated to obtain 5-(tetrahydro-2H-pyran-4-yl)-3-(5-(trifluoromethyl)-4H-1,2,4-triazol-3-yl)picolinaldehyde (13 mg, yield: 89%), which was used directly with no further purification. MS Calcd: 326.1. MS Found: 327.1 [M+H]⁺.

### Step G: (S)-2-((4-(2-(4-chloro-2-fluorophenyl)-2-methylbenzo[d][1,3]dioxol-4-yl)piperidin-1-yl)methyl)-3-(tetrahydro-2H-pyran-4-yl)-5-(5-(trifluoromethyl)-4H-1,2,4-triazol-3-yl)pyridine (F-6)

A mixture of (S)-4-(2-(4-chloro-2-fluorophenyl)-2-methylbenzo[d][1,3]dioxol-4-yl)piperidine (21 mg, 0.041 mmol), TEA (0.028 mL, 0.20 mmol) in DCM (4 mL) was stirred at 0° C. for 30 minutes. Then the mixture 5-(tetrahydro-2H-pyran-4-yl)-3-(5-(trifluoromethyl)-4H-1,2,4-triazol-3-yl)picolinaldehyde (13 mg, 0.040 mmol) and NaBH(OAc)₃ (34 mg, 0.16 mol) were added slowly for stirring 15 mins at 0° C. Then the mixture was warmed to rt for stirring 3 h. The water and DCM were added to the mixture, the organic phase was washed by brine, dried over Na₂SO₄. The solvent was concentrated to dryness. The residue was purified by pre-HPLC (0.1% NH₃·H₂O) to obtain (S)-2-((4-(2-(4-chloro-2-fluorophenyl)-2-methylbenzo[d][1,3]dioxol-4-yl)piperidin-1-yl)methyl)-3-(tetrahydro-2H-pyran-4-yl)-5-(5-(trifluoromethyl)-4H-1,2,4-triazol-3-yl)pyridine (1.0 mg, yield: 3.8%). MS Calcd: 657.2. MS Found: 658.0 [M+H]⁺.

¹H NMR (400 MHz, MeOD-d₄): δ 9.09 (s, 1H), 8.45 (s, 1H), 7.63-7.58 (t, J=16.4 Hz, 1H), 7.29 (dd, J=2.0 Hz/J=10.8 Hz, 2H), 7.22 (dd, J=2.0 Hz/J=8.4 Hz, 1H), 6.82-6.75 (m, 3H), 4.58 (s, 2H), 4.12-4.09 (dd, J=3.2 Hz/J=11.2 Hz, 2H), 3.67 (s, 1H), 3.64-3.61 (dd, J=1.6 Hz/J=12 Hz, 1H), 3.48 (m, 1H), 3.30 (m, 1H), 3.13 (m, 1H), 2.05-1.93 (m, 2H), 1.83-1.79 (m, 2H), 1.33-1.29 (m, 6). ¹⁹F NMR (377 MHz, MeOD-d₄): δ 65.01, −112.37.

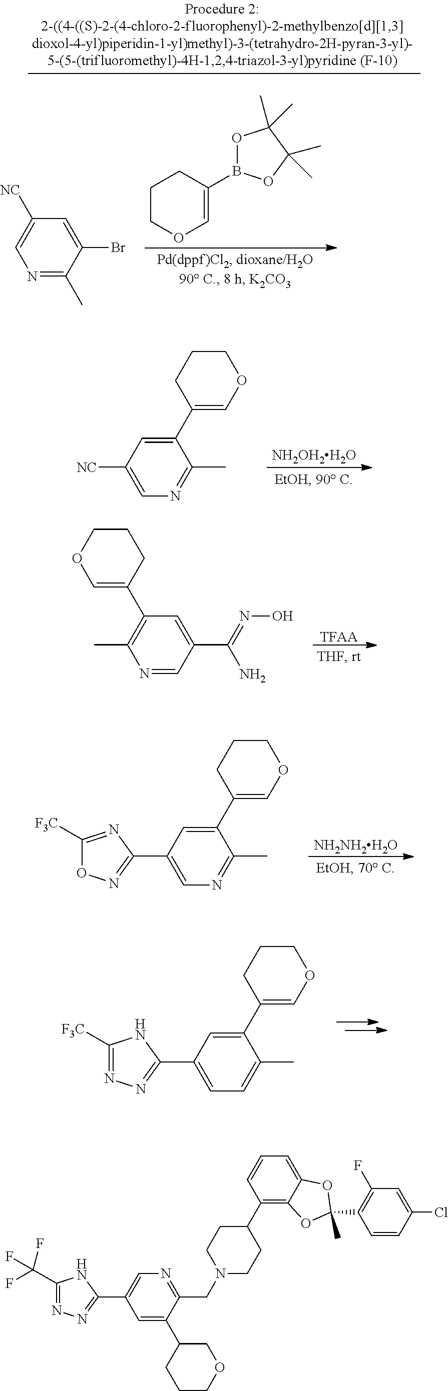

### Step A: 5-(3,4-dihydro-2H-pyran-5-yl)-6-methylnicotinonitrile

A mixture of 2-(3,4-dihydro-2H-pyran-5-yl)-4,4,5,5-tetramethyl-1,3,2-dioxaborolane (200 mg, 0.95 mmol) in dioxane/H₂O (4 mL/1 mL) was added 5-bromo-6-methylnicotinonitrile (186 mg, 0.95 mmol), Pd(dppf)Cl₂ (70 mg, 0.01 mmol) and K₂CO₃ (393 mg, 2.8 mmol) under N₂. The mixture was stirred at 90° C. for 8 hours. The water and EtOAc were added to the mixture, the organic phase was washed by brine, dried with Na₂SO₄, the residue was purified by prep-TLC to obtain 5-(3,4-dihydro-2H-pyran-5-yl)-6-methylnicotinonitrile (60 mg, yield: 31.6%). MS Calcd: 200.0. MS Found: 201.0[M+H]⁺.

### Step B: (Z)-5-(3,4-dihydro-2H-pyran-5-yl)-N′-hydroxy-6-methylnicotinimidamide

A mixture of 5-(3,4-dihydro-2H-pyran-5-yl)-6-methylnicotinonitrile (60 mg, 0.3 mmol) and 50% NH₂OH aq in water (198 mg, 3 mmol) in EtOH (3 mL) was stirred at 90° C. for 1 h. The mixture was concentrated to obtain (Z)-5-(3,4-dihydro-2H-pyran-5-yl)-N′-hydroxy-6-methylnicotinimidamide (70 mg). MS Calcd: 233.0. MS Found: 234.1 [M+H]⁺

### Step C: 3-(5-(3,4-dihydro-2H-pyran-5-yl)-6-methylpyridin-3-yl)-5-(trifluoromethyl)-1,2,4-oxadiazole

To a solution of (Z)-5-(3,4-dihydro-2H-pyran-5-yl)-N′-hydroxy-6-methylnicotinimidamide (70 mg, 0.3 mmol) in THF (3 mL) was added dropwise TFAA (500 mg, 2.4 mmol) at 0° C. The mixture was warmed to rt for stirring 16 h. The NaHCO₃ aq and EtOAc were added to the mixture to adjust pH above 7, the organic phase was washed by brine, dried with Na₂SO₄, the solvent was concentrated to dryness to give the residue, which was purified by prep-TLC to obtain 3-(5-(3,4-dihydro-2H-pyran-5-yl)-6-methylpyridin-3-yl)-5-(trifluoromethyl)-1,2,4-oxadiazole (80 mg, yield: 86%). MS Calcd: 311.2; MS Found: 312.2 [M+H]⁺

### Step D: 3-(3-(3,4-dihydro-2H-pyran-5-yl)-4-methylphenyl)-5-(trifluoromethyl)-4H-1,2,4-triazole

A mixture of 3-(5-(3,4-dihydro-2H-pyran-5-yl)-6-methylpyridin-3-yl)-5-(trifluoromethyl)-1,2,4-oxadiazole (80 mg, 0.26 mmol) and 85% NH₂NH₂·H₂O aq in water (0.2 mL) in EtOH (0.5 mL) was stirred at 70° C. for 1 h. The crude product was obtained by freeze-drying with the solvent. The crude product was purified by prep-TLC to 3-(3-(3,4-dihydro-2H-pyran-5-yl)-4-methylphenyl)-5-(trifluoromethyl)-4H-1,2,4-triazole (30 mg, yield: 37.5%). MS Calcd:310.10. MS Found: 311.1 [M+H]⁺

### Step E: 2-((4-((S)-2-(4-chloro-2-fluorophenyl)-2-methylbenzo[d][1,3]dioxol-4-yl)piperidin-1-yl)methyl)-3-(tetrahydro-2H-pyran-3-yl)-5-(5-(trifluoromethyl)-4H-1,2,4-triazol-3-yl)pyridine

2-((4-((S)-2-(4-chloro-2-fluorophenyl)-2-methylbenzo[d][1,3]dioxol-4-yl)piperidin-1-yl)methyl)-3-(tetrahydro-2H-pyran-3-yl)-5-(5-(trifluoromethyl)-4H-1,2,4-triazol-3-yl)pyridine (13 mg) was obtained with the similar method of Example 1. MS Calcd: 657.2. MS Found: 658.2 [M+H]⁺.

¹H NMR (400 MHz, MeOD-d₄): δ 8.95 (d, J=1.6 Hz, 1H), 8.29 (d, J=2.0 Hz, 1H), 7.58-7.52 (m, 2H), 7.35-7.31 (m, 1H), 6.80-6.78 (m, 2H), 6.70 (dd, J₁=4.0 Hz, J₂=8.8 Hz, 1H), 3.99-3.82 (m, 4H), 3.49-3.40 (m, 3H), 2.93-2.91 (m, 2H), 2.68-2.64 (m, 1H), 2.33-2.26 (m, 2H), 2.04-1.97 (m, 5H), 1.75-1.67 (m, 6H). ¹⁹F NMR (377 MHz, MeOD-d₄): δ 65.01, −112.3

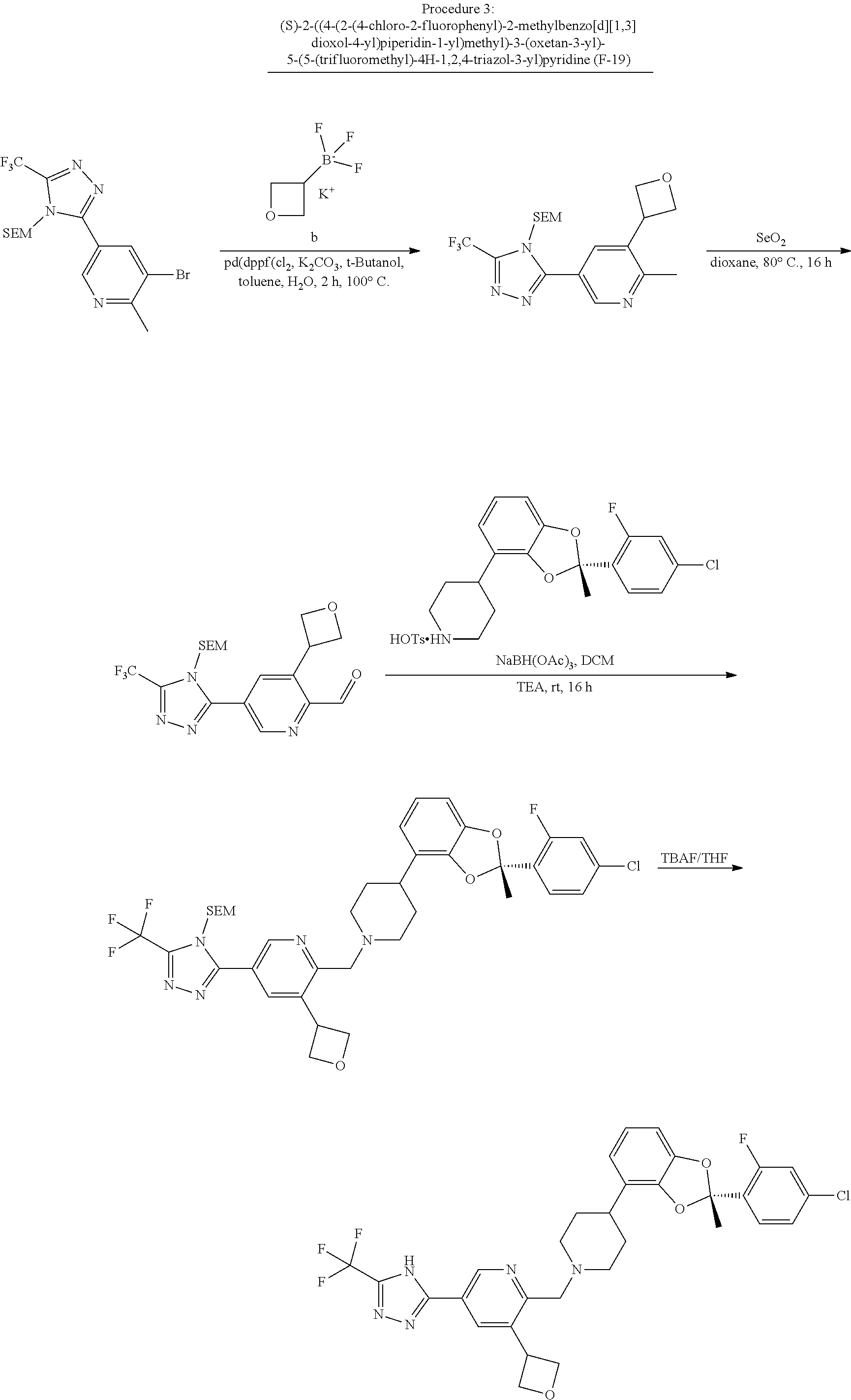

### Step A: 2-methyl-3-(oxetan-3-yl)-5-(5-(trifluoromethyl)-4-((2-(trimethylsilyl)ethoxy)methyl)-4H-1,2,4-triazol-3-yl)pyridine

A mixture of 3-bromo-2-methyl-5-(5-(trifluoromethyl)-4-((2-(trimethylsilyl)ethoxy)methyl)-4H-1,2,4-triazol-3-yl)pyridine (105 mg, 0.24 mmol), trifluoro(oxetan-3-yl)borate (118 mg, 0.72 mmol), Pd(dppf)Cl₂ (18 mg, 0.02 mmol), K₂CO₃ (133 mg, 0.96 mmol), water (0.5 ml) and t-Butanol (1 ml) in toluene (10 mL) was stirred at 100° C. in microwave cube for 16 h. The mixture was poured into water (30 mL) and extracted with EtOAc (2×30 mL), the combined organic layer was washed with brine, dried over sodium sulfate, filtered and the residue was purified by prep-TLC (PE:EA 4:1) to obtain 2-methyl-3-(oxetan-3-yl)-5-(5-(trifluoromethyl)-4-((2-(trimethylsilyl)ethoxy)methyl)-4H-1,2,4-triazol-3-yl)pyridine (22 mg, yield: 22.2). MS Calcd: 414.2. MS Found: 415.3 [M+H]⁺

### Step B: 3-(oxetan-3-yl)-5-(5-(trifluoromethyl)-4-((2-(trimethylsilyl)ethoxy)methyl)-4H-1,2,4-triazol-3-yl)picolinaldehyde

A mixture of 2-methyl-3-(oxetan-3-yl)-5-(5-(trifluoromethyl)-4-((2-(trimethylsilyl)ethoxy)methyl)-4H-1,2,4-triazol-3-yl)pyridine (22 mg, 0.05 mmol) and SeO₂ (59 mg, 0.53 mmol) in 1,4-dioxane (2 mL) was stirred at 80° C. for 16 h. The mixture was filtered, and the residue was concentrated to obtain 3-(oxetan-3-yl)-5-(5-(trifluoromethyl)-4-((2-(trimethylsilyl)ethoxy)methyl)-4H-1,2,4-triazol-3-yl)picolinaldehyde (35 mg). MS Calcd: 428.1. MS Found: 429.0 [M+H]⁺

### Step C: (S)-2-((4-(2-(4-chloro-2-fluorophenyl)-2-methylbenzo[d][1,3]dioxol-4-yl)piperidin-1-yl)methyl)-3-(oxetan-3-yl)-5-(5-(trifluoromethyl)-4-((2-(trimethylsilyl)ethoxy)methyl)-4H-1,2,4-triazol-3-yl)pyridine

A mixture of (S)-4-(2-(4-chloro-2-fluorophenyl)-2-methylbenzo[d][1,3]dioxol-4-yl)piperidine 4-methylbenzenesulfonate (47 mg, 0.09 mmol) and TEA (42 mg, 0.42 mmol) in DCM (5 mL) was stirred at rt for 0.5 h. The mixture was added 3-(oxetan-3-yl)-5-(5-(trifluoromethyl)-4-((2-(trimethylsilyl)ethoxy)methyl)-4H-1,2,4-triazol-3-yl)picolinaldehyde (35 mg, 0.08 mmol) and the mixture was stirred for 2 h. The mixture was added NaBH(OAC)₃ (69 mg, 0.33 mmol) and the mixture was stirred at rt for 16 h. The mixture was poured into water (30 mL) and extracted with DCM (2×30 mL), the combined organic layer was washed with brine, dried over sodium sulfate, filtered and the residue was concentrated to obtain (S)-2-((4-(2-(4-chloro-2-fluorophenyl)-2-methylbenzo[d][1,3]dioxol-4-yl)piperidin-1-yl)methyl)-3-(oxetan-3-yl)-5-(5-(trifluoromethyl)-4-((2-(trimethylsilyl)ethoxy)methyl)-4H-1,2,4-triazol-3-yl)pyridine (70 mg). MS Calcd: 759.3. MS Found: 760.3 [M+H]⁺

### Step D: (S)-2-((4-(2-(4-chloro-2-fluorophenyl)-2-methylbenzo[d][1,3]dioxol-4-yl)piperidin-1-yl)methyl)-3-(oxetan-3-yl)-5-(5-(trifluoromethyl)-4H-1,2,4-triazol-3-yl)pyridine

A mixture of (S)-2-((4-(2-(4-chloro-2-fluorophenyl)-2-methylbenzo[d][1,3]dioxol-4-yl)piperidin-1-yl)methyl)-3-(oxetan-3-yl)-5-(5-(trifluoromethyl)-4-((2-(trimethylsilyl)ethoxy)methyl)-4H-1,2,4-triazol-3-yl)pyridine (70 mg, 0.09 mmol) and TABF (1 M in THF, 2 mL) in THF (1 mL) was stirred at 60° C. for 6 h. The mixture was purified by prep-HPLC to obtain (S)-2-((4-(2-(4-chloro-2-fluorophenyl)-2-methylbenzo[d][1,3]dioxol-4-yl)piperidin-1-yl)methyl)-3-(oxetan-3-yl)-5-(5-(trifluoromethyl)-4H-1,2,4-triazol-3-yl)pyridine (0.9 mg, yield: 1.6%). MS Calcd: 629.2. MS Found: 630.2 [M+H]⁺

¹H NMR (400 MHz, CD₃OD): δ 8.97 (s, 1H), 8.24 (s, 1H), 7.61 (t, J=7.4 Hz, 1H), 7.25 (dd, J=6.8 Hz/J=23.6 Hz, 2H), 6.84-6.74 (m, 3H), 3.67-3.52 (m, 11H), 3.07-2.91 (m, 1H), 2.26-2.20 (m, 7H). ¹⁹F NMR (377 MHz, CD₃OD): δ −73.9), −75.85, −112.05.

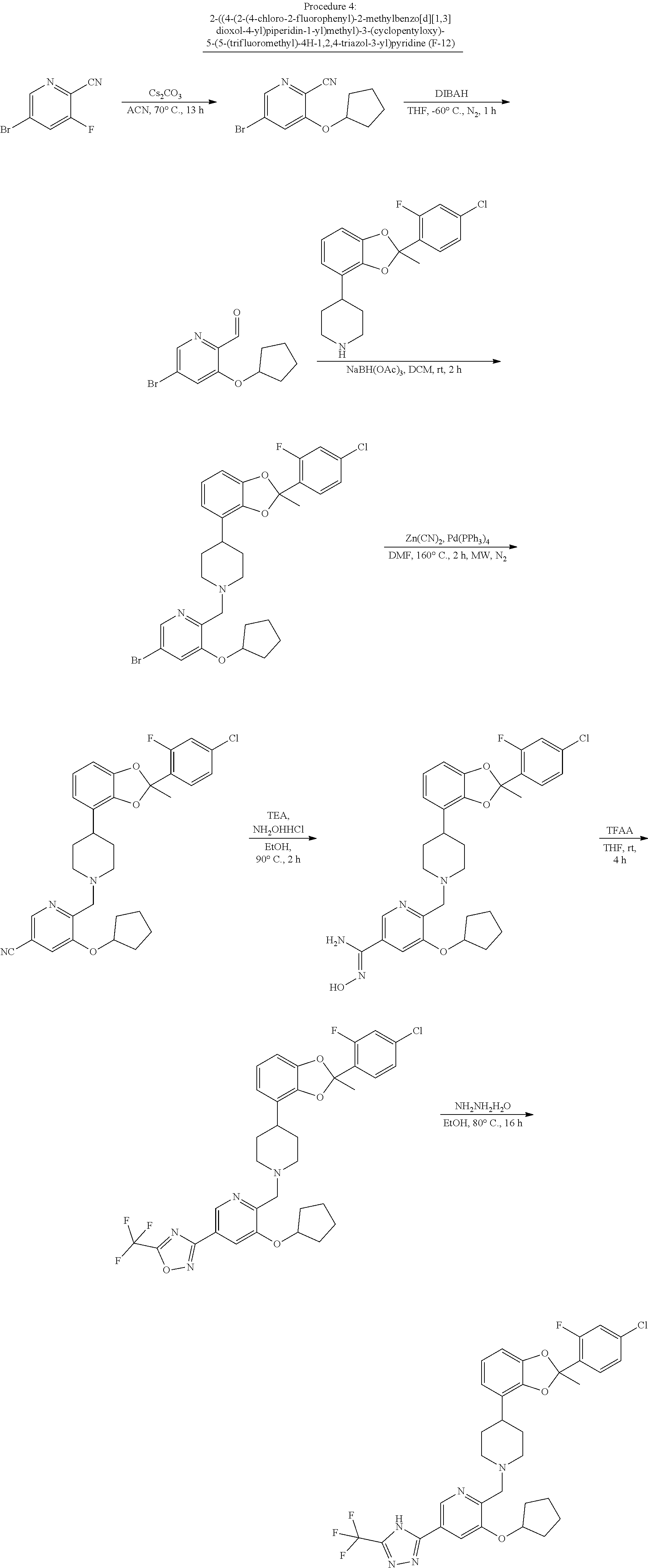

### Step A: 5-bromo-3-(cyclopentyloxy)picolinonitrile

A mixture of 5-bromo-3-fluoropicolinonitrile (2.0 g, 9.9 mmol), cyclopentanol (943 mg, 10.9 mmol) and Cs₂CO₃ (9.7 g, 29.8 mmol) in ACN (20 mL) was stirred at 70° C. for 13 hours. The reaction mixture was cooled to room temperature. The mixture was diluted with H₂O (100 mL), extracted with EA (3×200 mL). The combined organic layers were washed with brine (150 mL). The organic layer was dried over Na₂SO₄, filtered, and concentrated under reduced pressure. The residue was purified by silica gel column chromatography, eluted with PE/EA (5/1) to give 5-bromo-3-(cyclopentyloxy)picolinonitrile (2.0 g, yield: 76.2%). MS Calcd.: 266.0. MS Found: 267.0 [M+H]⁺.

### Step B: 5-bromo-3-(cyclopentyloxy)picolinaldehyde

To a stirred solution of 5-bromo-3-(cyclopentyloxy)picolinonitrile (1.0 g, 3.7 mmol) in dry THF (10 mL) under Nitrogen was added dropwise DIBAH (1.0 M in THF, 5.6 mL) at −78° C. The reaction mixture was stirred at −60° C. for 1 hours. The mixture was diluted with Sat. sodium potassium tartrate solution (100 mL), extracted with EA (3×200 mL). The combined organic layers were washed with brine (150 mL). The organic layer was dried over Na₂SO₄, filtered, and concentrated under reduced pressure. The residue was purified by silica gel column chromatography, eluted with PE/EA (3/1) to give 5-bromo-3-(cyclopentyloxy)picolinaldehyde (390 mg, yield: 38.5%). MS Calcd.: 269.01. MS Found: 269.9 [M+H]⁺.

### Step C: 5-bromo-2-((4-(2-(4-chloro-2-fluorophenyl)-2-methylbenzo[d][1,3]dioxol-4-yl)piperidin-1-yl)methyl)-3-(cyclopentyloxy)pyridine

To a mixture of 5-bromo-3-(cyclopentyloxy)picolinaldehyde (390 mg, 1.44 mmol), 4-(2-(4-chloro-2-fluorophenyl)-2-methylbenzo[d][1,3]dioxol-4-yl)piperidine (502 mg, 1.44 mmol) in DCM (4 mL) was added NaBH(OAc)₃ (916 mg, 4.32 mmol). The reaction mixture was stirred at rt for 2 hours. The mixture was diluted with H₂O (50 mL), extracted with DCM (3×100 mL). The combined organic layers were washed with brine (150 mL). The organic layer was dried over Na₂SO₄, filtered, and concentrated under reduced pressure. The residue was purified by silica gel column chromatography, eluted with DCM/MeOH (50/1) to give 5-bromo-2-((4-(2-(4-chloro-2-fluorophenyl)-2-methylbenzo[d][1,3]dioxol-4-yl)piperidin-1-yl)methyl)-3-(cyclopentyloxy)pyridine (650 mg, yield: 75.0%). MS Calcd.: 600.1. MS Found: 602.9 [M+H+2]⁺.

### Step D: 6-((4-(2-(4-chloro-2-fluorophenyl)-2-methylbenzo[d][1,3]dioxol-4-yl)piperidin-1-yl)methyl)-5-(cyclopentyloxy)nicotinonitrile

A mixture of 5-bromo-2-((4-(2-(4-chloro-2-fluorophenyl)-2-methylbenzo[d][1,3]dioxol-4-yl)piperidin-1-yl)methyl)-3-(cyclopentyloxy)pyridine (200 mg, 0.33 mmol), Zn(CN)₂ (117 mg, 1.00 mmol), Pd(PPh₃)₄ (34 mg, 0.03 mmol) in DMF (2 mL) was stirred under N₂ and microwave at 160° C. for 2 hours. The mixture was diluted with H₂O (50 mL), extracted with EtOAc (3×100 mL). The combined organic layers were washed with brine (150 mL). The organic layer was dried over Na₂SO₄, filtered, and concentrated under reduced pressure. The residue was purified by silica gel column chromatography, eluted with DCM/MeOH (30/1) to give 6-((4-(2-(4-chloro-2-fluorophenyl)-2-methylbenzo[d][1,3]dioxol-4-yl)piperidin-1-yl)methyl)-5-(cyclopentyloxy)nicotinonitrile (114 mg, yield: 63.6%). MS Calcd.: 547.20. MS Found: 548.1 [M+H]⁺.

### Step E: (E)-6-((4-(2-(4-chloro-2-fluorophenyl)-2-methylbenzo[d][1,3]dioxol-4-yl)piperidin-1-yl)methyl)-5-(cyclopentyloxy)-N′-hydroxynicotinimidamide

A mixture of 6-((4-(2-(4-chloro-2-fluorophenyl)-2-methylbenzo[d][1,3]dioxol-4-yl)piperidin-1-yl)methyl)-5-(cyclopentyloxy)nicotinonitrile (144 mg, 0.21 mmol), TEA (210 mg, 2.08 mmol), NH₂OH—HCl (87 mg, 1.25 mmol) in EtOH (2 mL). The reaction mixture was stirred at 90° C. for 2 hours. The mixture was diluted with H₂O (50 mL), extracted with EA (3×100 mL). The combined organic layers were washed with brine (150 mL). The organic layer was dried over Na₂SO₄, filtered, and concentrated under reduced pressure. The residue was purified by silica gel column chromatography, eluted with DCM/MeOH (10/1) to give (E)-6-((4-(2-(4-chloro-2-fluorophenyl)-2-methylbenzo[d][1,3]dioxol-4-yl)piperidin-1-yl)methyl)-5-(cyclopentyloxy)-N′-hydroxynicotinimidamide (115 mg, yield: 95.2%). MS Calcd.: 580.23. MS Found: 581.0 [M+H]⁺.

### Step F: 3-(6-((4-(2-(4-chloro-2-fluorophenyl)-2-methylbenzo[d][1,3]dioxol-4-yl)piperidin-1-yl)methyl)-5-(cyclopentyloxy)pyridin-3-yl)-5-(trifluoromethyl)-1,2,4-oxadiazole

A mixture of (E)-6-((4-(2-(4-chloro-2-fluorophenyl)-2-methylbenzo[d][1,3]dioxol-4-yl)piperidin-1-yl)methyl)-5-(cyclopentyloxy)-N′-hydroxynicotinimidamide (115 mg, 0.20 mmol), TFAA (166 mg, 0.79 mmol) in THF (2 mL). The reaction mixture was stirred at rt for 4 hours. The mixture was diluted with H₂O (50 mL), extracted with EA (3×100 mL). The combined organic layers were washed with brine (150 mL). The organic layer was dried over Na₂SO₄, filtered, and concentrated under reduced pressure. The residue was purified by silica gel column chromatography, eluted with DCM/MeOH (10/1) to give 3-(6-((4-(2-(4-chloro-2-fluorophenyl)-2-methylbenzo[d][1,3]dioxol-4-yl)piperidin-1-yl)methyl)-5-(cyclopentyloxy)pyridin-3-yl)-5-(trifluoromethyl)-1,2,4-oxadiazole (130 mg, yield: 98.6%). MS Calcd.: 658.20. MS Found: 659.0 [M+H]⁺.

### Step G: 2-((4-(2-(4-chloro-2-fluorophenyl)-2-methylbenzo[d][1,3]dioxol-4-yl)piperidin-1-yl)methyl)-3-(cyclopentyloxy)-5-(5-(trifluoromethyl)-4H-1,2,4-triazol-3-yl)pyridine (F-12)

A mixture of 3-(6-((4-(2-(4-chloro-2-fluorophenyl)-2-methylbenzo[d][1,3]dioxol-4-yl)piperidin-1-yl)methyl)-5-(cyclopentyloxy)pyridin-3-yl)-5-(trifluoromethyl)-1,2,4-oxadiazole (130 mg, 0.20 mmol) and NH₂NH₂·H₂O (29.6 mg, 0.59 mmol) in EtOH (3 mL) was stirred at 80° C. for 16 hours. After the reaction was completed. The reaction mixture was purified by Prep-HPLC to give 2-((4-(2-(4-chloro-2-fluorophenyl)-2-methylbenzo[d][1,3]dioxol-4-yl)piperidin-1-yl)methyl)-3-(cyclopentyloxy)-5-(5-(trifluoromethyl)-4H-1,2,4-triazol-3-yl)pyridine (14.9 mg, yield: 11.3%). MS Calcd.: 657.2. MS Found: 658.2 [M+H]⁺.

¹H NMR (400 MHz, DMSO-d₆): 8.76 (d, J=1.6 Hz, 1H), 7.94 (s, J=1.2 Hz, 1H), 7.53-7.59 (m, 2H), 7.34 (dd, J=8.4 Hz, 1.6 Hz, 1H), 6.79-6.80 (m, 2H), 6.71-6.75 (m, 1H), 5.04-5.05 (m, 1H), 3.91 (s, 2H), 3.16-3.18 (m, 2H), 2.67-2.71 (m, 1H), 2.55-2.61 (m, 2H), 1.89-2.02 (m, 5H), 1.65-1.83 (m, 10H).

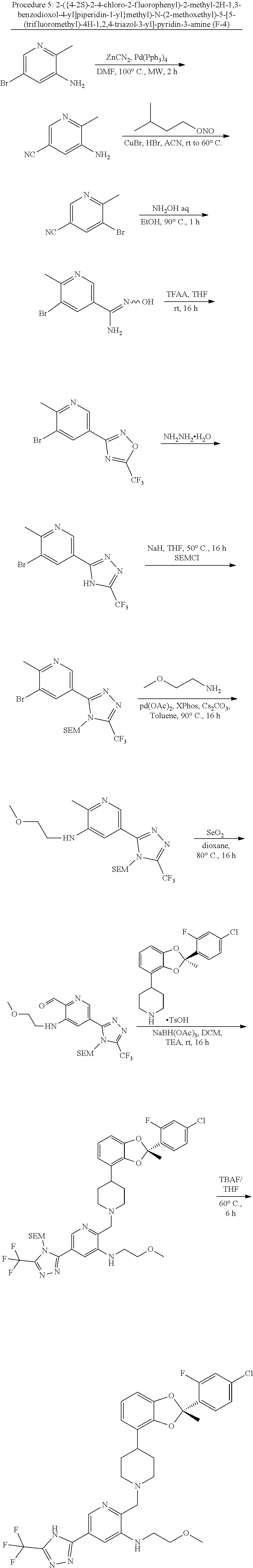

### Step A: 5-amino-6-methylnicotinonitrile

A mixture of 5-bromo-2-methylpyridin-3-amine (8.0 g, 42.8 mmol), Zn(CN)₂ (15.0 g, 128.2 mmol) and Pd(PPh₃)₄ (2.48 g, 2.15 mmol) in DMF (20 mL) was stirred at 100° C. under Ar with microwave irradiation for 2 h. The mixture reaction was filtered, the filter cake was washed with a small amount of DMF, then the filtrate was purified by flash reversed-phase chromatography column and concentrated under vacuum to give 5-amino-6-methylnicotinonitrile (3.0 g, yield: 52.8%).

### Step B: 5-bromo-6-methylnicotinonitrile

To a solution of 5-amino-6-methylnicotinonitrile (3.0 g, 21.9 mmol) and isopentyl nitrite (15.5 g, 87.6 mmol) in ACN (50 ml). The mixture was stirred at rt for 2 h, then heated to 60° C. and stirred for 2 h. HBr in water (48% in water, 140 mL) and CuBr (4.7 g, 32.8 mmol) was added, and the mixture was stirred at 60° C. under Ar with for 2 h. The mixture was poured into aqueous NaHCO₃ (50 mL) and extracted with EtOAc (2×50 mL), the combined organic layer was washed with brine, dried over sodium sulfate, filtered and the filtrate was concentrated. The residue was purified by column chromatography on silica gel (PE:EA=20:1) to give 5-bromo-6-methylnicotinonitrile (2.8 g, yield: 65.1%).

MS Calcd: 196.0. MS Found: 197.2 [M+H]⁺

### Step C: 5-bromo-N′-hydroxy-6-methylnicotinimidamide

A mixture of 5-bromo-6-methylnicotinonitrile (2.8 g, 14.3 mmol) and NH₂OH aqueous solution (50%) (9.1 g, 142.9 mmol) in EtOH (30 mL) was stirred at 90° C. for 1 h. The mixture was concentrated to give 5-bromo-N′-hydroxy-6-methylnicotinimidamide (3.0 g, crude).

MS Calcd: 229.0. MS Found: 230.0 [M+H]⁺.

### Step D: 3-(5-bromo-6-methylpyridin-3-yl)-5-(trifluoromethyl)-1,2,4-oxadiazole

To a solution of 5-bromo-N′-hydroxy-6-methylnicotinimidamide (3.0 g, crude) in THF (10 mL) were added TFAA (25.7 g, 131.0 mmol) in THF (20 mL) in a dropwise manner at 0° C. The mixture was stirred at rt for 16 h. The mixture was poured into sodium bicarbonate aqueous solution (50 mL) and extracted with EtOAc (2×50 mL). The combined organic layer was washed with brine, dried over sodium sulfate, filtered and the filtrate was concentrated to give 3-(5-bromo-6-methylpyridin-3-yl)-5-(trifluoromethyl)-1,2,4-oxadiazole (4.0 g).

MS Calcd: 307.0. MS Found: 308.1 [M+H]⁺

### Step E: 3-bromo-2-methyl-5-(5-(trifluoromethyl)-4H-1,2,4-triazol-3-yl)pyridine

A mixture of 3-(5-bromo-6-methylpyridin-3-yl)-5-(trifluoromethyl)-1,2,4-oxadiazole (4.0 g, 13.0 mmol) and NH₂NH₂·H₂O (6 ml) in EtOH (30 mL) was stirred at 70° C. for 1 h. The mixture was purified by prep-HPLC to give 3-bromo-2-methyl-5-(5-(trifluoromethyl)-4H-1,2,4-triazol-3-yl)pyridine (4.5 g, crude).

MS Calcd: 306.0. MS Found: 307.1 [M+H]⁺

### Step F: 3-bromo-2-methyl-5-(5-(trifluoromethyl)-4-((2-(trimethylsilyl)ethoxy)methyl)-4H-1,2,4-triazol-3-yl)pyridine

To a solution of 3-bromo-2-methyl-5-(5-(trifluoromethyl)-4H-1,2,4-triazol-3-yl)pyridine (4.5 g, 14.7 mmol) in THF (150 mL). Then NaH (60%, w/w 2.4 g, 58.8 mmol) was added and the mixture was stirred at 50° C. for 0.5 h. Then SEMCl (7.4 g, 44.10 mmol) was added and stirred at 50° C. for 16 h. The mixture was poured water (100 mL) and extracted with EtOAc (2×100 mL), the combined organic layer was washed with brine, dried over sodium sulfate, filtered and the filtrate was concentrated. The residue was purified by column chromatography on silica gel (PE:EA=8:1) to give 3-bromo-2-methyl-5-(5-(trifluoromethyl)-4-((2-(trimethylsilyl)ethoxy)methyl)-4H-1,2,4-triazol-3-yl)pyridine (4.3 g, yield: 67.5%).

MS Calcd: 436.0. MS Found: 437.1 [M+H]⁺

Several regio-isomers maybe exist. We choose A-1 as a representative structure.

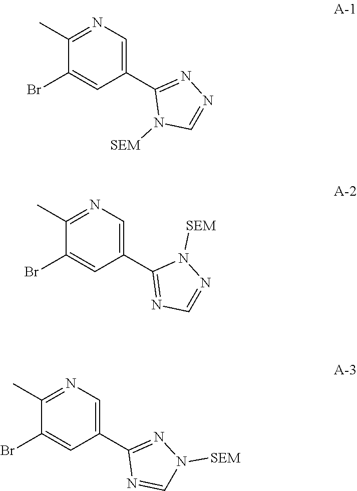 [SMILES: Cc1ncc(-c2nncn2COCC[Si](C)(C)C)cc1Br ; Cc1ncc(-c2ncnn2COCC[Si](C)(C)C)cc1Br ; Cc1ncc(-c2ncn(COCC[Si](C)(C)C)n2)cc1Br]

### Step G: N-(2-methoxyethyl)-2-methyl-5-(5-(trifluoromethyl)-4-((2-(trimethylsilyl)ethoxy)methyl)-4H-1,2,4-triazol-3-yl)pyridin-3-amine

A mixture of 3-bromo-2-methyl-5-(5-(trifluoromethyl)-4-((2-(trimethylsilyl)ethoxy)methyl)-4H-1,2,4-triazol-3-yl)pyridine (60 mg, 0.14 mmol), 2-methoxyethan-1-amine (20 mg, 0.28 mmol), Pd(OAc)₂ (3 mg, 0.01 mmol), Xphos (13 mg, 0.03 mmol) and Cs₂CO₃ (134 mg, 0.41 mmol) in toluene (5 mL) was stirred at 90° C. under Argon for 16 hours. The mixture was filtered, and the filtrate was concentrated. The residue was purified by prep-TLC (PE:EA=2:1) to give N-(2-methoxyethyl)-2-methyl-5-(5-(trifluoromethyl)-4-((2-(trimethylsilyl)ethoxy)methyl)-4H-1,2,4-triazol-3-yl)pyridin-3-amine (32 mg, yield: 53.0%).

MS Calcd: 431.2. MS Found: 432.2 [M+H]⁺

### Step H: 3-((2-methoxyethyl)amino)-5-(5-(trifluoromethyl)-4-((2-(trimethylsilyl)ethoxy)methyl)-4H-1,2,4-triazol-3-yl)picolinaldehyde

A mixture of N-(2-methoxyethyl)-2-methyl-5-(5-(trifluoromethyl)-4-((2-(trimethylsilyl)ethoxy)methyl)-4H-1,2,4-triazol-3-yl)pyridin-3-amine (32 mg, 0.07 mmol) and SeO₂ (82 mg, 0.74 mmol) in 1,4-dioxane (2 mL) was stirred at 80° C. for 16 hours. The mixture was filtered, and the filtrate was concentrated to give 3-((2-methoxyethyl)amino)-5-(5-(trifluoromethyl)-4-((2-(trimethylsilyl)ethoxy)methyl)-4H-1,2,4-triazol-3-yl)picolinaldehyde (33 mg, crude). MS Calcd: 445.2; MS Found: 446.3 [M+H]⁺

### Step I: (S)-2-((4-(2-(4-chloro-2-fluorophenyl)-2-methylbenzo[d][1,3]dioxol-4-yl)piperidin-1-yl)methyl)-N-(2-methoxyethyl)-5-(5-(trifluoromethyl)-4-((2-(trimethylsilyl)ethoxy)methyl)-4H-1,2,4-triazol-3-yl)pyridin-3-amine

A mixture of (R)-4-(2-(4-chloro-2-fluorophenyl)-2-methylbenzo[d][1,3]dioxol-4-yl)piperidine (43 mg, 0.08 mmol) and TEA (38 mg, 0.38 mmol) in DCM (5 mL) was stirred at rt for 0.5 h. 3-((2-methoxyethyl)amino)-5-(5-(trifluoromethyl)-4-((2-(trimethylsilyl)ethoxy)methyl)-4H-1,2,4-triazol-3-yl)picolinaldehyde (33 mg, 0.07 mmol) was added and the mixture was stirred for 2 hours. NaBH(OAc)₃ (63 mg, 0.30 mmol) was added and the mixture was stirred at rt for 16 h. The mixture was poured into water (30 mL) and extracted with DCM (2×30 mL), the combined organic layer was washed with brine, dried over sodium sulfate, filtered and the filtrate was concentrated to give (S)-2-((4-(2-(4-chloro-2-fluorophenyl)-2-methylbenzo[d][1,3]dioxol-4-yl)piperidin-1-yl)methyl)-N-(2-methoxyethyl)-5-(5-(trifluoromethyl)-4-((2-(trimethylsilyl)ethoxy)methyl)-4H-1,2,4-triazol-3-yl)pyridin-3-amine (50 mg, yield: 92.0%).

MS Calcd: 776.3. MS Found: 777.4 [M+H]⁺

### Step J: 2-({4-[(2S)-2-(4-chloro-2-fluorophenyl)-2-methyl-2H-1,3-benzodioxol-4-yl]piperidin-1-yl}methyl)-N-(2-methoxyethyl)-5-[5-(trifluoromethyl)-4H-1,2,4-triazol-3-yl]pyridin-3-amine (F-4)

A mixture of (S)-2-((4-(2-(4-chloro-2-fluorophenyl)-2-methylbenzo[d][1,3]dioxol-4-yl)piperidin-1-yl)methyl)-N-(2-methoxyethyl)-5-(5-(trifluoromethyl)-4-((2-(trimethylsilyl)ethoxy)methyl)-4H-1,2,4-triazol-3-yl)pyridin-3-amine (50 mg, 0.06 mmol) and TABF (1 M, 2 mL) in THE (1 mL) was stirred at 60° C. for 6 h. The mixture was purified by prep-HPLC to give 2-({4-[(2S)-2-(4-chloro-2-fluorophenyl)-2-methyl-2H-1,3-benzodioxol-4-yl]piperidin-1-yl}methyl)-N-(2-methoxyethyl)-5-[5-(trifluoromethyl)-4H-1,2,4-triazol-3-yl]pyridin-3-amine (F-4) (5.3 mg, yield: 13.8%).

MS Calcd: 646.2. MS Found: 647.3 [M+H]⁺

¹H NMR (400 MHz, CD₃OD): δ 8.37 (s, 1H), 7.63-7.55 (m, 2H), 7.34-7.17 (m, 2H), 6.83-6.67 (m, 3H), 3.86 (s, 2H), 3.77-3.68 (m, 2H), 3.50-3.37 (m, 6H), 3.08-2.97 (m, 2H), 2.83-2.70 (m, 1H), 2.40-2.25 (m, 2H), 2.03 (s, 3H), 1.99-1.80 (m, 4H). ¹⁹F-NMR (377 MHz, CD₃OD): δ −66.36, −112.28.

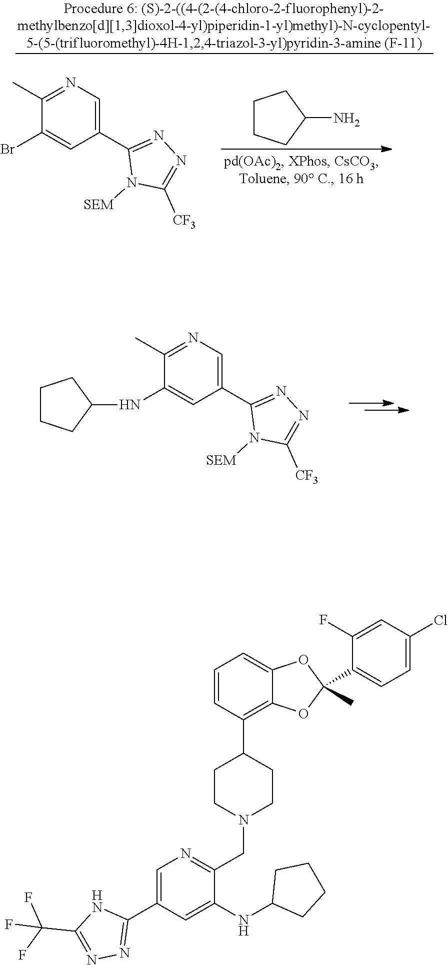 [SMILES: Cc1ncc(-c2nnc(C(F)(F)F)n2COCC[Si](C)(C)C)cc1Br ; NC1CCCC1 ; Cc1ncc(-c2nnc(C(F)(F)F)n2COCC[Si](C)(C)C)cc1NC1CCCC1 ; C[C@]1(c2ccc(Cl)cc2F)Oc2cccc(C3CCN(Cc4ncc(-c5nnc(C(F)(F)F)[nH]5)cc4NC4CCCC4)CC3)c2O1]

### Step A: N-cyclopentyl-2-methyl-5-(5-(trifluoromethyl)-4-((2-(trimethylsilyl)ethoxy)methyl)-4H-1,2,4-triazol-3-yl)pyridin-3-amine

A mixture of 3-bromo-2-methyl-5-(5-(trifluoromethyl)-4-((2-(trimethylsilyl)ethoxy)methyl)-4H-1,2,4-triazol-3-yl)pyridine (60 mg, 0.14 mmol), cyclopentanamine (23 mg, 0.28 mmol), Pd(OAc)₂ (3 mg, 0.014 mmol), XPhos (13 mg, 0.027 mmol) and Cs₂CO₃ (134 mg, 0.41 mmol) in Toluene (5 mL). The mixture was stirred at 90° C. under Argon for 16 h. The mixture was poured into water (100 mL) and extracted with EtOAC (2×100 mL), the combined organic layer was washed with brine, dried over sodium sulfate, filtered and the residue was concentrated. The residue was removed of volatiles under vacuum and purified by prep-TLC to obtain N-cyclopentyl-2-methyl-5-(5-(trifluoromethyl)-4-((2-(trimethylsilyl)ethoxy)methyl)-4H-1,2,4-triazol-3-yl)pyridin-3-amine (32 mg, yield: 52.5%). MS Calcd: 441.2. MS Found: 442.2 [M+H]⁺

### Step B: (S)-2-((4-(2-(4-chloro-2-fluorophenyl)-2-methylbenzo[d][1,3]dioxol-4-yl)piperidin-1-yl)methyl)-N-cyclopentyl-5-(5-(trifluoromethyl)-4H-1,2,4-triazol-3-yl)pyridin-3-amine (F-11)

(S)-2-((4-(2-(4-chloro-2-fluorophenyl)-2-methylbenzo[d][1,3]dioxol-4-yl)piperidin-1-yl)methyl)-N-cyclopentyl-5-(5-(trifluoromethyl)-4H-1,2,4-triazol-3-yl)pyridin-3-amine (2.3 mg) was obtained by a similar method to Procedure 5. MS Calcd: 656.2. MS Found: 659.5 [M+H]⁺

¹H NMR (400 MHz, CD₃OD): δ 8.33 (s, 1H), 7.63-7.55 (m, 2H), 7.31-7.18 (m, 2H), 6.80-6.74 (m, 1H), 6.71-6.66 (m, 2H), 3.96-3.91 (m, 1H), 3.84-3.79 (m, 2H), 3.06-2.95 (m, 2H), 2.76-2.65 (m, 1H), 2.33-2.24 (m, 2H), 2.16-2.04 (m, 2H), 2.04 (s, 3H), 1.92-1.57 (m, 10H). ¹⁹F NMR (377 MHz, CD₃OD): δ −66.22, −112.06.

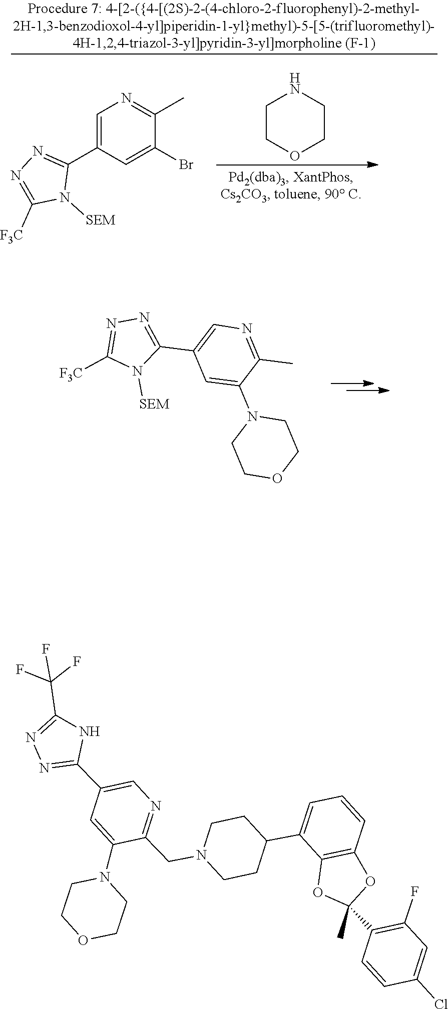 [SMILES: Cc1ncc(-c2nnc(C(F)(F)F)n2COCC[Si](C)(C)C)cc1Br ; C1COCCN1 ; Cc1ncc(-c2nnc(C(F)(F)F)n2COCC[Si](C)(C)C)cc1N1CCOCC1 ; C[C@]1(c2ccc(Cl)cc2F)Oc2cccc(C3CCN(Cc4ncc(-c5nnc(C(F)(F)F)[nH]5)cc4N4CCOCC4)CC3)c2O1]

### Step A: 4-(2-methyl-5-(5-(trifluoromethyl)-4-((2-(trimethylsilyl)ethoxy)methyl)-4H-1,2,4-triazol-3-yl)pyridin-3-yl)morpholine

A mixture of 3-bromo-2-methyl-5-(5-(trifluoromethyl)-4-((2-(trimethylsilyl)ethoxy)methyl)-4H-1,2,4-triazol-3-yl)pyridine (50 mg, 0.11 mmol), morpholine (20 mg, 0.23 mmol), Pd(OAc)₂ (3 mg, 0.01 mmol), Xphos (11 mg, 0.02 mmol) and Cs₂CO₃ (112 mg, 0.34 mmol) in toluene (8 mL) was stirred at 90° C. under Argon for 16 hours. The mixture was filtered, and the filtrate was concentrated. The residue was purified by prep-TLC (PE:EA=2:1) to give 4-(2-methyl-5-(5-(trifluoromethyl)-4-((2-(trimethylsilyl)ethoxy)methyl)-4H-1,2,4-triazol-3-yl)pyridin-3-yl)morpholine (40 mg, yield: 78.7%).

MS Calcd: 443.2. MS Found: 444.6 [M+H]⁺

### Step B: 4-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]morpholine (F-1)

4-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]morpholine (F-1) (5.0 mg) was obtained by a similar method to Procedure 5.

MS Calcd: 658.2. MS Found: 659.3 [M+H]⁺

¹H NMR (400 MHz, CD₃OD): δ 9.05 (d, J=1.6 Hz, 1H), 8.29 (d, J=1.6 Hz, 1H), 7.62 (t, J=8.4 Hz, 1H), 7.33-7.20 (m, 2H), 6.89-6.73 (m, 3H), 4.61 (s, 2H), 3.91 (t, J=4.4 Hz, 4H), 3.70-3.60 (m, 2H), 3.30-3.21 (m, 2H), 3.09-3.00 (m, 5H), 2.35-2.05 (m, 4H), 2.08 (s, 3H). ¹⁹F-NMR (377 MHz, CD₃OD): δ −65.47, −112.21.

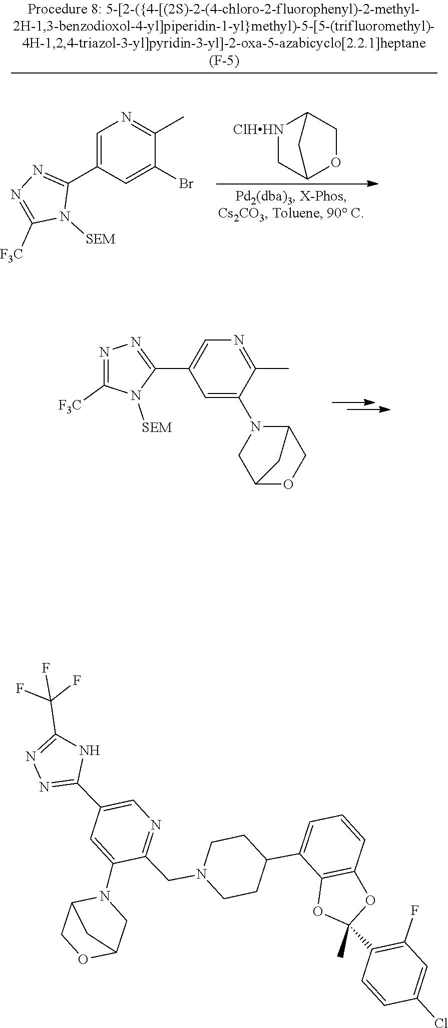

### Step A: 5-(2-methyl-5-(5-(trifluoromethyl)-4-((2-(trimethylsilyl)ethoxy)methyl)-4H-1,2,4-triazol-3-yl)pyridin-3-yl)-2-oxa-5-azabicyclo[2.2.1]heptane

A mixture of 3-bromo-2-methyl-5-(5-(trifluoromethyl)-4-((2-(trimethylsilyl)ethoxy)methyl)-4H-1,2,4-triazol-3-yl)pyridine (50 mg, 0.11 mmol), 2-oxa-5-azabicyclo[2.2.1]heptane hydrochloride (15 mg, 0.11 mmol), Pd(OAc)₂ (3 mg, 0.01 mmol), Xphos (11 mg, 0.02 mmol) and Cs₂CO₃ (150 mg, 0.46 mmol) in toluene (8 mL) was stirred at 90° C. under Argon for 16 hours. The mixture was filtered, and the filtrate was concentrated. The residue was purified by prep-TLC (PE:EA=2:1) to give 5-(2-methyl-5-(5-(trifluoromethyl)-4-((2-(trimethylsilyl)ethoxy)methyl)-4H-1,2,4-triazol-3-yl)pyridin-3-yl)-2-oxa-5-azabicyclo[2.2.1]heptane (46 mg, yield: 88.2%).

MS Calcd: 455.2. MS Found: 456.3 [M+H]⁺

### Step B: 5-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]-2-oxa-5-azabicyclo[2.2.1]heptane (F-5)

5-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]-2-oxa-5-azabicyclo[2.2.1]heptane (F-5) (1.0 mg) was obtained by a similar method to Procedure 5.

MS Calcd: 670.2. MS Found: 671.4 [M+H]⁺

¹H NMR (400 MHz, CD₃OD): δ 8.82 (s, 1H), 8.00 (s, 1H), 7.62 (t, J=8.4 Hz, 1H), 7.34-7.18 (m, 2H), 6.88-6.72 (m, 3H), 4.67 (s, 1H), 4.60-4.49 (m, 1H), 4.44 (s, 1H), 4.39-4.30 (m, 1H), 4.11 (d, J=8 Hz, 1H), 3.90 (d, J=8 Hz, 1H), 3.73-3.50 (m, 4H), 3.30-3.15 (m, 2H), 3.08-2.98 (m, 1H), 2.35-2.13 (m, 2H), 2.13-1.98 (m, 7H). ¹⁹F-NMR (377 MHz, CD₃OD): δ −64.99, −112.25.

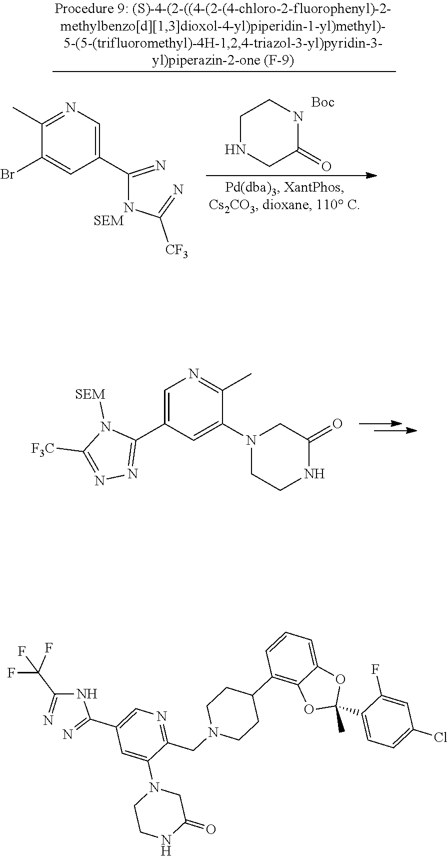

### Step A: 4-(2-methyl-5-(5-(trifluoromethyl)-4-((2-(trimethylsilyl)ethoxy)methyl)-4H-1,2,4-triazol-3-yl)pyridin-3-yl)piperazin-2-one

A mixture of 3-bromo-2-methyl-5-(5-(trifluoromethyl)-4-((2-(trimethylsilyl)ethoxy)methyl)-4H-1,2,4-triazol-3-yl)pyridine (50 mg, 0.115 mmol), tert-butyl 2-oxopiperazine-1-carboxylate (23 mg, 0.115 mmol) and Pd₂(dba)₃ (11 mg, 0.011 mmol) in toluene (5 mL) was stirred with Xantphos (13 mg, 0.023 mmol) and Cs₂CO₃ (112 mg, 0.344 mmol) at 110° C. under Argon for 16 hours. The mixture was poured into water (50 mL) and extracted with EA (2×50 mL), the combined organic layer was washed with brine, dried over sodium sulfate, filtered and the residue was concentrated. The residue was purified by TLC gel to obtain 4-(2-methyl-5-(5-(trifluoromethyl)-4-((2-(trimethylsilyl)ethoxy)methyl)-4H-1,2,4-triazol-3-yl)pyridin-3-yl)piperazin-2-one (50 mg, yield: 78.10%). MS Calcd: 456.2. MS Found: 457.3 [M+H]⁺

### Step D: (S)-4-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)piperazin-2-one

(S)-4-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)piperazin-2-one (0.5 mg) was obtained by a similar method to Procedure 5. MS Calcd: 671.2. MS Found: 672.3 [M+H]⁺

¹H NMR (400 MHz, CD₃OD): δ 9.06 (s, 1H), 8.34 (s, 1H), 7.65-7.59 (m, 1H), 7.30-7.21 (m, 2H), 6.84-6.73 (m, 3H), 4.54 (s, 2H), 3.73 (s, 2H), 3.58-3.48 (m, 2H), 3.43-3.41 (m, 2H), 3.07-3.03 (m, 4H), 3.01 (s, 1H), 2.28-2.13 (m, 2H), 2.02-2.00 (m, 5H). ¹⁹F NMR (377 MHz, CD₃OD-d₄): δ −64.96, −112.31.

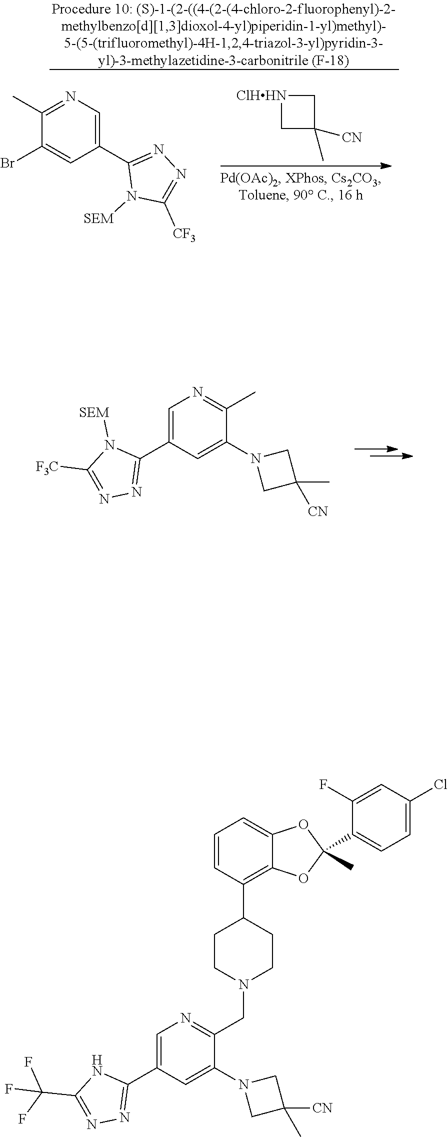

### Step A: 3-methyl-1-(2-methyl-5-(5-(trifluoromethyl)-4-((2-(trimethylsilyl)ethoxy)methyl)-4H-1,2,4-triazol-3-yl)pyridin-3-yl)azetidine-3-carbonitrile

A mixture of 3-bromo-2-methyl-5-(5-(trifluoromethyl)-4-((2-(trimethylsilyl)ethoxy)methyl)-4H-1,2,4-triazol-3-yl)pyridine (40 mg, 0.09 mmol), 3-methylazetidine-3-carbonitrile hydrochloride (24 mg, 0.18 mmol), Pd(OAC)₂ (2 mg, 0.01 mmol), Xphos (9 mg, 0.02 mmol) and Cs₂CO₃ (120 mg, 0.37 mmol) in toluene (5 mL) was stirred at 90° C. under Argon for 16 h. The mixture was filtered, and the residue was concentrated. The residue was purified by prep-TLC to obtain 3-methyl-1-(2-methyl-5-(5-(trifluoromethyl)-4-((2-(trimethylsilyl)ethoxy)methyl)-4H-1,2,4-triazol-3-yl)pyridin-3-yl)azetidine-3-carbonitrile (40 mg, yield: 96.5%). MS Calcd: 452.2. MS Found: 453.3 [M+H]⁺

### Step B: (S)-1-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-3-methylazetidine-3-carbonitrile (F-18)

(S)-1-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-3-methylazetidine-3-carbonitrile (13 mg) was obtained by a similar method to Procedure 5. MS Calcd: 667.2. MS Found: 668.4 [M+H]⁺

¹H NMR (400 MHz, CD₃OD): δ 8.80 (d, J=1.6 Hz, 1H), 7.67-7.59 (m, 2H), 7.33-7.20 (m, 2H), 6.89-6.74 (m, 3H), 4.53 (s, 2H), 4.46 (d, J=7.2 Hz, 2H), 4.10 (d, J=7.2 Hz, 2H), 3.79 (d, J=10.0 Hz, 2H), 3.40-3.32 (m, 2H), 3.16-3.14 (m, 1H), 2.39-2.27 (m, 2H), 2.21-2.01 (m, 2H), 2.06 (s, 3H), 1.79 (s, 3H). ¹⁹F NMR (377 MHz, CD₃OD): δ −66.74, −77.13, −112.22.

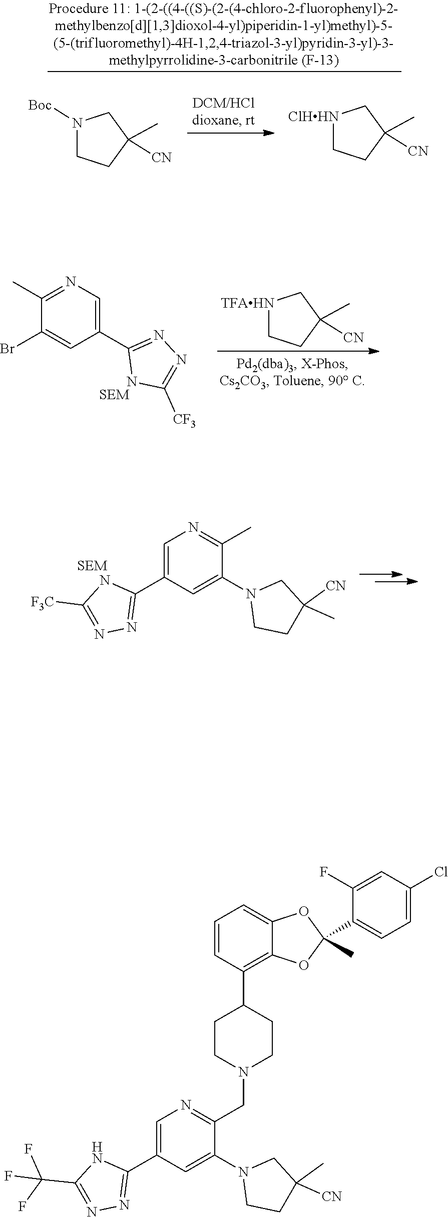

### Step A: 3-methylpyrrolidine-3-carbonitrile hydrochloride

A mixture of tert-butyl 3-cyano-3-methylpyrrolidine-1-carboxylate (150 mg, 0.71 mmol) and HCl·dioxane (1.5 mL) in DCM (4 mL) was stirred at rt for 1 h. The mixture was concentrated to obtain 3-methylpyrrolidine-3-carbonitrile hydrochloride (79 mg, yield: 96.8%). MS Calcd: 110.1. MS Found: 111.3 [M+H]⁺

### Step B: 3-methyl-1-(2-methyl-5-(5-(trifluoromethyl)-4-((2-(trimethylsilyl)ethoxy)methyl)-4H-1,2,4-triazol-3-yl)pyridin-3-yl)pyrrolidine-3-carbonitrile

A mixture of 3-bromo-2-methyl-5-(5-(trifluoromethyl)-4-((2-(trimethylsilyl)ethoxy)methyl)-4H-1,2,4-triazol-3-yl)pyridine (40 mg, 0.092 mmol), 3-methylpyrrolidine-3-carbonitrile hydrochloride (79 mg, 0.46 mmol) and Pd₂(dba)₃ (8 mg, 0.009 mmol) in toluene (5 mL) was stirred with BINAP (11 mg, 0.018 mmol) and t-BuOK (31 mg, 0.27 mmol) at 120° C. under Argon for 16 hours. The mixture was poured into water (50 mL) and extracted with EA (2×50 mL), the combined organic layer was washed with brine, dried over sodium sulfate, filtered and the residue was concentrated. The residue was purified by pre-TLC gel to obtain 3-methyl-1-(2-methyl-5-(5-(trifluoromethyl)-4-((2-(trimethylsilyl)ethoxy)methyl)-4H-1,2,4-triazol-3-yl)pyridin-3-yl)pyrrolidine-3-carbonitrile (33 mg, yield: 76.7%). MS Calcd: 466.2. MS Found: 467.4 [M+H]⁺

### Step C: 1-(2-((4-((S)-2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-3-methylpyrrolidine-3-carbonitrile

1-(2-((4-((S)-2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-3-methylpyrrolidine-3-carbonitrile (10.7 mg) was obtained by a similar method to Procedure 5. MS Calcd: 681.2. MS Found: 682.4 [M+H]⁺

¹H NMR (400 MHz, CD₃OD-d₄): δ 8.88 (s, 1H), 8.09 (s, 1H), 7.61 (t, J=8.4 Hz, 1H), 7.29 (d, J=11.2 Hz, 1H), 7.22 (d, J=9.6 Hz, 1H), 6.83-6.76 (m, 3H), 4.52-4.48 (m, 2H), 3.78-3.61 (m, 4H), 3.39-3.37 (m, 1H), 3.24-3.15 (m, 2H), 3.04-2.98 (m, 1H), 2.58-2.50 (m, 1H), 2.21-2.05 (m, 9H), 1.62 (s, 3H). ¹⁹F NMR (377 MHz, CD₃OD-d₄): δ: (−65.22), (−112.23).

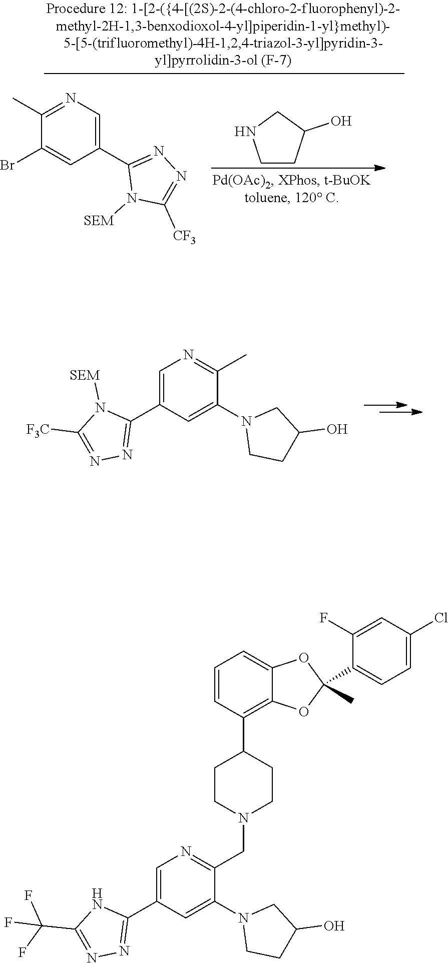 [SMILES: Cc1ncc(-c2nnc(C(F)(F)F)n2COCC[Si](C)(C)C)cc1Br ; OC1CCNC1 ; Cc1ncc(-c2nnc(C(F)(F)F)n2COCC[Si](C)(C)C)cc1N1CCC(O)C1 ; C[C@]1(c2ccc(Cl)cc2F)Oc2cccc(C3CCN(Cc4ncc(-c5nnc(C(F)(F)F)[nH]5)cc4N4CCC(O)C4)CC3)c2O1]

### Step A: 1-(2-methyl-5-(5-(trifluoromethyl)-4-((2-(trimethylsilyl)ethoxy)methyl)-4H-1,2,4-triazol-3-yl)pyridin-3-yl)pyrrolidin-3-ol

A mixture of 3-bromo-2-methyl-5-(5-(trifluoromethyl)-4-((2-(trimethylsilyl)ethoxy)methyl)-4H-1,2,4-triazol-3-yl)pyridine (20.00 mg, 0.459 mmol), pyrrolidin-3-ol (14.00 mg, 0.115 mmol) and Pd₂(dba)₃ (4 mg, 0.005 mmol) in toluene (5 mL) was stirred with BINAP (6 mg, 0.009 mmol) and t-BuOK(15 mg, 0.138 mmol) at 120° C. under Argon for 16 hours. The mixture was poured into water (50 mL) and extracted with DCM (2×50 mL), the combined organic layer was washed with brine, dried over sodium sulfate, filtered and the filtrate was concentrated. The residue was purified by TLC gel to give 1-(2-methyl-5-(5-(trifluoromethyl)-4-((2-(trimethylsilyl)ethoxy)methyl)-4H-1,2,4-triazol-3-yl)pyridin-3-yl)pyrrolidin-3-ol (24 mg, yield: 82.3%).

MS Calcd: 443.2. MS Found: 444.0 [M+H]⁺

### Step B: 1-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]pyrrolidin-3-ol (F-7)

1-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]pyrrolidin-3-ol (F-7) (0.8 mg) was obtained by a similar method to Procedure 5.

MS Calcd: 658.2. MS Found: 659.0 [M+H]⁺

¹H NMR (400 MHz, CD₃OD): δ 8.80 (s, 1H), 8.48 (s, 1H), 7.99 (s, 1H), 7.65-7.60 (m, 1H), 7.30 (dd, J=1.2 Hz/J=10.0 Hz, 1H), 7.22 (dd, J=2.4 Hz/J=4.8 Hz, 1H), 6.86-6.75 (m, 3H), 5.37-5.32 (m, 1H), 4.57-4.53 (m, 4H), 3.70-3.55 (m, 4H), 3.23-3.20 (m, 2H), 3.08-2.99 (m, 1H), 2.38-2.17 (m, 2H), 2.17-1.99 (m, 7H). ¹⁹F-NMR (377 MHz, CD₃OD): δ −65.28, −112.25.

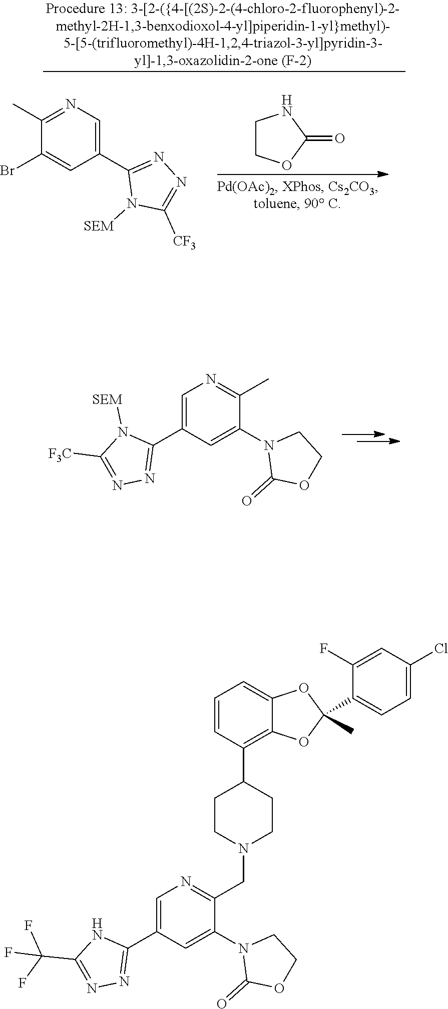 [SMILES: Cc1ncc(-c2nnc(C(F)(F)F)n2COCC[Si](C)(C)C)cc1Br ; O=C1NCCO1 ; Cc1ncc(-c2nnc(C(F)(F)F)n2COCC[Si](C)(C)C)cc1N1CCOC1=O ; C[C@]1(c2ccc(Cl)cc2F)Oc2cccc(C3CCN(Cc4ncc(-c5nnc(C(F)(F)F)[nH]5)cc4N4CCOC4=O)CC3)c2O1]

### Step A: 3-(2-methyl-5-(5-(trifluoromethyl)-4-((2-(trimethylsilyl)ethoxy)methyl)-4H-1,2,4-triazol-3-yl)pyridin-3-yl)oxazolidin-2-one

A mixture of 3-bromo-2-methyl-5-(5-(trifluoromethyl)-4-((2-(trimethylsilyl)ethoxy)methyl)-4H-1,2,4-triazol-3-yl)pyridine (40 mg, 0.09 mmol), oxazolidin-2-one (40 mg, 0.46 mmol), Pd(OAc)₂ (2 mg, 0.01 mmol), Xphos (9 mg, 0.01 mmol) and Cs₂CO₃ (89 mg, 0.27 mmol) in toluene (5 mL) was stirred at 90° C. under Argon for 16 hours. The mixture was filtered, and the filtrate was concentrated. The residue was purified by prep-TLC (DCM:MeOH=30:1) to give 3-(2-methyl-5-(5-(trifluoromethyl)-4-((2-(trimethylsilyl)ethoxy)methyl)-4H-1,2,4-triazol-3-yl)pyridin-3-yl)oxazolidin-2-one (35 mg, yield: 87.8%).

MS Calcd: 443.2. MS Found: 444.3 [M+H]⁺

### Step B: 3-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]-1,3-oxazolidin-2-one (F-2)

3-[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]-1,3-oxazolidin-2-one (F-2) (7.1 mg) was obtained by a similar method to Procedure 5.

MS Calcd: 658.2. MS Found: 659.4 [M+H]⁺

¹H NMR (400 MHz, CD₃OD): δ 9.26 (s, 1H), 8.51 (s, 1H), 7.62 (t, J=8.2 Hz, 1H), 7.29 (d, J=10.8 Hz, 1H), 7.23 (d, J=8.0 Hz, 1H), 6.89-6.74 (m, 3H), 4.67 (t, J=8.0 Hz, 2H), 4.58 (s, 2H), 4.24 (t, J=7.8 Hz, 2H), 3.78-3.68 (m, 2H), 3.15-3.01 (m, 2H), 2.40-2.05 (m, 8H). ¹⁹F-NMR (377 MHz, CD₃OD): δ −66.18, −112.43.

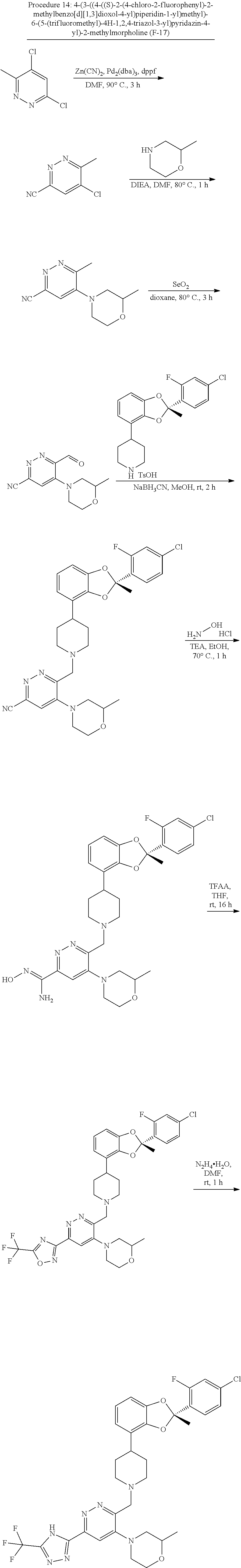

### Step A: 5-chloro-6-methylpyridazine-3-carbonitrile

A mixture of 4,6-dichloro-3-methylpyridazine (1.89 g, 11.6 mmol), zinc cyanide (0.68 g, 5.8 mmol), Pd₂(dba)₃ (0.53 g, 0.58 mmol) and dppf (0.51 g, 0.9 mmol) in DMF (20 mL) was stirred for 3 hours at 90° C. under nitrogen atmosphere. The reaction mixture was cooled to room temperature. The mixture was diluted with water (40 mL), extracted with EtOAc (3×30 mL). The combined organic layers were washed with brine (2×20 mL) and dried over anhydrous Na₂SO₄. After filtration, the filtrate was concentrated under reduced pressure. The residue was purified by silica gel column chromatography, eluted with (PE/EA=10/1) to afford 5-chloro-6-methylpyridazine-3-carbonitrile (1.59 g, yield: 89.3%).

### Step B: 6-methyl-5-(2-methylmorpholino)pyridazine-3-carbonitrile

A mixture of 5-chloro-6-methylpyridazine-3-carbonitrile (300 mg, 1.96 mmol), 2-methylmorpholine (238 mg, 2.35 mmol) and DIEA (379 mg, 2.94 mmol) in DMF (5 mL) was stirred at 80° C. for an hour under N₂ atmosphere. The reaction mixture was cooled to room temperature. The mixture was quenched with H₂O (10 mL), extracted with EA (3×20 mL). The combined organic layers were washed with brine (10 mL). The organic layer was dried over Na₂SO₄, filtered, and concentrated under reduced pressure. The residue was purified by silica gel column chromatography, eluted with PE/EA (1/1) to give 6-methyl-5-(2-methylmorpholino)pyridazine-3-carbonitrile (440 mg, yield: 92.7%, 90% purity). MS Calcd.: 218.1. MS Found: 219.1 [M+H]⁺.

### Step C: 6-formyl-5-(2-methylmorpholino)pyridazine-3-carbonitrile

A mixture of 6-methyl-5-(2-methylmorpholino)pyridazine-3-carbonitrile (400 mg, 1.83 mmol) and SeO₂ (407 mg, 3.67 mmol) in dioxane (6 mL) was stirred at 80° C. for 3 hours under an atmosphere of nitrogen. The reaction mixture was cooled to room temperature, quenched with H₂O (10 mL) and extracted with EA (3×20 mL). The combined organic layers were washed with brine (10 mL). The organic layer was dried over Na₂SO₄, filtered, and concentrated under reduced pressure. The residue was purified by silica gel column chromatography, eluted with PE/EA (1/1) to give 6-formyl-5-(2-methylmorpholino)pyridazine-3-carbonitrile (330 mg, yield: 66%, 85% purity). MS Calcd.: 232.1. MS Found: 233.1 [M+H]⁺.

### Step D: 6-((4-((S)-2-(4-chloro-2-fluorophenyl)-2-methylbenzo[d][1,3]dioxol-4-yl)piperidin-1-yl)methyl)-5-(2-methylmorpholino)pyridazine-3-carbonitrile

To a solution of 6-formyl-5-(2-methylmorpholino)pyridazine-3-carbonitrile (330 mg, 1.4 mmol) and (S)-4-(2-(4-chloro-2-fluorophenyl)-2-methylbenzo[d][1,3]dioxol-4-yl)piperidine (740 mg, 1.4 mmol, TsOH salt) in MeOH (8 mL) was added sodium cyanoborohydride (268 mg, 4.3 mmol). The reaction mixture was stirred at room temperature for 2 hours. The reaction mixture was quenched with H₂O (20 mL), extracted with EA (3×40 mL). The organic layers were combined, washed with brine (20 mL). The organic layer was dried over Na₂SO₄, filtered, and concentrated under reduced pressure. The residue was purified by Prep-TLC (PE/EA=5/1) to afford 6-((4-((S)-2-(4-chloro-2-fluorophenyl)-2-methylbenzo[d][1,3]dioxol-4-yl)piperidin-1-yl)methyl)-5-(2-methylmorpholino)pyridazine-3-carbonitrile (110 mg, yield: 13.7%). MS Calcd.: 563.2. MS Found: 564.2 [M+H]⁺.

### Step E: (Z)-6-((4-((S)-2-(4-chloro-2-fluorophenyl)-2-methylbenzo[d][1,3]dioxol-4-yl)piperidin-1-yl)methyl)-N′-hydroxy-5-(2-methylmorpholino)pyridazine-3-carboximidamide

To a solution of 6-((4-((S)-2-(4-chloro-2-fluorophenyl)-2-methylbenzo[d][1,3]dioxol-4-yl)piperidin-1-yl)methyl)-5-(2-methylmorpholino)pyridazine-3-carbonitrile (110 mg, 0.19 mmol) in ethyl alcohol (3 mL) was added hydroxylamine hydrochloride (27 mg, 0.39 mmol) and TEA (59 mg, 0.58 mmol). The resulting mixture was stirred at 70° C. for an hour. The reaction mixture was diluted with EA (20 mL), washed with H₂O (10 mL). The organic layer was dried over Na₂SO₄ and filtered. The filtrate was concentrated under reduced pressure to afford (Z)-6-((4-((S)-2-(4-chloro-2-fluorophenyl)-2-methylbenzo[d][1,3]dioxol-4-yl)piperidin-1-yl)methyl)-N′-hydroxy-5-(2-methylmorpholino)pyridazine-3-carboximidamide (110 mg, yield: 94%). The crude product was used in the next step directly without further purification. MS Calcd.: 596.2. MS Found: 597.2 [M+H]⁺.

### Step F: 4-(3-((4-((S)-2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyridazin-4-yl)-2-methylmorpholine

To a solution of (Z)-6-((4-((S)-2-(4-chloro-2-fluorophenyl)-2-methylbenzo[d][1,3]dioxol-4-yl)piperidin-1-yl)methyl)-N′-hydroxy-5-(2-methylmorpholino)pyridazine-3-carboximidamide (110 mg, 0.18 mmol) in THF (4 mL) was added TFAA (155 mg, 0.74 mmol) at room temperature. The resulting mixture was stirred at room temperature for 12 hours. The reaction mixture was quenched with saturated sodium bicarbonate aqueous solution (10 mL), and then extracted with EA (3×20 mL). The organic layers were combined, washed with brine (20 mL). The organic layer was dried over Na₂SO₄, filtered, and concentrated under reduced pressure. The residue was purified by Prep-TLC (PE/EA=2/1) to afford 4-(3-((4-((S)-2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyridazin-4-yl)-2-methylmorpholine (80 mg, yield: 64%). MS Calcd.: 674.2. MS Found: 675.2 [M+H]⁺.

### Step G: 4-(3-((4-((S)-2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)-2-methylmorpholine (F-17)

To a solution of 4-(3-((4-((S)-2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyridazin-4-yl)-2-methylmorpholine (80 mg, 0.12 mmol) in DMF (1.5 mL) was added hydrazine hydrate (24 mg, 0.47 mmol). The resulting mixture was stirred at room temperature for an hour. The solvent was removed in vacuo. The residue was purified by Prep-HPLC to afford 4-(3-((4-((S)-2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)-2-methylmorpholine (20.4 mg, yield: 25%). MS Calcd.: 673.2. MS Found: 674.0 [M+H]⁺.

¹H NMR (400 MHz, DMSO-d₆): δ 10.27 (brs, 1H), 7.77 (s, 1H), 7.57-7.65 (m, 2H), 7.35 (dd, J=8.4 Hz, 2.0 Hz, 1H), 6.84-6.92 (m, 2H), 6.73-6.80 (m, 1H), 4.71-4.89 (m, 2H), 3.93-3.96 (m, 1H), 3.73-3.85 (m, 2H), 3.53-3.68 (m, 2H), 3.28-3.45 (m, 4H), 2.93-3.13 (m, 2H), 2.74-2.84 (m, 1H), 2.10-2.26 (m, 2H), 1.92-2.05 (m, 5H), 1.19 (d, J=6.4 Hz, 3H). ¹⁹F NMR (377 MHz, DMSO-d6): δ −63.72, −110.51.

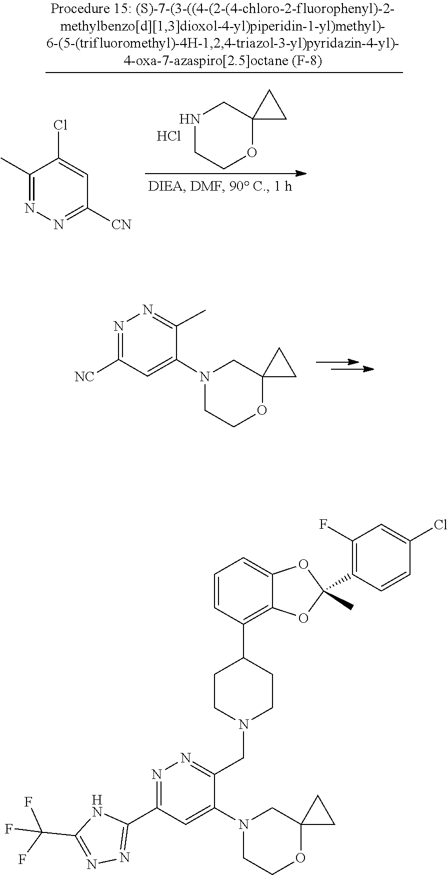

### Step A: 6-methyl-5-(4-oxa-7-azaspiro[2.5]octan-7-yl)pyridazine-3-carbonitrile

A mixture of 5-chloro-6-methylpyridazine-3-carbonitrile (450 mg, 2.94 mmol), 4-oxa-7-azaspiro[2.5]octane HCl salt (657 mg, 4.41 mmol), DIEA (1.1 g, 8.8 mmol) in DMF (7.0 mL) was stirred at 90° C. for 1 hour. After the reaction was completed, the mixture was diluted with H₂O (40 mL), extracted with EA (30 mL×2). The combined organic layer was dried over anhydrous sodium sulfate, filtered, and evaporated to dryness. The residue was purified by column chromatography on silica gel (EA/PE=½) to give 6-methyl-5-(4-oxa-7-azaspiro[2.5]octan-7-yl)pyridazine-3-carbonitrile (535 mg, yield: 79%). MS Calcd.: 230.1 MS Found: 231.2 [M+H]⁺.

### Step B: (S)-7-(3-((4-(2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)-4-oxa-7-azaspiro[2.5]octane

(S)-7-(3-((4-(2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)-4-oxa-7-azaspiro[2.5]octane (11 mg) was obtained by a similar method to Procedure 14. MS Calcd.: 685.2. MS Found: 686.0 [M+H]⁺.

¹H NMR (400 MHz, DMSO-d6): δ 7.51-7.61 (m, 3H), 7.33 (d, J=10.0 Hz, 1H), 6.75-6.81 (m, 2H), 6.68-6.75 (m, 1H), 3.85-3.92 (m, 2H), 3.81 (s, 2H), 3.61-3.70 (m, 2H), 3.51-3.57 (m, 2H), 2.93-3.04 (m, 2H), 2.60-2.71 (m, 1H), 2.20-2.35 (m, 2H), 2.01 (s, 3H), 1.64-1.78 (m, 4H), 0.71-0.78 (m, 2H), 0.58-0.66 (m, 2H). ¹⁹F NMR (377 MHz, DMSO-d6): δ −62.95, −110.74.

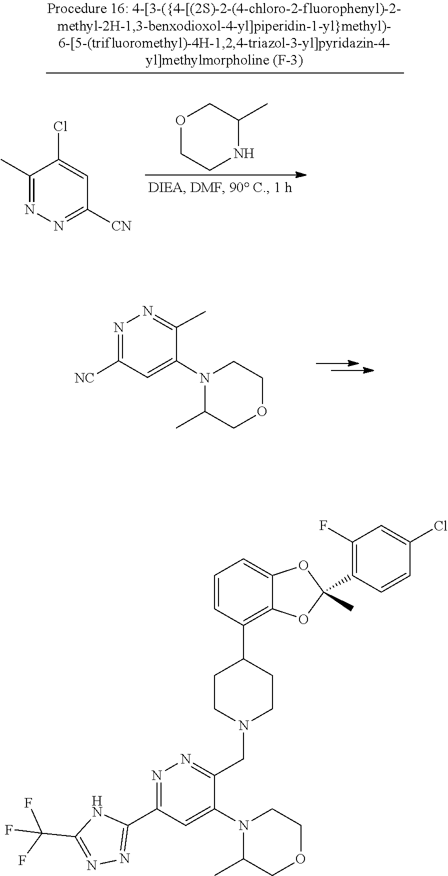

### Step A: 6-methyl-5-(3-methylmorpholino)pyridazine-3-carbonitrile

A mixture of 5-chloro-6-methylpyridazine-3-carbonitrile (300 mg, 1.96 mmol), 3-methylmorpholine (397 mg, 3.92 mmol), DIEA (379 mg, 2.94 mmol) in DMF (6.0 mL) was stirred at 90° C. for 1 hour. After the reaction was completed, the mixture was diluted with H₂O (40 mL), extracted with EA (30 mL×2). The combined organic layer was dried over anhydrous sodium sulfate, filtered, and evaporated to dryness. The residue was purified by column chromatography on silica gel (EA/PE=1/1) to give 6-methyl-5-(3-methylmorpholino)pyridazine-3-carbonitrile (128 mg, yield: 30%).

MS Calcd.: 218.1 MS Found: 219.1 [M+H]⁺.

### Step B: 4-[3-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]pyridazin-4-yl]-3-methylmorpholine (F-3)

4-[3-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]pyridazin-4-yl]-3-methylmorpholine (47.5 mg) was obtained with the similar method disclosed herein.

MS Calcd.: 673.22. MS Found: 674.4 [M+H]⁺.

¹H NMR (400 MHz, DMSO-d6): δ 7.51-7.63 (m, 3H), 7.30-7.37 (m, 1H), 6.69-6.82 (m, 3H), 4.24-4.38 (m, 1H), 3.79-4.02 (m, 4H), 3.53-3.72 (m, 3H), 3.13-3.21 (m, 1H), 3.00-3.08 (m, 1H), 2.89-2.98 (m, 1H), 2.60-2.71 (m, 1H), 2.20-2.35 (m, 2H), 2.01 (d, J=1.6 Hz, 3H), 1.66-1.85 (m, 4H), 1.07 (d, J=6.0 Hz, 3H). ¹⁹F NMR (377 MHz, DMSO-d6): δ −63.41, −110.77, −110.78.

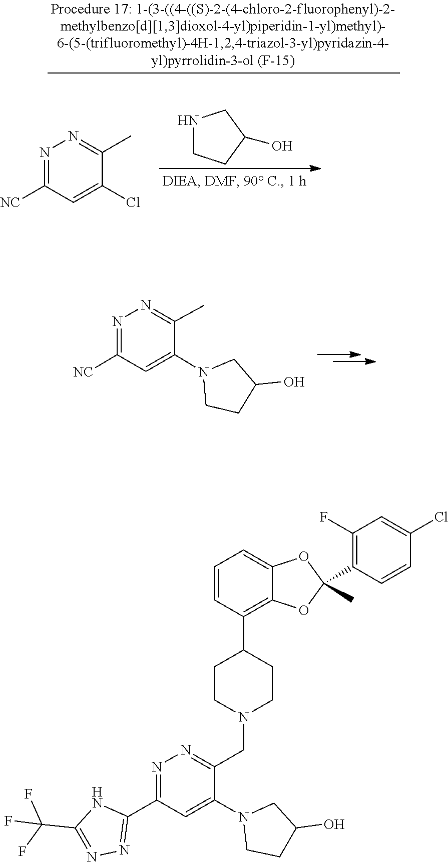

### Step A: 5-(3-hydroxypyrrolidin-1-yl)-6-methylpyridazine-3-carbonitrile

A mixture of 5-chloro-6-methylpyridazine-3-carbonitrile (400 mg, 2.6 mmol), pyrrolidin-3-ol (454 mL, 5.2 mmol), DIEA (1.7 g, 13.0 mmol) in DMF (5 mL) was stirred for 2 hours at 90° C. under nitrogen atmosphere. The mixture was allowed to cool down to room temperature. The resulting mixture was diluted with water (50 mL), extracted with EtOAc (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE to afford 5-(3-hydroxypyrrolidin-1-yl)-6-methylpyridazine-3-carbonitrile (300 mg, yield: 56.4%). MS Calcd.: 204.10. MS Found: 205.2 [M+H]⁺.

### Step B: 1-(3-((4-((S)-2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)pyrrolidin-3-ol (F-15)

1-(3-((4-((S)-2-(4-chloro-2-fluorophenyl)-2-methylbenzo[d][1,3]dioxol-4-yl)piperidin-1-yl)methyl)-6-(5-(trifluoromethyl)-4H-1,2,4-triazol-3-yl)pyridazin-4-yl)pyrrolidin-3-ol (75.0 mg) was obtained with the similar method to Procedure 14. MS Calcd.: 659.20. MS Found: 660.1 [M+H]⁺.

¹H NMR (400 MHz, DMSO-d6): δ 7.51-7.61 (m, 2H), 7.33 (d, J=8.4 Hz, 1H), 7.13 (s, 1H), 6.69-6.80 (m, 3H), 4.39-4.51 (m, 1H), 3.69-4.06 (m, 7H), 2.89-3.00 (m, 2H), 2.65-2.72 (m, 1H), 2.14-2.25 (m, 2H), 1.92-2.05 (m, 5H), 1.65-1.79 (m, 4H). ¹⁹F NMR (377 MHz, DMSO-d6): δ −63.18, −110.82.

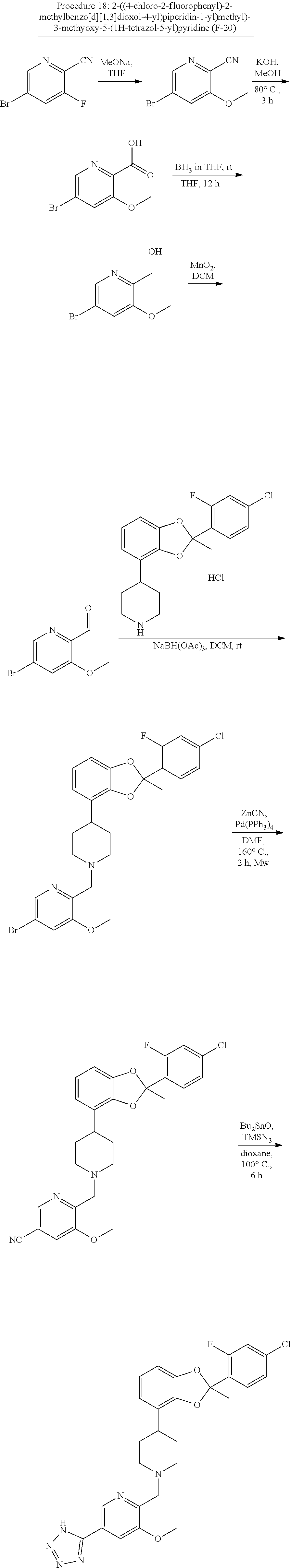

### Step A: 5-bromo-3-methoxypicolinonitrile

To a solution of 5-bromo-3-fluoropicolinonitrile (10.0 g, 49.7 mmol) in THF (100 mL) was added MeONa (3.2 g, 59.7 mmol) at 0° C., the reaction was stirred at room temperature for 1 h. The resulting mixture was quenched with water (200 mL) and extracted with EA (200 mL*3). The combined organic layer was dried over Na₂SO₄, filtered, concentrated and the residue purified by column chromatography on silica gel (PE:EA=4:1) to give 5-bromo-3-methoxypicolinonitrile (9.7 g, 91.5% yield). MS Calcd.: 212.0. MS Found: 212.9 [M+H]⁺.

### Step B: 5-bromo-3-methoxypicolinic acid

To a solution of 5-bromo-3-methoxypicolinonitrile (200 mg, 0.94 mmol) in MeOH (5 mL) and H₂O (2 mL) was added KOH (2.5 g, 47 mmol) at room temperature. The mixture was heated to 80° C. and stirred for 3 h. The mixture was poured onto ice and adjusted to pH=7 with 1 N HCl. The aqueous phase was extracted with EA (50 mL*2). The combined organic layer was dried over Na₂SO₄, filtered, and concentrated to give 5-bromo-3-methoxypicolinic acid (170 mg, 74% yield). MS Calcd.: 230.9; MS Found: 231.9 [M+H]⁺.

### Step C: (5-bromo-3-methoxypyridin-2-yl)methanol

To a solution of 5-bromo-3-methoxypicolinic acid (800 mg, 3.5 mmol) in THF (8 mL) was added BH₃ in THF (7 mL, 7.0 mmol, 1 M in THF) at room temperature and the mixture stirred for 12 hours. After the reaction was completed, the mixture was quenched by MeOH (4 mL) and diluted with EA (50 mL), washed with brine (50 mL). The organic layer was dried over Na₂SO₄, filtered and concentrated. The residue was purified by column chromatography on silica gel (PE/EA=1/1) to give (5-bromo-3-methoxypyridin-2-yl)methanol (400 mg, 52.6% yield). MS Calcd.: 216.97. MS Found: 218.0 [M+H]⁺.

### Step D: 5-bromo-3-methoxypicolinaldehyde

A mixture of (5-bromo-3-methoxypyridin-2-yl)methanol (400 mg, 1.8 mmol), MnO₂ (1.6 g, 18.3 mmol) in DCM (10 mL) was stirred at room temperature overnight. The mixture was filtered and concentrated to give 5-bromo-3-methoxypicolinaldehyde (401 mg, crude).

### Step E: 5-bromo-2-((4-(2-(4-chloro-2-fluorophenyl)-2-methylbenzo[d][1,3]dioxol-4-yl)piperidin-1-yl)methyl)-3-methoxypyridine

To a solution 5-bromo-3-methoxypicolinaldehyde (300 mg, 1.4 mmol), 4-(2-(4-chloro-2-fluorophenyl)-2-methylbenzo[d][1,3]dioxol-4-yl)piperidine HCl salt (483 mg, 1.4 mmol) in DCM (10 mL) was added NaBH(OAc)₃ (884 mg, 4.17 mmol) at room temperature and stirred for 2 h. The resulting mixture was diluted with EA (40 mL) and washed with water (30 mL*2). The organic layer was dried over Na₂SO₄, filtered, concentrated and the residue purified by column chromatography on silica gel (PE:EA=1:1) to give 5-bromo-2-((4-(2-(4-chloro-2-fluorophenyl)-2-methylbenzo[d][1,3]dioxol-4-yl)piperidin-1-yl)methyl)-3-methoxypyridine (400 mg, 52.7% yield). MS Calcd.: 546.1. MS Found: 547.1 [M+H]⁺.

### Step F: 6-((4-(2-(4-chloro-2-fluorophenyl)-2-methylbenzo[d][1,3]dioxol-4-yl)piperidin-1-yl)methyl)-5-methoxynicotinonitrile

A mixture of 5-bromo-2-((4-(2-(4-chloro-2-fluorophenyl)-2-methylbenzo[d][1,3]dioxol-4-yl)piperidin-1-yl)methyl)-3-methoxypyridine (400 mg, 0.73 mmol), Zn(CN)₂ (250 mg, 2.19 mmol), Pd(PPh₃)₄ (80 mg, 0.07 mmol) in DMF (5 mL) was stirred at 160° C. for 2 hours under microwave. After the reaction was completed, the mixture was diluted with EA (70 mL), washed with H₂O (40 mL*3). The organic layer was dried over anhydrous sodium sulfate, filtered, and evaporated to dryness. The residue was purified by column chromatography on silica gel (PE/EA=1/1) to give 6-((4-(2-(4-chloro-2-fluorophenyl)-2-methylbenzo[d][1,3]dioxol-4-yl)piperidin-1-yl)methyl)-5-methoxynicotinonitrile (120 mg, 33.10% yield). MS Calcd.: 493.2. MS Found: 494.2 [M+H]⁺.

### Step G: 2-((4-(2-(4-chloro-2-fluorophenyl)-2-methylbenzo[d][1,3]dioxol-4-yl)piperidin-1-yl)methyl)-3-methoxy-5-(1H-tetrazol-5-yl)pyridine

A mixture of 6-((4-(2-(4-chloro-2-fluorophenyl)-2-methylbenzo[d][1,3]dioxol-4-yl)piperidin-1-yl)methyl)-5-methoxynicotinonitrile (60 mg, 0.12 mmol), Bu₂SnO (89 mg, 0.36 mmol), TMSN₃ (70 mg, 0.60 mmol) in dioxane (1 mL) was stirred at 100° C. for 6 hours. After the reaction was completed, the mixture was filtered and the filtrate was purified by Prep-HPLC to afford 2-((4-(2-(4-chloro-2-fluorophenyl)-2-methylbenzo[d][1,3]dioxol-4-yl)piperidin-1-yl)methyl)-3-methoxy-5-(1H-tetrazol-5-yl)pyridine (16.3 mg, yield: 24.0%). MS Calcd.: 536.2. MS Found: 537.2 [M+H]⁺.

¹H NMR (400 MHz, DMSO-d₆): δ 8.91 (d, J=1.2 Hz, 1H), 8.07 (d, J=1.6 Hz, 1H), 7.68 (t, J=8.4 Hz, 1H), 7.57 (dd, J=11.2 Hz, 2.0 Hz, 1H), 7.40 (dd, J=8.4 Hz, 1.2 Hz, 1H), 6.87-6.95 (m, 2H), 6.80-6.83 (m, 1H), 4.40 (br s, 2H), 4.05 (s, 3H), 3.52-3.59 (m, 2H), 3.11-3.15 (m, 2H), 2.96-3.03 (m, 1H), 1.97-2.18 (m, 7H). ¹⁹F NMR (377 MHz, DMSO-d6): δ −110.45.

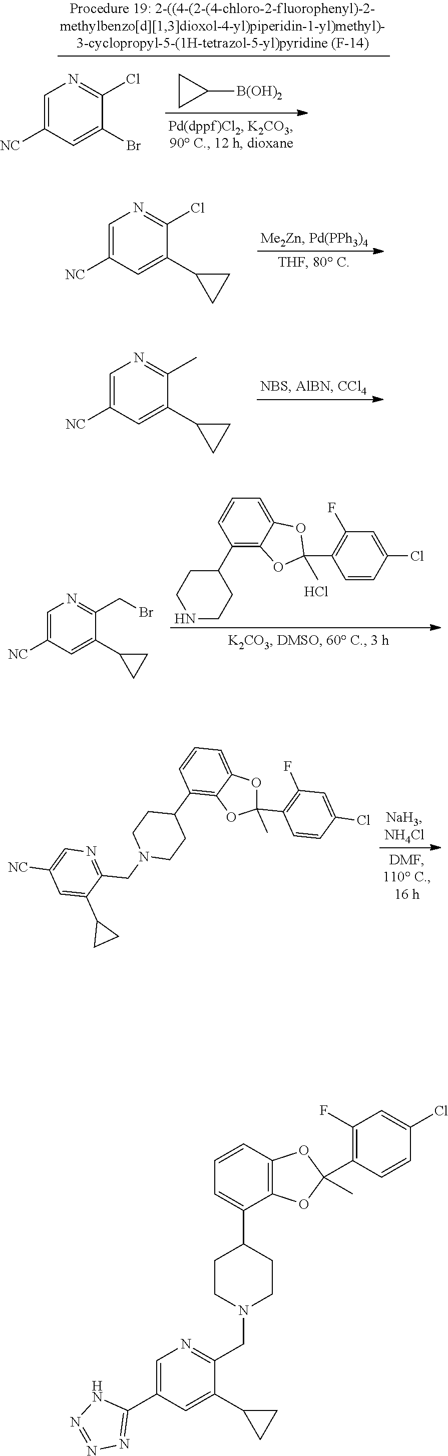

### Step A: 6-chloro-5-cyclopropylnicotinonitrile

A mixture of 5-bromo-6-chloronicotinonitrile (200 mg, 0.92 mmol), cyclopropylboronic acid (119 mg, 1.38 mmol), Pd(dppf)Cl₂ (50 mg, 0.07 mmol) and K₂CO₃ (381 mg, 2.76 mmol) in 1,4-dioxane (2 mL) was stirred at 90° C. under Argon for 12 hours. The mixture was poured into water (30 mL) and extracted with EtOAC (2×30 mL), the combined organic layer was washed with brine, dried over sodium sulfate, filtered and the residue was concentrated. The residue was purified by column chromatography on silica gel to obtain 6-chloro-5-cyclopropylnicotinonitrile (90 mg, yield: 55%). MS Calcd: 178.0. MS Found: 179.1 [M+H]⁺.

### Step B: 5-cyclopropyl-6-methylnicotinonitrile

A mixture of 6-chloro-5-cyclopropylnicotinonitrile (90 mg, 0.51 mmol), toluene solution of Me₂Zn (1.01 mL, 1M, 1.01 mmol) and Pd(PPh₃)₄ (58 mg, 0.05 mmol) in THE (6 mL) was stirred at 80° C. under Ar for 16 h. The mixture was poured into water (30 mL) and extracted with EtOAC (2×30 mL), the combined organic layer was washed with brine, dried over sodium sulfate, filtered and the residue was concentrated. The residue was purified by column chromatography on silica gel to obtain 5-cyclopropyl-6-methylnicotinonitrile (60 mg, yield: 75.1%). MS Calcd: 158.1. MS Found: 159.2 [M+H]⁺.

### Step C: 6-(bromomethyl)-5-cyclopropylnicotinonitrile

A mixture of 5-cyclopropyl-6-methylnicotinonitrile (60 mg, 0.38 mmol), NBS (74 mg, 0.42 mmol) and AIBN (13 mg, 0.08 mmol) in CCl₄ (5 mL) was stirred at 80° C. for 16 h. The mixture was poured into water (30 mL) and extracted with EtOAC (2×30 mL), the combined organic layer was washed with brine, dried over sodium sulfate, filtered and the residue was concentrated. The residue was purified by column chromatography on silica gel to obtain 6-(bromomethyl)-5-cyclopropylnicotinonitrile (25 mg, yield: 27.9%). MS Calcd: 236.0. MS Found: 239.0 [M+H]⁺.

### Step D: 6-((4-(2-(4-chloro-2-fluorophenyl)-2-methylbenzo[d][1,3]dioxol-4-yl)piperidin-1-yl)methyl)-5-cyclopropylnicotinonitrile

A mixture of 6-(bromomethyl)-5-cyclopropylnicotinonitrile (25 mg, 0.11 mmol), 4-(2-(4-chloro-2-fluorophenyl)-2-methylbenzo[d][1,3]dioxol-4-yl)piperidine hydrochloric acid salt (41 mg, 0.11 mmol) and K₂CO₃ (44 mg, 0.32 mmol) in DMSO (3 mL) was stirred at 60° C. for 3 h. The mixture was poured into water (30 mL) and extracted with EtOAC (2×30 mL), the combined organic layer was washed with brine, dried over sodium sulfate, filtered and the residue was concentrated. The residue was purified by column chromatography on silica gel to obtain 6-((4-(2-(4-chloro-2-fluorophenyl)-2-methylbenzo[d][1,3]dioxol-4-yl)piperidin-1-yl)methyl)-5-cyclopropylnicotinonitrile (25 mg, yield: 47.2%). MS Calcd: 503.2. MS Found: 504.1 [M+H]⁺.

### Step F: 2-((4-(2-(4-chloro-2-fluorophenyl)-2-methylbenzo[d][1,3]dioxol-4-yl)piperidin-1-yl)methyl)-3-cyclopropyl-5-(1H-tetrazol-5-yl)pyridine (F-14)

A mixture of 6-((4-(2-(4-chloro-2-fluorophenyl)-2-methylbenzo[d][1,3]dioxol-4-yl)piperidin-1-yl)methyl)-5-cyclopropylnicotinonitrile (25 mg, 0.05 mmol), NaN₃ (5 mg, 0.077 mmol) and NH₄Cl (4 mg, 0.077 mmol) in DMF (1 mL) was stirred at 110° C. in a sealed tube for 16 h. The mixture was filtered, and the residue was purified by prep-HPLC to obtain 2-((4-(2-(4-chloro-2-fluorophenyl)-2-methylbenzo[d][1,3]dioxol-4-yl)piperidin-1-yl)methyl)-3-cyclopropyl-5-(1H-tetrazol-5-yl)pyridine (4.4 mg, yield: 16.3%). MS Calcd: 546.2. MS Found: 547.1 [M+H]⁺

¹H NMR (400 MHz, CD₃OD): δ 9.08 (s, 1H), 8.12 (s, 1H), 7.62 (t, J=8.4 Hz, 1H), 7.31-7.22 (m, 2H), 6.83-6.75 (m, 3H), 4.61-4.50 (m, 2H), 3.66-3.58 (m, 2H), 3.11-2.97 (m, 2H), 2.23-2.13 (m, 3H), 2.05 (s, 5H), 1.14-1.12 (m, 2H), 0.87-0.86 (m, 2H). ¹⁹F-NMR (377 MHz, CD₃OD): δ −112.25.

The compounds of Table 1 can or were synthesized according to the general synthesis or procedures described herein using the appropriate starting materials.

| | | MS (m/z) |
|---|---|---|
| No. | Structure | [M + H]⁺ |
| A-1 | 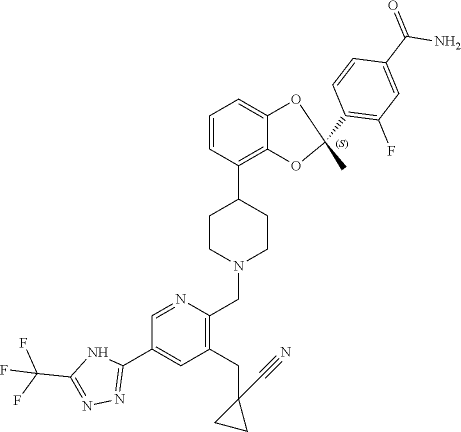 | MS Calcd: 661.2; MS Found: 662.4 |
| A-3 | 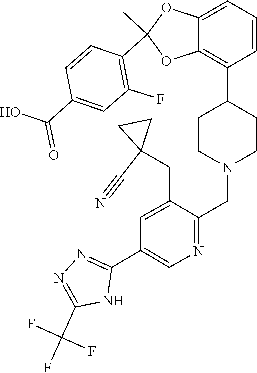 [SMILES: CC1(c2ccc(C(=O)O)cc2F)Oc2cccc(C3CCN(Cc4ncc(-c5nnc(C(F)(F)F)[nH]5)cc4CC4(C#N)CC4)CC3)c2O1] | MS Calcd: 662.2; MS Found: 663.1 |
| B-1 | 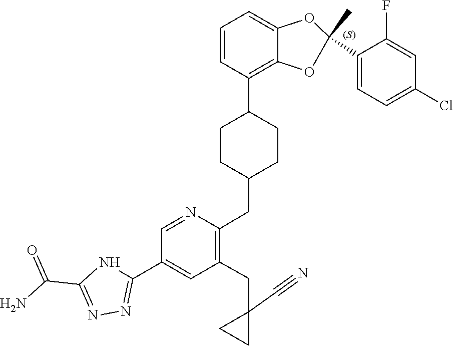 | MS Calcd: 627.2; MS Found: 628.3 |
| C-1 | 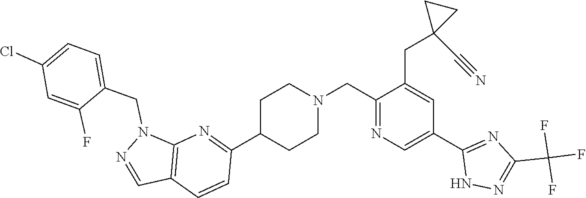 [SMILES: N#CC1(Cc2cc(-c3nc(C(F)(F)F)n[nH]3)cnc2CN2CCC(c3ccc4cnn(Cc5ccc(Cl)cc5F)c4n3)CC2)CC1] | MS Calcd: 649.2; MS Found: 650.3 |
| D-1 | 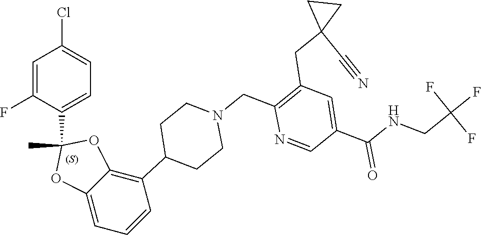 | N.A. |
| D-2 | 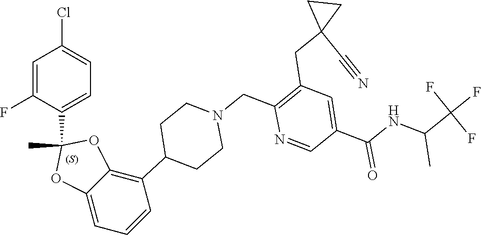 | N.A. |
| D-3 | 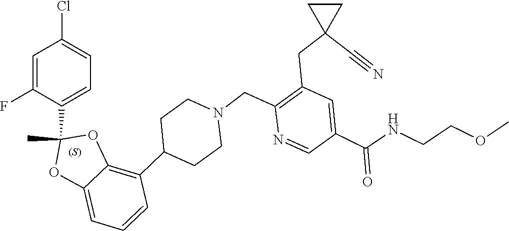 | MS Calcd: 618.2; MS Found: 619.3 |
| D-4 | 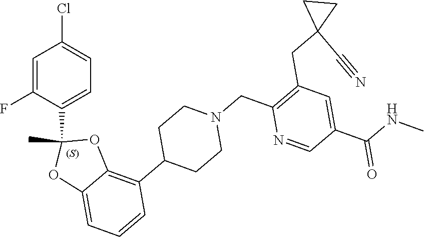 | MS Calcd: 574.2; MS Found: 575.2 |
| D-5 | 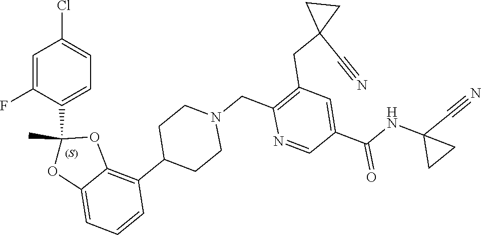 | N.A. |
| D-6 | 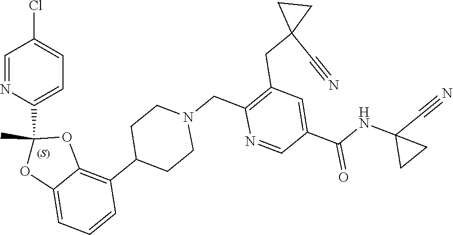 | MS Calcd: 608.2; MS Found: 609.4 |
| D-7 | 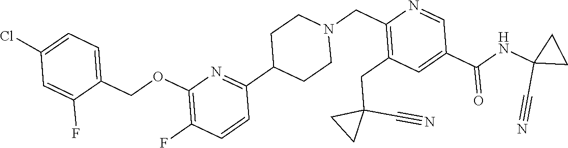 [SMILES: N#CC1(Cc2cc(C(=O)NC3(C#N)CC3)cnc2CN2CCC(c3ccc(F)c(OCc4ccc(Cl)cc4F)n3)CC2)CC1] | MS Calcd: 616.2; MS Found: 617.2 |
| E-3 | 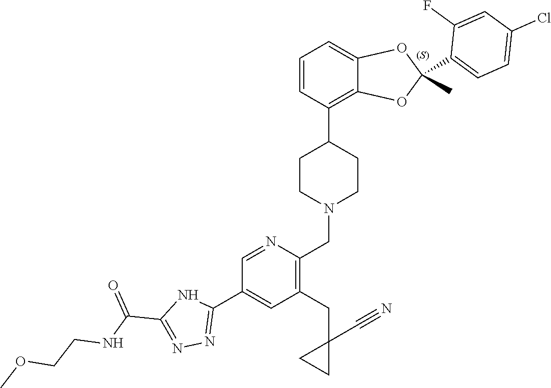 | MS Calcd: 685.2; MS Found: 686.4 |
| E-4 | 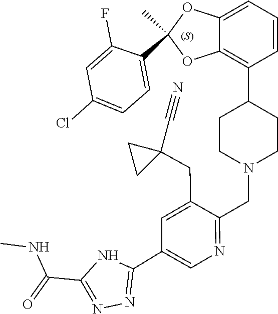 | MS Calcd: 641.2; MS Found: 642.3 |
| E-5 | 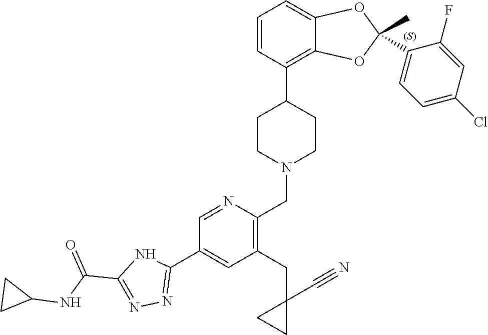 | MS Calcd: 667.2; MS Found: 668.3 |
| E-6 | 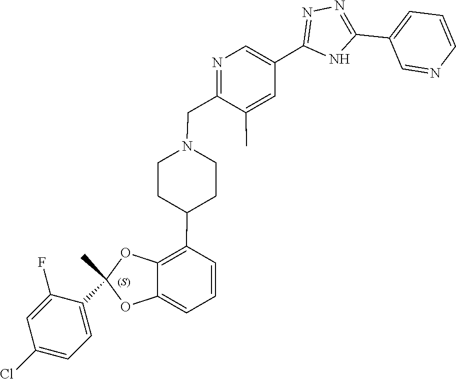 | MS Calcd: 596.2; MS Found: 597.2 |
| E-7 | 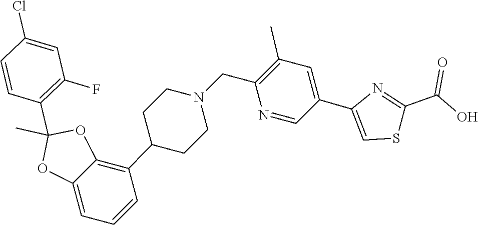 [SMILES: Cc1cc(-c2csc(C(=O)O)n2)cnc1CN1CCC(c2cccc3c2OC(C)(c2ccc(Cl)cc2F)O3)CC1] | MS Calcd: 579.1; MS Found: 580.2 |
| E-8 | 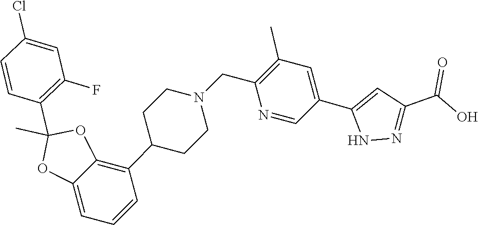 [SMILES: Cc1cc(-c2cc(C(=O)O)n[nH]2)cnc1CN1CCC(c2cccc3c2OC(C)(c2ccc(Cl)cc2F)O3)CC1] | MS Calcd: 562.2; MS Found: 563.3 |
| E-9 | 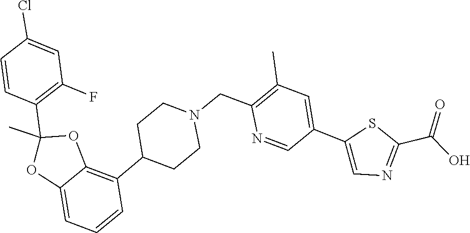 [SMILES: Cc1cc(-c2cnc(C(=O)O)s2)cnc1CN1CCC(c2cccc3c2OC(C)(c2ccc(Cl)cc2F)O3)CC1] | MS Calcd: 579.1; MS Found: 580.3 |
| E-10 | 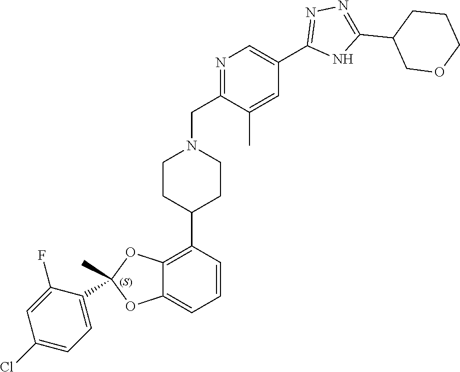 | MS Calcd: 603.2; MS Found: 604.2 |
| E-11 | 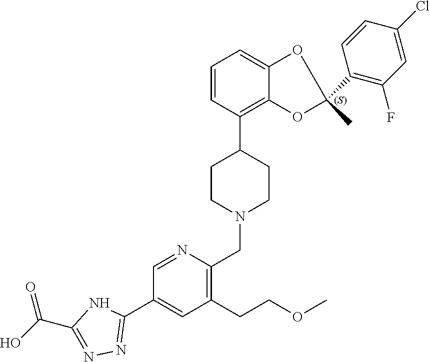 | MS Calcd: 607.2; MS Found: 608.2 |
| E-12 | 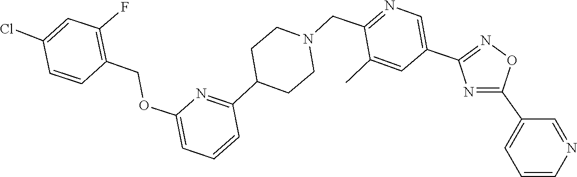 [SMILES: Cc1cc(-c2noc(-c3cccnc3)n2)cnc1CN1CCC(c2cccc(OCc3ccc(Cl)cc3F)n2)CC1] | MS Calcd: 570.2; MS Found: 571.2 |
| E-13 | 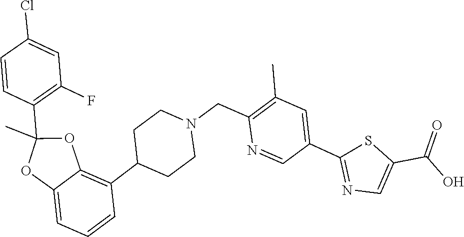 [SMILES: Cc1cc(-c2ncc(C(=O)O)s2)cnc1CN1CCC(c2cccc3c2OC(C)(c2ccc(Cl)cc2F)O3)CC1] | MS Calcd: 579.1; MS Found: 580.3 |
| E-14 | 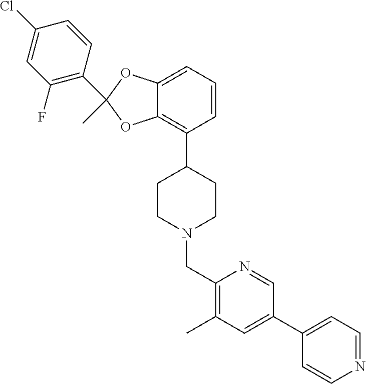 [SMILES: Cc1cc(-c2ccncc2)cnc1CN1CCC(c2cccc3c2OC(C)(c2ccc(Cl)cc2F)O3)CC1] | MS Calcd: 529.2; MS Found: 530.2 |
| E-15 | 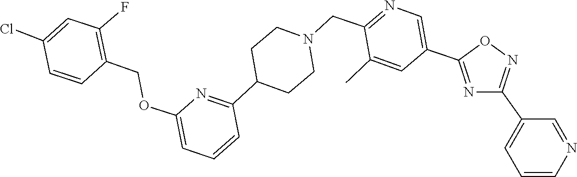 [SMILES: Cc1cc(-c2nc(-c3cccnc3)no2)cnc1CN1CCC(c2cccc(OCc3ccc(Cl)cc3F)n2)CC1] | MS Calcd: 570.2; MS Found: 571.1 |
| E-16 | 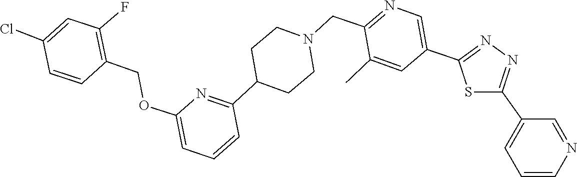 [SMILES: Cc1cc(-c2nnc(-c3cccnc3)s2)cnc1CN1CCC(c2cccc(OCc3ccc(Cl)cc3F)n2)CC1] | MS Calcd: 586.2; MS Found: 587.2 |
| E-17 | 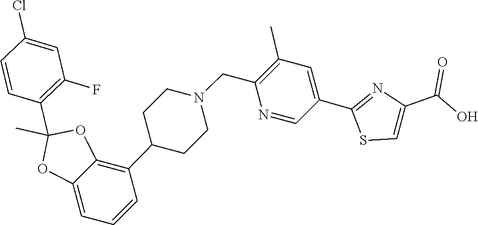 [SMILES: Cc1cc(-c2nc(C(=O)O)cs2)cnc1CN1CCC(c2cccc3c2OC(C)(c2ccc(Cl)cc2F)O3)CC1] | MS Calcd: 579.1; MS Found: 580.3 |
| E-18 | 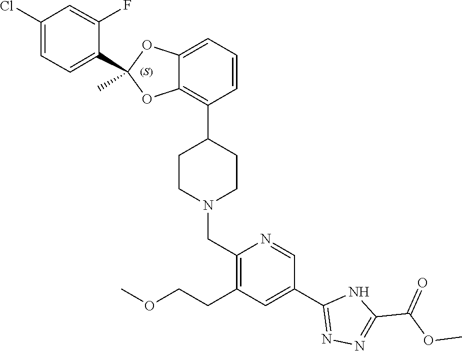 | MS Calcd: 621.2; MS Found: 622.2 |
| E-19 | 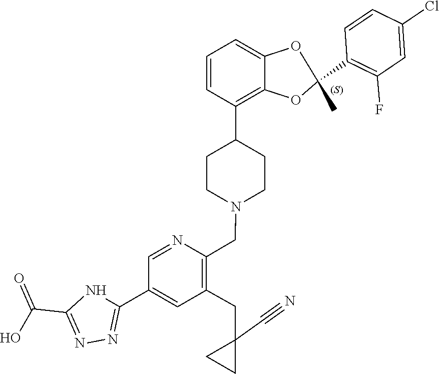 | MS Calcd: 628.2; MS Found: 629.3 |
| F-1 | 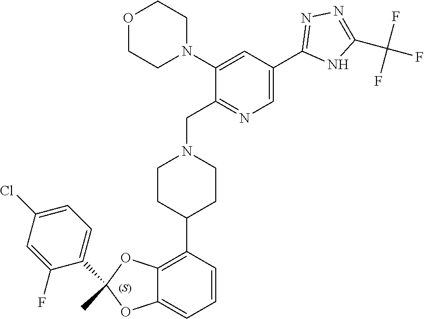 | MS Calcd: 658.2; MS Found: 659.3 |
| F-2 | 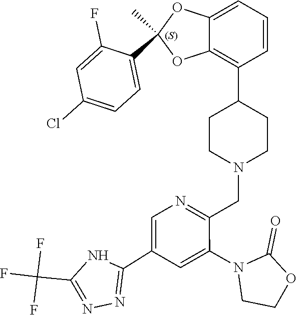 | MS Calcd: 658.2; MS Found: 659.4 |
| F-3 | 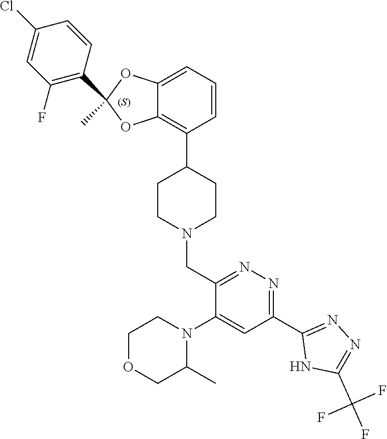 | MS Calcd.: 673.2; MS Found: 674.4 |
| F-4 | 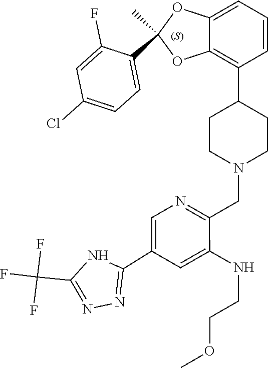 | MS Calcd: 646.2; MS Found: 647.3 |
| F-5 | 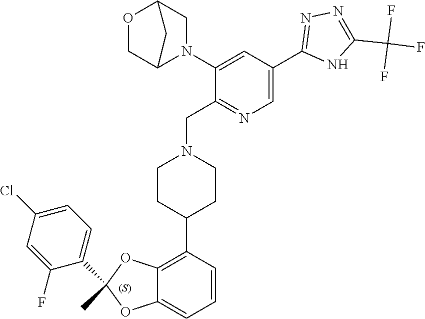 | MS Calcd: 670.2; MS Found: 671.4 |
| F-6 | 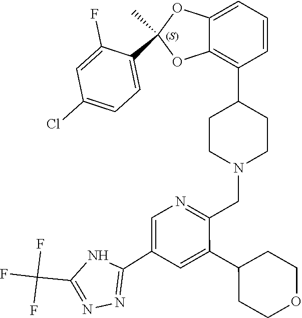 | MS Calcd: 657.2; MS Found: 658.0 |
| F-7 | 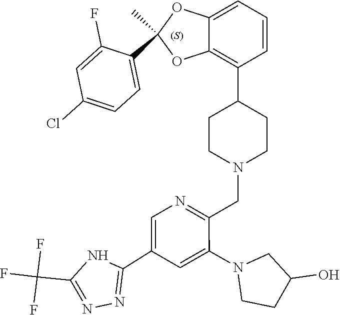 | MS Calcd: 658.2; MS Found: 659.0 |
| F-8 | 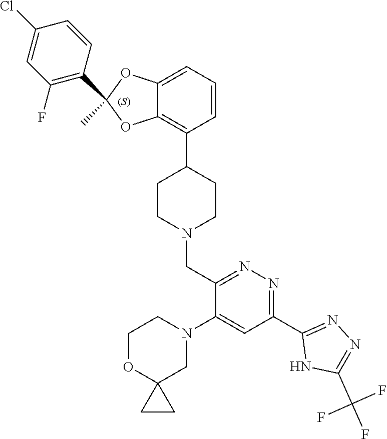 | MS Calcd.: 685.2; MS Found: 686.0 |
| F-9 | 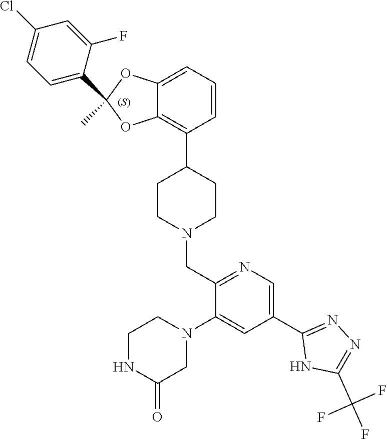 | MS Calcd: 671.2; MS Found: 672.3 |
| F-10 | 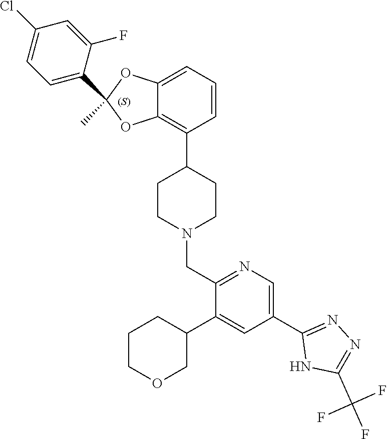 | MS Calcd: 657.2; MS Found: 658.2 |
| F-11 | 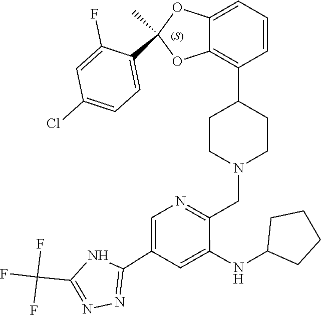 | MS Calcd: 656.2; MS Found: 659.5 |
| F-12 | 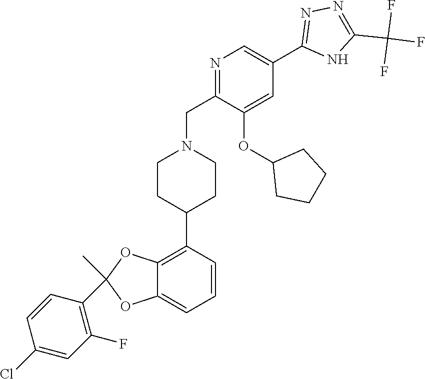 [SMILES: CC1(c2ccc(Cl)cc2F)Oc2cccc(C3CCN(Cc4ncc(-c5nnc(C(F)(F)F)[nH]5)cc4OC4CCCC4)CC3)c2O1] | MS Calcd.: 657.2; MS Found: 658.2 |
| F-13 | 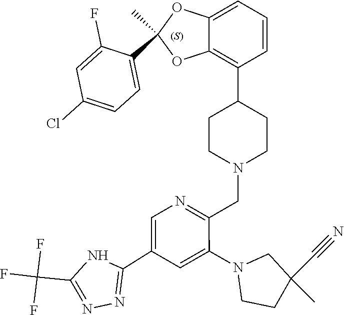 | MS Calcd: 681.2; MS Found: 682.4 |
| F-14 | 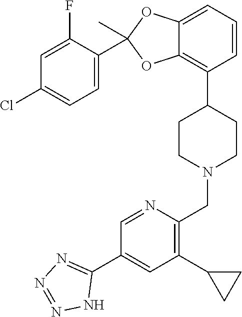 [SMILES: CC1(c2ccc(Cl)cc2F)Oc2cccc(C3CCN(Cc4ncc(-c5nnn[nH]5)cc4C4CC4)CC3)c2O1] | MS Calcd: 546.2; MS Found: 547.1 |
| F-15 | 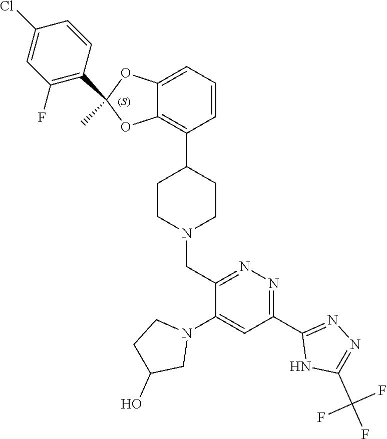 | MS Calcd.: 659.2; MS Found: 660.1 |
| F-16 | 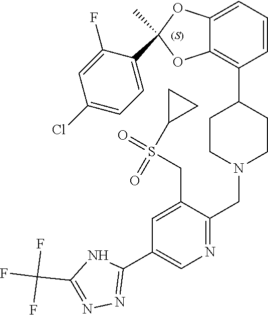 | Calcd: 691.2; MS Found: 692.2 |
| F-17 | 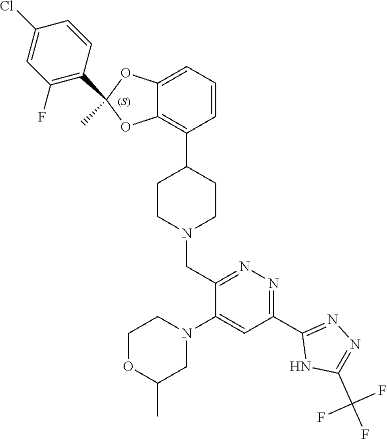 | MS Calcd.: 673.2; MS Found: 674.0 |
| F-18 | 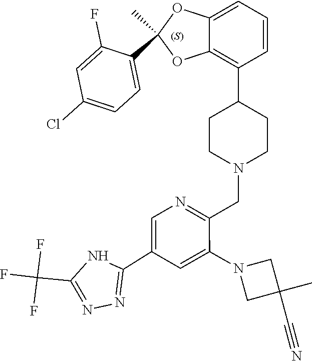 | MS Calcd: 667.2; MS Found: 668.4 |
| F-19 | 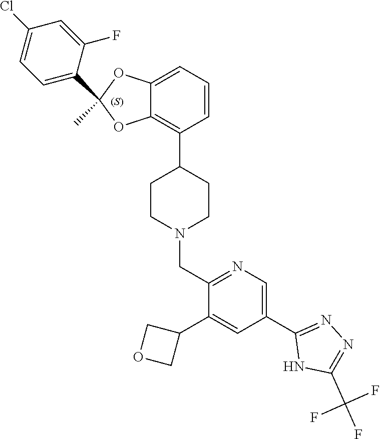 | MS Calcd: 629.2; MS Found: 630.2 |
| F-20 | 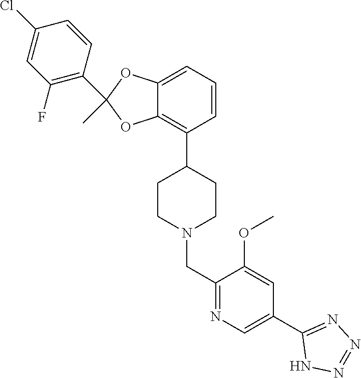 [SMILES: COc1cc(-c2nnn[nH]2)cnc1CN1CCC(c2cccc3c2OC(C)(c2ccc(Cl)cc2F)O3)CC1] | MS Calcd.: 536.2; MS Found: 537.2 |

### BIOLOGICAL EXAMPLES

### Biological Example 1: cAMP Assays

Activation of GLP-1 receptor is known to stimulate cyclic AMP (cAMP) production in cells which indicates primary coupling to the Gas subunit of the G protein heterotrimeric complex. Evidence suggests signaling through Gas induced cAMP stimulation elicits the desired pharmacological response regarding insulin release from pancreatic P-cells.

To optimize functional activity directed toward Gas coupling, a HEK293/CRE-Luc cell line developed by HDB stably expressing the GLP-1 Receptor was used. 200× concentration of compound working solutions were prepared (Agilent Technologies Bravo) with ½ log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550. 1×105 cells/mL HEK293/GLPIR/CRE-LUC (HD Biosciences) cell suspensions prepared with assay buffer [DPBS containing 0.5 mM IBMX (Sigma, Cat #15879) and 0.10% BSA (GENVIEW, Cat #FA016-100g)], 10 μL cell suspensions were added to each well of previous generated assay plate which already contains 50 nL compound at 200× concentration using ThermoFisher Multidrop Combi (1000 cells/well). Seal the plate and incubate at 37° C. with 5% CO₂ for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

$$\% Activity =$$

$$100 \% \times ( mean RLU of test sample - mean RLU of vehicle control ) /$$

$$( mean RLU of MAX control - mean RLU of vehicle control ) )$$

Table 3 shows the biological activity of compounds in GLP-1R agonist cAMP stimulation assay (EC₅₀). Activity of the tested compounds is provided in Table 3 below as follows: +++=EC₅₀<1 nM; ++=EC₅₀ 1-100 nM; +=EC₅₀ >100 nM.

TABLE 3

| No. | Activity |
|---|---|
| A-1 | +++ |
| A-3 | ++ |
| B-1 | +++ |
| C-1 | +++ |
| D-3 | ++ |
| D-4 | ++ |
| D-6 | + |
| D-7 | + |
| E-3 | +++ |
| E-4 | +++ |
| E-5 | +++ |
| E-6 | ++ |
| E-7 | ++ |
| E-8 | ++ |
| E-9 | ++ |
| E-10 | + |
| E-11 | + |
| E-12 | + |
| E-13 | + |
| E-14 | + |
| E-15 | + |
| E-16 | + |
| E-17 | + |
| E-18 | ++ |
| E-19 | +++ |
| F-1 | +++ |
| F-2 | ++ |
| F-3 | ++ |
| F-4 | ++ |
| F-5 | ++ |
| F-6 | ++ |
| F-7 | ++ |
| F-8 | ++ |
| F-9 | ++ |
| F-10 | ++ |
| F-11 | ++ |
| F-12 | ++ |
| F-13 | ++ |
| F-14 | ++ |
| F-15 | ++ |
| F-16 | ++ |
| F-17 | ++ |
| F-18 | ++ |
| F-19 | + |
| F-20 | + |

1. A compound of Formula I: 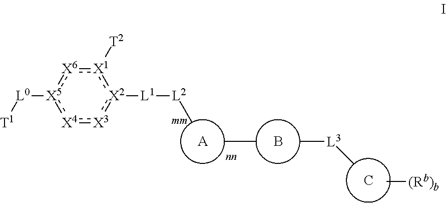
or a pharmaceutically acceptable salt or solvate thereof, wherein:
X¹, X², and X⁵ are independently C or N;
X³ and X⁴ are independently selected from the group consisting of: N, NR^{x}, CR^{y}, C(O), O, and S;
X⁶ is selected from the group consisting of: a bond, N, NR^{x}, CR^{y}, and C(O);
each [symbol] is a single bond or a double bond, provided that at least one of X¹—X⁶ is an independently selected heteroatom or heteroatomic group; at least one of X¹—X⁶ is C or CR^{y}; and the ring including X¹—X⁶ is aromatic;
each R^{x} is independently selected from the group consisting of: hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
each R^{y} is independently selected from the group consisting of: hydrogen, —OH, (C₁-C₆)alkyl, (C₁-C₆)haloalkyl, (C₁-C₆)alkoxy, (C₁-C₆)haloalkoxy, CN, and halogen;
L⁰ is a bond or #—P⁰-P¹, wherein # represents the point of attachment to X⁵;
—P⁰ is a bond, —NH—, —N(C₁-C₆ alkyl)-, —O—, or S(O)₀₋₂;
—P¹ is selected from the group consisting of: (C₁-C₆)alkylene, (C₂-C₆)alkenylene, (C₂-C₆)alkynylene, (C₃-C₅)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH or a carboxylic acid bioisostere; or
the moiety -L⁰-T¹ is:
—C(O)NH—(C₁-C₆)alkyl or —C(O)NH—(C₃-C₆)cycloalkyl; wherein each is optionally substituted with 1-4 R^{T}; or
a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
T² is:
hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
—NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl;
wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆ alkyl)-C(O)—C₁-C₆ alkyl, S(O)₂(C₁-C₆ alkyl), and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of: 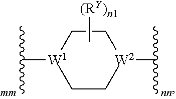
wherein n1 is 0, 1, or 2, W¹ is CR^{Y1} or N, and W² is CR^{Y2} or N; 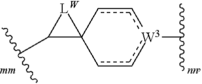
wherein W³ is C, CR^{Y3}, or N, L^{w} is (C₁-C₃)alkylene, and each [symbol] is independently a single bond or a double bond, as allowed by valence;
phenylene optionally substituted with 1-4 R^{Y};
5- to 6-membered heteroarylene optionally substituted with 1-3 R^{Y};
(C₅-C₅)cycloalkylene optionally substituted with 1-4 R^{Y};
partially unsaturated monocyclic (C₅-C₅)cycloalkylene optionally substituted with 1-4 R^{Y}; and
partially unsaturated monocyclic 5- to 8-membered heterocycloalkylene optionally substituted with 1-4 R^{Y};
wherein mm represents the point of attachment to L², and nn represents the point of attachment to Ring B;
each occurrence of R is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1}, R^{Y2}, and R^{Y3} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together can form (C₁-C₄)alkylene, wherein one of the CH₂ units of the (C₁-C₄)alkylene is optionally replaced by a heteroatom selected from the group consisting of O, S, NH, and N(C₁₋₃)alkyl;
Ring B is selected from the group consisting of: (B-I), (B-II), (B-III), (B-IV), (B-V), (B-VI), and (B-VII): 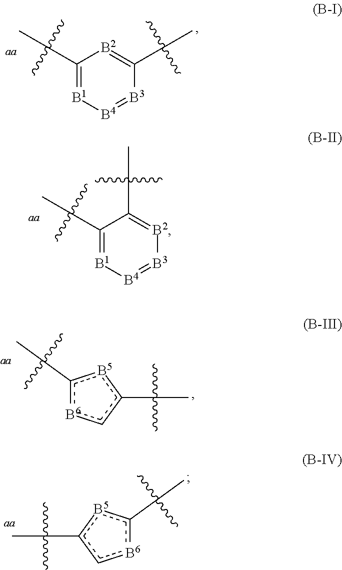
wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl; 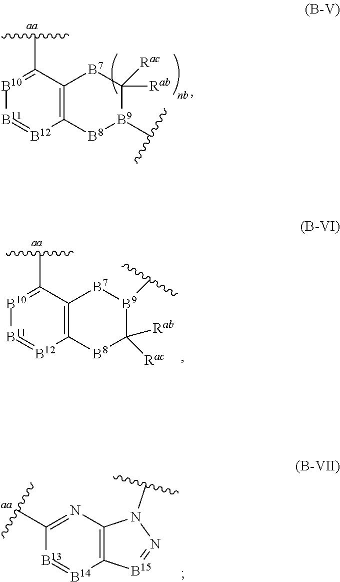
wherein aa represents the point of attachment to Ring A;
B⁷ and B⁸ are independently selected from the group consisting of: —O—, —NR^{N}—, and —C(R′)₂—;
B⁹ is N or CR^{aa};
nb is 0 or 1;
B¹⁰, B¹¹, and B¹² are independently selected from the group consisting of CR¹ and N;
B¹³, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or —Z¹-Z²—*, wherein * represents the point of attachment to Ring C;
—Z¹ is a bond, NH, N(C₁-C₆ alkyl), O, or S(O)₀₋₂;
—Z² is C₁₋₃ alkylene optionally substituted with 1-2 R^{e};
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3;
provided at least one of the following occurs:
1) at least one R is —C(O)NH₂ or —C(O)OH;
2) Ring A is (C₅-C₈)cycloalkylene optionally substituted with 1-4 R^{Y};
3) Ring B is 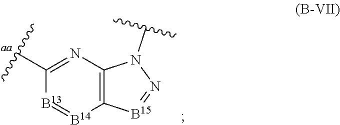
4) the moiety -L⁰-T¹ is:
—C(O)NH—(C₁-C₆)alkyl or —C(O)NH—(C₃-C₆)cycloalkyl; wherein each is optionally substituted with 1-4 R^{T}; or
a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}; provided the compound is not:

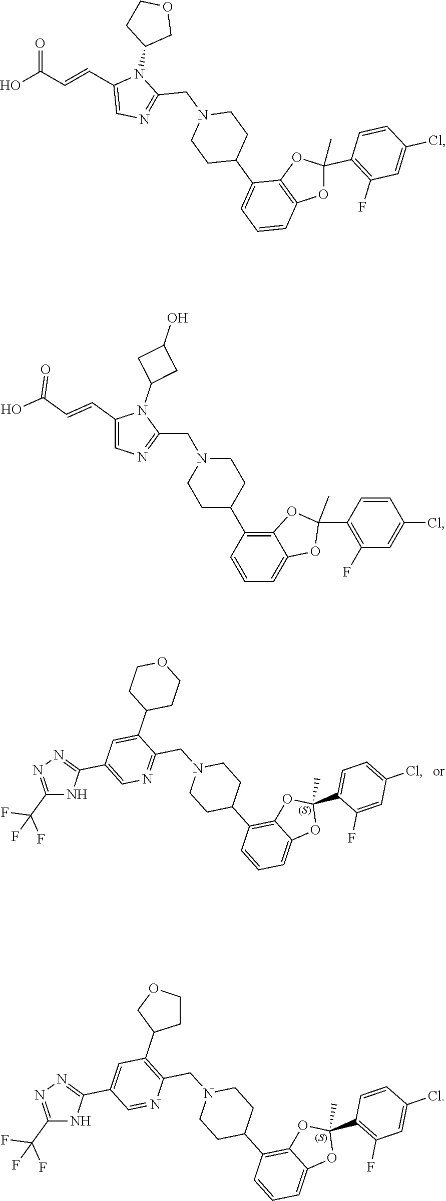

2. A compound of Formula II: 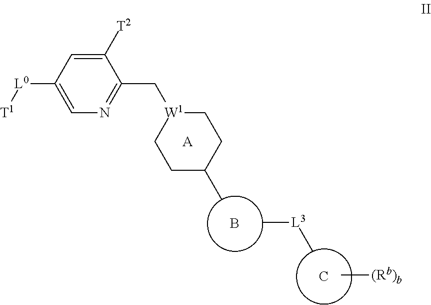
or a pharmaceutically acceptable salt or solvate thereof, wherein:
L⁰ is a bond or —P⁰-P¹;
—P⁰ is a bond, —NH—, —N(C₁-C₆ alkyl)-, —O—, or S(O)₀₋₂;
—P¹ is selected from the group consisting of: (C₁-C₆)alkylene, (C₂-C₆)alkenylene, (C₂-C₆)alkynylene, (C₃-C₅)cycloalkylene, and 4- to 8-membered heterocycloalkylene, each of which is optionally substituted with 1-3 R⁰;
each R⁰ is independently selected from the group consisting of: halogen, CN, (C₁-C₆)alkyl, (C₁-C₆)haloalkyl, (C₁-C₆)alkoxy, and (C₁-C₆)haloalkoxy;
T¹ is C(O)OH or a carboxylic acid bioisostere; or
the moiety -L⁰-T¹ is:
—C(O)NH—(C₁-C₆)alkyl or —C(O)NH—(C₃-C₆)cycloalkyl; wherein each is optionally substituted with 1-4 R^{T}; or
a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
T² is:
hydrogen, CN, (C₁-C₆)haloalkyl, (C₁-C₆)hydroxyalkyl or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), —N(C₁-C₆ alkyl)-S(O)₂(C₁-C₆ alkyl), —NH—S(O)₂(C₁-C₆ alkyl), (C₃-C₆)cycloalkyl, (C₃-C₆)cycloalkoxy, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}; or
—NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl;
wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
each R^{T} is independently selected from the group consisting of OH, SH, CN, NO₂, halogen, ═O, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, (C₁-C₆)alkylamino(C₁-C₆alkyl)-C(O)— C₁-C₆ alkyl, S(O)₂(C₁-C₆ alkyl), and di(C₁-C₆)alkylamino;
W¹ is CH or N;
Ring B is selected from the group consisting of: (B-I), (B-II), (B-III), (B-IV), (B-V), (B-VI), and (B-VII): 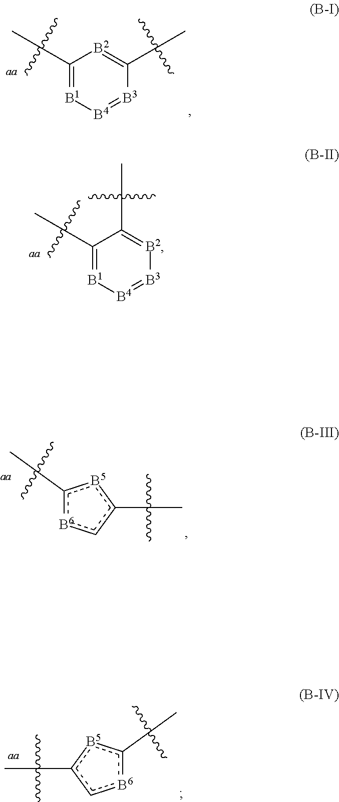
wherein aa represents the point of attachment to Ring A;
each of B¹, B², B³, and B⁴ is independently selected from the group consisting of CR¹ and N;
each of B⁵ and B⁶ is independently selected from the group consisting of N, NR¹, C, CR¹, O, and S, provided that the ring containing B⁵ and B⁶ is heteroaryl; 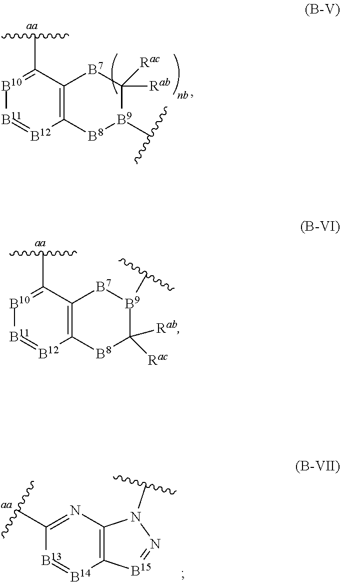
wherein aa represents the point of attachment to Ring A;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
B¹³, B¹⁴, and B¹⁵ are each independently CR¹;
each R¹ is independently selected from the group consisting of hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of H and (C₁-C₆)alkyl;
each R^{N} is independently selected from the group consisting of hydrogen, (C₁-C₆)alkyl, (C₁-C₆)haloalkyl, C(O)(C₁-C₆)alkyl, S(O)₂(C₁-C₆)alkyl, and C(O)O(C₁-C₆)alkyl;
R^{aa}, R^{ab}, and R^{ac} are each independently selected from the group consisting of H, (C₁-C₆)alkyl, and (C₁-C₆)haloalkyl;
L³ is a bond or —Z¹-Z²—*, wherein * represents the point of attachment to Ring C;
—Z¹ is a bond, NH, N(C₁-C₆ alkyl), 0, or S(O)₀₋₂;
—Z² is C₁₋₃ alkylene optionally substituted with 1-2 R^{c};
each R^{c} is independently selected from the group consisting of halogen, (C₁-C₆)alkyl, and (C₁-C₃)haloalkyl;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, CN, —C(O)NH₂, or —C(O)OH; and
b is an integer selected from 0-3;
provided at least one of the following occurs:
1) at least one R is —C(O)NH₂ or —C(O)OH;
2) W¹ is CH;
3) Ring B is 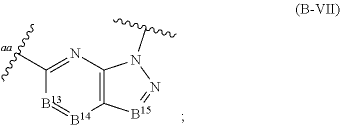
4) the moiety -L⁰-T¹ is:
—C(O)NH—(C₁-C₆)alkyl or —C(O)NH—(C₃-C₆)cycloalkyl; wherein each is optionally substituted with 1-4 R^{T}; or
a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)O(C₁-C₆)alkyl, —C(O)NH(C₁-C₆)alkyl, —C(O)NH—(C₃-C₆)cycloalkyl, 3- to 6-membered heterocycloalkyl, and 6-membered heteroaryl is optionally substituted with 1-4 R^{T};
or
5) T² is —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T};
provided the compound is not:

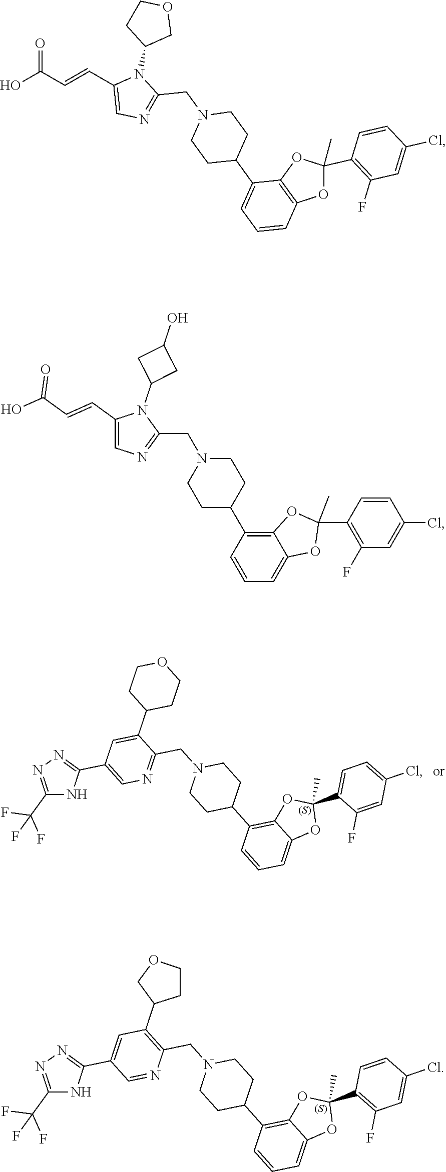

3. The compound of claim 2, wherein at least one R^{b} is —C(O)NH₂ or —C(O)OH.

4. The compound of claim 2, represented by Formula IIA: 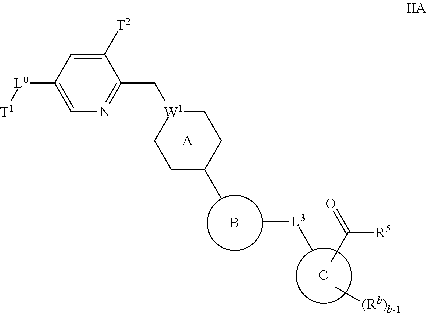
or a pharmaceutically acceptable salt or solvate thereof, wherein R⁵ is —OH or —NH₂.

5. The compound of claim 4, having the structure:

| No. | Structure |
|---|---|
| A-1 | 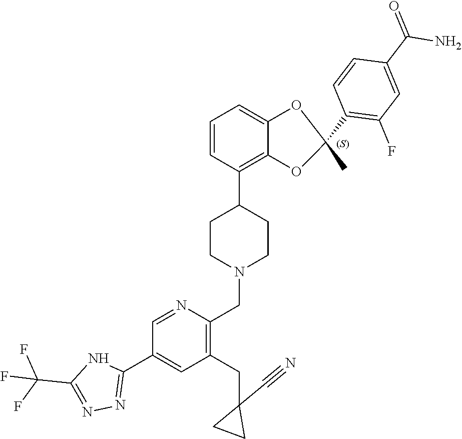 |
| A-3 | 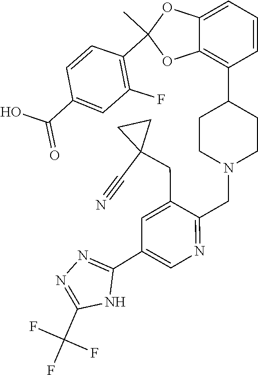 [SMILES: CC1(c2ccc(C(=O)O)cc2F)Oc2cccc(C3CCN(Cc4ncc(-c5nnc(C(F)(F)F)[nH]5)cc4CC4(C#N)CC4)CC3)c2O1] |

or a pharmaceutically acceptable salt or solvate thereof.

6. The compound of claim 2, wherein Ring A is (C₅-C₈)cycloalkylene optionally substituted with 1-4 R^{Y}.

7. The compound of claim 2, represented by Formula IIB: 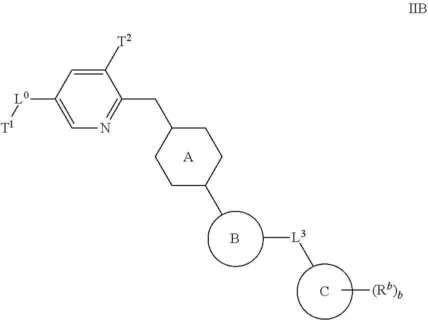
or a pharmaceutically acceptable salt or solvate thereof.

8. The compound of claim 7, having the structure:

| No. | Structure |
|---|---|
| B-1 | 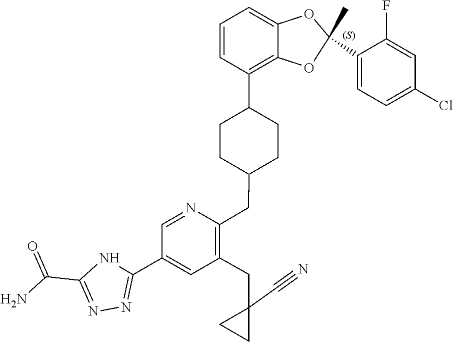 |

or a pharmaceutically acceptable salt or solvate thereof.

9. The compound of claim 2, wherein Ring B is

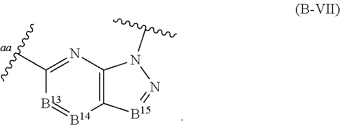

10. The compound of claim 2, represented by Formula IIC: 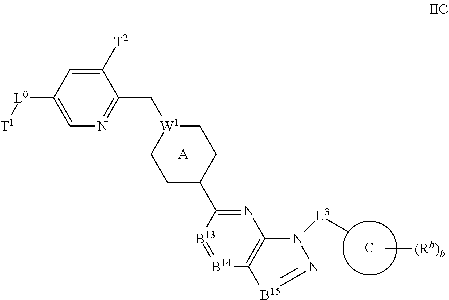
or a pharmaceutically acceptable salt or solvate thereof.

11. The compound of claim 10, having the structure:

| No. | Structure |
|---|---|
| C-1 | 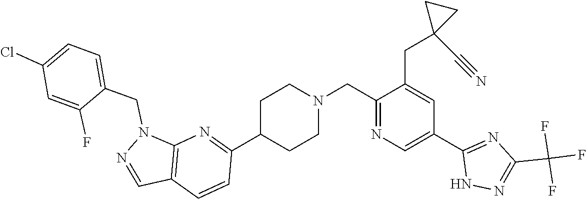 [SMILES: N#CC1(Cc2cc(-c3nc(C(F)(F)F)n[nH]3)cnc2CN2CCC(c3ccc4cnn(Cc5ccc(Cl)cc5F)c4n3)CC2)CC1] |

or a pharmaceutically acceptable salt or solvate thereof.

12. The compound of claim 2, wherein the moiety -L⁰-T¹ is —C(O)NH—(C₁-C₆)alkyl or —C(O)NH—(C₃-C₆)cycloalkyl; wherein each is optionally substituted with 1-4 R^{T}.

13. The compound of claim 2, represented by Formula IID: 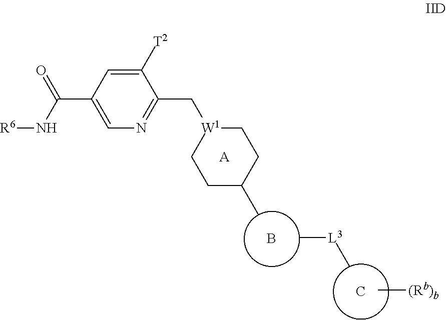
or a pharmaceutically acceptable salt or solvate thereof, wherein R⁶ is —(C₁-C₆)alkyl or —(C₃-C₆)cycloalkyl; and wherein the —(C₁-C₆)alkyl or —(C₃-C₆)cycloalkyl is optionally substituted with 1-4 R^{T}.

14. The compound of claim 13, having the structure:

| No. | Structure |
|---|---|
| D-1 | 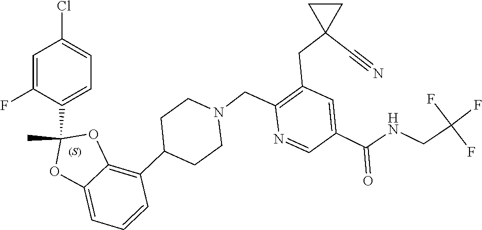 |
| D-2 | 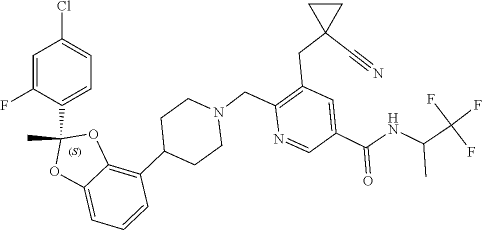 |
| D-3 | 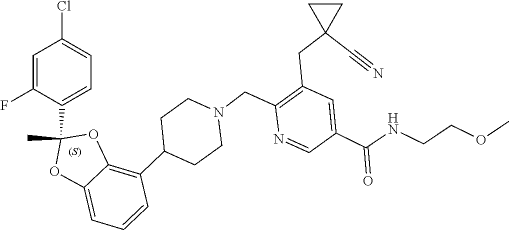 |
| D-4 | 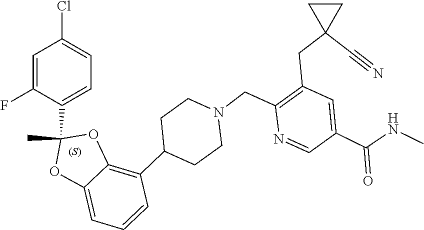 |
| D-5 | 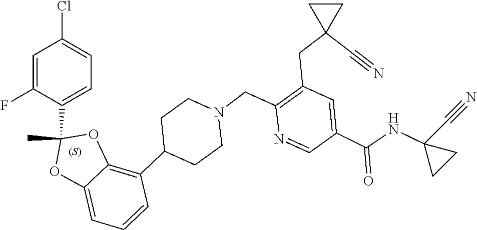 |
| D-6 | 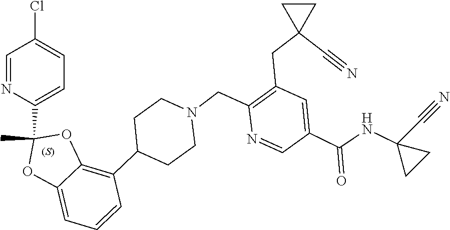 |
| D-7 | 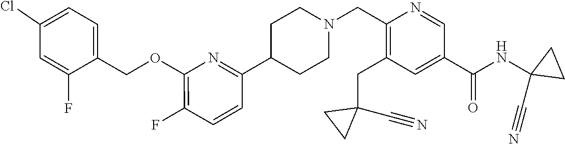 [SMILES: N#CC1(Cc2cc(C(=O)NC3(C#N)CC3)cnc2CN2CCC(c3ccc(F)c(OCc4ccc(Cl)cc4F)n3)CC2)CC1] |

or a pharmaceutically acceptable salt or solvate thereof.

15. The compound of claim 1, wherein the moiety -L⁰-T¹ is a 5- to 6-membered heteroaryl optionally substituted with —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein each —C(O)NH(C₁-C₆)alkyl, —C(O)O(C₁-C₆)alkyl, —C(O)NH—(C₃-C₆)cycloalkyl, 3- to 6-membered heterocycloalkyl, and 6-membered heteroaryl is optionally substituted with 1-4 R^{T}.

16. The compound of claim 2, represented by Formula IIE: 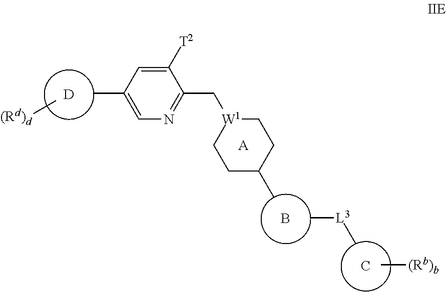
or a pharmaceutically acceptable salt or solvate thereof, wherein:
Ring D is a 5- to 6-membered heteroaryl;
d is 0 or 1; and
R^{d} is —C(O)OH, —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl; wherein the —C(O)O(C₁-C₆)alkyl, —C(O)NH(C₁-C₆)alkyl, —C(O)NH—(C₃-C₆)cycloalkyl, 3- to 6-membered heterocycloalkyl, or 6-membered heteroaryl is optionally substituted with 1-4 R^{T}.

17. The compound of claim 16, having the structure:

| No. | Structure |
|---|---|
| E-2 | 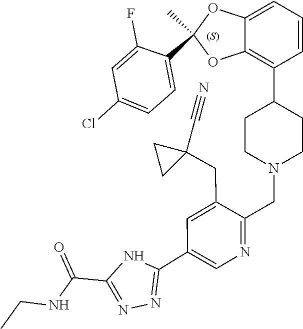 |
| E-3 | 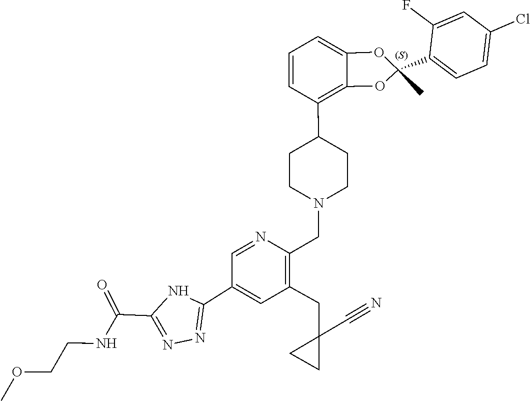 |
| E-4 | 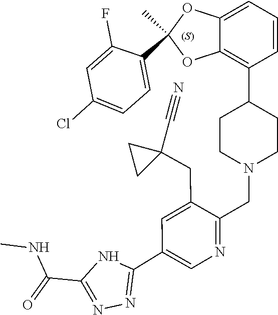 |
| E-5 | 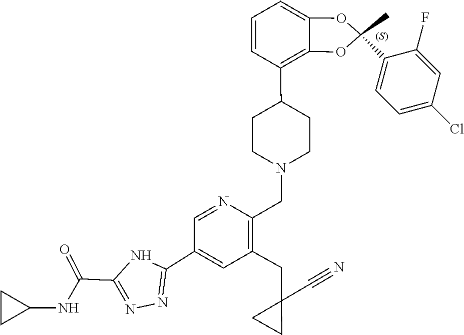 |
| E-6 | 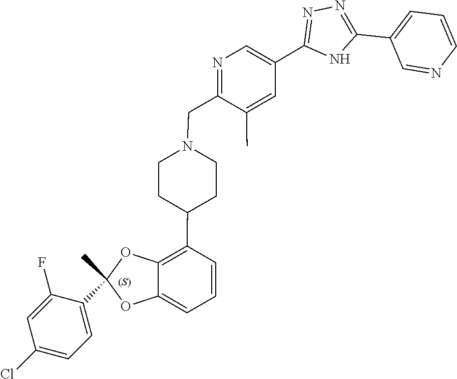 |
| E-7 | 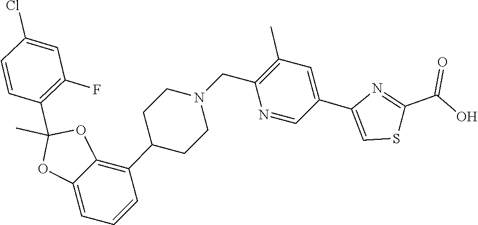 [SMILES: Cc1cc(-c2csc(C(=O)O)n2)cnc1CN1CCC(c2cccc3c2OC(C)(c2ccc(Cl)cc2F)O3)CC1] |
| E-8 | 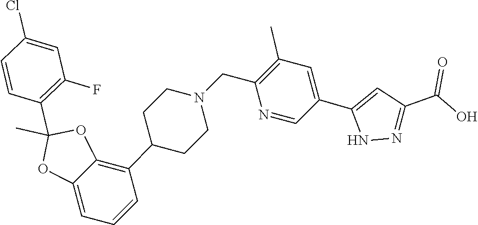 [SMILES: Cc1cc(-c2cc(C(=O)O)n[nH]2)cnc1CN1CCC(c2cccc3c2OC(C)(c2ccc(Cl)cc2F)O3)CC1] |
| E-9 | 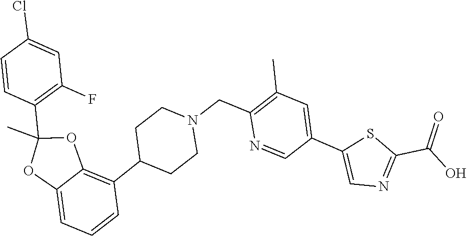 [SMILES: Cc1cc(-c2cnc(C(=O)O)s2)cnc1CN1CCC(c2cccc3c2OC(C)(c2ccc(Cl)cc2F)O3)CC1] |
| E-10 | 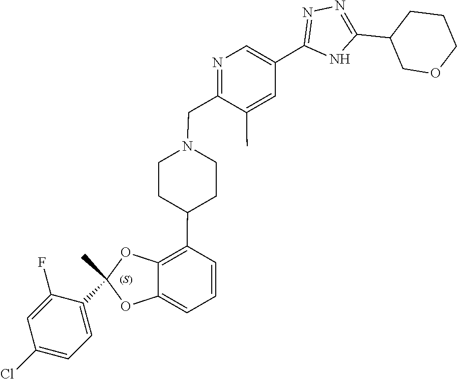 |
| E-11 | 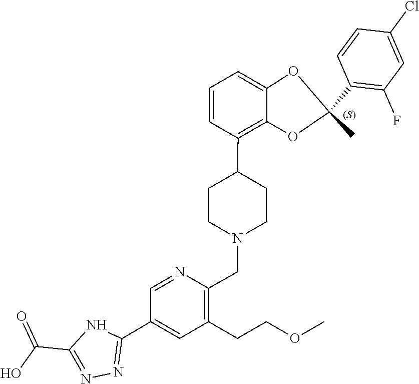 |
| E-12 | 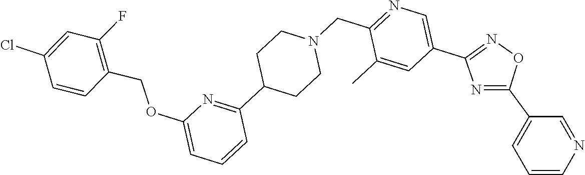 [SMILES: Cc1cc(-c2noc(-c3cccnc3)n2)cnc1CN1CCC(c2cccc(OCc3ccc(Cl)cc3F)n2)CC1] |
| E-13 | 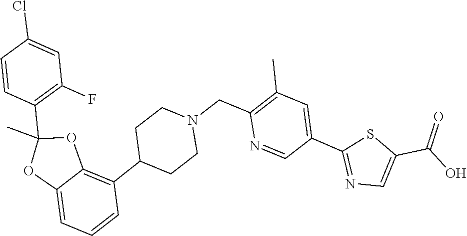 [SMILES: Cc1cc(-c2ncc(C(=O)O)s2)cnc1CN1CCC(c2cccc3c2OC(C)(c2ccc(Cl)cc2F)O3)CC1] |
| E-14 | 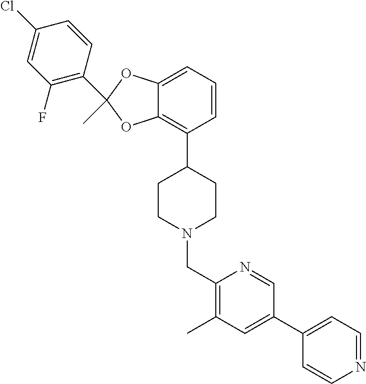 [SMILES: Cc1cc(-c2ccncc2)cnc1CN1CCC(c2cccc3c2OC(C)(c2ccc(Cl)cc2F)O3)CC1] |
| E-15 | 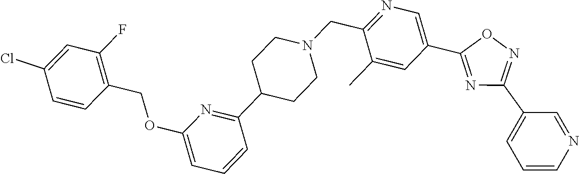 [SMILES: Cc1cc(-c2nc(-c3cccnc3)no2)cnc1CN1CCC(c2cccc(OCc3ccc(Cl)cc3F)n2)CC1] |
| E-16 | 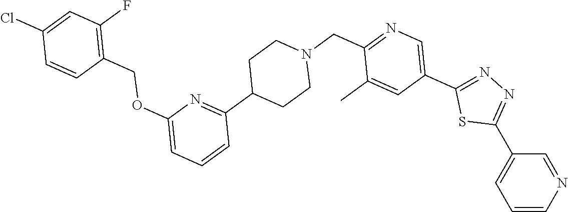 [SMILES: Cc1cc(-c2nnc(-c3cccnc3)s2)cnc1CN1CCC(c2cccc(OCc3ccc(Cl)cc3F)n2)CC1] |
| E-17 | 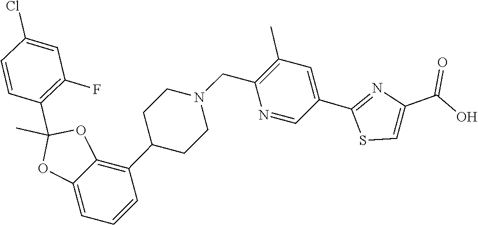 [SMILES: Cc1cc(-c2nc(C(=O)O)cs2)cnc1CN1CCC(c2cccc3c2OC(C)(c2ccc(Cl)cc2F)O3)CC1] |
| E-18 | 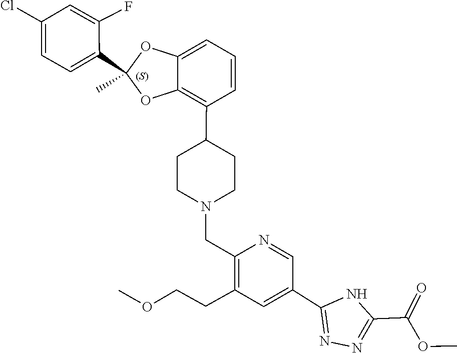 |
| E-19 | 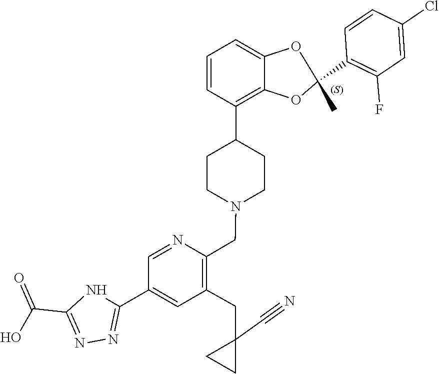 |

or a pharmaceutically acceptable salt or solvate thereof.

18. The compound of claim 2, wherein T² is —NH(C₁-C₆)alkyl, —NH—(C₃-C₆)cycloalkyl,
—CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl; wherein each —NH(C₁-C₆)alkyl, —NH—(C₃-C₆)cycloalkyl, —CH₂—S(O)₂—(C₃-C₆)cycloalkyl, —O—(C₃-C₆)cycloalkyl, (C₁-C₆)alkoxy, (C₃-C₆)cycloalkyl, or 3-10 membered heterocycloalkyl is optionally substituted with 1-4 R^{T}.

19. The compound of claim 2, represented by Formula IIF: 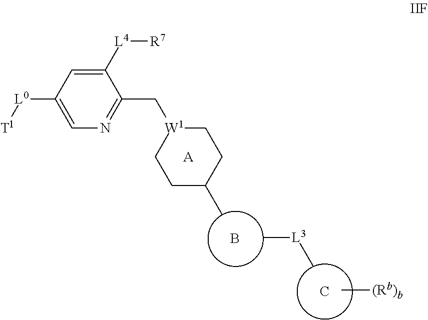
or a pharmaceutically acceptable salt or solvate thereof, wherein:
L⁴ is a bond, —NH—, —CH₂—S(O)₂, or —O—; and
R⁷ is (C₁-C₆)alkyl, (C₃-C₆)cycloalkyl, or 3-10 membered heterocycloalkyl, wherein each (C₁-C₆)alkyl, (C₃-C₆)cycloalkyl, or 3-10 membered heterocycloalkyl is optionally substituted with 1-4 R^{T}; provided that when L⁴ is a bond, then R⁷ is substituted or unsubstituted (C₃-C₆)cycloalkyl, or substituted or unsubstituted 3-10 membered heterocycloalkyl.

20. The compound of claim 19, having the structure:

| No. | Structure |
|---|---|
| F-1 | 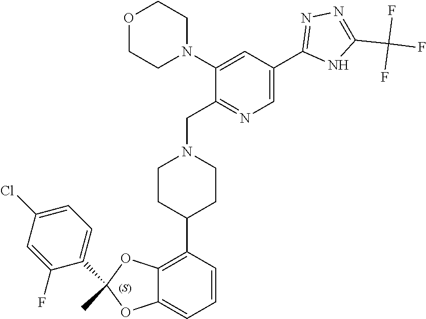 |
| F-2 | 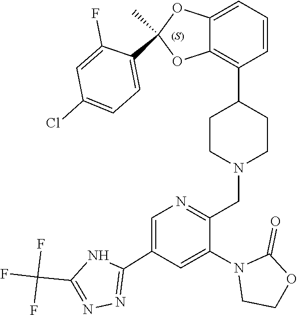 |
| F-3 | 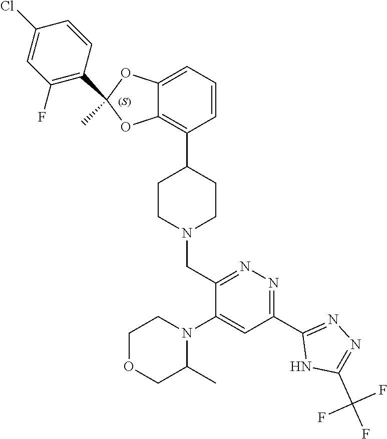 |
| F-4 | 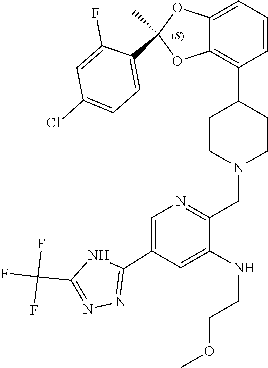 |
| F-5 | 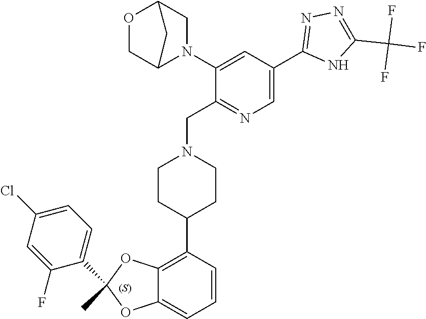 |
| F-6 | 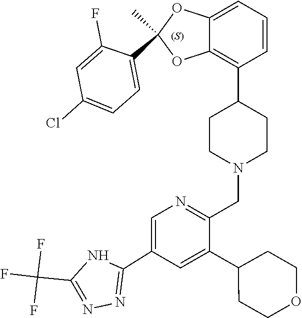 |
| F-7 | 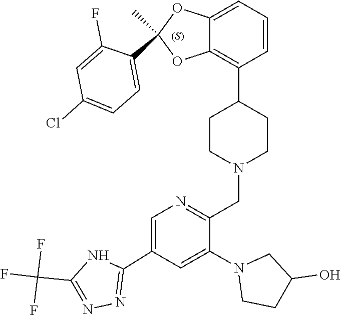 |
| F-8 | 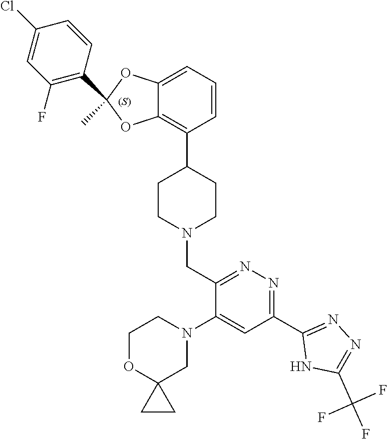 |
| F-9 | 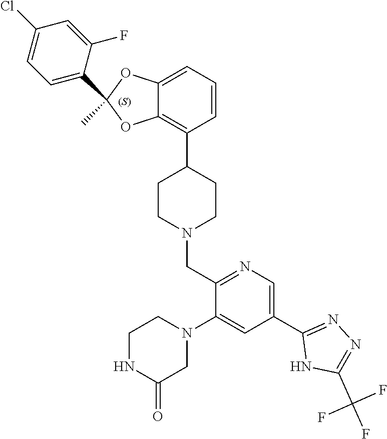 |
| F-10 | 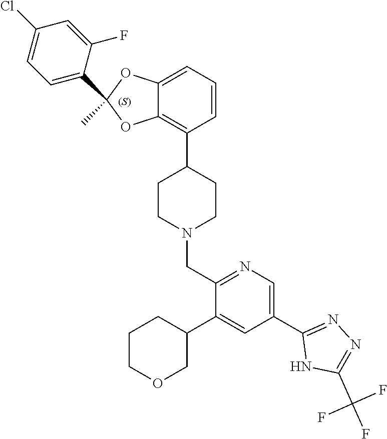 |
| F-11 | 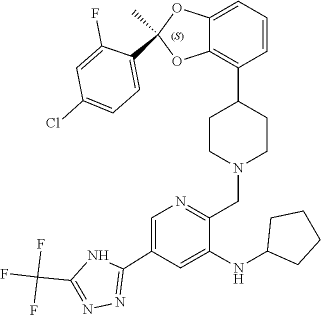 |
| F-12 | 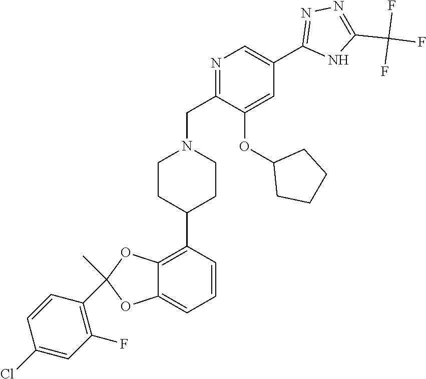 [SMILES: CC1(c2ccc(Cl)cc2F)Oc2cccc(C3CCN(Cc4ncc(-c5nnc(C(F)(F)F)[nH]5)cc4OC4CCCC4)CC3)c2O1] |
| F-13 | 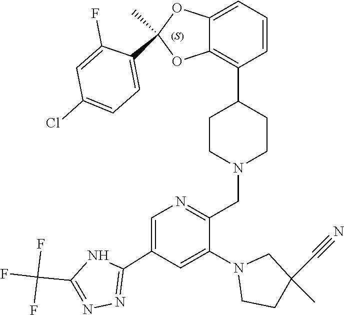 |
| F-14 | 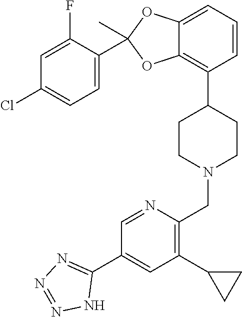 [SMILES: CC1(c2ccc(Cl)cc2F)Oc2cccc(C3CCN(Cc4ncc(-c5nnn[nH]5)cc4C4CC4)CC3)c2O1] |
| F-15 | 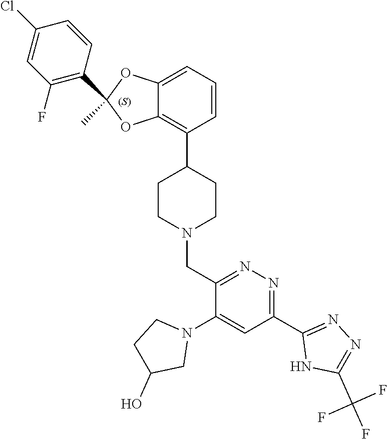 |
| F-16 | 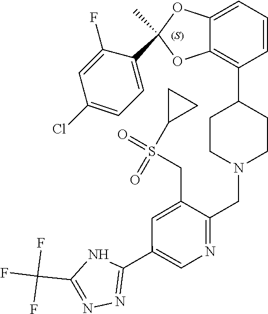 |
| F-17 | 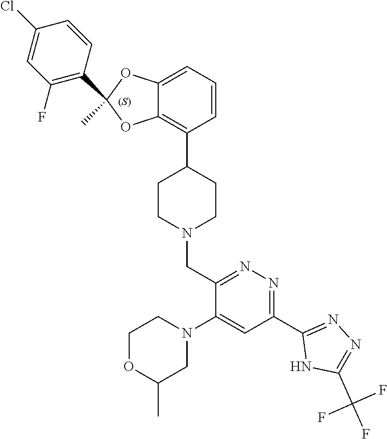 |
| F-18 | 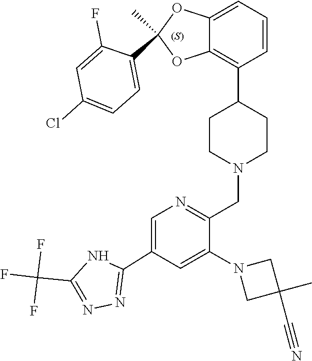 |
| F-19 | 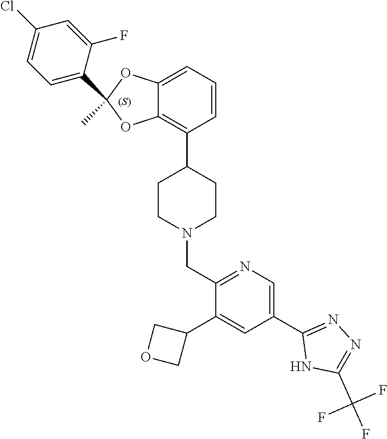 |
| F-20 | 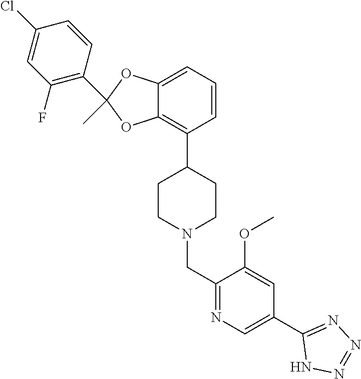 [SMILES: COc1cc(-c2nnn[nH]2)cnc1CN1CCC(c2cccc3c2OC(C)(c2ccc(Cl)cc2F)O3)CC1] |

or a pharmaceutically acceptable salt or solvate thereof.

21. A compound selected from: 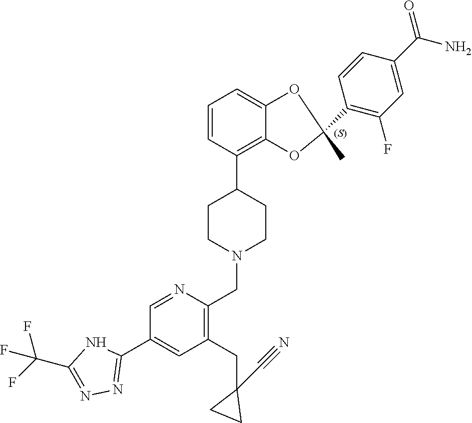 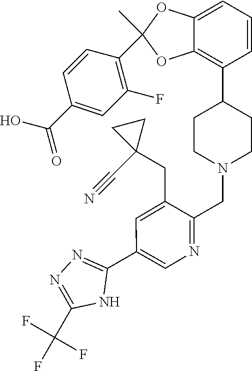 [SMILES: CC1(c2ccc(C(=O)O)cc2F)Oc2cccc(C3CCN(Cc4ncc(-c5nnc(C(F)(F)F)[nH]5)cc4CC4(C#N)CC4)CC3)c2O1] 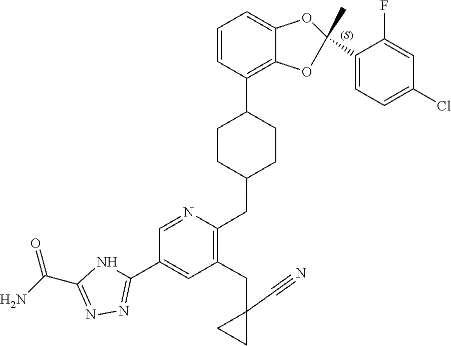 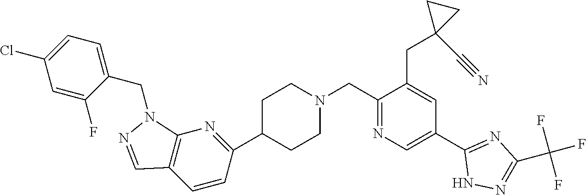 [SMILES: N#CC1(Cc2cc(-c3nc(C(F)(F)F)n[nH]3)cnc2CN2CCC(c3ccc4cnn(Cc5ccc(Cl)cc5F)c4n3)CC2)CC1] 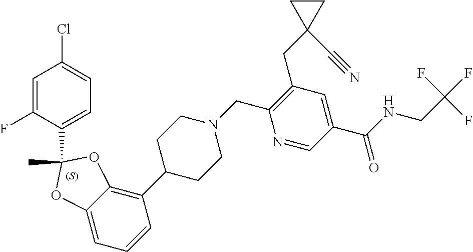 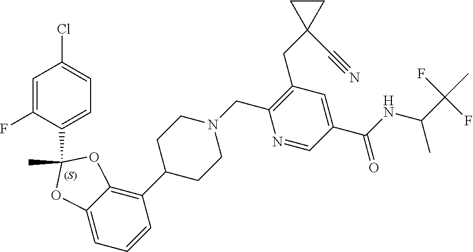 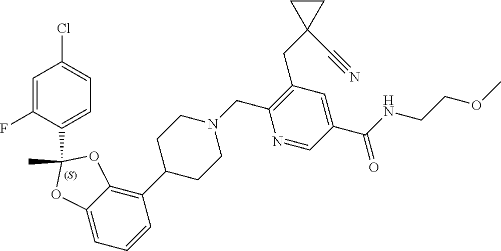 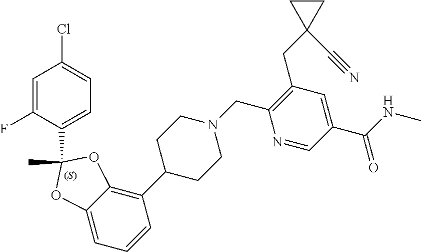 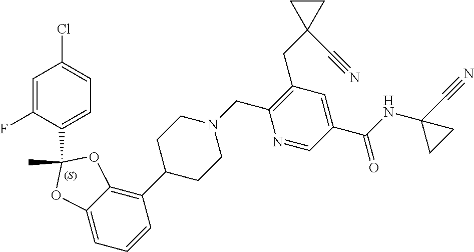 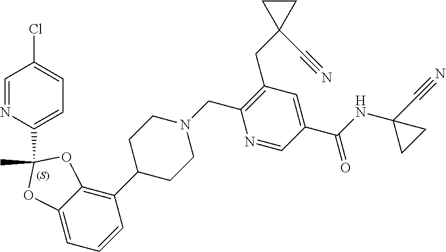 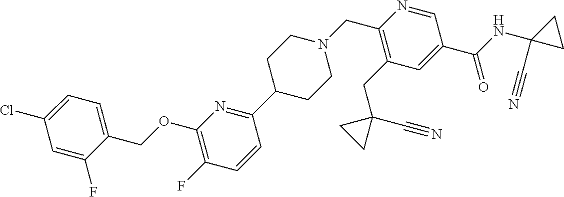 [SMILES: N#CC1(Cc2cc(C(=O)NC3(C#N)CC3)cnc2CN2CCC(c3ccc(F)c(OCc4ccc(Cl)cc4F)n3)CC2)CC1] 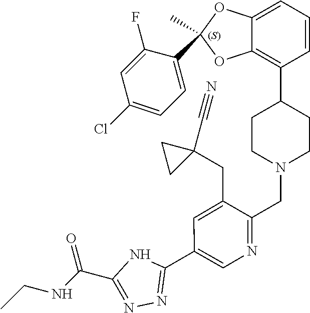 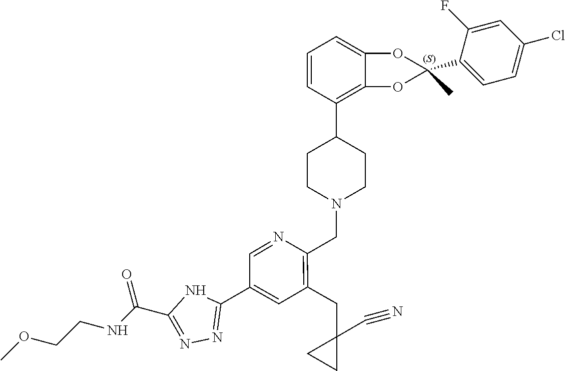 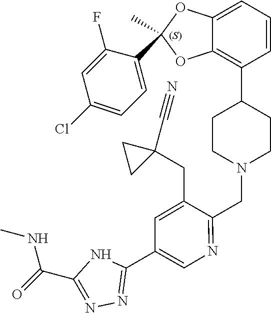 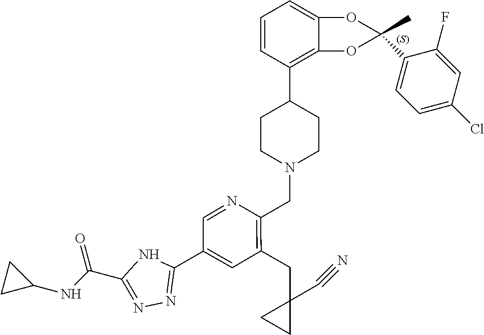 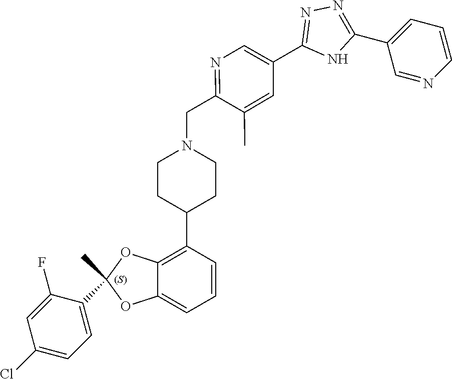 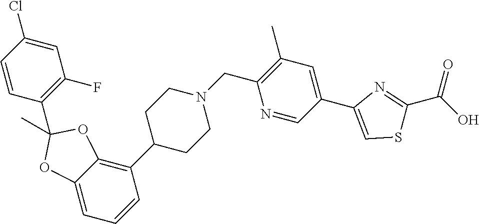 [SMILES: Cc1cc(-c2csc(C(=O)O)n2)cnc1CN1CCC(c2cccc3c2OC(C)(c2ccc(Cl)cc2F)O3)CC1] 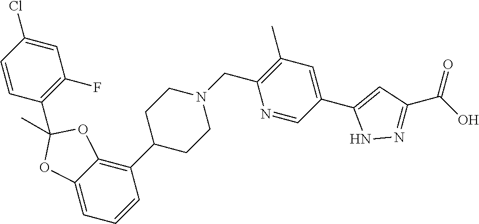 [SMILES: Cc1cc(-c2cc(C(=O)O)n[nH]2)cnc1CN1CCC(c2cccc3c2OC(C)(c2ccc(Cl)cc2F)O3)CC1] 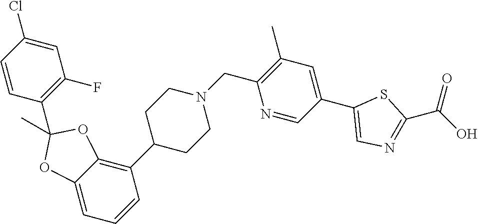 [SMILES: Cc1cc(-c2cnc(C(=O)O)s2)cnc1CN1CCC(c2cccc3c2OC(C)(c2ccc(Cl)cc2F)O3)CC1] 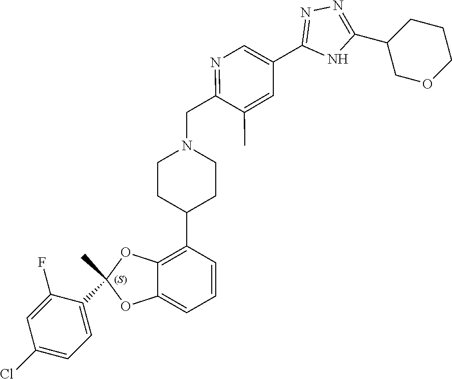 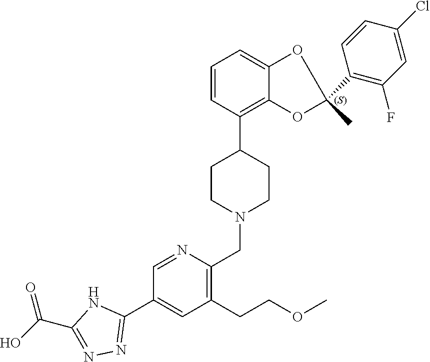 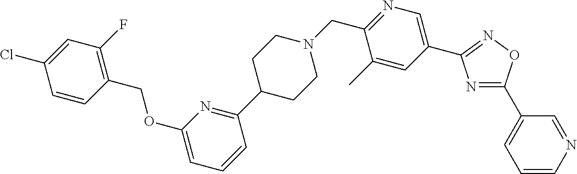 [SMILES: Cc1cc(-c2noc(-c3cccnc3)n2)cnc1CN1CCC(c2cccc(OCc3ccc(Cl)cc3F)n2)CC1] 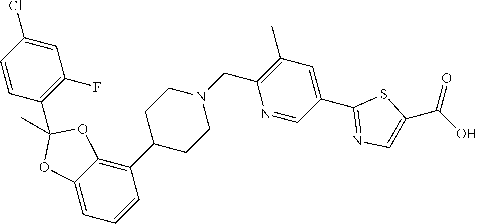 [SMILES: Cc1cc(-c2ncc(C(=O)O)s2)cnc1CN1CCC(c2cccc3c2OC(C)(c2ccc(Cl)cc2F)O3)CC1] 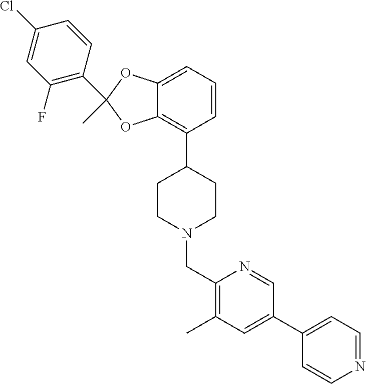 [SMILES: Cc1cc(-c2ccncc2)cnc1CN1CCC(c2cccc3c2OC(C)(c2ccc(Cl)cc2F)O3)CC1] 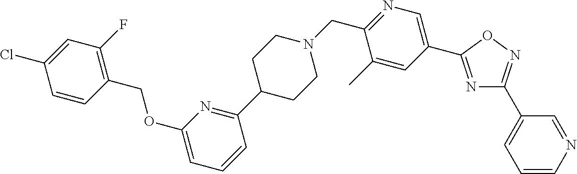 [SMILES: Cc1cc(-c2nc(-c3cccnc3)no2)cnc1CN1CCC(c2cccc(OCc3ccc(Cl)cc3F)n2)CC1] 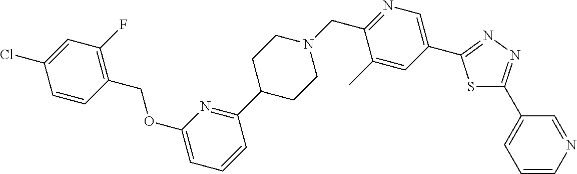 [SMILES: Cc1cc(-c2nnc(-c3cccnc3)s2)cnc1CN1CCC(c2cccc(OCc3ccc(Cl)cc3F)n2)CC1] 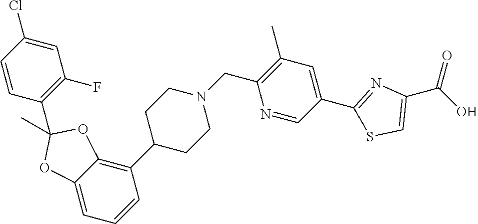 [SMILES: Cc1cc(-c2nc(C(=O)O)cs2)cnc1CN1CCC(c2cccc3c2OC(C)(c2ccc(Cl)cc2F)O3)CC1] 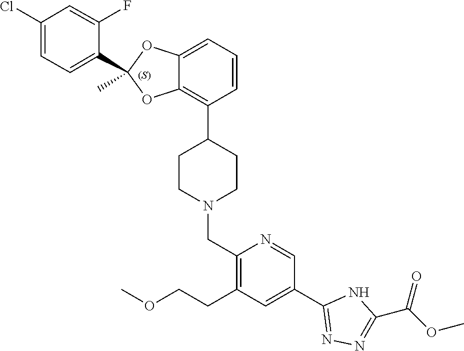 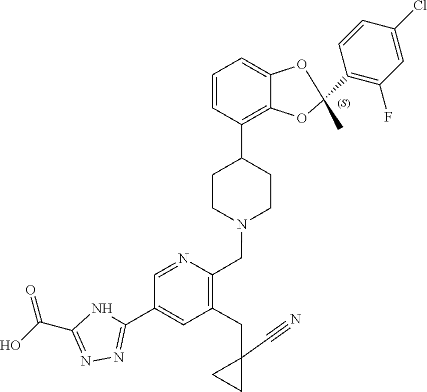 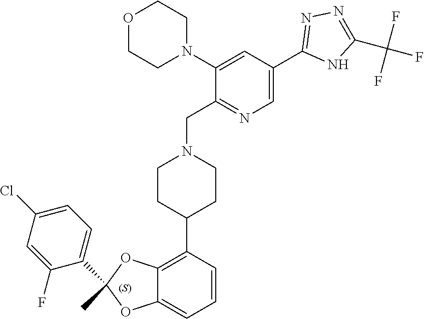 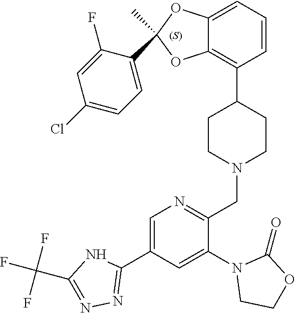 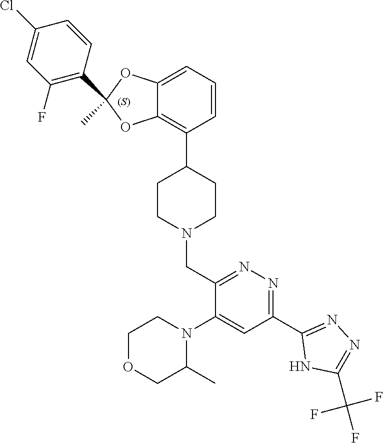 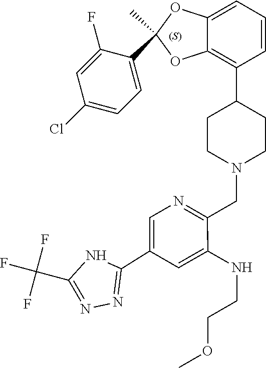 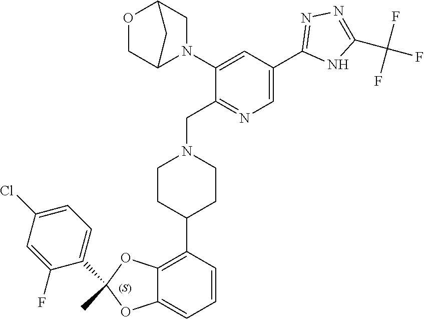 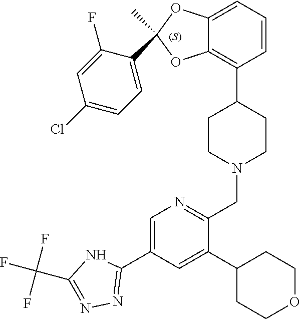 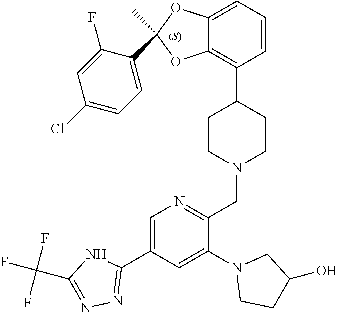 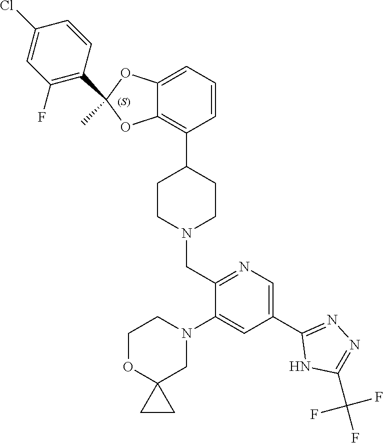 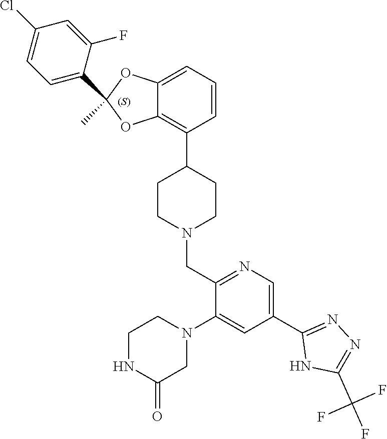 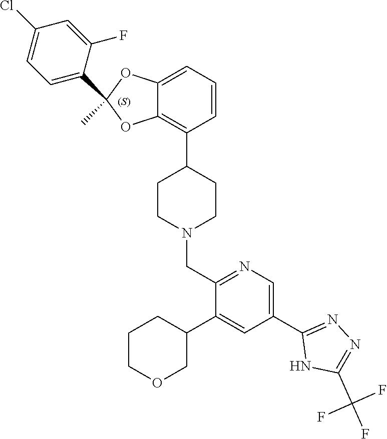 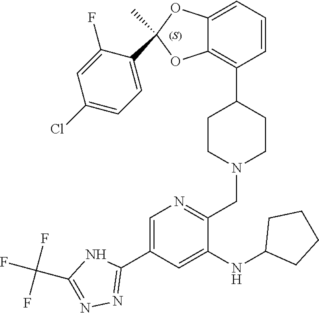 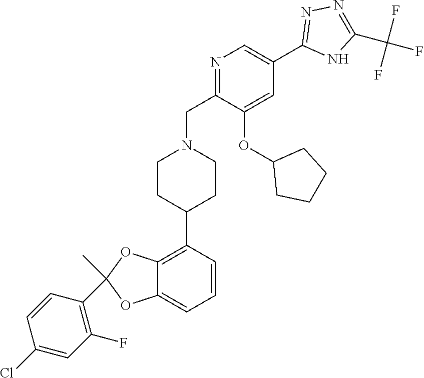 [SMILES: CC1(c2ccc(Cl)cc2F)Oc2cccc(C3CCN(Cc4ncc(-c5nnc(C(F)(F)F)[nH]5)cc4OC4CCCC4)CC3)c2O1] 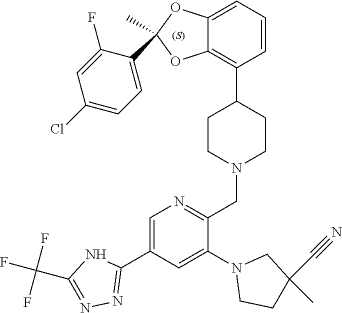 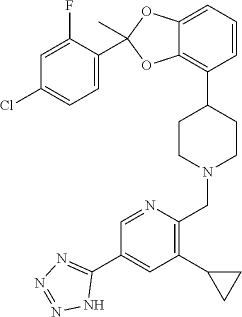 [SMILES: CC1(c2ccc(Cl)cc2F)Oc2cccc(C3CCN(Cc4ncc(-c5nnn[nH]5)cc4C4CC4)CC3)c2O1] 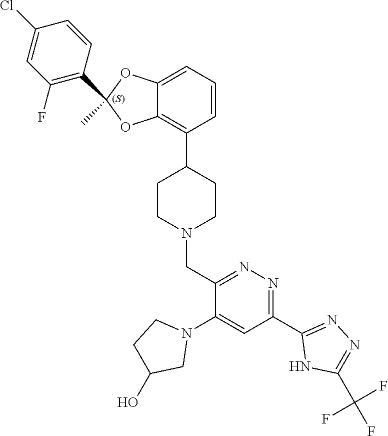 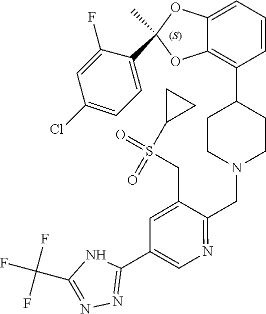 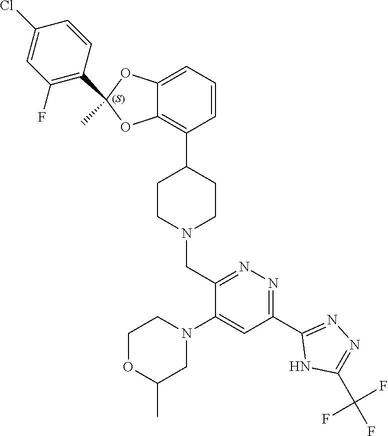 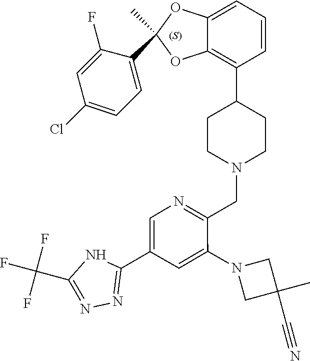 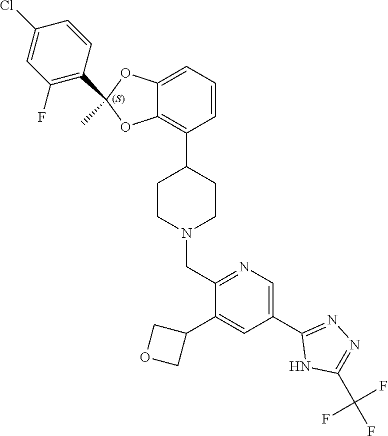 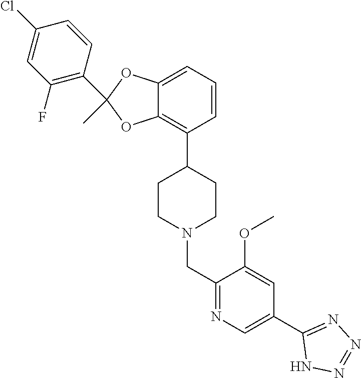 [SMILES: COc1cc(-c2nnn[nH]2)cnc1CN1CCC(c2cccc3c2OC(C)(c2ccc(Cl)cc2F)O3)CC1] 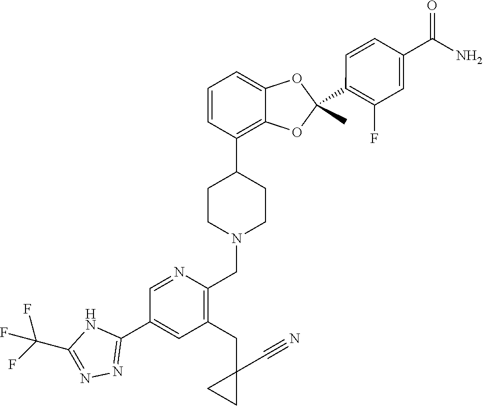 [SMILES: C[C@]1(c2ccc(C(N)=O)cc2F)Oc2cccc(C3CCN(Cc4ncc(-c5nnc(C(F)(F)F)[nH]5)cc4CC4(C#N)CC4)CC3)c2O1] 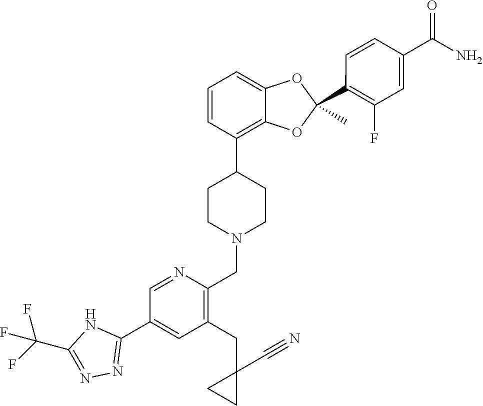 [SMILES: C[C@@]1(c2ccc(C(N)=O)cc2F)Oc2cccc(C3CCN(Cc4ncc(-c5nnc(C(F)(F)F)[nH]5)cc4CC4(C#N)CC4)CC3)c2O1] 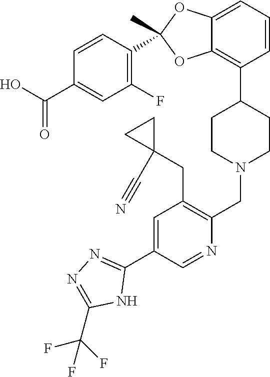 [SMILES: C[C@@]1(c2ccc(C(=O)O)cc2F)Oc2cccc(C3CCN(Cc4ncc(-c5nnc(C(F)(F)F)[nH]5)cc4CC4(C#N)CC4)CC3)c2O1] 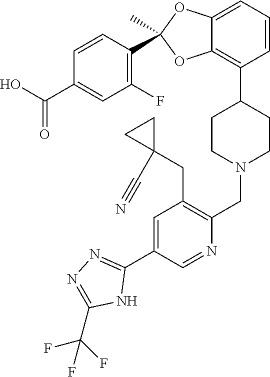 [SMILES: C[C@]1(c2ccc(C(=O)O)cc2F)Oc2cccc(C3CCN(Cc4ncc(-c5nnc(C(F)(F)F)[nH]5)cc4CC4(C#N)CC4)CC3)c2O1] 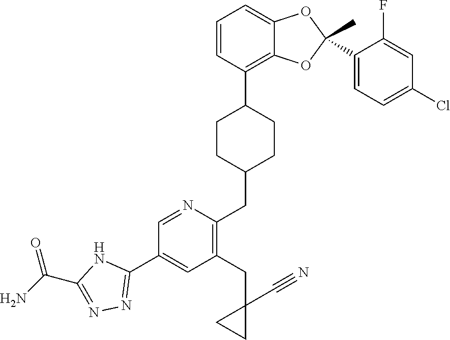 [SMILES: C[C@]1(c2ccc(Cl)cc2F)Oc2cccc(C3CCC(Cc4ncc(-c5nnc(C(N)=O)[nH]5)cc4CC4(C#N)CC4)CC3)c2O1] 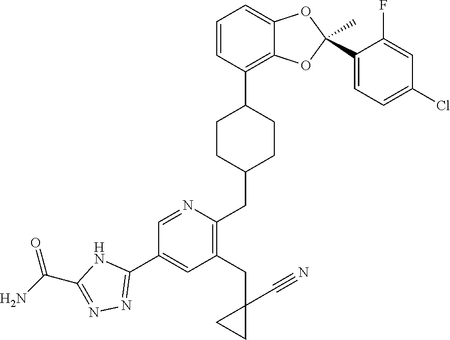 [SMILES: C[C@@]1(c2ccc(Cl)cc2F)Oc2cccc(C3CCC(Cc4ncc(-c5nnc(C(N)=O)[nH]5)cc4CC4(C#N)CC4)CC3)c2O1] 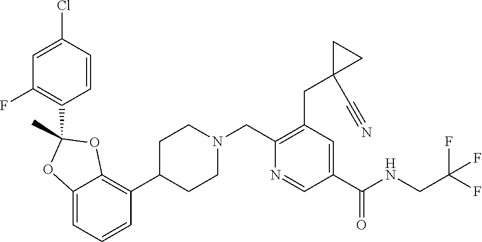 [SMILES: C[C@]1(c2ccc(Cl)cc2F)Oc2cccc(C3CCN(Cc4ncc(C(=O)NCC(F)(F)F)cc4CC4(C#N)CC4)CC3)c2O1] 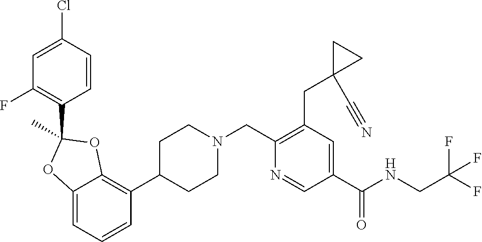 [SMILES: C[C@@]1(c2ccc(Cl)cc2F)Oc2cccc(C3CCN(Cc4ncc(C(=O)NCC(F)(F)F)cc4CC4(C#N)CC4)CC3)c2O1] 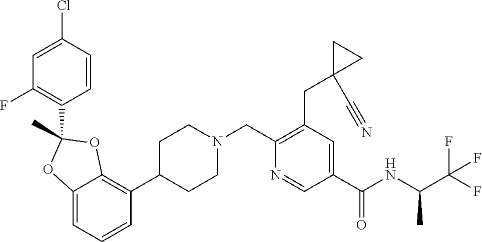 [SMILES: C[C@@H](NC(=O)c1cnc(CN2CCC(c3cccc4c3O[C@@](C)(c3ccc(Cl)cc3F)O4)CC2)c(CC2(C#N)CC2)c1)C(F)(F)F] 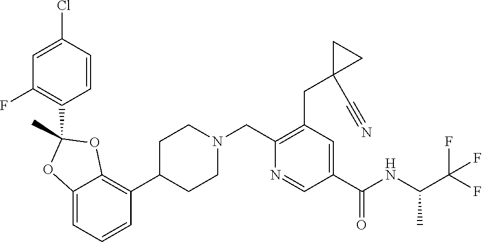 [SMILES: C[C@H](NC(=O)c1cnc(CN2CCC(c3cccc4c3O[C@@](C)(c3ccc(Cl)cc3F)O4)CC2)c(CC2(C#N)CC2)c1)C(F)(F)F] 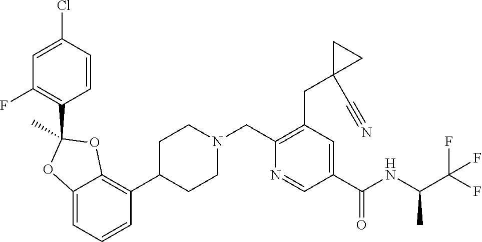 [SMILES: C[C@@H](NC(=O)c1cnc(CN2CCC(c3cccc4c3O[C@](C)(c3ccc(Cl)cc3F)O4)CC2)c(CC2(C#N)CC2)c1)C(F)(F)F] 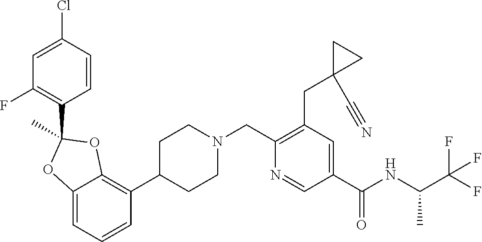 [SMILES: C[C@H](NC(=O)c1cnc(CN2CCC(c3cccc4c3O[C@](C)(c3ccc(Cl)cc3F)O4)CC2)c(CC2(C#N)CC2)c1)C(F)(F)F] 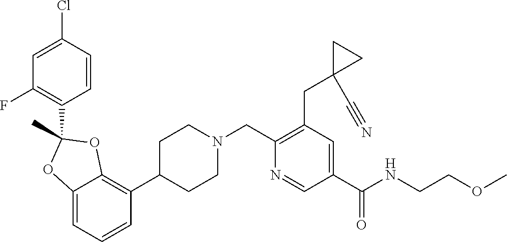 [SMILES: COCCNC(=O)c1cnc(CN2CCC(c3cccc4c3O[C@@](C)(c3ccc(Cl)cc3F)O4)CC2)c(CC2(C#N)CC2)c1] 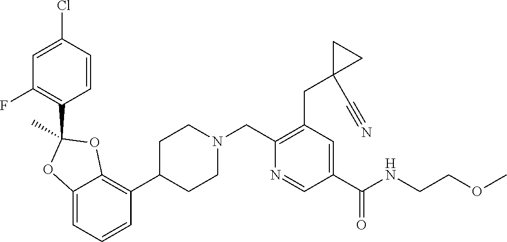 [SMILES: COCCNC(=O)c1cnc(CN2CCC(c3cccc4c3O[C@](C)(c3ccc(Cl)cc3F)O4)CC2)c(CC2(C#N)CC2)c1] 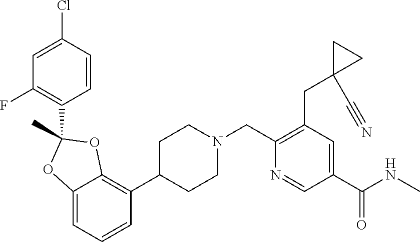 [SMILES: CNC(=O)c1cnc(CN2CCC(c3cccc4c3O[C@@](C)(c3ccc(Cl)cc3F)O4)CC2)c(CC2(C#N)CC2)c1] 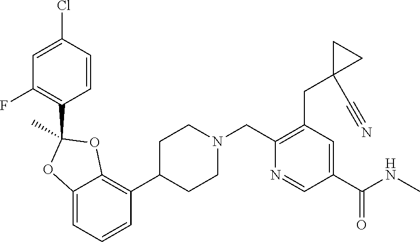 [SMILES: CNC(=O)c1cnc(CN2CCC(c3cccc4c3O[C@](C)(c3ccc(Cl)cc3F)O4)CC2)c(CC2(C#N)CC2)c1] 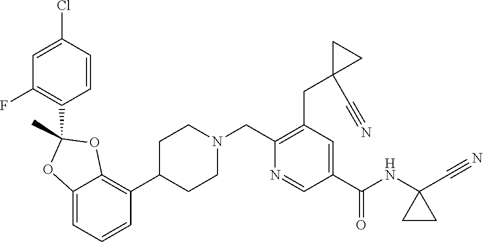 [SMILES: C[C@]1(c2ccc(Cl)cc2F)Oc2cccc(C3CCN(Cc4ncc(C(=O)NC5(C#N)CC5)cc4CC4(C#N)CC4)CC3)c2O1] 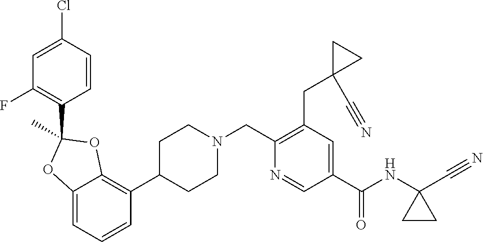 [SMILES: C[C@@]1(c2ccc(Cl)cc2F)Oc2cccc(C3CCN(Cc4ncc(C(=O)NC5(C#N)CC5)cc4CC4(C#N)CC4)CC3)c2O1] 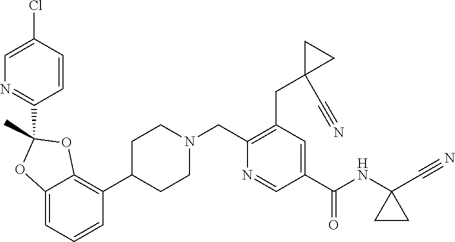 [SMILES: C[C@]1(c2ccc(Cl)cn2)Oc2cccc(C3CCN(Cc4ncc(C(=O)NC5(C#N)CC5)cc4CC4(C#N)CC4)CC3)c2O1] 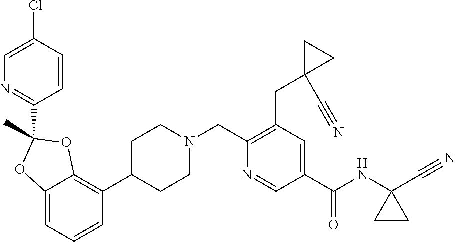 [SMILES: C[C@]1(c2ccc(Cl)cn2)Oc2cccc(C3CCN(Cc4ncc(C(=O)NC5(C#N)CC5)cc4CC4(C#N)CC4)CC3)c2O1] 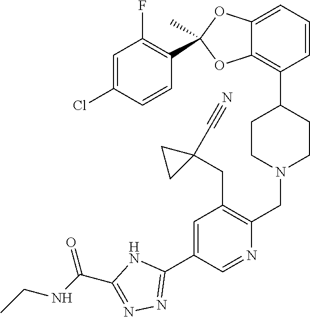 [SMILES: CCNC(=O)c1nnc(-c2cnc(CN3CCC(c4cccc5c4O[C@@](C)(c4ccc(Cl)cc4F)O5)CC3)c(CC3(C#N)CC3)c2)[nH]1] 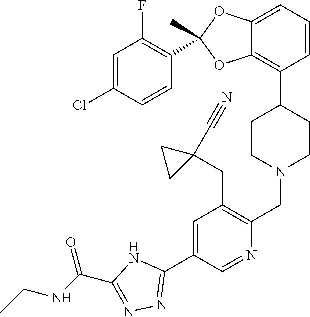 [SMILES: CCNC(=O)c1nnc(-c2cnc(CN3CCC(c4cccc5c4O[C@](C)(c4ccc(Cl)cc4F)O5)CC3)c(CC3(C#N)CC3)c2)[nH]1] 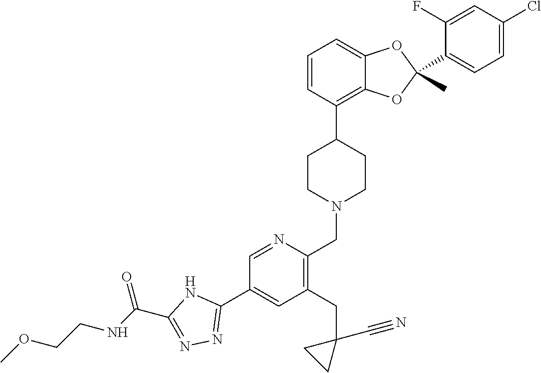 [SMILES: COCCNC(=O)c1nnc(-c2cnc(CN3CCC(c4cccc5c4O[C@@](C)(c4ccc(Cl)cc4F)O5)CC3)c(CC3(C#N)CC3)c2)[nH]1] 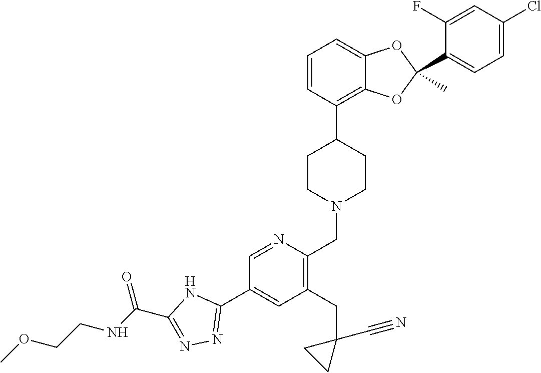 [SMILES: COCCNC(=O)c1nnc(-c2cnc(CN3CCC(c4cccc5c4O[C@](C)(c4ccc(Cl)cc4F)O5)CC3)c(CC3(C#N)CC3)c2)[nH]1] 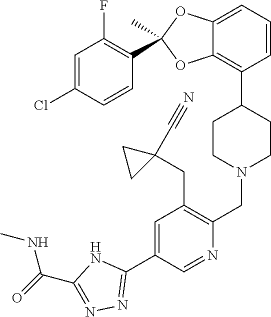 [SMILES: CNC(=O)c1nnc(-c2cnc(CN3CCC(c4cccc5c4O[C@@](C)(c4ccc(Cl)cc4F)O5)CC3)c(CC3(C#N)CC3)c2)[nH]1] 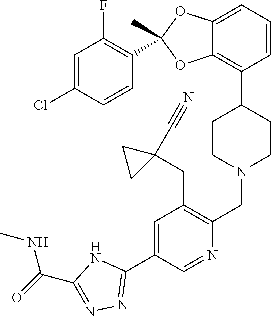 [SMILES: CNC(=O)c1nnc(-c2cnc(CN3CCC(c4cccc5c4O[C@](C)(c4ccc(Cl)cc4F)O5)CC3)c(CC3(C#N)CC3)c2)[nH]1] 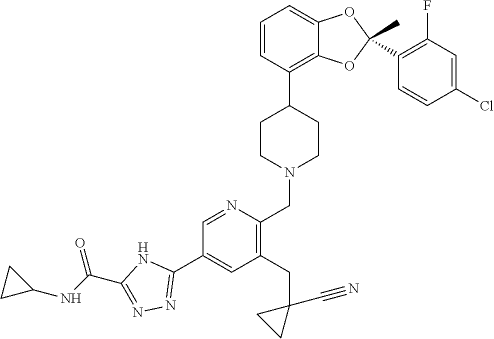 [SMILES: C[C@]1(c2ccc(Cl)cc2F)Oc2cccc(C3CCN(Cc4ncc(-c5nnc(C(=O)NC6CC6)[nH]5)cc4CC4(C#N)CC4)CC3)c2O1] 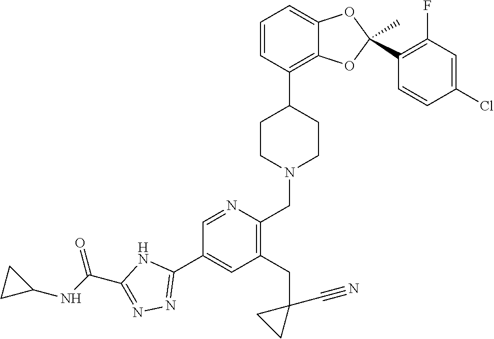 [SMILES: C[C@@]1(c2ccc(Cl)cc2F)Oc2cccc(C3CCN(Cc4ncc(-c5nnc(C(=O)NC6CC6)[nH]5)cc4CC4(C#N)CC4)CC3)c2O1] 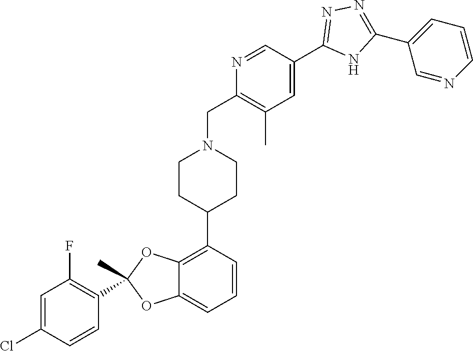 [SMILES: Cc1cc(-c2nnc(-c3cccnc3)[nH]2)cnc1CN1CCC(c2cccc3c2O[C@@](C)(c2ccc(Cl)cc2F)O3)CC1] 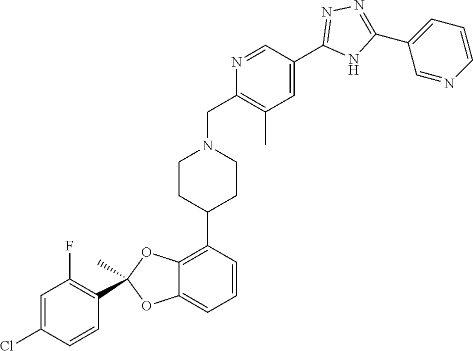 [SMILES: Cc1cc(-c2nnc(-c3cccnc3)[nH]2)cnc1CN1CCC(c2cccc3c2O[C@](C)(c2ccc(Cl)cc2F)O3)CC1] 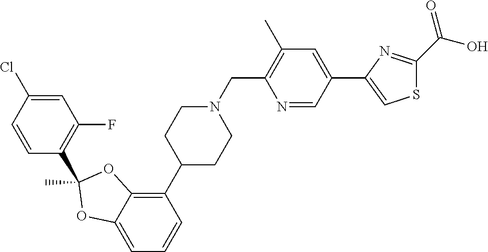 [SMILES: Cc1cc(-c2csc(C(=O)O)n2)cnc1CN1CCC(c2cccc3c2O[C@](C)(c2ccc(Cl)cc2F)O3)CC1] 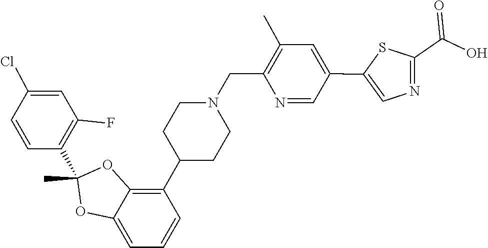 [SMILES: Cc1cc(-c2cnc(C(=O)O)s2)cnc1CN1CCC(c2cccc3c2O[C@@](C)(c2ccc(Cl)cc2F)O3)CC1] 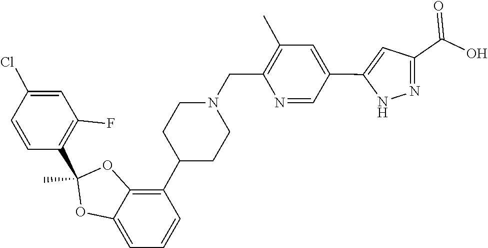 [SMILES: Cc1cc(-c2cc(C(=O)O)n[nH]2)cnc1CN1CCC(c2cccc3c2O[C@](C)(c2ccc(Cl)cc2F)O3)CC1] 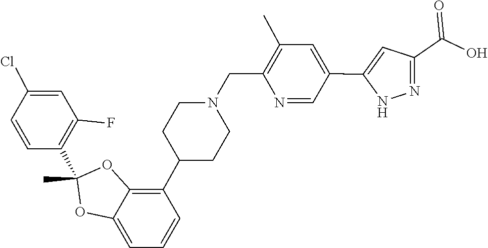 [SMILES: Cc1cc(-c2cc(C(=O)O)n[nH]2)cnc1CN1CCC(c2cccc3c2O[C@@](C)(c2ccc(Cl)cc2F)O3)CC1] 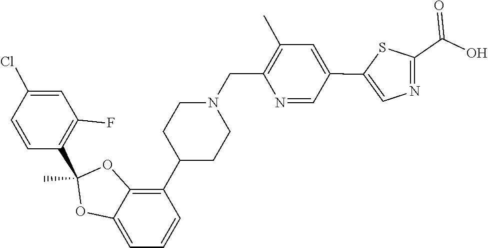 [SMILES: Cc1cc(-c2cnc(C(=O)O)s2)cnc1CN1CCC(c2cccc3c2O[C@](C)(c2ccc(Cl)cc2F)O3)CC1] 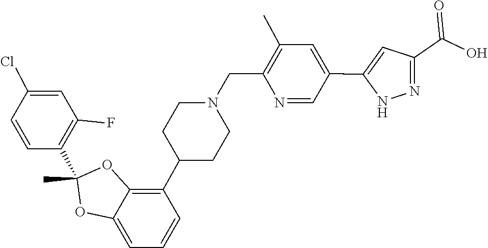 [SMILES: Cc1cc(-c2cc(C(=O)O)n[nH]2)cnc1CN1CCC(c2cccc3c2O[C@@](C)(c2ccc(Cl)cc2F)O3)CC1] 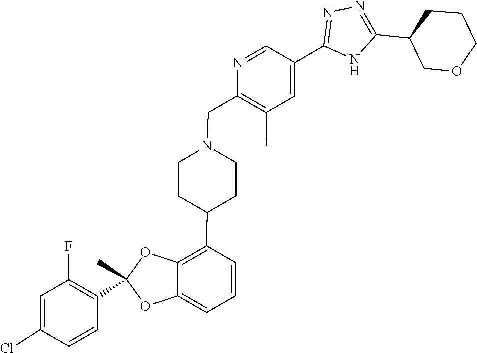 [SMILES: Cc1cc(-c2nnc([C@H]3CCCOC3)[nH]2)cnc1CN1CCC(c2cccc3c2O[C@@](C)(c2ccc(Cl)cc2F)O3)CC1] 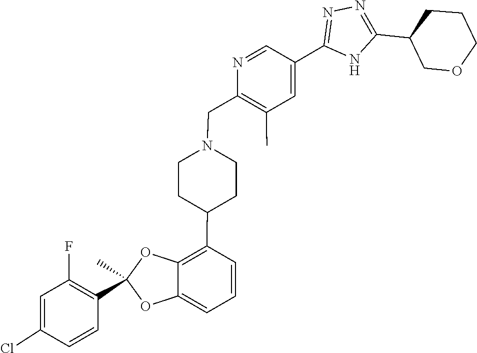 [SMILES: Cc1cc(-c2nnc([C@H]3CCCOC3)[nH]2)cnc1CN1CCC(c2cccc3c2O[C@](C)(c2ccc(Cl)cc2F)O3)CC1] 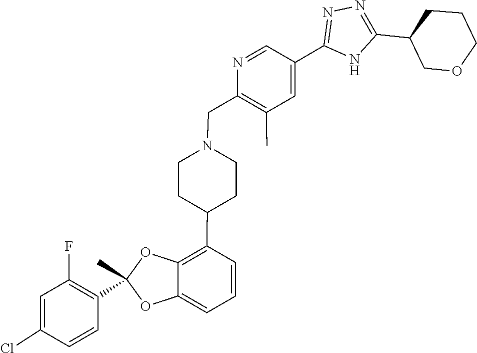 [SMILES: Cc1cc(-c2nnc([C@H]3CCCOC3)[nH]2)cnc1CN1CCC(c2cccc3c2O[C@@](C)(c2ccc(Cl)cc2F)O3)CC1] 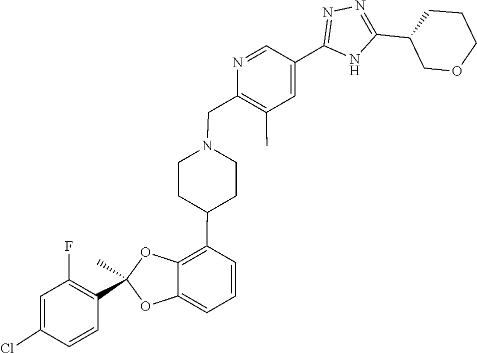 [SMILES: Cc1cc(-c2nnc([C@@H]3CCCOC3)[nH]2)cnc1CN1CCC(c2cccc3c2O[C@](C)(c2ccc(Cl)cc2F)O3)CC1] 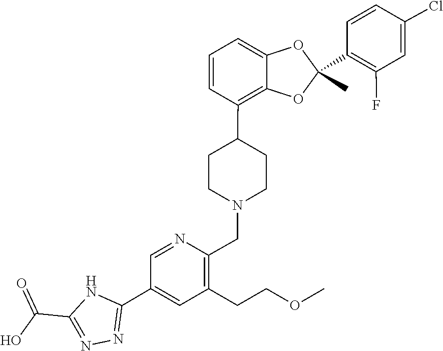 [SMILES: COCCc1cc(-c2nnc(C(=O)O)[nH]2)cnc1CN1CCC(c2cccc3c2O[C@@](C)(c2ccc(Cl)cc2F)O3)CC1] 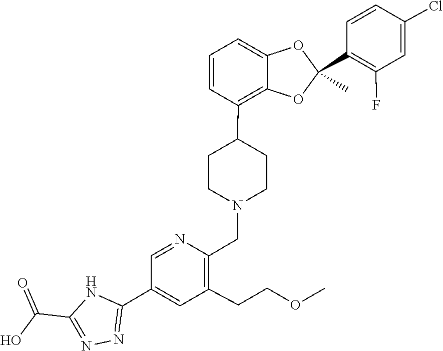 [SMILES: COCCc1cc(-c2nnc(C(=O)O)[nH]2)cnc1CN1CCC(c2cccc3c2O[C@](C)(c2ccc(Cl)cc2F)O3)CC1] 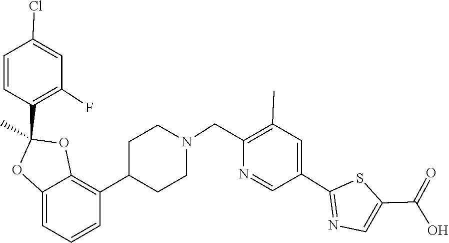 [SMILES: Cc1cc(-c2ncc(C(=O)O)s2)cnc1CN1CCC(c2cccc3c2O[C@](C)(c2ccc(Cl)cc2F)O3)CC1] 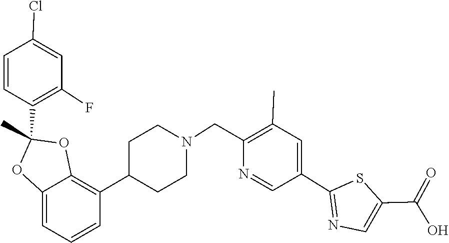 [SMILES: Cc1cc(-c2ncc(C(=O)O)s2)cnc1CN1CCC(c2cccc3c2O[C@@](C)(c2ccc(Cl)cc2F)O3)CC1] 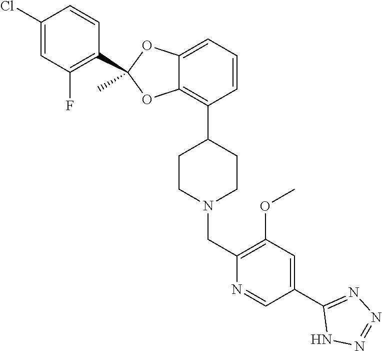 [SMILES: COc1cc(-c2nnn[nH]2)cnc1CN1CCC(c2cccc3c2O[C@@](C)(c2ccc(Cl)cc2F)O3)CC1] 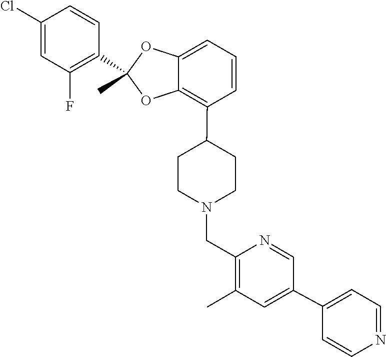 [SMILES: Cc1cc(-c2ccncc2)cnc1CN1CCC(c2cccc3c2O[C@](C)(c2ccc(Cl)cc2F)O3)CC1] 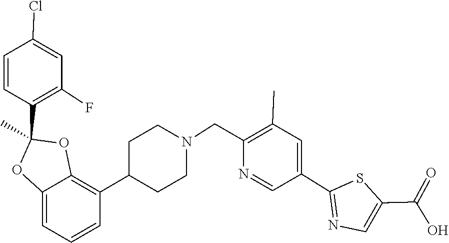 [SMILES: Cc1cc(-c2ncc(C(=O)O)s2)cnc1CN1CCC(c2cccc3c2O[C@](C)(c2ccc(Cl)cc2F)O3)CC1] 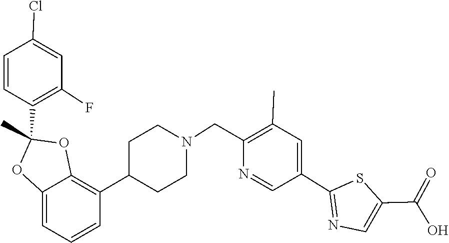 [SMILES: Cc1cc(-c2ncc(C(=O)O)s2)cnc1CN1CCC(c2cccc3c2O[C@@](C)(c2ccc(Cl)cc2F)O3)CC1] 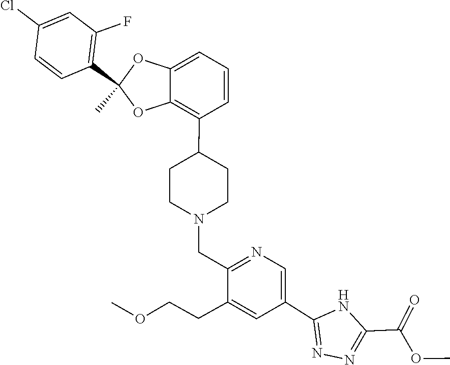 [SMILES: COCCc1cc(-c2nnc(C(=O)OC)[nH]2)cnc1CN1CCC(c2cccc3c2O[C@@](C)(c2ccc(Cl)cc2F)O3)CC1] 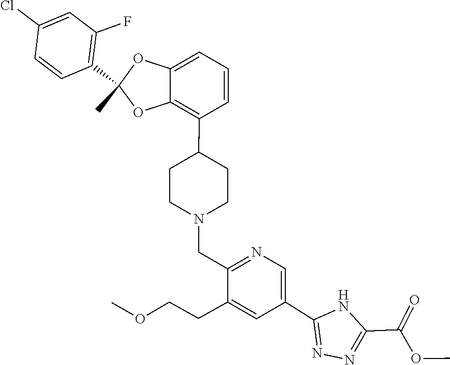 [SMILES: COCCc1cc(-c2nnc(C(=O)OC)[nH]2)cnc1CN1CCC(c2cccc3c2O[C@](C)(c2ccc(Cl)cc2F)O3)CC1] 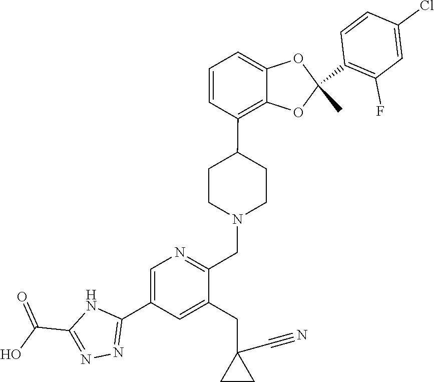 [SMILES: C[C@]1(c2ccc(Cl)cc2F)Oc2cccc(C3CCN(Cc4ncc(-c5nnc(C(=O)O)[nH]5)cc4CC4(C#N)CC4)CC3)c2O1] 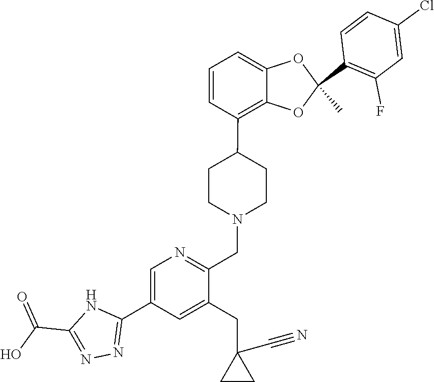 [SMILES: C[C@@]1(c2ccc(Cl)cc2F)Oc2cccc(C3CCN(Cc4ncc(-c5nnc(C(=O)O)[nH]5)cc4CC4(C#N)CC4)CC3)c2O1] 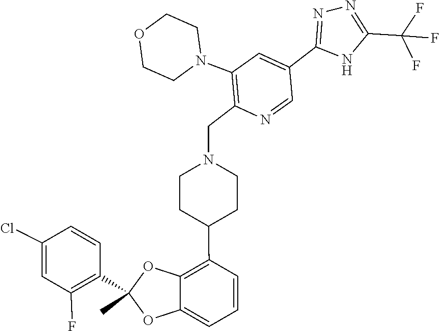 [SMILES: C[C@]1(c2ccc(Cl)cc2F)Oc2cccc(C3CCN(Cc4ncc(-c5nnc(C(F)(F)F)[nH]5)cc4N4CCOCC4)CC3)c2O1] 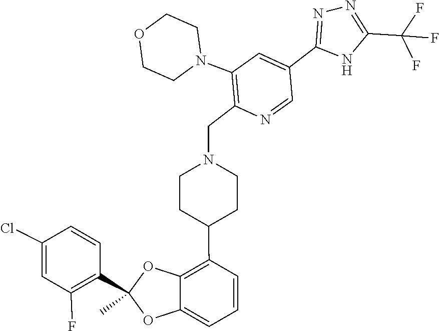 [SMILES: C[C@@]1(c2ccc(Cl)cc2F)Oc2cccc(C3CCN(Cc4ncc(-c5nnc(C(F)(F)F)[nH]5)cc4N4CCOCC4)CC3)c2O1] 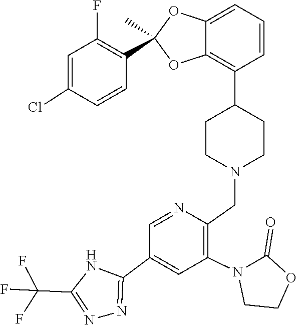 [SMILES: C[C@]1(c2ccc(Cl)cc2F)Oc2cccc(C3CCN(Cc4ncc(-c5nnc(C(F)(F)F)[nH]5)cc4N4CCOC4=O)CC3)c2O1] 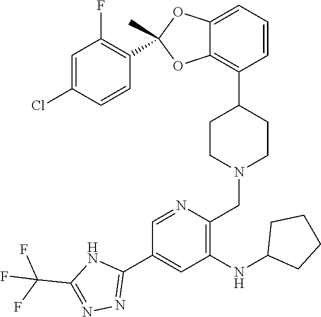 [SMILES: C[C@@]1(c2ccc(Cl)cc2F)Oc2cccc(C3CCN(Cc4ncc(-c5nnc(C(F)(F)F)[nH]5)cc4NC4CCCC4)CC3)c2O1] 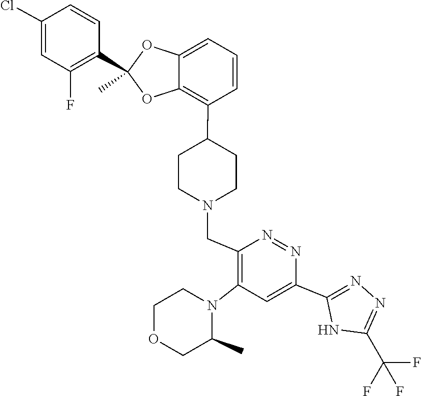 [SMILES: C[C@H]1COCCN1c1cc(-c2nnc(C(F)(F)F)[nH]2)nnc1CN1CCC(c2cccc3c2O[C@@](C)(c2ccc(Cl)cc2F)O3)CC1] 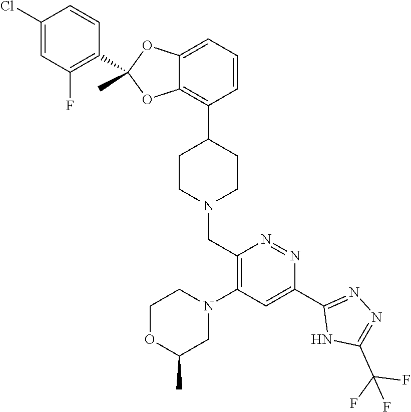 [SMILES: C[C@@H]1CN(c2cc(-c3nnc(C(F)(F)F)[nH]3)nnc2CN2CCC(c3cccc4c3O[C@](C)(c3ccc(Cl)cc3F)O4)CC2)CCO1] 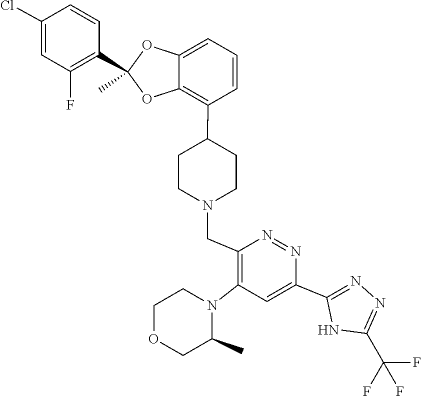 [SMILES: C[C@H]1COCCN1c1cc(-c2nnc(C(F)(F)F)[nH]2)nnc1CN1CCC(c2cccc3c2O[C@@](C)(c2ccc(Cl)cc2F)O3)CC1] 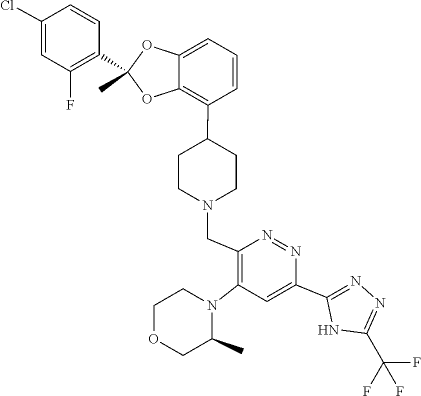 [SMILES: C[C@H]1COCCN1c1cc(-c2nnc(C(F)(F)F)[nH]2)nnc1CN1CCC(c2cccc3c2O[C@](C)(c2ccc(Cl)cc2F)O3)CC1] 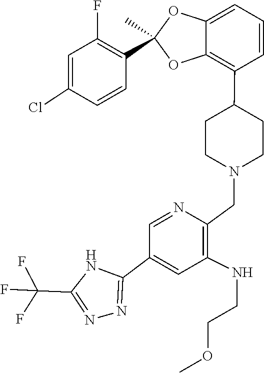 [SMILES: COCCNc1cc(-c2nnc(C(F)(F)F)[nH]2)cnc1CN1CCC(c2cccc3c2O[C@@](C)(c2ccc(Cl)cc2F)O3)CC1] 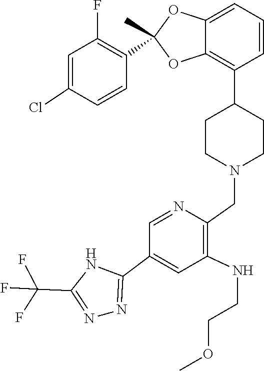 [SMILES: COCCNc1cc(-c2nnc(C(F)(F)F)[nH]2)cnc1CN1CCC(c2cccc3c2O[C@](C)(c2ccc(Cl)cc2F)O3)CC1] 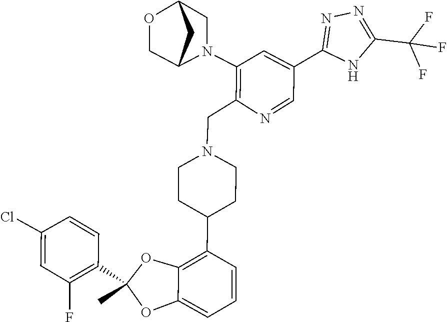 [SMILES: C[C@]1(c2ccc(Cl)cc2F)Oc2cccc(C3CCN(Cc4ncc(-c5nnc(C(F)(F)F)[nH]5)cc4N4C[C@@H]5C[C@H]4CO5)CC3)c2O1] 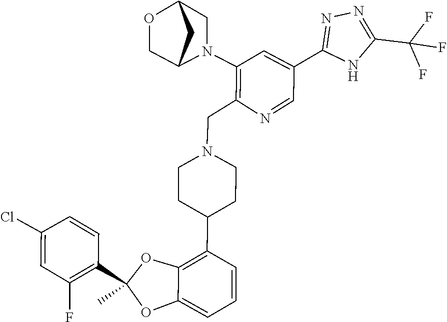 [SMILES: C[C@@]1(c2ccc(Cl)cc2F)Oc2cccc(C3CCN(Cc4ncc(-c5nnc(C(F)(F)F)[nH]5)cc4N4C[C@@H]5C[C@H]4CO5)CC3)c2O1] 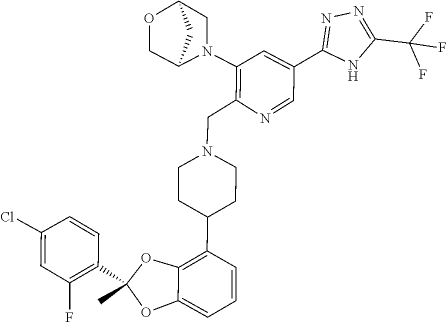 [SMILES: C[C@]1(c2ccc(Cl)cc2F)Oc2cccc(C3CCN(Cc4ncc(-c5nnc(C(F)(F)F)[nH]5)cc4N4C[C@H]5C[C@@H]4CO5)CC3)c2O1] 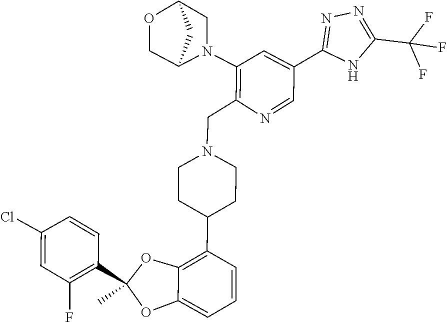 [SMILES: C[C@@]1(c2ccc(Cl)cc2F)Oc2cccc(C3CCN(Cc4ncc(-c5nnc(C(F)(F)F)[nH]5)cc4N4C[C@H]5C[C@@H]4CO5)CC3)c2O1] 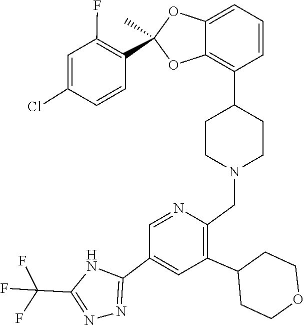 [SMILES: C[C@]1(c2ccc(Cl)cc2F)Oc2cccc(C3CCN(Cc4ncc(-c5nnc(C(F)(F)F)[nH]5)cc4C4CCOCC4)CC3)c2O1] 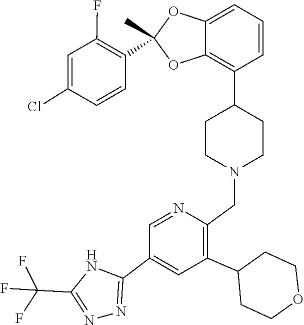 [SMILES: C[C@@]1(c2ccc(Cl)cc2F)Oc2cccc(C3CCN(Cc4ncc(-c5nnc(C(F)(F)F)[nH]5)cc4C4CCOCC4)CC3)c2O1] 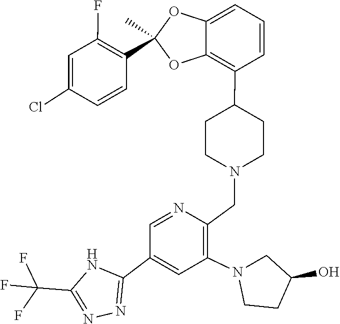 [SMILES: C[C@]1(c2ccc(Cl)cc2F)Oc2cccc(C3CCN(Cc4ncc(-c5nnc(C(F)(F)F)[nH]5)cc4N4CC[C@H](O)C4)CC3)c2O1] 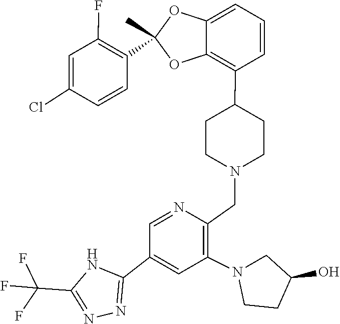 [SMILES: C[C@@]1(c2ccc(Cl)cc2F)Oc2cccc(C3CCN(Cc4ncc(-c5nnc(C(F)(F)F)[nH]5)cc4N4CC[C@H](O)C4)CC3)c2O1] 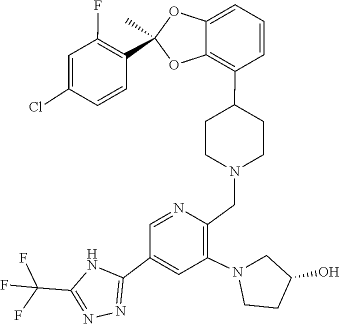 [SMILES: C[C@]1(c2ccc(Cl)cc2F)Oc2cccc(C3CCN(Cc4ncc(-c5nnc(C(F)(F)F)[nH]5)cc4N4CC[C@@H](O)C4)CC3)c2O1] 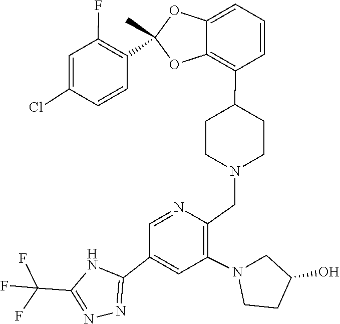 [SMILES: C[C@@]1(c2ccc(Cl)cc2F)Oc2cccc(C3CCN(Cc4ncc(-c5nnc(C(F)(F)F)[nH]5)cc4N4CC[C@@H](O)C4)CC3)c2O1] 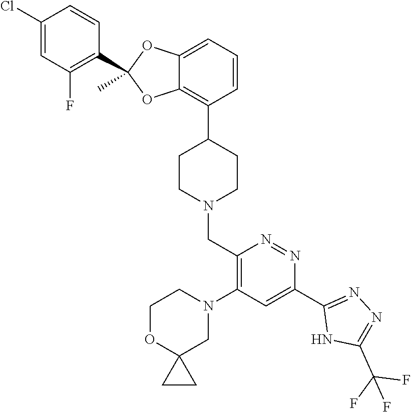 [SMILES: C[C@]1(c2ccc(Cl)cc2F)Oc2cccc(C3CCN(Cc4nnc(-c5nnc(C(F)(F)F)[nH]5)cc4N4CCOC5(CC5)C4)CC3)c2O1] 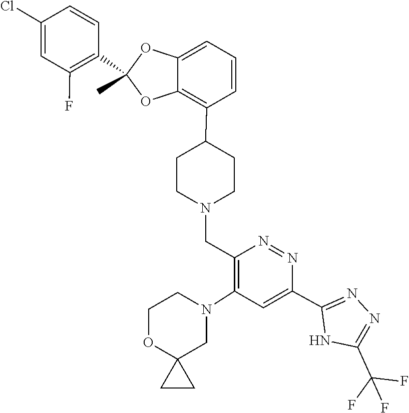 [SMILES: C[C@@]1(c2ccc(Cl)cc2F)Oc2cccc(C3CCN(Cc4nnc(-c5nnc(C(F)(F)F)[nH]5)cc4N4CCOC5(CC5)C4)CC3)c2O1] 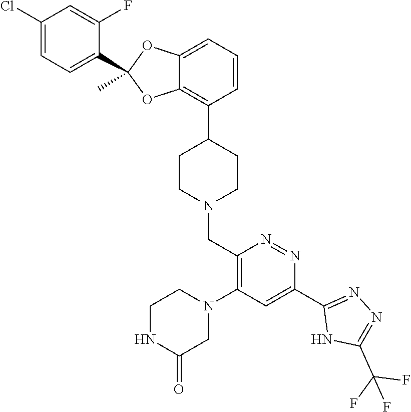 [SMILES: C[C@]1(c2ccc(Cl)cc2F)Oc2cccc(C3CCN(Cc4nnc(-c5nnc(C(F)(F)F)[nH]5)cc4N4CCNC(=O)C4)CC3)c2O1] 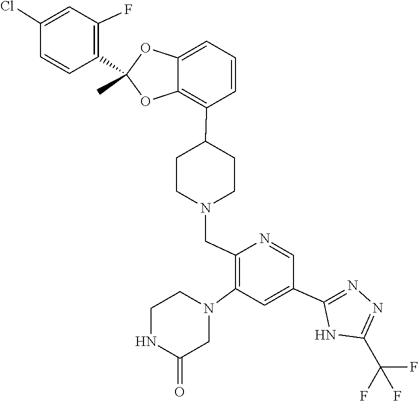 [SMILES: C[C@@]1(c2ccc(Cl)cc2F)Oc2cccc(C3CCN(Cc4ncc(-c5nnc(C(F)(F)F)[nH]5)cc4N4CCNC(=O)C4)CC3)c2O1] 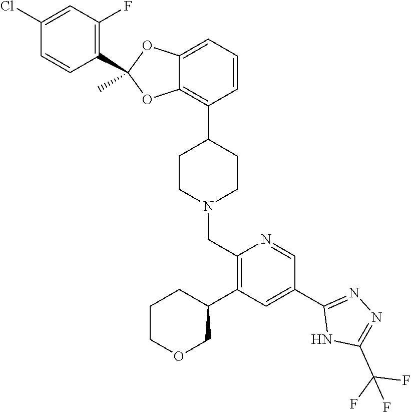 [SMILES: C[C@]1(c2ccc(Cl)cc2F)Oc2cccc(C3CCN(Cc4ncc(-c5nnc(C(F)(F)F)[nH]5)cc4[C@@H]4CCCOC4)CC3)c2O1] 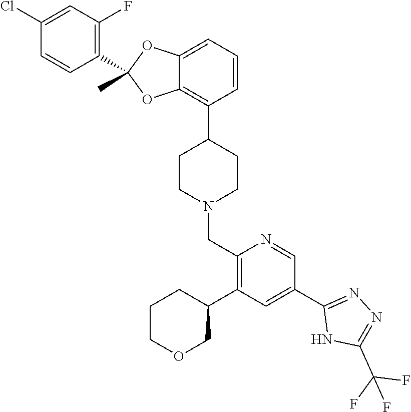 [SMILES: C[C@@]1(c2ccc(Cl)cc2F)Oc2cccc(C3CCN(Cc4ncc(-c5nnc(C(F)(F)F)[nH]5)cc4[C@@H]4CCCOC4)CC3)c2O1] 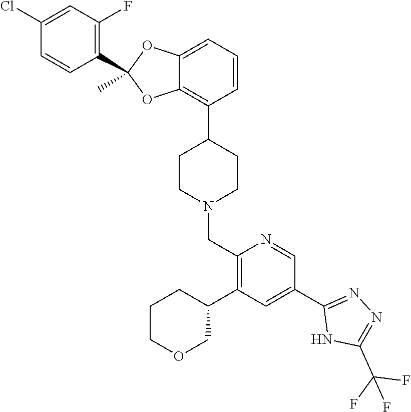 [SMILES: C[C@]1(c2ccc(Cl)cc2F)Oc2cccc(C3CCN(Cc4ncc(-c5nnc(C(F)(F)F)[nH]5)cc4[C@H]4CCCOC4)CC3)c2O1] 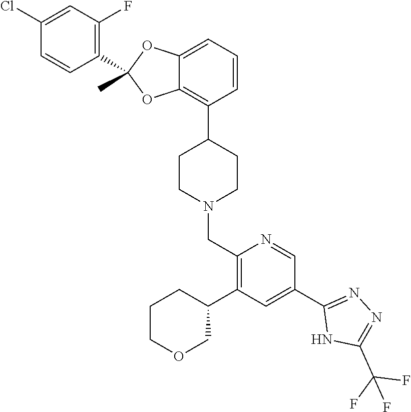 [SMILES: C[C@@]1(c2ccc(Cl)cc2F)Oc2cccc(C3CCN(Cc4ncc(-c5nnc(C(F)(F)F)[nH]5)cc4[C@H]4CCCOC4)CC3)c2O1] 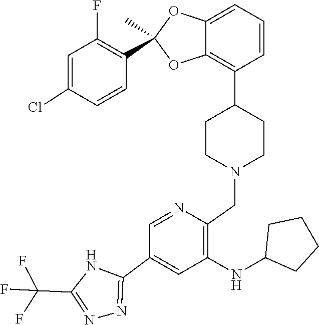 [SMILES: C[C@]1(c2ccc(Cl)cc2F)Oc2cccc(C3CCN(Cc4ncc(-c5nnc(C(F)(F)F)[nH]5)cc4NC4CCCC4)CC3)c2O1] 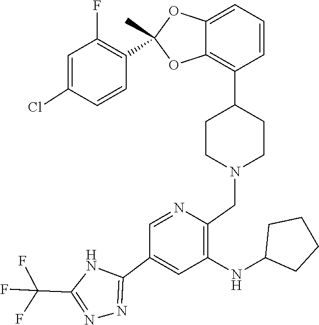 [SMILES: C[C@@]1(c2ccc(Cl)cc2F)Oc2cccc(C3CCN(Cc4ncc(-c5nnc(C(F)(F)F)[nH]5)cc4NC4CCCC4)CC3)c2O1] 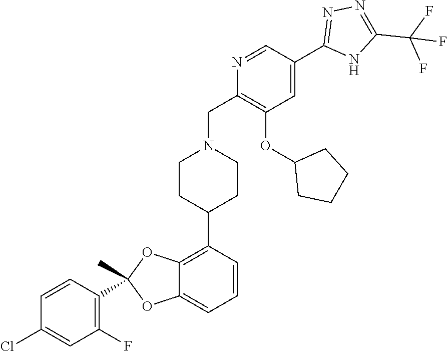 [SMILES: C[C@]1(c2ccc(Cl)cc2F)Oc2cccc(C3CCN(Cc4ncc(-c5nnc(C(F)(F)F)[nH]5)cc4OC4CCCC4)CC3)c2O1] 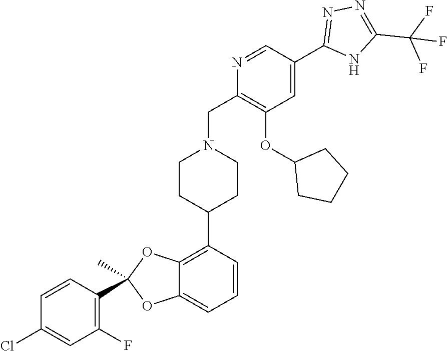 [SMILES: C[C@@]1(c2ccc(Cl)cc2F)Oc2cccc(C3CCN(Cc4ncc(-c5nnc(C(F)(F)F)[nH]5)cc4OC4CCCC4)CC3)c2O1] 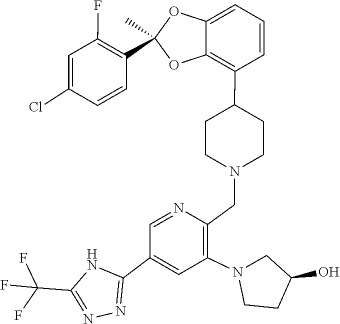 [SMILES: C[C@]1(c2ccc(Cl)cc2F)Oc2cccc(C3CCN(Cc4ncc(-c5nnc(C(F)(F)F)[nH]5)cc4N4CC[C@H](O)C4)CC3)c2O1] 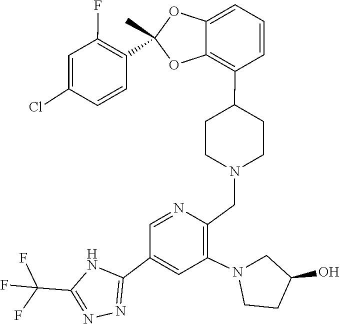 [SMILES: C[C@@]1(c2ccc(Cl)cc2F)Oc2cccc(C3CCN(Cc4ncc(-c5nnc(C(F)(F)F)[nH]5)cc4N4CC[C@H](O)C4)CC3)c2O1] 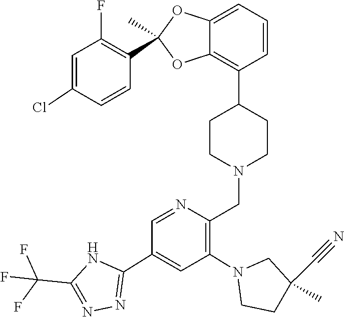 [SMILES: C[C@]1(C#N)CCN(c2cc(-c3nnc(C(F)(F)F)[nH]3)cnc2CN2CCC(c3cccc4c3O[C@@](C)(c3ccc(Cl)cc3F)O4)CC2)C1] 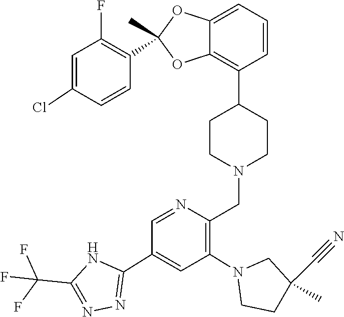 [SMILES: C[C@]1(C#N)CCN(c2cc(-c3nnc(C(F)(F)F)[nH]3)cnc2CN2CCC(c3cccc4c3O[C@](C)(c3ccc(Cl)cc3F)O4)CC2)C1] 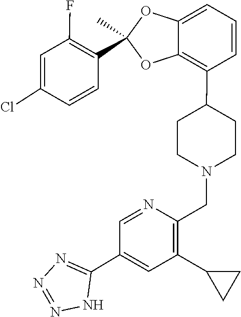 [SMILES: C[C@]1(c2ccc(Cl)cc2F)Oc2cccc(C3CCN(Cc4ncc(-c5nnn[nH]5)cc4C4CC4)CC3)c2O1] 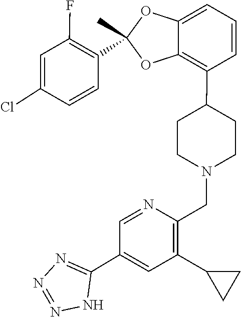 [SMILES: C[C@@]1(c2ccc(Cl)cc2F)Oc2cccc(C3CCN(Cc4ncc(-c5nnn[nH]5)cc4C4CC4)CC3)c2O1] 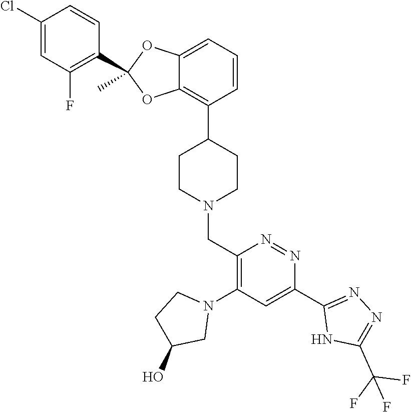 [SMILES: C[C@]1(c2ccc(Cl)cc2F)Oc2cccc(C3CCN(Cc4nnc(-c5nnc(C(F)(F)F)[nH]5)cc4N4CC[C@H](O)C4)CC3)c2O1] 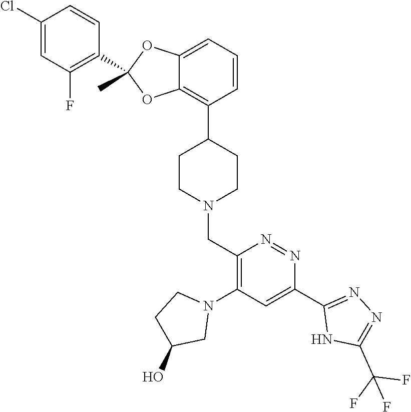 [SMILES: C[C@@]1(c2ccc(Cl)cc2F)Oc2cccc(C3CCN(Cc4nnc(-c5nnc(C(F)(F)F)[nH]5)cc4N4CC[C@H](O)C4)CC3)c2O1] 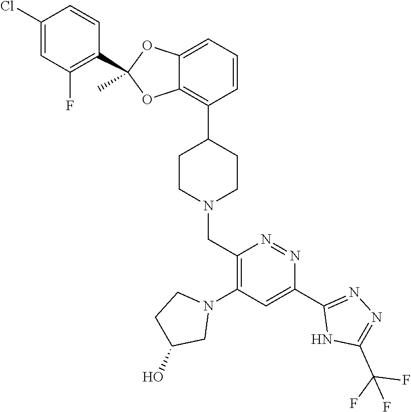 [SMILES: C[C@]1(c2ccc(Cl)cc2F)Oc2cccc(C3CCN(Cc4nnc(-c5nnc(C(F)(F)F)[nH]5)cc4N4CC[C@@H](O)C4)CC3)c2O1] 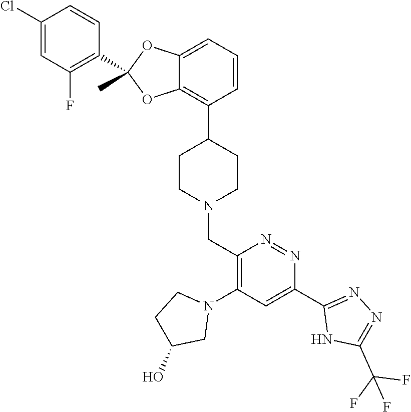 [SMILES: C[C@@]1(c2ccc(Cl)cc2F)Oc2cccc(C3CCN(Cc4nnc(-c5nnc(C(F)(F)F)[nH]5)cc4N4CC[C@@H](O)C4)CC3)c2O1] 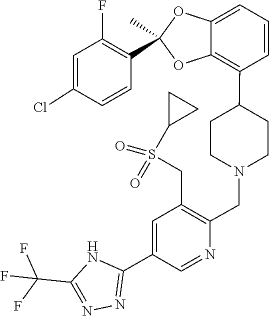 [SMILES: C[C@]1(c2ccc(Cl)cc2F)Oc2cccc(C3CCN(Cc4ncc(-c5nnc(C(F)(F)F)[nH]5)cc4CS(=O)(=O)C4CC4)CC3)c2O1] 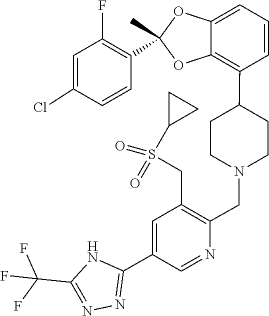 [SMILES: C[C@@]1(c2ccc(Cl)cc2F)Oc2cccc(C3CCN(Cc4ncc(-c5nnc(C(F)(F)F)[nH]5)cc4CS(=O)(=O)C4CC4)CC3)c2O1] 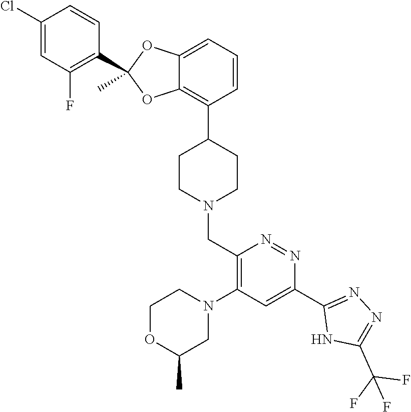 [SMILES: C[C@@H]1CN(c2cc(-c3nnc(C(F)(F)F)[nH]3)nnc2CN2CCC(c3cccc4c3O[C@@](C)(c3ccc(Cl)cc3F)O4)CC2)CCO1] 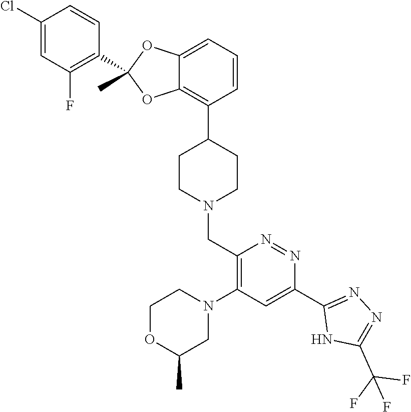 [SMILES: C[C@@H]1CN(c2cc(-c3nnc(C(F)(F)F)[nH]3)nnc2CN2CCC(c3cccc4c3O[C@](C)(c3ccc(Cl)cc3F)O4)CC2)CCO1] 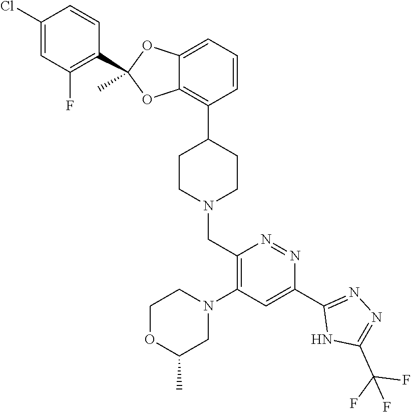 [SMILES: C[C@H]1CN(c2cc(-c3nnc(C(F)(F)F)[nH]3)nnc2CN2CCC(c3cccc4c3O[C@@](C)(c3ccc(Cl)cc3F)O4)CC2)CCO1] 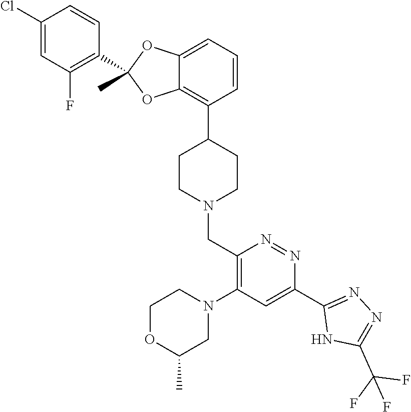 [SMILES: C[C@H]1CN(c2cc(-c3nnc(C(F)(F)F)[nH]3)nnc2CN2CCC(c3cccc4c3O[C@](C)(c3ccc(Cl)cc3F)O4)CC2)CCO1] 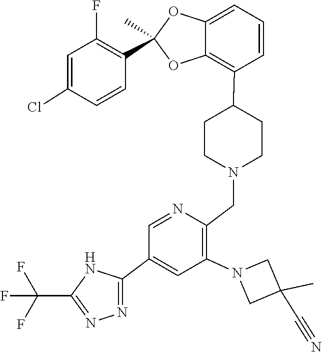 [SMILES: CC1(C#N)CN(c2cc(-c3nnc(C(F)(F)F)[nH]3)cnc2CN2CCC(c3cccc4c3O[C@@](C)(c3ccc(Cl)cc3F)O4)CC2)C1] 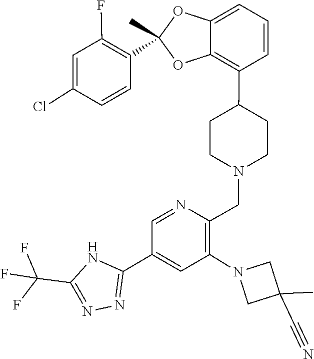 [SMILES: CC1(C#N)CN(c2cc(-c3nnc(C(F)(F)F)[nH]3)cnc2CN2CCC(c3cccc4c3O[C@](C)(c3ccc(Cl)cc3F)O4)CC2)C1] 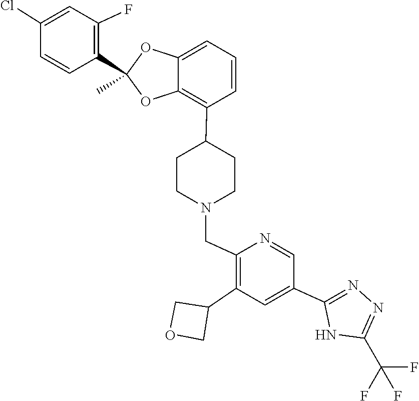 [SMILES: C[C@]1(c2ccc(Cl)cc2F)Oc2cccc(C3CCN(Cc4ncc(-c5nnc(C(F)(F)F)[nH]5)cc4C4COC4)CC3)c2O1] 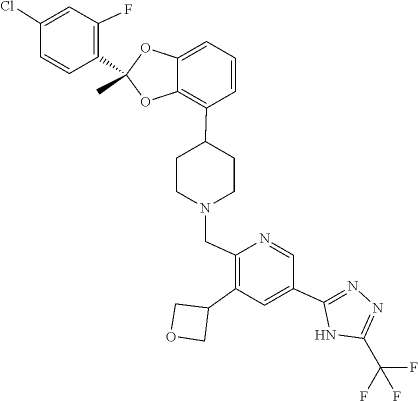 [SMILES: C[C@@]1(c2ccc(Cl)cc2F)Oc2cccc(C3CCN(Cc4ncc(-c5nnc(C(F)(F)F)[nH]5)cc4C4COC4)CC3)c2O1] 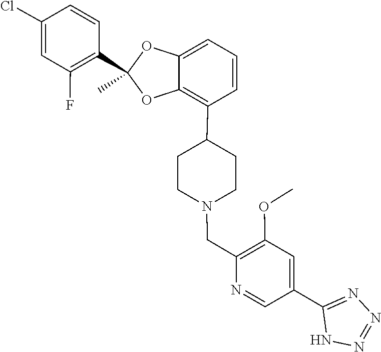 [SMILES: COc1cc(-c2nnn[nH]2)cnc1CN1CCC(c2cccc3c2O[C@@](C)(c2ccc(Cl)cc2F)O3)CC1] 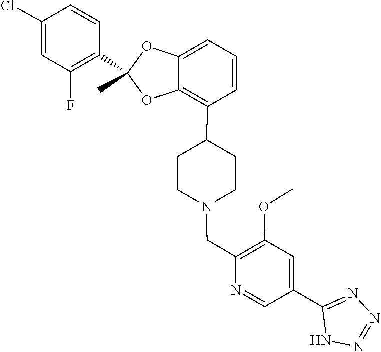 [SMILES: COc1cc(-c2nnn[nH]2)cnc1CN1CCC(c2cccc3c2O[C@](C)(c2ccc(Cl)cc2F)O3)CC1]
or a pharmaceutically acceptable salt solvate thereof.

22. A pharmaceutical composition comprising a compound of claim 2, or a pharmaceutically acceptable salt or solvate thereof, and a pharmaceutically acceptable excipient.

23. A method for treating a GLP-1 associated disease, disorder, or condition, the method comprising administering to a patient in need thereof an effective amount of a compound of claim 2, or a pharmaceutically acceptable salt or solvate thereof.

24. The method of claim 23, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or a combination thereof.

25. A method of treating type 2 diabetes mellitus in a patient in need thereof, the method comprising administering to a patient in need thereof an effective amount of a compound of claim 2, or a pharmaceutically acceptable salt or solvate thereof.

26. A method for modulating insulin levels in a patient in need of such modulating, the method comprising administering to a patient in need thereof an effective amount of a compound of claim 2, or a pharmaceutically acceptable salt or solvate thereof.

27. A method for modulating glucose levels in a patient in need of such modulating, the method comprising administering to a patient in need thereof an effective amount of a compound of claim 2, or a pharmaceutically acceptable salt or solvate thereof.

28. The method of claim 23, further comprising administering an additional therapy or therapeutic agent to the patient.

29. The method of claim 28, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.
